(19) United States
(12) Patent Application Publication
(10) Pub. No.: US 2004/0191291 A1
(43) Pub. Date: Sep. 30, 2004

(54) Composition and method for nerve regeneration

(76) Inventors: Masaya Tohyama, Toyonaka-shi (JP); Toshihide Yamashita, Minoh-shi (JP)

Correspondence Address: PERKINS COIE LLP, P.O. BOX 2168, MENLO PARK, CA, 94026, US

(21) Appl. No.: 10/427,741

(22) Filed: Apr. 30, 2003

(30) Foreign Application Priority Data
Mar. 28, 2003 (JP) 2003-92923

(51) Int. Cl. A61F 2/00

(52) U.S. Cl. 424/426

(57) ABSTRACT

The present invention provides a method for regenerating nerves, comprising the step of inhibiting a p75 signal transduction pathway. The inhibition of the p75 signal transduction pathway is selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

### BACKGROUND OF THE INVENTION

1. Field of the Invention

The present invention relates to a pharmaceutical composition and method for treating neurological diseases, and a pharmaceutical composition and method for regenerating nerves. Specifically, the present invention relates to a pharmaceutical composition and method for treating neurological diseases by disrupting inhibition of neurite outgrowth.

2. Description of the Related Art

The neurotrophin receptor p75 (p75^{NTR}) mediates surprisingly diverse biological effects (e.g., cell death, Schwann cell migration, modulation of the synaptic transmission, and functional regulation of sensory neurons and calcium currents) (e.g., see Dechant, G. & Barde, Y. A., Nat Neurosci. 5, 1131-1136 (2002)). Recent work also implicates p75^{NTR} in the regulation of axon elongation. Nerve growth factor (NGF) stimulates neurite outgrowth from embryonic rat hippocampal neurons and chick ciliary neurons, which express only p₇₅NTR for NGF receptors (e.g., Yamashita, T., Tucker, K. L. & Barde, Y. A., Neuron 24, 585-593 (1999)). These effects can be accounted for the modulation of Rho activity by p75^{NTR} Rho is a small GTPase that regulates the state of actin polymerization. In its active GTP-bound form, Rho rigidifies the actin cytoskeleton, thereby inhibiting axonal elongation and mediating growth cone collapse (e.g., see Davies, A. M., Curr. Biol. 10, R198-200 (2000) and Schmidt, A. & Hall, A., Genes Dev. 16, 1587-1609 (2002)). Neurotrophin binding to p75^{NTR} inactivates RhoA in HN10e cells as well as cerebellar neurons, whereas the over-expression of RhoA in the transfected 293 cells results in the activation of RhoA, suggesting that p75^{NTR} elicits bi-directional signals (e.g., see Yamashita et al. supra). Indeed, subsequent study shows that myelin-associated glycoprotein (MAG), a glycoprotein derived from myelin, activates RhoA by a p75^{NTR}-dependent mechanism, thus inhibiting neurite outgrowth from postnatal sensory neurons and cerebellar neurons (e.g., see Yamashita, T., Higuchi, H. & Tohyama, M., J. Cell Biol. 157, 565-570 (2002)). Furthermore, Nogo and oligodendrocyte myelin glycoprotein (OMgp), the other myelin-derived inhibitors of the neurite outgrowth, act on neurons via p75^{NTR} (e. g., see Wang, K. C. & Kim, J. A., Sivasankaran, R., Segal, R. & He, Z., Nature 420, 74-78 (2002)). p75^{NTR} in complex with the Nogo receptor is suggested to form a receptor for all the myelin-derived inhibitors found so far (e.g., see Wang et al. supra, and Wong, S. T. et al., Nat Neurosci. 5, 1302-1308 (2002)). However, precise mechanism of the regulation of Rho activity by p75^{NTR} remained to be elucidated.

RhoA was shown to interact with p75^{NTR} by the yeast two-hybrid system and co-immunoprecipitation (e.g., see Yamashita, T., Tucker, K. L. & Barde, Y. A., supra). As only the wild type of RhoA, which is predominantly in a GDP-bound form, but not the constitutive active form of RhoA, interacts with p75^{NTR}, it is suggested that the activation of RhoA is dependent on a direct interaction of RhoA and p75^{NTR}. Rho proteins in the GDP-bound form interact with Rho GDP dissociation inhibitor (Rho GDI), which plays a role in inhibiting nucleotide dissociation as well as the shuttling of Rho proteins between the cytoplasm and membranes (e.g., see Sasaki, T. & Takai, Y., Biochem Biophys Res Commun. 245, 641-645 (1998)). Rho GDI prevents Rho family proteins from being converted to the active, GTP-bound form that is translocated to the membrane. In addition, after the active forms of Rho proteins are converted to the inactive forms at the membrane, Rho GDI forms a complex with them and translocates them to the cytosol. The Rho GDI family comprises at least three isoforms: Rho GDIα, Rho GDIβ and Rho GDIγ. Rho GDIα is ubiquitously expressed and binds to all of the Rho family proteins thus far examined, whereas Rho GDIβ and Rho GDIγ show unique tissue expression patterns and their substrate specificities have not been exactly determined.

Considering the above-described discussion, an object of the present invention is to elucidate the relationship between p75^{NTR}, which is involved in inhibition of neurite outgrowth, and agents capable of interacting therewith, thereby leading to regeneration of nerves and further treating neurological diseases based on the nerve regeneration.

### SUMMARY OF THE INVENTION

The present inventors achieved the above-described object in part by completely uncovering the signal transduction pathway via p75^{NTR} (or herein referred to also as p75).

The present inventors report the precise mechanism of the regulation of Rho activity by p75^{NTR}. Interestingly, p75^{NTR} shows activity of displacing the GDP-bound form of RhoA from Rho GDIα. A peptide (Pep5), that was shown to specifically associate with p75^{NTR}, efficiently inhibits the signal mediated by p75^{NTR}, and may be a useful therapeutic agent in reversing the growth inhibition elicited by myelin-derived inhibitors.

The neurotrophin receptor p75^{NTR} is involved in the regulation of axonal elongation by neurotrophins as well as several myelin components (e.g., myelin-associated glycoprotein, Nogo and oligodendrocyte myelin glycoprotein). Neurotrophins stimulate neurite outgrowth by inhibiting Rho activity, whereas myelin-derived proteins activate RhoA, both through a p75^{NTR}-dependent mechanism. Here, the present inventors show that direct interaction of the Rho GDP dissociation inhibitor with p75^{NTR} initiates the activation of RhoA. The interaction of p75^{NTR} with Rho GDI is strengthened by myelin-associated glycoprotein or Nogo. p75^{NTR} facilitates the release of prenylated RhoA from Rho GDP dissociation inhibitor. The peptide ligand that was shown to be associated with the fifth of the six α-helices of p75^{NTR} inhibits the interaction between Rho GDP dissociation inhibitor and p75^{NTR}, thus silencing the action mediated by p75^{NTR}. This peptide has potential as a therapeutic agent against the inhibitory cues that contribute to the lack of regeneration of the central nervous system, i.e., an agent extinguishing the interaction between p75^{NTR} and Rho GDI has the therapeutic potential for spinal cord injury, Alzheimer's disease, cerebral infarction, cerebral hemorrhage, brain injury, and the like.

Therefore, the present invention provides the following.

According to an aspect of the present invention, a method for regenerating nerves is provided, which comprises the step of inhibiting a p75 signal transduction pathway.

In one embodiment of this invention, the p75 signal transduction pathway is present in a neuron at a site desired for nerve regeneration.

In one embodiment of this invention, the inhibition of the p75 signal transduction pathway is achieved by providing a transduction agent in the p75 signal transduction pathway or a variant or fragment thereof, or an agent capable of specifically interacting with the transduction agent in the p75 signal transduction pathway in an amount effective for regeneration.

In one embodiment of this invention, the transduction agent in the p75 signal transduction pathway is at least one transduction agent selected from the group consisting of MAG, GT1b, p75, Rho GDI, Rho, p21, and Rho kinase.

In one embodiment of this invention, the inhibition of the p75 signal transduction pathway is selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the inhibition of the p75 signal transduction pathway is achieved by providing at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase, in an amount effective for regeneration.

In one embodiment of this invention, the nerve regeneration is carried out in vivo or in vitro.

In one embodiment of this invention, the nerve is in a condition including spinal cord injury, cerebrovascular disorder, or brain injury.

In one embodiment of this invention, the step of inhibiting the p75 signal transduction pathway comprises the step of providing a composition comprising at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase, an agent capable of specifically interacting with a nucleic acid molecule encoding the Rho kinase, and variants and fragments thereof, to the nerve in an amount effective for regeneration.

In one embodiment of this invention, the agent is bound to a PTD domain.

According to another aspect of the present invention, a method for treatment, prophylaxis, diagnosis or prognosis of nervous diseases, nervous disorders and/or nervous conditions is provided, which comprises the step of modulating a p75 signal transduction pathway in a subject in need of or suspected of being in need of the treatment, prophylaxis, diagnosis or prognosis.

In one embodiment of this invention, the step of modulating the p75 signal transduction pathway comprises the step of administering a transduction agent in the p75 signal transduction pathway or a variant or fragment thereof, or an agent capable of specifically interacting with the transduction agent in the p75 signal transduction pathway in an amount effective for regeneration to the subject in need of or suspected of being in need of the treatment, prophylaxis, diagnosis or prognosis.

In one embodiment of this invention, the transduction agent in the p75 signal transduction pathway is at least one transduction agent selected from the group consisting of MAG, GT1b, p75, Rho GDI, Rho, p21, and Rho kinase.

In one embodiment of this invention, the modulation of the p75 signal transduction pathway comprises at least one modulation selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase, in the subject in need of or suspected of being in need of the treatment, prophylaxis, diagnosis or prognosis.

In one embodiment of this invention, the modulation of the p75 signal transduction pathway comprises the step of administering at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase, in an amount effective for regeneration to the subject in need of or suspected of being in need of the treatment, prophylaxis, diagnosis or prognosis.

In one embodiment of this invention, the nerve regeneration is carried out in vivo or in vitro.

In one embodiment of this invention, the nerve is in a condition including spinal cord injury, cerebrovascular disorder, or brain injury.

In one embodiment of this invention, the step of modulating the p75 signal transduction pathway comprises the step of providing a composition comprising at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof, in an amount effective for the diagnosis, prophylaxis, treatment or prognosis to the nerve.

In one embodiment of this invention, the method further comprises the step of providing one or more drugs.

In one embodiment of this invention, the agent is bound to a PTD domain.

According to another aspect of the present invention, a composition is provided, which comprises an agent capable of inhibiting a p75 signal transduction pathway.

In one embodiment of this invention, the agent capable of inhibiting the p75 signal transduction pathway is in a form appropriate for delivery to a neuron at a site desired for nerve regeneration.

In one embodiment of this invention, the agent capable of inhibiting the p75 signal transduction pathway comprises a transduction agent in the p75 signal transduction pathway or a variant or fragment thereof, or an agent capable of specifically interacting with the transduction agent in the p75 signal transduction pathway.

In one embodiment of this invention, the transduction agent in the p75 signal transduction pathway comprises at least one transduction agent selected from the group consisting of MAG, GT1b, p75, Rho GDI, Rho, p21, and Rho kinase.

In one embodiment of this invention, the agent capable of inhibiting the p75 signal transduction pathway has at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the agent capable of inhibiting the p75 signal transduction pathway comprises at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase. The agent capable of inhibiting the p75 signal transduction pathway is present in an amount effective for regeneration.

In one embodiment of this invention, the composition is suitable for in vivo or in vitro administration forms.

In one embodiment of this invention, the nerve is in a condition including spinal cord injury, cerebrovascular disorder, or brain injury.

In one embodiment of this invention, the agent capable of inhibiting the p75 signal transduction pathway comprises at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof.

In one embodiment of this invention, the agent is bound to a PTD domain.

According to another aspect of the present invention, a composition for treatment, prophylaxis, diagnosis or prognosis of nervous diseases, nervous disorders and/or nervous conditions is provided, which comprises an agent capable of modulating a p75 signal transduction pathway.

In one embodiment of this invention, the agent capable of modulating the p75 signal transduction pathway comprises a transduction agent in the p75 signal transduction pathway or a variant or fragment thereof, or an agent capable of specifically interacting with the transduction agent in the p75 signal transduction pathway.

In one embodiment of this invention, the transduction agent in the p75 signal transduction pathway comprises at least one transduction agent selected from the group consisting of MAG, GT1b, p75, Rho GDI, Rho, p21, and Rho kinase.

In one embodiment of this invention, the modulation of the p75 signal transduction pathway is selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the agent capable of modulating the p75 signal transduction pathway comprises at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase.

In one embodiment of this invention, the composition is in a form suitable for oral or parenteral administration.

In one embodiment of this invention, the nerve is in a condition including spinal cord injury, cerebrovascular disorder, or brain injury.

In one embodiment of this invention, the agent capable of modulating the p75 signal transduction pathway comprises at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof.

In one embodiment of this invention, the composition further comprises one or more drugs.

In one embodiment of this invention, the agent is bound to a PTD domain.

According to another aspect of the present invention, a composition for regenerating nerves is provided, which comprises a Pep5 polypeptide.

In one embodiment of this invention, the Pep5 polypeptide comprises (a) a polypeptide encoded by a nucleic acid sequence as set forth in SEQ ID NO. 1 or a fragment thereof; (b) a polypeptide having an amino acid sequence as set forth in SEQ ID NO. 2 or a fragment thereof; (c) a variant polypeptide having an amino acid sequence as set forth in SEQ ID NO. 2 having at least one mutation selected from the group consisting of one or more amino acid substitutions, additions, and deletions, wherein the variant polypeptide has a biological activity; or (d) a polypeptide consisting of an amino acid sequence having at least 70% identity to any one of the polypeptides of (a) to (c), wherein the polypeptide has a biological activity.

In one embodiment of this invention, the Pep5 polypeptide comprises the whole amino acid sequence as set forth in SEQ ID NO. 2.

In one embodiment of this invention, the nerve is in a condition including spinal cord injury, cerebrovascular disorder, or brain injury.

In one embodiment of this invention, the Pep5 polypeptide further comprises a PTD domain.

According to another aspect of the present invention, a composition for regenerating nerves is provided, which comprises a nucleic acid molecule encoding a Pep5 polypeptide.

In one embodiment of this invention, the nucleic acid molecule encoding the Pep5 polypeptide comprises: (a) a polynucleotide having a base sequence as set forth in SEQ ID NO. 1 or a fragment thereof; (b) a polynucleotide encoding an amino acid sequence as set forth in SEQ ID NO. 2 or a fragment thereof; (c) a polynucleotide encoding a variant polypeptide having the amino acid sequence as set forth in SEQ ID NO. 2 having at least one mutation selected from the group consisting of one or more amino acid substitutions, additions, and deletions, wherein the variant polypeptide has a biological activity; (d) a polynucleotide encoding a polypeptide hybridizable to any one of the polynucleotides of (a) to (c) under stringent conditions, wherein the polypeptide has a biological activity; or (e) a polynucleotide consisting of a base sequence having at least 70% identity to any one of the polynucleotides of (a) to (c) or a complementary sequence thereof, wherein the polynucleotide encodes a polypeptide having a biological activity.

In one embodiment of this invention, the nucleic acid molecule encoding the Pep5 polypeptide comprises the whole nucleotide sequence in the nucleic acid sequence as set forth in SEQ ID NO. 1.

In one embodiment of this invention, the nerve is in a condition including spinal cord injury, cerebrovascular disorder, or brain injury.

In one embodiment of this invention, the nucleic acid molecule encoding the Pep5 polypeptide comprises a sequence encoding a PTD domain.

According to another aspect of the present invention, a composition for regenerating nerves is provided, which comprises an agent capable of specifically interacting with a p75 polypeptide.

In one embodiment of this invention, the p75 polypeptide comprises: (a) a polypeptide encoded by a nucleic acid sequence as set forth in SEQ ID NO. 3 or 16 or a fragment thereof; (b) a polypeptide having an amino acid sequence as set forth in SEQ ID NO. 4 or 17 or a fragment thereof; (c) a variant polypeptide having the amino acid sequence as set forth in SEQ ID NO. 4 or 17 having at least one mutation selected from the group consisting of one or more amino acid substitutions, additions, and deletions, wherein the variant polypeptide has a biological activity; (d) a polypeptide encoded by a splice variant or allelic variant of the base sequence as set forth in SEQ ID NO. 3 or 16; (e) a species homolog polypeptide of a polypeptide having the amino acid sequence as set forth in SEQ ID NO. 4 or 17; or (f) a polypeptide consisting of an amino acid sequence having at least 70% identity to the amino acid sequence of any one of the polypeptides of (a) to (e), wherein the polypeptide has a biological activity.

In one embodiment of, this invention, the p75 polypeptide comprises amino acids 273 to 427 or 274 to 425 of the amino acid sequence as set forth in SEQ ID NO. 4 or 17, respectively.

In one embodiment of this invention, the nerve is in a condition including spinal cord injury, cerebrovascular disorder, or brain injury.

In one embodiment of this invention, the agent comprises an antibody.

According to another aspect of the present invention, a composition for regenerating nerves is provided, which comprises an agent capable of specifically interacting with a nucleic acid molecule encoding a p75 polypeptide.

In one embodiment of this invention, a nucleic acid molecule encoding the p75 polypeptide is a polynucleotide selected from the group consisting of: (a) a polynucleotide having a base sequence as set forth in SEQ ID NO. 3 or 16 or a fragment sequence thereof; (b) a polynucleotide encoding an amino acid sequence as set forth in SEQ ID NO. 4 or 17 or a fragment thereof; (c) a polynucleotide encoding a variant polypeptide havingthe amino acid sequence as set forth in SEQ ID NO. 4 or 17 having at least one mutation selected from the group consisting of one or more amino acid substitutions, additions, and deletions, wherein the variant polypeptide has a biological activity; (d) a polynucleotide which is a splice variant or allelic variant of the base sequence as set forth in SEQ ID NO. 3 or 16; (e) a polynucleotide encoding a species homolog of a polypeptide consisting of the amino acid sequence as set forth in SEQ ID NO. 4 or 17; (f) a polynucleotide hybridizable to any one of the polynucleotides of (a) to (e) under stringent conditions, wherein the polynucleotide encodes a polypeptide having a biological activity; or (g) a polynucleotide consisting of a base sequence having at least 70% identity to any one of the polynucleotides of (a) to (e) or a complementary sequence thereof, wherein the polynucleotide encodes a polypeptide having a biological activity.

In one embodiment of this invention, the nucleic acid molecule encoding the p75 polynucleotide comprises nucleotides 1110 to 1283 or 1113 to 1277 of the nucleic acid sequence as set forth in SEQ ID NO. 3 or 16, respectively.

In one embodiment of this invention, the nerve is in a condition including spinal cord injury, cerebrovascular disorder, or brain injury.

In one embodiment of this invention, the agent is an antisense or RNAi of the nucleic acid molecule encoding the p75 polypeptide.

According to another aspect of the present invention, a composition for regenerating nerves is provided, which comprises a p75 extracellular domain polypeptide.

In one embodiment of this invention, the p75 extracellular domain comprises: (a) a polypeptide encoded by nucleotides 198 to 863 or 201 to 866 of a nucleic acid sequence as set forth in SEQ ID NO. 3 or 16, respectively, or a fragment thereof; (b) a polypeptide having amino acids 29 to 250 or 30 to 251 of an amino acid sequence as set forth in SEQ ID NO. 4 or 17, respectively, or a fragment thereof; (c) a variant polypeptide having amino acids 29 to 250 or 30 to 251 of the amino acid sequence as set forth in SEQ ID NO. 4 or 17, respectively, having at least one mutation selected from the group consisting of one or more amino acid substitutions, additions, and deletions, wherein the variant polypeptide has a biological activity; (d) a polypeptide encoded by a sequence of a splice variant or allelic variant of nucleotides 198 to 863 or 201 to 866 of the base sequence as set forth in SEQ ID NO. 3 or 16, respectively; (e) a species homolog polypeptide of a polypeptide having amino acids 29 to 250 or 30 to 251 of the amino acid sequence as set forth in SEQ ID NO. 4 or 17, respectively; or (f) a polypeptide consisting of an amino acid sequence having at least 70% identity to any one of the polypeptides of (a) to (e), wherein the polypeptide has a biological activity.

In one embodiment of this invention, the p75 extracellular domain polypeptide comprises amino acids 29 to 250 or 30 to 251 of the amino acid sequence as set forth in SEQ ID NO. 4 or 17, respectively.

In one embodiment of this invention, the nerve is in a condition including spinal cord injury, cerebrovascular disorder, or brain injury.

In one embodiment of this invention, the p75 extracellular domain polypeptide is soluble.

According to another aspect of the present invention, a composition for regenerating nerves is provided, which comprises a nucleic acid molecule encoding the p75 extracellular domain polypeptide.

In one embodiment of this invention, the nucleic acid molecule encoding the p75 extracellular domain polypeptide is a polynucleotide selected from the group consisting of:. (a) a polynucleotide having nucleotides 198 to 863 or 201 to 866 of a base sequence as set forth in SEQ ID NO. 3 or 16, respectively, or a fragment thereof; (b) a polynucleotide encoding amino acids 29 to 250 or 30 to 251 of an amino acid sequence as set forth in SEQ ID. NO. 4 or 17, respectively, or a fragment thereof; (c) a polynucleotide encoding a variant polypeptide having amino acids. 29 to 250 or 30 to 251 of the amino acid sequence as set forth in SEQ ID NO. 4 or 17, respectively, having at least one mutation selected from the group consisting of one or more amino acid substitutions, additions, and deletions, wherein the variant polypeptide has a biological activity; (d) a polynucleotide which is a splice variant or allelic variant of nucleotides 198 to 863 or 201 to 866 of the base sequence as set forth in SEQ ID NO. 3 or 16, respectively; (e) a polynucleotide encoding a species homolog of a polypeptide consisting of amino acid 29 to 250 or 30 to 251 of the amino acid sequence as set forth in SEQ ID NO. 4 or 17, respectively; (f) a polynucleotide hybridizable to any one of the polynuleotides of (a) to (e) under stringent conditions, wherein the polynucleotide encodes a polypeptide having a biological activity; and (g) a polynucleotide consisting of a base sequence having at least 70% identity to any one of the polynucleotides of (a) to (e) or a complementary sequence thereof, wherein the polypeptide has a biological activity.

In one embodiment of this invention, the nucleic acid molecule encoding the p75 extracellular domain polypeptide comprises nucleotides 198 to 863 or 201 to 866 of the nucleic acid sequence as set forth in SEQ ID NO. 3 or 16, respectively.

In one embodiment of this invention, the nerve is in a condition including spinal cord injury, cerebrovascular disorder, or brain injury.

In one embodiment of this invention, the p75 extracellular domain polypeptide is soluble.

According to another aspect of the present invention, a composition for regenerating nerves is provided, which comprises an agent capable of specifically interacting with a Rho GDI polypeptide.

In one embodiment of this invention, the Rho GDI polypeptide comprises: (a) a polypeptide encoded by a nucleic acid sequence as set forth in SEQ ID NO. 5 or a fragment thereof; (b) a polypeptide having an amino acid sequence SEQ ID NO. 6 or a fragment thereof; (c) a variant polypeptide having the amino acid sequence as set forth in SEQ ID NO. 6 having at least one mutation selected from the group consisting of one or more amino acid substitutions, additions, and deletions, wherein the variant peptide has a biological activity; (d) a polypeptide encoded by a splice variant or allelic variant of the base sequence as set forth in SEQ ID NO. 5; (e) a species homolog polypeptide of a polypeptide having the amino acid sequence as set forth in SEQ ID NO. 6; or (f) a polypeptide consisting of an amino acid sequence having at least 70% identity to any one of the polypeptides of (a) to (e), wherein the polypeptide has a biological activity.

In one embodiment of this invention, the Rho GDI polypeptide comprises the entire amino acid sequence as set forth in SEQ ID NO. 6.

In one embodiment of this invention, the nerve is in a condition including spinal cord injury, cerebrovascular disorder, or brain injury.

In one embodiment of this invention, the agent comprises an antibody.

According to another aspect of the present invention, a composition for regenerating nerves is provided, which comprises an agent capable of specifically interacting with a nucleic acid molecule encoding a Rho GDI polypeptide.

In one embodiment of this invention, the nucleic acid encoding the Rho GDI polypeptide is a polynucleotide selected from the group consisting of: (a) a polynucleotide having a base sequence as set forth in SEQ ID NO. 5 or a fragment sequence thereof; (b) a polynucleotide encoding an amino acid of an amino acid sequence as set forth in SEQ ID NO. 6 or a fragment thereof; (c) a polynucleotide encoding a variant polypeptide having the amino acid of the amino acid sequence as set forth in SEQ ID NO. 6 having at least one mutation selected from the group consisting of one or more amino acid substitutions, additions, and deletions, wherein the variant polypeptide has a biological activity;. (d) a polynucleotide which is a splice variant or allelic variant of the base sequence as set forth in SEQ ID NO. 5; (e) a polynucleotide encoding a species homolog of a polypeptide consisting of the amino acid sequence as set forth in SEQ ID NO. 6; (f) a polynucleotide hybridizable to any one of the polynucleotides of (a) to (e) under stringent conditions, wherein the polynucleotide encodes a polypeptide having a biological activity; and (g) a polynucleotide consisting of a base sequence having at least 70% identity to any one of the polynucleotides of (a) to (e) or a complementary sequence thereof, and wherein the polynucleotide encodes a polypeptide having a biological activity.

In one embodiment of this invention, the Rho GDI comprises the entire nucleic acid sequence as set forth in SEQ ID NO. 5.

In one embodiment of this invention, the nerve is in a condition including spinal cord injury, cerebrovascular disorder, or brain injury.

In one embodiment of this invention, the agent comprises an antisense molecule or RNAi.

According to another aspect of the present invention, a composition for regenerating nerves is provided, which comprises an agent capable of specifically interacting with a MAG polypeptide.

In one embodiment of this invention, the MAG polypeptide comprises: (a) a polypeptide encoded by a nucleic acid molecule as set forth in SEQ ID NO. 7 or a fragment thereof; (b) a polypeptide having an amino acid sequence as set forth in SEQ ID NO. 8 or a fragment thereof; (c) a variant polypeptide having the amino acid sequence as set forth in SEQ ID NO. 8 having at least one mutation selected from the group consisting of one or more amino acid substitutions, additions, and deletions, wherein the variant polypeptide has a biological activity; (d) a polypeptide encoded by a splice variant or allelic variant of the base sequence as set forth in SEQ ID NO. 7; (e) a species homolog polypeptide of a polypeptide having the amino acid sequence as set forth in SEQ ID NO. 8; or (f) a polypeptide consisting of an amino acid sequence having at least 70% identity to any one of the polypeptides of (a) to (e) and wherein the polypeptide has a biological activity.

In one embodiment of this invention, the MAG polypeptide comprises amino acids 1 to 626 of the amino acid sequence as set forth in SEQ ID NO. 8.

In one embodiment of this invention, the nerve is in a condition including spinal cord injury, cerebrovascular disorder, or brain injury.

In one embodiment of this invention, the agent comprises an antibody.

According to another aspect of the present invention, a composition for regenerating nerves is provided, which comprises an agent capable of specifically interacting with a nucleic acid molecule encoding a MAG polypeptide.

In one embodiment of this invention, the nucleic acid molecule encoding the MAG polypeptide is a polynucleotide selected from the group consisting of: (a) a polynucleotide having a base sequence as set forth in SEQ ID NO. 7 or a fragment sequence thereof; (b) a polynucleotide encoding an amino acid sequence as set forth in SEQ ID NO. 8 or a fragment thereof; (c) a polynucleotide encoding a variant polypeptide having the amino acid sequence as set forth in SEQ ID NO. 8 having at least one mutation selected from the group consisting of one or more amino acid substitutions, additions, and deletions, wherein the variant polypeptide has a biological activity; (d) a polynucleotide which is a splice variant or allelic variant of the base sequence as set forth in SEQ ID NO. 7; (e) a polynucleotide encoding a species homolog of a polypeptide consisting of the amino acid having the amino acid sequence as set forth in SEQ ID NO. 8; (f) a polynucleotide hybridizable to any one of the polynucleotides of (a) to (e) under stringent conditions, wherein the polynucleotide has a biological activity; or (g) a polynucleotide consisting of a base sequence having at least 70% identity to any one of the polynucleotides of (a) to (e) or a complementary sequence thereof, wherein the polypeptide has a biological activity.

In one embodiment of this invention, the nucleic acid molecule encoding the MAG polypeptide comprises nucleotides 1 to 2475 of the nucleic acid sequence as set forth in SEQ ID NO. 7.

In one embodiment of this invention, the nerve is in a condition including spinal cord injury, cerebrovascular disorder, or brain injury.

In one embodiment of this invention, the agent is an antisense or RNAi of the nucleic acid molecule encoding the MAG polypeptide.

According to another aspect of the present invention, a composition for regenerating nerves is provided, which comprises an agent capable of specifically interacting with a Rho polypeptide.

In one embodiment of this invention, the Rho polypeptide comprises: (a) a polypeptide encoded by a nucleic acid sequence as set forth in SEQ ID NO. 11 or a fragment thereof; (b) a polypeptide having an amino acid sequence SEQ ID NO. 12 or a fragment thereof; (c) a variant polypeptide having the amino acid sequence as set forth in SEQ ID NO. 12 having at least one mutation selected from the group consisting of one or more amino acid substitutions, additions, and deletions, wherein the variant peptide has a biological activity; (d) a polypeptide encoded by a splice variant or allelic variant of the base sequence as set forth in SEQ ID NO. 11; (e) a species homolog polypeptide of a polypeptide having the amino acid sequence as set forth in SEQ ID NO. 12; or (f) a polypeptide consisting of an amino acid sequence having at least 70% identity to any one of the polypeptides of (a) to (e), wherein the polypeptide has a biological activity.

In one embodiment of this invention, the Rho polypeptide comprises amino acids 1 to 193 of the amino acid sequence as set forth in SEQ ID NO. 12.

In one embodiment of this invention, the nerve is in a condition including spinal cord injury, cerebrovascular disorder, or brain injury.

In one embodiment of this invention, the agent comprises an antibody.

According to another aspect of the present invention, a composition for regenerating nerves is provided, which comprises an agent capable of specifically interacting with a nucleic acid molecule encoding a Rho polypeptide.

In one embodiment of this invention, the nucleic acid molecule encoding the Rho polypeptide is a polynucleotide selected from the group consisting of: (a) a polynucleotide having a base sequence as set forth in SEQ ID NO. 11 or a fragment sequence thereof; (b) a polynucleotide encoding an amino acid sequence as set forth in SEQ ID NO. 12 or a fragment thereof; (c) a polynucleotide encoding a variant polypeptide having the amino acid sequence as set forth in SEQ ID NO. 12 having at least one mutation selected from the group consisting of one or more amino acid substitutions, additions, and deletions, wherein the variant polypeptide has a biological activity; (d) a polynucleotide which is a splice variant or allelic variant of the base sequence as set forth in SEQ ID NO. 11; (e) a polynucleotide encoding a species homolog of a polypeptide consisting of the amino acid having the amino acid sequence as set forth in SEQ ID NO. 12; (f) a polynucleotide hybridizable to any one of the polynucleotides of (a) to (e) under stringent conditions, wherein the polynucleotide has a biological activity; or (g) a polynucleotide consisting of a base sequence having at least 70% identity to any one of the polynucleotides of (a) to (e) or a complementary sequence thereof, wherein the polypeptide has a biological activity.

In one embodiment of this invention, the nucleic acid molecule encoding the Rho polypeptide comprises nucleotides 1 to 579 of the nucleic acid sequence as set forth in SEQ ID NO. 11.

In one embodiment of this invention, the nerve is in a condition including spinal cord injury, cerebrovascular disorder, or brain injury.

In one embodiment of this invention, the agent comprises an antisense molecule or RNAi.

According to another aspect of the present invention, a composition for regenerating nerves is provided, which comprises an agent capable of specifically interacting with a Rho kinase polypeptide.

In one embodiment of this invention, the Rho kinase polypeptide comprises: (a) a polypeptide encoded by a nucleic acid sequence as set forth in SEQ ID NO. 18 or a fragment thereof; (b) a polypeptide having an amino acid sequence SEQ ID NO. 19 or a fragment thereof; (c) a variant polypeptide having the amino acid sequence as set forth in SEQ ID NO. 19 having at least one mutation selected from the group consisting of one or more amino acid substitutions, additions, and deletions, wherein the variant peptide has a biological activity; (d) a polypeptide encoded by a splice variant or allelic variant of the base sequence as set forth in SEQ ID NO. 18; (e) a species homolog polypeptide of a polypeptide having the amino acid sequence as set forth in SEQ ID NO. 19; or (f) a polypeptide consisting of an amino acid sequence having at least 70% identity to any one of the polypeptides of (a) to (e), wherein the polypeptide has a biological activity.

In one embodiment of this invention, the Rho kinase polypeptide comprises amino acids 1 to 1388 of the amino acid sequence as set forth in SEQ ID NO. 19.

In one embodiment of this invention, the nerve is in a condition including spinal cord injury, cerebrovascular disorder, or brain injury.

In one embodiment of this invention, the agent comprises an antibody.

According to another aspect of the present invention, a composition for regenerating nerves is provided, whch comprises an agent capable of specifically interacting with a nucleic acid molecule encoding a Rho kinase polypeptide.

In one embodiment of this invention, the nucleic acid molecule encoding the Rho kinase polypeptide is a polynucleotide selected from the group consisting of: (a) a polynucleotide having a base sequence as set forth in SEQ ID NO. 18 or a fragment sequence thereof; (b) a polynucleotide encoding an amino acid sequence as set forth in SEQ ID NO. 19 or a fragment thereof; (c) a polynucleotide encoding a variant polypeptide having the amino acid sequence as set forth in SEQ ID NO. 19 having at least one mutation selected from the group consisting of one or more amino acid substitutions, additions, and deletions, wherein the variant polypeptide has a biological activity; (d) a polynucleotide which is a splice variant or allelic variant of the base sequence as set forth in SEQ ID NO. 18; (e) a polynucleotide encoding a species homolog of a polypeptide consisting of the amino acid having the amino acid sequence as set forth in SEQ ID NO. 19;

(f) a polynucleotide hybridizable to any one of the polynucleotides of (a) to (e) under stringent conditions, wherein the polynucleotide has a biological activity; or (g) a polynucleotide consisting of a base sequence having at least 70% identity to any one of the polynucleotides of (a) to (e) or a complementary sequence thereof, wherein the polypeptide has a biological activity.

In one embodiment of this invention, the nucleic acid molecule encoding the Rho kinase polypeptide comprises nucleotides 1 to 4164 of the nucleic acid sequence as set forth in SEQ ID NO. 18.

In one embodiment of this invention, the nerve is in a condition including spinal cord injury, cerebrovascular disorder, or brain injury.

In one embodiment of this invention, the agent comprises an antisense molecule or RNAi.

According to anther aspect of the present invention, a composition for regenerating nerves is provided, which comprises a p21 polypeptide.

In one embodiment of this invention, the p21 polypeptide comprises: (a) a polypeptide encoded by a nucleic acid sequence as set forth in SEQ ID NO. 13 or 22 or a fragment thereof; (b) a polypeptide having an amino acid sequence SEQ ID NO. 14 or 23 or a fragment thereof; (c) a variant polypeptide having the amino acid sequence as set forth in SEQ ID NO. 14 or 23 having at least one mutation selected from the group consisting of one or more amino acid substitutions, additions, and deletions, wherein the variant peptide has a biological activity; (d) a polypeptide encoded by a splice variant or allelic variant of the base sequence as set forth in SEQ ID NO. 13 or 22; (e) a species homolog polypeptide of a polypeptide having the amino acid sequence as set forth in SEQ ID NO. 14 or 23; or (f) a polypeptide consisting of an amino acid sequence having at least 70% identity to any one of the polypeptides of (a) to (e), wherein the polypeptide has a biological activity.

In one embodiment of this invention, the p21 polypeptide comprises amino acids 1 to 140 of the amino acid as set forth in SEQ ID NO. 14 or 23.

In one embodiment of this invention, the nerve is in a condition including spinal cord injury, cerebrovascular disorder, or brain injury.

In one embodiment of this invention, the p21 polypeptide further comprises a PTD domain.

In one embodiment of this invention, the PTD domain comprises an amino acid sequence of YGRKKRRQRRR or the amino acid sequence having one or more substitutions, additions and/or deletions.

In one embodiment of this invention, the PTD domain is located at the C-terminus or the N-terminus of the p21 polypeptide.

In one embodiment of this invention, the p21 polypeptide is substantially free of a nuclear localization domain.

In one embodiment of this invention, the p21 polypeptide further comprises a PTD domain and is substantially free of a nuclear localization domain.

In one embodiment of this invention, the p21 polypeptide further comprises a PTD domain and is substantially free of a nuclear localization domain, and the PTD domain is located at the C-terminus of the p21 polypeptide.

According to another aspect of the present invention, a composition for regenerating nerves is provided, which comprises a nucleic acid molecule encoding a p21 polypeptide.

In one embodiment of this invention, the nucleic acid molecule encoding the p21 polypeptide is a polynucleotide selected from the group consisting of: (a) a polynucleotide having a base sequence as set forth in SEQ ID NO. 13 or 22 or a fragment sequence thereof; (b) a polynucleotide encoding an amino acid sequence as set forth in SEQ ID NO. 14 or 23 or a fragment thereof; (c) a polynucleotide encoding a variant polypeptide having the amino acid sequence as set forth in SEQ ID NO. 14 or 23 having at least one mutation selected from the group consisting of one or more amino acid substitutions, additions, and deletions, wherein the variant polypeptide has a biological activity; (d) a polynucleotide which is a splice variant or allelic variant of the base sequence as set forth in SEQ ID NO. 13 or 22; (e) a polynucleotide encoding a species homolog of a polypeptide consisting of the amino acid having the amino acid sequence as set forth in SEQ ID NO. 14 or 23; (f) a polynucleotide hybridizable to any one of the polynucleotides of (a) to (e) under stringent conditions, wherein the polynucleotide has a biological activity; or (g) a polynucleotide consisting of a base sequence having at least 70% identity to any one of the polynucleotides of (a) to (e) or a complementary sequence thereof, wherein the polypeptide has a biological activity.

In one embodiment of this invention, the nucleic acid molecule encoding the p21 polypeptide comprises nucleotides 1 to 140 of the base sequence as set forth in SEQ ID NO. 13 or 22.

In one embodiment of this invention, the nerve is in a condition including spinal cord injury, cerebrovascular disorder, or brain injury.

In one embodiment of this invention, the nucleic acid molecule further comprises an agent encoding a PTD domain.

In one embodiment of this invention, the PTD domain comprises an amino acid sequence of YGRKKRRQRRR or the amino acid sequence having one or more substitutions, additions and/or deletions.

In one embodiment of this invention, a sequence encoding the PTD domain is located at the 5′-terminus or the 3′-terminus of a sequence encoding the p21 polypeptide.

In one embodiment of this invention, the nucleic acid molecule encoding the p21 polypeptide is substantially free of a sequence encoding a nuclear localization domain.

In one embodiment of this invention, the nucleic acid molecule encoding the p21 polypeptide further comprises a sequence encoding a PTD domain and is substantially free of a sequence encoding a nuclear localization domain.

In one embodiment of this invention, the nucleic acid molecule encoding the p21 polypeptide further comprises a sequence encoding a PTD domain and is substantially free of a sequence encoding a nuclear localization domain, and the sequence encoding the PTD domain is located at the 3′-terminus of the nucleic acid molecule encoding the p21 polypeptide.

According to another aspect of the present invention, a composition for regenerating nerves is provided, which comprises a PTD domain and a nerve regeneration agent.

In one embodiment of this invention, the nerve regeneration agent inhibits a p75 signal transduction pathway. In one embodiment of this invention, the nerve regeneration agent comprises a transduction agent in the p75 signal transduction pathway or a variant or fragment thereof, or an agent capable of specifically interacting with the transduction agent in the p75 signal transduction pathway.

In one embodiment of this invention, the transduction agent in the p75 signal transduction pathway comprises at least one transduction agent selected from the group consisting of MAG, GT1b, p75, Rho GDI, Rho, p21, and Rho kinase.

In one embodiment of this invention, the nerve regeneration agent has at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the nerve regeneration agent comprises at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase.

In one embodiment of this invention, the nerve regeneration agent comprises an agent selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof.

In one embodiment of this invention, the PTD domain comprises an amino acid sequence of YGRKKRRQRRR or the amino acid sequence having one or more substitutions, additions and/or deletions.

In one embodiment of this invention, the PTD domain is located at the C-terminus or the N-terminus of the p21 polypeptide.

In one embodiment of this invention, the nerve regeneration agent is capable of residing in the cytoplasm.

According to another aspect of the present invention, a composition for regenerating nerves is provided, which comprises a nucleic acid molecule comprising a nucleic acid sequence encoding a PTD domain and a nucleic acid sequence encoding a nerve regeneration agent.

In one embodiment of this invention, the nerve regeneration agent inhibits a p75 signal transduction pathway.

In one embodiment of this invention, the nerve regeneration agent comprises a transduction agent in the p75 signal transduction pathway or a variant or fragment thereof, or an agent capable of specifically interacting with the transduction agent in the p75 signal transduction pathway.

In one embodiment of this invention, the transduction agent in the p75 signal transduction pathway comprises at least one transduction agent selected from the group consisting of MAG, GT1b, p75, Rho GDI, Rho, p21 and Rho kinase.

In one embodiment of this invention, the nerve regeneration agent has at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the nerve regeneration agent comprises at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase.

In one embodiment of this invention, the nerve regeneration agent comprises an agent selected from the group consisting of a Pep5 polypeptide, an agent capable of specifically interacting with a p75 polypeptide, an agent capable of specifically interacting with a nucleic acid molecule encoding the p75 polypeptide, a p75 extracellular domain polypeptide, the Rho GDI polypeptide, an agent capable of specifically interacting with a MAG polypeptide, an agent capable of specifically interacting with a nucleic acid molecule encoding the MAG polypeptide, a p21 polypeptide, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof.

In one embodiment of this invention, the PTD domain comprises an amino acid sequence of YGRKKRRQRRR or the amino acid sequence having one or more substitutions, additions and/or deletions.

In one embodiment of this invention, the nucleic acid sequence encoding the PTD domain is located at the 5′-terminus or the 3′-terminus of the p21 polypeptide.

In one embodiment of this invention, the nerve regeneration agent is capable of residing in the cytoplasm.

According to another aspect of the present invention, a method for disrupting or reducing inhibition of neurite outgrowth is provided, which comprises the step of: inhibiting a p75 signal transduction pathway.

In one embodiment of this invention, the inhibition of the p75 signal transduction pathway is achieved by providing a transduction agent in the p75 signal transduction pathway or a variant or fragment thereof, or an agent capable of specifically interacting with the transduction agent in the p75 signal transduction pathway in an amount effective for regeneration.

In one embodiment of this invention, the transduction agent in the p75 signal transduction pathway comprises at least one transduction agent selected from the group consisting of MAG, GT1b, p75, Rho GDI, Rho, p21, and Rho kinase.

In one embodiment of this invention, the inhibition of the p75 signal transduction pathway is selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the inhibition of the p75 signal transduction pathway is achieved by providing at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase, in an amount effective for regeneration.

In one embodiment of this invention, the step of inhibiting the p75 signal transduction pathway comprises the step of: providing at least one molecule selected from the group consisting of a Pep5 polypeptide, a nucleic acid molecule encoding the Pep5 polypeptide, an agent capable of specifically interacting with a p75 polypeptide, an agent capable of specifically interacting with a, nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof, to the nerve in an amount effective for regeneration.

In one embodiment of this invention, the agent is bound to a PTD domain.

According to another aspect of the present invention, a composition for disrupting or reducing inhibition of neurite outgrowth is provided, which comprises an agent capable of inhibiting a p75 signal transduction pathway.

In one embodiment of this invention, the agent capable of inhibiting the p75 signal transduction pathway is in a form appropriate for delivery to a neuron at a site desired for nerve regeneration.

In one embodiment of this invention, the agent capable of inhibiting the p75 signal transduction pathway comprises a transduction agent in the p75 signal transduction pathway or a variant or fragment thereof, or an agent capable of specifically interacting with the transduction agent in the p75 signal transduction pathway.

In one embodiment of this invention, the transduction agent in the p75 signal transduction pathway comprises at least one transduction agent selected from the group consisting of MAG, GT1b, p75, Rho GDI, Rho, p21, and Rho kinase.

In one embodiment of this invention, the agent capable of inhibiting the p75 signal transduction pathway has at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the agent capable of inhibiting the p75 signal transduction pathway comprises at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase. The agent capable of inhibiting the p75 signal transduction pathway is present in an amount effective for regeneration.

In one embodiment of this invention, the agent capable of inhibiting the p75 signal transduction pathway comprises at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof.

In one embodiment of this invention, the agent is bound to a PTD domain.

According to another aspect of the present invention, a method for constructing a network of neurons is provided, which comprises the step of: inhibiting a p75 signal transduction pathway in the neuron.

In one embodiment of this invention, the inhibition of the p75 signal transduction pathway is achieved by providing a transduction agent in the p75 signal transduction pathway or a variant or fragment thereof, or an agent capable of specifically interacting with the transduction agent in the p75 signal transduction pathway to the neuron in an amount effective for regeneration.

In one embodiment of this invention, the transduction agent in the p75 signal transduction pathway is at least one transduction agent selected from the group consisting of MAG, GT1b, p75, Rho GDI, Rho, p21, and Rho kinase.

In one embodiment of this invention, the inhibition of the p75 signal transduction pathway is selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the inhibition of the p75 signal transduction pathway is achieved by providing at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase, in an amount effective for regeneration.

In one embodiment of this invention, the step of inhibiting the p75 signal transduction pathway comprises the step of: providing a composition comprising at least one molecule selected from the group consisting of a Pep5 polypeptide, a nucleic acid molecule encoding the Peps polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase, an agent capable of specifically interacting with a nucleic acid molecule encoding the Rho kinase, and variants and fragments thereof, to the neuron in an amount effective for regeneration.

In one embodiment of this invention, the agent is bound to a PTD domain.

According to another aspect of the present invention, a composition for constructing a network of neurons is provided, which comprises an agent capable of inhibiting a p75 signal transduction pathway.

In one embodiment of this invention, the agent capable of inhibiting the p75 signal transduction pathway comprises a transduction agent in the p75 signal transduction pathway or a variant or fragment thereof, or an agent capable of specifically interacting with the transduction agent in the p75 signal transduction pathway.

In one embodiment of this invention, the transduction agent in the p75 signal transduction pathway comprises at least one transduction agent selected from the group consisting of MAG, GT1b, p75, Rho GDI, Rho, p21, and Rho kinase.

In one embodiment of this invention, the agent capable of inhibiting the p75 signal transduction pathway has at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the agent capable of inhibiting the p75 signal transduction pathway comprises at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase. The agent capable of inhibiting the p75 signal transduction pathway is present in an amount effective for regeneration.

In one embodiment of this invention, the agent capable of inhibiting the p75 signal transduction pathway comprises at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof.

In one embodiment of this invention, the agent is bound to a PTD domain.

According to another aspect of the present invention, a kit for treatment of neurological diseases is provided, which comprises: (A) a cell population regenerated with a composition comprising an agent capable of inhibiting a p75 signal transduction pathway; and (B) a container for preserving the cell population.

In one embodiment of this invention, the agent capable of inhibiting the p75 signal transduction pathway comprises a transduction agent in the p75 signal transduction pathway or a variant or fragment thereof, or an agent capable of specifically interacting with the transduction agent in the p75 signal transduction pathway.

In one embodiment of this invention, the transduction agent in the p75 signal transduction pathway comprises at least one transduction agent selected from the group consisting of MAG, GT1b, p75, Rho GDI, Rho, p21 and Rho kinase.

In one embodiment of this invention, the agent capable of inhibiting the p75 signal transduction pathway has at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the agent capable of inhibiting the p75 signal transduction pathway comprises at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase. The agent capable of inhibiting the p75 signal transduction pathway is present in an amount effective for regeneration.

In one embodiment of this invention, the agent capable of inhibiting the p75 signal transduction pathway comprises at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof.

In one embodiment of this invention, the agent is bound to a PTD domain.

According to another aspect of the present invention, a method for treating neurological diseases is provided, whch comprises the steps of: (a) providing a cell population regenerated with a composition comprising an agent capable of inhibiting a p75 signal transduction pathway; and (b) transplanting the cell population to a patient.

In one embodiment of this invention, the inhibition of the p75 signal transduction pathway is achieved by providing a transduction agent in the p75 signal transduction pathway or a variant or fragment thereof, or an agent capable of specifically interacting with the transduction agent in the p75 signal transduction pathway to the neuron in an amount effective for regeneration.

In one embodiment of this invention, the transduction agent in the p75 signal transduction pathway is at least one transduction agent selected from the group consisting of MAG, GT1b, p75, Rho GDI, Rho, p21, and Rho kinase.

In one embodiment of this invention, the inhibition of the p75 signal transduction pathway is selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In one embodiment of this invention, the inhibition of the p75 signal transduction pathway is achieved by providing at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase, in an amount effective for regeneration.

In one embodiment of this invention, the step of inhibiting the p75 signal transduction pathway comprises the step of: providing a composition comprising at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase, an agent capable of specifically interacting with a nucleic acid molecule encoding the Rho kinase, and variants and fragments thereof, to the neuron in an amount effective for regeneration.

In one embodiment of this invention, the agent is bound to a PTD domain.

According to another aspect of the present invention, a screening method for identifying an agent which induces nerve regeneration is provided, which comprises the steps of: (a) contacting at least two agents capable of interacting with each other in a p75 signal transduction pathway in the presence of a test agent; and (b) comparing a level of an interaction between the at least two agents in the presence of a test agent with a level of an interaction of the at least two agents in the absence of the test agent. The test agent is identified as an agent for regenerating nerves when the level of the interaction in the presence of the test agent is reduced as compared to the level of the interaction in the absence of the test agent.

In one embodiment of this invention, the interaction includes at least one interaction selected from the group consisting of an interaction between MAG and GT1b, an interaction between GT1b and p75, an interaction between p75 and Rho, an interaction between p75 and Rho GDI, interaction between Rho and Rho GDI, conversion from Rho GDP to Rho GTP, an interaction between Rho and Rho kinase, and an activity of Rho kinase, and the reduction of the interaction includes at least one action selected from the group consisting of inhibition of the interaction between MAG and GT1b, inhibition of the interaction between GT1b and p75, inhibition of the interaction between p75 and Rho, inhibition of the interaction between p75 and Rho GDI, maintenance or enhancement of the interaction between Rho and Rho GDI, inhibition of the conversion from Rho GDP to Rho GTP, inhibition of the interaction between Rho and Rho kinase, and inhibition of the activity of Rho kinase.

In one embodiment of this invention, the at least two agents comprise a first polypeptide having an amino acid sequence having at least 70% homology to SEQ ID NO. 4 or 17 or a fragment thereof and a second polypeptide having an amino acid sequence having at least 70% homology to SEQ ID NO. 6 or a fragment thereof, and the comparing step comprises comparing a binding level of the first polypeptide and the second polypeptide in the presence of the test agent with a binding level of the first polypeptide and the second polypeptide in the absence of the test agent.

According to another aspect of the present invention, a modulating agent identified by the above-described method is provided.

According to another aspect of the present invention, a pharmaceutical composition comprising the above-described modulating agent is provided.

According to another aspect of the present invention, a method for prophylaxis or treatment of neurological diseases, disorders or conditions is provided, which comprises the step of: administering the above-described pharmaceutical composition to a subject.

According to another aspect of the present invention, a vector is provided, which comprises at least one nucleic acid molecule selected from the group consisting of a nucleic acid molecule encoding a MAG polypeptide, a nucleic acid molecule encoding a p75 polypeptide, a nucleic acid encoding a Rho GDI polypeptide, a nucleic acid molecule encoding Rho, a nucleic molecule encoding p21, and a nucleic acid molecule encoding Rho kinase, wherein the at least one nucleic acid molecule has a sequence comprising an introduced sequence different from a sequence of a wild type of the at least one nucleic acid molecule.

According to another aspect of the present invention, a cell is provided, which comprises the above-described vector.

According to another aspect of the present invention, a tissue is provided, which comprises the above-described vector.

According to another aspect of the present invention, an organ is provided, which comprises the above-described vector.

According to another aspect of the present invention, an organism is provided, which comprises the above-described vector.

According to another aspect of the present invention, a nerve-modified transgenic animal is provided, which is transformed with the above-described vector.

According to another aspect of the present invention, a nerve-modified knockout animal is provided, wherein at least one nucleic acid molecule selected from the group consisting of a nucleic acid molecule encoding a MAG polypeptide, a nucleic acid molecule encoding a p75 polypeptide, a nucleic acid encoding a Rho GDI polypeptide, a nucleic acid molecule encoding Rho, a nucleic molecule encoding p21, and a nucleic acid molecule encoding a Rho kinase, is deleted.

These and other advantages of the present invention will become apparent to those skilled in the art upon reading and understanding the following detailed description with reference to the accompanying figures.

### BRIEF DESCRIPTION OF THE DRAWINGS

FIG. 1 shows the effects of MAG on neurons which are dependent on p75^{NTR}. (A) Dissociated DRG neurons were incubated for 24 h with or without MAG-Fc, and then were immunostained with monoclonal antibody (TuJ1) recognizing the neuron-specific β-tubulin III protein. p75(+/+), wild type; p75(−/−), mice carrying a mutation in the p75^{NTR} gene. (B) Mean length of the longest neurite per neuron. Data are mean±S.E.M. An asterisk indicates statistical significance, *; p<0.01 (Student's t-test). (C) Mean length of the longest neurite per neuron. Dissociated cerebellar neurons were incubated for 24 h with or without MAG-Fc.

FIG. 2 shows that MAG activates RhoA through a p75^{NTR} dependent mechanism. (A) The effect of C3 transferase on MAG-treated DRG neurons from wild type mice. Mean length of the longest neurite per neuron. Data are mean±S.E.M. Asterisks indicate statistical significance, *; p<0.01 (Student's t-test). (B) Binding of MAG-Fc to 293 cells was visualized by incubation with a FITC-tagged anti-human IgG. (C) Affinity precipitation of RhoA in transfected 293 cells. MAG-Fc (25 μg/ml) elicits activation of RhoA only when 293 cells express p75^{NTR}.

FIG. 3 shows affinity precipitation of RhoA in postnatal cerebellar neurons. (A) RhoA activity was increased after the addition of MAG-Fc (25 μg/ml). RhoA activity is indicated by the amount of RBD-bound RhoA normalized to the amount of RhoA in the lysates. Values represent RhoA activity relative to the cells at time 0. Results are means±SE from three experiments. Asterisks indicate statistical significance, *; p<0.01 (Student's t-test). (B) NGF rapidly inhibits RhoA activity (˜10 min). (C) shows dose response. (D) The activation was lost in the neurons from mice carrying a mutation in the p75^{NTR} gene.

FIG. 4 shows co-localization of p75^{NTR} and MAG binding. (A) DRG neurons were stained with the anti-p75^{NTR} antibody and an Alexa fluor™ 568-conjugated secondary antibody. Binding of MAG-Fc was visualized by incubation with the FITC-tagged anti-human IgG. Confocal microscopy was performed on a Zeiss LSM-510 laser scanning microscope. Representative single optical sections for p75^{NTR} (left), MAG binding (middle) and overlay images (right) are shown. Close association of these markers on the neurites was seen in almost all the neurons with p75^{NTR} immunoreactivity. (B) shows binding of MAG-Fc to DRG neurons from mice carrying a mutation in the p75^{NTR} gene.

FIG. 5 shows association of MAG, p75^{NTR} and GT1b. (A) shows co-precipitation of p75^{NTR} and MAG-Fc using lysates prepared from P9 cerebellum. In the MAG-Fc precipitates, the anti-p75^{NTR} antibody revealed the presence of a protein corresponding to p75^{NTR} (B) shows co-precipitation of recombinant p75^{NTR} and GT1b. Association was examined by the present inventorsstern blot analysis of the precipitates produced with protein A sepharose and Fc fused protein of p75^{NTR}-GT1b antibody revealed the presence of a 100-kDa protein (left), which was shown to be p75^{NTR} by the anti-p75^{NTR} antibody (right). (C) shows co-precipitation of recombinant p75^{NTR} and other gangliosides. (D) shows co-immunoprecipitation of p75^{NTR} and GT1b using lysates prepared from P9 cerebellum. In the GT1b immunoprecipitates, the anti-p75^{NTR} antibody revealed the presence of a protein corresponding to p75^{NTR} The lower bands correspond to the Ig of the antibodies used. (E) shows co-immunoprecipitation of p75^{NTR} and GT1b using transfected 293 cells. In the p75^{NTR} immunoprecipitates, the anti-GT1b antibody revealed the presence of a protein (left), which was shown to be p75^{NTR} by the anti-p75^{NTR} antibody (right).

FIG. 6 shows co-immunoprecipitation of p75^{NTR} with Rho GDI. (A) shows co-immunoprecipitation of p75^{NTR} with Rho GDI or RhoA using lysates prepared from the transfected 293T cells. In the p75^{NTR} immunoprecipitates, the anti-Rho GDI antibody revealed the presence of a protein corresponding to Rho GDI. (B) shows the effects of MAG and Nogo on the interaction of p75^{NTR} with Rho GDI or RhoA in the transfected N1E-115 cells. Data are mean±S.E. Asterisks indicate statistical significance, *; p<0.01 (Student's t-test). (C) shows co-immunoprecipitation of p75^{NTR} and Rho GDI using lysates prepared from cerebellar neurons. Association was observed in MAG- and Nogo-treated cells.

FIG. 7 shows that p75^{NTR} directly associates with Rho GDI. (A) shows co-precipitation of p75^{NTR} with recombinant GST-Rho GDI or GST-RhoA. Association was examined by the present inventorsstern blot analysis of the precipitates produced with the purified p75^{NTR} and protein A sepharose. The anti-GST antibody revealed the presence of a Rho GDI in the complex. (B) shows co-precipitation of Rho GDI with the deletion mutants of p75^{NTR}. A schematic representation of the constructs for the deleted mutants is shown. The indicated numbers correspond to residues of the mutants. (C) shows affinity precipitation of RhoA in the transfected 293T cells. Overexpression of the full-length of p75 NT or p75^{NTR} ICD elicits activation of RhoA, while the mutated p75^{NTR} that lacks the fifth helix fails to activate RhoA.

FIG. 8 shows that p75^{NTR} reduces the Rho GDI activity. (A) shows that p75^{NTR} is not a guanine nucleotide exchange factor for RhoA. The ability of the proteins to induce the dissociation of ³H-labeled GDP from RhoA in 30 min was measured. GST protein or the incubation buffer was used as a control. The graph represents the average of relative amount of initial ³H-GDP remaining bound±S.E. from three individual experiments. *, p<0.01; (Student's t-test). (B) shows that p75^{NTR} HD inhibits the Rho GDI activity in vitro. The GDP/GTP exchange reaction of RhoA in complex with Rho GDI was determined in the presence or absence of p75^{NTR} HD. In the [³H] GDP dissociation assay, the dissociation of [³H]GDP from [³H] GDP-RhoA complexed with Rho GDI was assayed by measuring the radioactivity of [³H] GDP bound to RhoA. In the [³⁵S] GTPγS binding assay, the binding of [³⁵S] GTPγS to GDP-RhoA complexed with Rho GDI was assayed by measuring the activity of [³⁵S] GTPγS bound to RhoA. Closed circle, GST-p75^{NTR} HD; Open square, GST. *, p<0.01; (Student's t-test). (C) shows that p75^{NTR} inhibits the Rho GDI activity. The GDP/GTP exchange reaction of RhoA stimulated with Dbl was determined. The [³H] GDP-RhoA-Rho GDI complex (50 nM) was incubated with 90 nM GST-Dbl and GST-fused proteins at the indicated concentrations. Closed circle, GST-p75^{NTR} HD; Open square, GST; Open triangle, GST-p75^{NTR} ICD. *, p<0.01; (Student's t-test). (C) shows that overexpression of Rho GDI abolishes the effect of MAG and Nogo. The effect of Rho GDI on the neurite outgrowth of dissociated cerebellar neurons was assessed. Left; images of representative cells transiently transfected with the control or Rho GDI plasmid. MAG, MAG-Fc (25 μg/ml); Nogo, the Nogo peptide (4 μM); Rho GDI, cells transfected with myc-tagged Rho GDI. Data are mean±S.E. An asterisk indicates statistical significance, *; p<0.01 (Student's t-test).

FIG. 9 shows that Pep5 inhibits interaction of Rho GDI with p75^{NTR}. (A) shows co-precipitation of p75^{NTR} with recombinant GST-Pep5. (B) shows that Pep5 inhibits the binding of p75^{NTR} with Rho GDI dose dependently. (C) shows co-immunoprecipitation of p75^{NTR} and Rho GDI using lysates prepared from cerebellar neurons. The interaction was diminished by TAT-Pep5.

FIG. 10 shows that Pep5 silences the inhibitory action of p75^{NTR}. (A) shows that dissociated DRG neurons were incubated for 24 h with or without the Nogo peptide, and then were immunostained with monoclonal antibody (TuJ1) recognizing the neuron-specific β-tubulin III protein. Nogo, the Nogo peptide; Pep5, TAT-Pep5. (B) shows neurite outgrowth of DRG neurons. MAG, MAG-Fc; HD, the peptide corresponding to the p75^{NTR} HD (residues 368-381); p75(+/+), wild type; p75(−/−), mice carrying a mutation in the p75^{NTR} gene. Data are mean±S.E. Asterisks indicate statistical significance, *; p<0.01 (Student's t-test). (C) shows that dissociated cerebellar neurons were incubated for 24 h with or without the Nogo peptide. (D) shows neurite outgrowth of cerebellar neurons. Data are mean±S.E. Asterisks indicate statistical significance, *; p<0.01 (Student's t-test). (E) shows affinity precipitation of RhoA in cerebellar neurons. The Nogo peptide. (4 μM) and MAG-Fc (25 μg/ml) elicit activation of RhoA, whereas TAT-Pep5 (1 μM) completely abolishes these effects.

FIG. 11 shows inhibition of myelin signal by the antibody to the p75^{NTR}. (A) shows dissociated cerebellar neurons were incubated for 24 h with or without myelin-derived inhibitors. Mean length of the longest neurite per neuron. Data are mean±S.E.M. Asterisks indicate statistical significance; *, p<0.01 (Student's t-test). Nogo, GST-Nogo; Fc-p75, the extracellular domain of the p75^{NTR} fused with Fc; p75-Ab, the antibody to the p75^{NTR}; MAG, MAG-Fc. (B) shows affinity precipitation of RhoA in cerebellar neurons. (C) shows co-immunoprecipitation of the endogenous p75^{NTR} and the NgR using lysates prepared from P9 cerebellum.

FIG. 12 shows that the antibody to the p75^{NTR} improves locomotor behavior and enhances sprouting of mouse CST fibers. (A) shows modified BBB scores of anti-p75 antibody-treated mice (n=12) revealed significantly higher recovery than those of control antibody-treated mice (n=12) from seven days after injury to 4 weeks. *, p<0.05 (Student's t-test), compared with control antibody-treated mice. SCI, Spinal cord injury. (B) shows that the anti-p75 antibody promotes axonal outgrowth after CST injury. Anterogradely BDA-labeled axons (arrows) in the anti-p75 antibody-treated mouse in a transverse section of the gray matter 2 mm caudal to the injured site 28days after injury. Scale bar: 25 μm. (C) shows the number of regenerating axons labeled with BDA per transverse section caudal to the CST region. Data represents mean±S.E. from five control or anti-p75 antibody-treated mice respectively. *, p<0.05 (Student's t-test), compared with control antibody-treated mice.

FIG. 13 shows that chick retinal neurons from E5 embryos display cytoplasmic p21^{Cip1/WAF1} expression. (A) shows that chick retinas from E5 embryos were immunostained with the anti-p21^{Cip1/WAF1} antibody. In every panel, the right side is the vitreous body and the left side is the pigment epithelium. (B) shows p21^{Cip1/WAF1} immunoreactivity in chick dissociated retinal cells from E5 embryos. The upper panels are the cells devoid of β-tubulin immunoreactivity, and the lower panels are the neurons.

FIG. 14 shows subcellular localization of p21^{Cip1/WAF1} in DMSO-induced differentiating N1E-115 cells and immunocytochemical staining of p21^{Cip1/WAF1} with the anti-p21^{Cip1/WAF1} antibody. Representative features of N1E-115 cells incubated without DMSO (A), or with DMSO for 1 day (B) and 4 days (C).

FIG. 15 shows morphological changes of N1E-115 cells by overexpression of p21^{Cip1/WAF1}. (A) shows growth of N1E-115 cells. Cells were seeded in 6-cm dishes, transfected, and were counted 1 and 2 days after transfection. The relative increases in the number of the cells are shown. The values are means±SEM of 3 independent experiments. *, p<0.01 compared with full-p21 (Student's t-test). There is no significant difference between GFP and GFP-ΔNLS-p21 transfected cells. (B) shows the western blot analysis of cyclinD3 and pRb. N1E-115 cells were treated with DMSO, or transfected with GFP-full-p21 or GFP-ΔNLS-p21, then were harvested at 1, 2, 3 and 4 days. Arrowheads indicate hyperphosphorylated pRb, and the arrow indicates underphosphorylated pRb. (C) shows expression levels of p21^{Cip1/WAF1} in N1E-115 cells treated with DMSO for 4 days or transfected with GFP-ΔNLS-p21. (D) shows that N1E-115 cells were transfected with GFP (control), GFP-full-p21 or GFP-ΔNLS-p21. Shown are photomicrographs of the cells transfected with each construct. (E) shows quantification of the morphology of the cells. N1E-115 cells exposed to Y-27632 (10 μM) for 30 minutes or expressing GFP, GFP-full-p21 or GFP-ΔNLS-p21 were categorized into 3 groups; the cells with long neurites (long neurite), cells with a round form (round), and cells with other forms (others). Data represent means±SEM of 3 independent experiments. *, p<0.05 compared with control. **, p<0.01 compared with control as well as full-p21 (Student's t-test).

FIG. 16 shows effects of cytoplasmic p21^{Cip1/WAF1} on the cytoskeletal organization. (A) show that NIH3T3 cells were transfected with GFP-ΔNLS-p21. After serum starvation for 16 hours, the cells were treated with 10% fetal bovine serum, fixed and stained with rhodamine-conjugated phalloidine. (B) shows quantification of the cells containing stress fibers. Data represent means±SEM of 3 independent experiments. *, p<0.01 compared with GFP (Student's t-test).

FIG. 17 shows cytoplasmic p21^{Cip1/WAF1}, but not p21^{Cip1/WAF1} in the nucleus, precipitates Rho-kinase. (A) shows subcellular localization of ectopically expressed proteins in 293T cells. Note the difference in the localization between GFP-full-p21 and GFP-ΔNLS-p21. (B) shows that 293T cells were cotransfected with myc-Rho-kinase in combination with GFP-full-p21 or GFP-ΔNLS-p21. The lysates were immunoprecipitated with the anti-p21^{Cip1/WAF1} antibody. Immunocomplexes were electrophorased and blotted with anti-myc antibody. Expression of Rho-kinase and p21^{Cip1/WAF1} in the lysates was determined. (C) shows interaction of p21^{Cip1/WAF1} with Rho-kinase using lysates prepared from differentiating N1E-115 cells with DMSO treatment. Immunoprecipitated p21^{Cip1/WAF1} was electrophorased and immunoblotted with anti-Rho-kinase antibody. Anti-mouse IgG antibody was used as a negative control. (D) shows in vitro interaction of recombinant full-length p21^{Cip1/WAF1} and the catalytic domain of Rho-kinase (GST-CAT). S6 kinase substrate peptide (AKRRRLSSLRA) and Y-27632 at the indicated concentrations were co-incubated.

FIG. 18 shows that p21^{Cip1/WAF1} inhibits Rho-kinase activity. (A) shows that the activity of Rho-kinase was assayed in the presence of the indicated concentrations of p21^{Cip1/WAF1}. The percentage was quantified compared to CPM in the absence of p21^{Cip1/WAF1}. Data represent means±SEM of 3 independent experiments. (B) shows that the activity of Rho-kinase was assayed with the cells exposed to Y-27632 (10 μM) for 30 minutes or cotransfected with myc-Rho-kinase and p21^{Cip1/WAF1} constructs. The expression of Rho-kinase was determined by the present inventorsstern blot to normalize the relative activities. The relative activities were quantified compared to CPM in the control cells cotransfected with myc-Rho-kinase and GFP. Data represent means±SEM of 3 independent experiments. *, p<0.001 compared with control (Student's t-test).

FIG. 19 shows neurite outgrowth and branching of hippocampal neurons by overexpression of cytoplasmic p21^{Cip1/WAF1}. (A) shows morphology of hippocampal neurons transfected with GFP or GFP-ΔNLS-p21 by computer tracing. Primary hippocampal neurons were transfected with GFP (control) or GFP-ΔNLS-p21 (ΔNLS-p21). Neurons were immunostained with anti-β-tubulin III antibody, and were traced with image analysis computer software. Scale bar; 10 μm. (B) shows morphological analysis of primary hippocampal neurons transfected with GFP or GFP-ΔNLS-p21. In neurons transfected with ΔNLS-p21, the total neurite length, the axonal length and the number of branch points per neuron were increased compared to those transfected with GFP. Data represent means±SEM of 3 independent experiments. *, p<0.001 compared with control (Student's t-test).

FIG. 20 schematically shows a construct in which p21 is fused with a TAT PTD domain (bottom) and a control construct (top).

FIG. 21 shows the functional recovery of a rat, whose spinal cord had been injured, due to the p21 construct. The rat was observed from day 2 after spinal cord injury for 6 weeks.

FIG. 22 shows a signal transduction pathway involved in inhibition of regeneration.

### DESCRIPTION OF SEQUENCE LISTING

SEQ ID NO. 1: the nucleic acid sequence of a Pep5 polypeptide. SEQ ID NO. 1 is a degenerate nucleic acid sequence of a Pep polypeptide as set forth in SEQ ID NO. 2.

Pep5 AA Sequence

C F F R G G F F N H N P R Y C
Cys Phe Phe Arg Gly Gly Phe Phe Asn His Asn Pro Arg Tyr Cys
tgy tty tty mgn ggn ggn tty tty aay cay aay ccn mgn tay tgy
tgt ttt cgt ggt aat cat cct tat
tgc ttc cgc ggc aac cac ccc tac
cga gga cca
cgg ggg ccg
aga
agg

SEQ ID NO. 1: Pep5 degenerate DNA tgyttyttymgnggnggnttyttyaaycayaayccnmgntaytgy

SEQ ID NO. 2: the amino acid sequence of a Pep5 polypeptide.

SEQ ID NO. 3: the nucleic acid sequence of a human p75 polypeptide.

SEQ ID NO. 4: the amino acid sequence of the human p75 polypeptide.

SEQ ID NO. 5: the nucleic acid sequence of a human Rho GDI polypeptide.

SEQ ID NO. 6: the amino acid sequence of the human the Rho GDI polypeptide.

SEQ ID NO. 7: the nucleic acid sequence of a MAG polypeptide.

SEQ ID NO. 8: the amino acid sequence of the MAG polypeptide.

SEQ ID NO. 9: the nucleic acid sequence of a Nogo polypeptide.

SEQ ID NO. 10: the amino acid sequence of the Nogo polypeptide.

SEQ ID NO. 11: the nucleic acid sequence of a Rho A polypeptide.

SEQ ID NO. 12: the amino acid sequence of the Rho A polypeptide.

SEQ ID NO. 13: the nucleic acid sequence of a p21 polypeptide.

SEQ ID NO. 14: the amino acid sequence of the p21 polypeptide.

SEQ ID NO. 15: a control peptide used in Examples.

SEQ ID NO. 16: the nucleic acid sequence of a rat p75 polypeptide.

SEQ ID NO. 17: the amino acid sequence of the rat p75 polypeptide.

SEQ ID NO. 18: the nucleic acid sequence of a human Rho kinase polypeptide.

SEQ ID NO. 19: the amino acid sequence of the human Rho kinase polypeptide.

SEQ ID NO. 20: the amino acid sequence of a TAT PTD domain.

SEQ ID NO. 21: the nucleic acid sequence of a HIV TAT PTD domain.

SEQ ID NO. 22: the nucleic acid sequence of a p21 polypeptide used in the Examples.

SEQ ID NO. 23: the amino acid sequence of the p21 polypeptide used in the Examples.

SEQ ID NO. 24: the amino acid sequence of ADB substrate peptide.

SEQ ID NO. 25: the full length amino acid sequence of a HIV TAT PTD domain.

### DETAILED DESCRIPTION OF THE INVENTION

It should be understood throughout the present specification that articles for singular forms include the concept of their plurality unless otherwise mentioned. Therefore, articles or adjectives for singular forms (e.g., “a”, “an”, “the”, and the like in English) include the concept of their plurality unless otherwise specified. It should be also understood that terms as used herein have definitions ordinarily used in the art unless otherwise mentioned. Therefore, all technical and scientific terms used herein have the same meanings as commonly understood by those skilled in the relevant art. Otherwise, the present application (including definitions) takes precedence.

(Definitions)

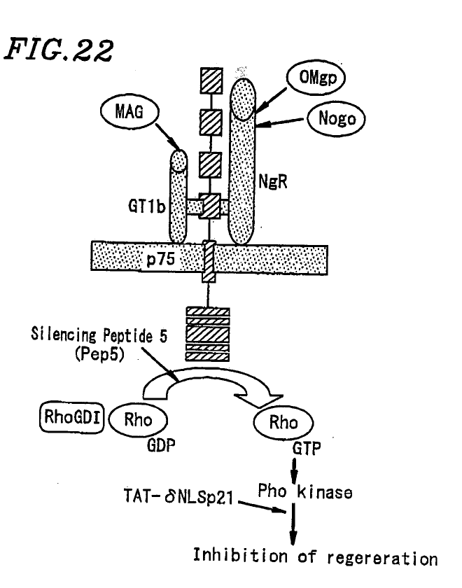

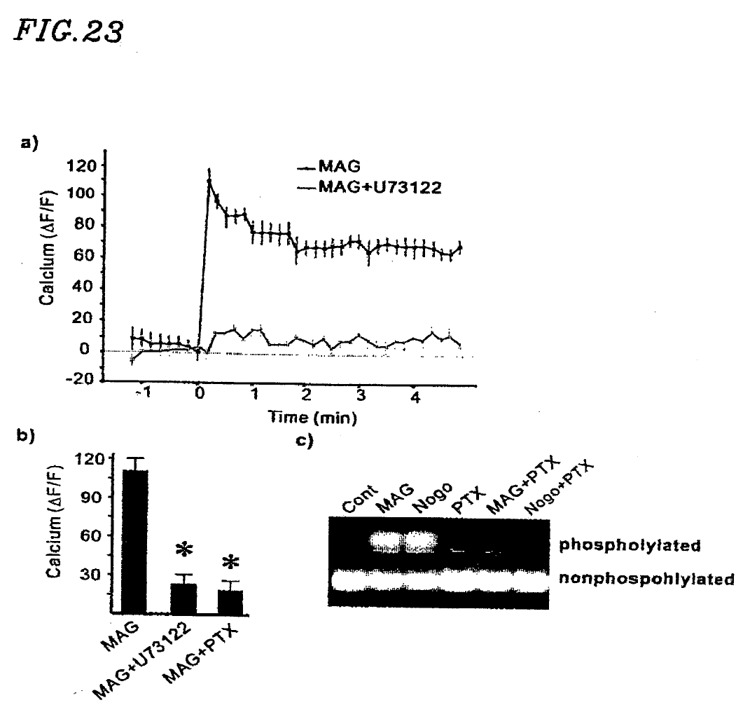

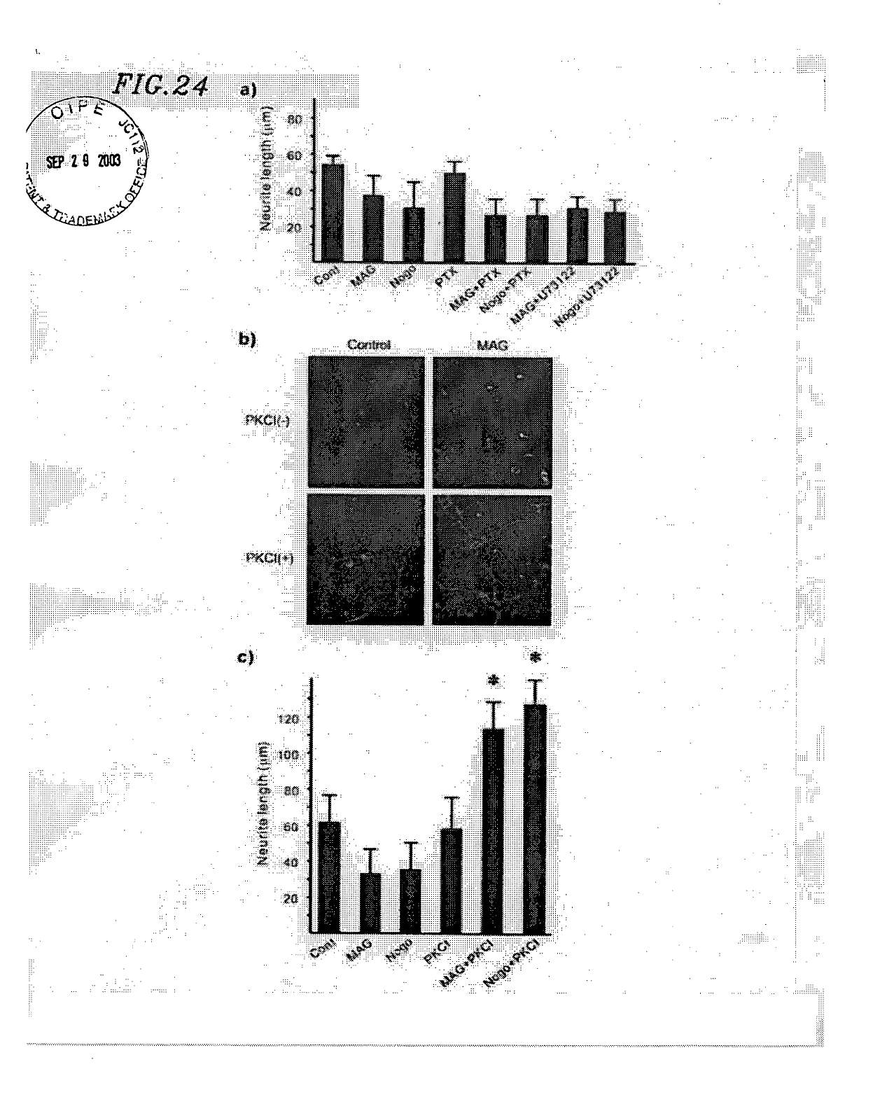

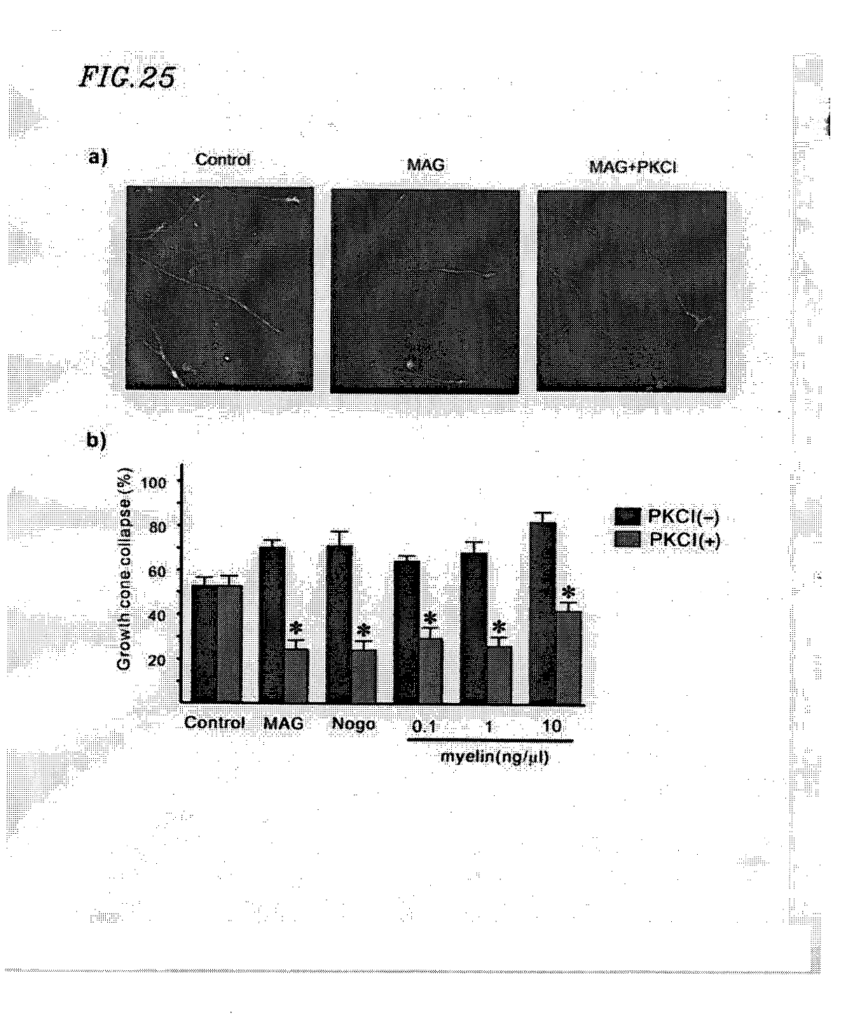

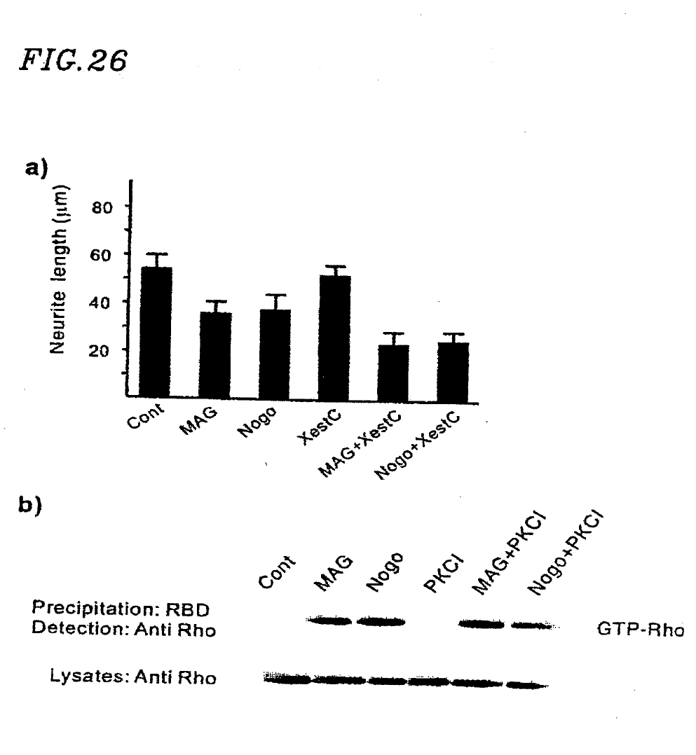

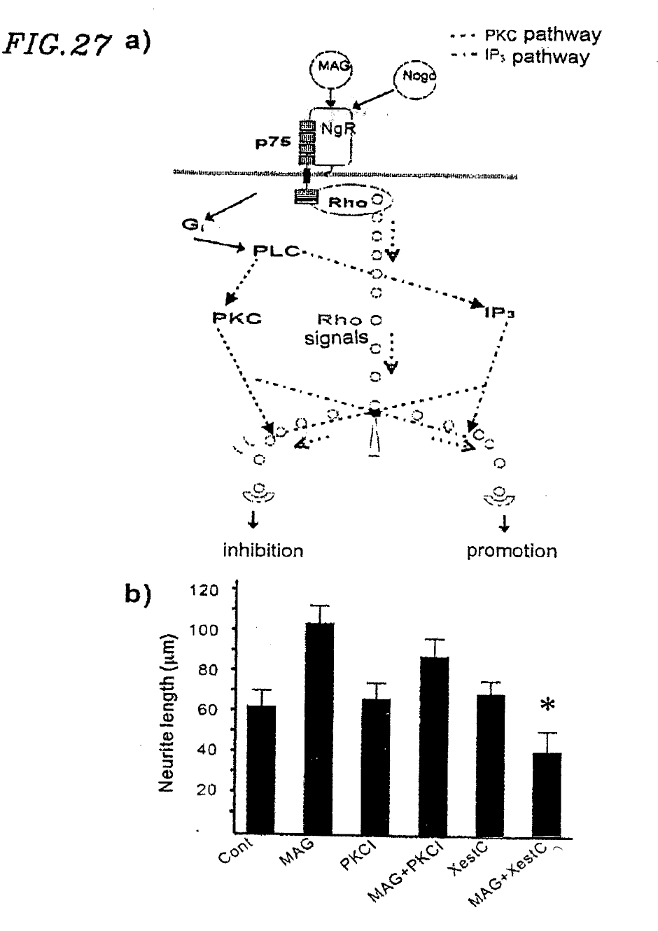

As used herein, “p75 signal transduction pathway” refers to a series of signal transduction pathways from activation of Rho by myelin-derived proteins via the p75 receptor on nerve membranes to inhibition of neurite outgrowth, i.e., a mechanism causing a phenomenon that once a central nerve axon is injured, the axon can no longer regenerated. Referring to FIG. 22, the p75 signal transduction pathway is a pathway in which when a myelin-derived protein acts on p75, Rho is activated via p75, so that neurite outgrowth is inhibited.

As used herein, “Pep5” refers to a peptide which binds to the intracellular domain of p75 to inhibit activation of Rho by p75. Representatively, Peps has sequences as set forth in SEQ ID NO. 1 (degenerate nucleic acid sequence) and SEQ ID NO. 2 (amino acid sequence). Variants and fragments of Pep5 are also included within the definition of Pep5 as long as they retain biological activity. Examples of the biological activity of Pep5 include, but are not limited to, blocking of neurite outgrowth inhibition by a myelin-derived protein. Such activity can be measured with a Rho activity assay which blocks activation of Rho by a myelin-derived protein, or the like.

As used herein, “p75^{NTR}” refers to a neurotrophin receptor which is involved in the regulation of axonal elongation by a neurotrophin and several myelin components (including myelin-binding glycoprotein, Nogo, and oligodendrocyte myelin glycoprotein). The neurotrophin receptor p75 (p75^{NTR}) mediates surprisingly diverse biological effects (e.g., see Dechant et al., supra) (e.g., cell death, Schwann cell migration, modulation of synaptic transmission, and functional regulation of sensory neurons and calcium currents). Recent work also implicates p75^{NTR} in the regulation of axon elongation.

As used herein, “p75” is used interchangeably with p75^{NTR} to refer to a single transmembrane receptor which mediates signal transduction of a myelin-derived protein where a neurotrophin is a ligand. Representatively, p75 has sequences as set forth in SEQ ID NO. 3 or 16 (human or rat nucleic acid sequences, respectively) and SEQ ID NO. 4 or 17 (human or rat amino acid sequences, respectively), and their variants and fragments are also included within the definition of p75 as long as they have biological activity. Examples of the biological activity of p75 include, but are not limited to, blocking of neurite outgrowth inhibition by a neurotrophin. Such activity can be measured with an assay which blocks activation of Rho by a myelin-derived protein, or the like.

As used herein, “p75 extracellular domain” refers to an extracellular portion (amino terminus) of p75 which is a single transmembrane receptor present on cell membranes. The p75 extracellular domain representatively has sequences indicated by positions 1110-1283 of SEQ ID NO. 3 (human nucleic acid sequence) or positions 1113-1277 of SEQ ID NO. 16 (rat nucleic acid sequence) and positions 273-427 of SEQ ID NO. 4 (human amino acid sequence) or positions 274-425 of SEQ ID NO. 17 (rat amino acid sequence), and their variants and fragments are also included within the definition of the p75 extracellular domain as long as they have biological activity. Examples of the biological activity of the p75 extracellular domain include, but are not limited to, blocking of neurite outgrowth inhibition by a myelin-derived protein. Such activity can be measured with an assay which blocks activation of Rho by a myelin-derived protein, or the like.

As used herein, the term “p75 extracellular domain” is also referred to as “soluble p75 polypeptide”. Therefore, a soluble p75 polypeptide is a p75 polypeptide which is not anchored in the membrane. Such a soluble polypeptide includes, but is not limited to, a p75 polypeptide such that, for example, the GPI anchor signal portion thereof which is sufficient for anchoring the polypeptide is deleted or the GPI anchor signal is modified so that the GPI anchor signal is not sufficient for replacement of the polypeptide with the GPI anchor. In a preferred embodiment, up to 5, 10, 20 or 25 amino acids are removed from the C terminus of p75, which makes the protein soluble.

A soluble p75 polypeptide may include the whole p75 protein including a putative GPI signal sequence. In another embodiment, the signal peptide of the protein may be deleted or truncated or shortened.

The terms “Rho GDP release inhibiting protein” and “Rho GDI” are used interchangeably to refer to a protein which has a role in inhibition of nucleotide release and the shuttling of Rho proteins between cytoplasm and membrane (e.g., see Sasaki et al., supra). Rho GDI prevents the Rho family proteins from being transformed into active GTP-bound forms which are translocated to membranes. After the Rho protein in the active form is transformed into an inactive form, Rho GDI and the Rho protein form a complex which is then translocated to the cytosol. The Rho GDI family includes at least three isoforms: Rho GDIα, Rho GDIβ, and Rho GDIγ. Rho GDIα is ubiquitously expressed and binds to all Rho family proteins which have been heretofore studied. Rho GDIβ and Rho GDIγ exhibit particular tissue expression patterns. Rho GDI representatively has sequences as set forth in SEQ ID NO. 5 (nucleic acid sequence) and SEQ ID NO. 6 (amino acid sequence), and their variants and fragments are also included within the definition of Rho GDI as long as they have biological activity. Examples of the biological activity of Rho GDI include, but are not limited to, binding to GDP-bound Rho. Such activity can be measured with an assay, such as a GDP-GTP exchange assay.

As used herein, “MAG” and “myelin-binding glycoprotein” are used interchangeably to refer to a glycoprotein present on oligodendrocyte and Schwann cell membranes (MAG is an abbreviation of myelin-associated glycoprotein). MAG representatively has sequences as set forth in SEQ ID NO. 7 (nucleic acid sequence) and SEQ ID NO. 8 (amino acid sequence), and their variants and fragments are also included within the definition of MAG as long as they have biological activity. Examples of the biological activity of MAG include, but are not limited to, blocking of neurite outgrowth inhibition. Such activity can be measured with an assay which observes activation of Rho in nerve cells.

As used herein, “Nogo” refers to a double transmembrane protein present on cell membranes of oligodendrocytes. Nogo representatively has sequences as set forth in SEQ ID NO. 9 (nucleic acid sequence) and SEQ ID NO. 10 (amino acid sequence), and their variants and fragments are also included within the definition of Nogo as long as they have biological activity. Examples of the biological activity of Nogo include, but are not limited to, inhibition of neurite outgrowth. Such activity can be measured with an assay which observes Rho activation in nerve cells, or the like.

The term “Rho” refers to a low molecular weight GTPase which regulates the state of actin polymerization. In its active GTP-bound form, Rho hardens the actin cytoskeleton, thereby inhibiting axonal elongation and mediating destruction of growth cones (e.g., see Davies et al., supra and Schmidt et al., supra). Rho representatively has sequences as set forth in SEQ ID NO. 11 (nucleic acid sequence) and SEQ ID NO. 12 (amino acid sequence) which are RhoA sequences described below. Their variants and fragments are also included within the definition of Rho as long as they have biological activity. Examples of the biological activity of Rho include, but are not limited to, control of neurite outgrowth. Such activity can be measured by an assay, such as affinity precipitation using an effector protein, or the like.

As used herein, “RhoA” refers to a molecule which is a member of the Rho family. RhoA representatively has sequences as set forth in SEQ ID NO. 11 (nucleic acid sequence) and SEQ ID NO. 12 (amino acid sequence), and their variants and fragments are also included within the definition of RhoA as long as they have biological activity. Examples of the biological activity of RhoA include, but are not limited to, control of neurite outgrowth. Such activity can be measured with an assay, such as affinity precipitation using an effector protein.

As used herein, the term “Rho kinase” refers to a biomolecule whose phosphorylation is regulated by Rho. A Rho kinase representatively has a nucleic acid sequence of SEQ ID NO. 18 and an amino acid sequence of SEQ ID NO. 19. The definition of the term “Rho kinase” encompasses variants and fragments of these sequences as long as they have a biological activity (representatively, phosphorylation activity, regulation by Rho, and the like).

As used herein, “GT1b” refers to a molecule which is a type of ganglioside and has the same meaning as defined in the art. Examples of the biological activity of GT1b include, but are not limited to, binding to MAG or p75. Such activity can be measured with an assay, such as a MAG or p75 binding assay.

A molecule having the same function as that of GT1b in the context of the binding to MAG includes, but is not limited to, GD1a, α-series gangliosides, and the like. Such gangliosides other than GT1b may have competitive inhibition against GT1b, and therefore, can be used as MAG inhibitors.

As used herein, “p21” refers to a cyclin-dependent protein kinase inhibitor (also known as WAF1 or Cip1). p21 representatively has sequences as set forth in SEQ ID NO. 13 or 22 (nucleic acid sequence) and SEQ ID NO. 14 or 23 (amino acid sequence), and their variants and fragments are also included within the definition of p21 as long as they have biological activity. p21 is also herein referred to as p21^{Cip1/WAF1}. Examples of the biological activity of p21 include, but are not limited to, cell cycle arrest. Such activity can be measured with an assay, such as molecular induction of nerve cells.

The p21^{Cip1/WAF1} gene was identified by its interaction with Cdk2, and its expression is induced by activation of wild-type p53, and during cellular senescence and differentiation. An NH₂ terminal domain of p21^{Cip1/WAF1} inhibits cyclin-Cdk kinases and a COOH-terminal domain of p21^{Cip1/WAF1} inhibits proliferating-cell nuclear antigen (Waga, S., et al., Nature. 369:574-578, 1994; Chen, J., et al., Nature. 374:386-388, 1995; Sherr, C. J., et al., Genes. Dev. 9:1149-1163, 1995; Luo, Y., et al., Nature. 375:159-161, 1995). These cell cycle inhibitory activities of p21^{Cip1/WAF1} are attributable to its nuclear localization (Goubin, F., et al., Oncogene. 10:2281-2287, 1995; Sherr, C. J., et al., Genes. Dev. 9:1149-1163, 1995). However, recent studies provide evidence showing that p21^{Cip1/WAF1} has other biological activities in the cytoplasm. During the process of monocytic differentiation of U937 cells and HL60 cells by treatment with vitamin D3, p21^{Cip1/WAF1} expression was induced in the cytoplasm and this cytoplasmic p21^{Cip1/WAF1} forms a complex with the apoptosis signal-regulating kinase 1 and inhibits the stress-activated MAPK cascade, thus contributing to the acquisition of resistance to various apoptogenic stimuli (Asada, M., et al., EMBO. J. 18:1223-1234, 1999). Cytoplasmic localization of p21^{Cip1/WAF1} was also observed in peripheral blood monocytes (Asada, M., et al., EMBO. J. 18:1223-1234, 1999). Several reports propose possible mechanisms of translocation of p21^{Cip1/WAF1} from the nucleus to the cytoplasm. It is reported that phosphatidylinositol-3 kinase/Akt phosphorylates threonine 145 in the COOH-terminal NLS of p21^{Cip1/WAF1} and phosphorylated p21^{Cip1/WAF1} loses its ability to localize to the nucleus (Zhou, B. P., et al., Nat. Cell. Biol. 3:245-252, 2001). Another paper shows that truncation of the COOH-terminus of p21^{Cip1/WAF1} by a member of the caspase family of proteases results in the loss of its NLS and its localization changes (Levkau, B., et al., Mol. Cell. 1:553-563, 1998).

During the course of differentiation of the neuronal cells, p21^{Cip1/WAF1} also plays important roles in regulating the cell cycle. In several cell lines during differentiation after nerve growth factor treatment, the expression of p21^{Cip1/WAF1} protein was increased (Decker, S. J. J. Biol. Chem. 270:30841-30844,1995; Dobashi, Y., et al., J. Biol. Chem. 270:23031-23037, 1995; Yan, G. Z., et al., J. Neurosci. 15:6200-6212, 1995; Poluha, W., et al., Mol. Cell. Biol. 16:1335-1341, 1996; van Grunsven, L. A., et al., Oncogene. 12:1347-1356, 1996; Gollapudi, L., et al., J. Neurosci. Res. 49:461-474, 1997; Erhardt, J. A., et al., J Biol Chem. 273: 23517-23523, 1998). However, neurons after differentiation seem to have special features, distinct from other cell types, as newborn neurons extend axons and dendrites to communicate with appropriate targets. For example, dorsal root ganglion neurons up to postnatal day 3-4 or embryonic retinal ganglion neurons can extend their neurites rapidly on myelin-associated glycoprotein, which is an effective neurite outgrowth inhibitor for adult neurons (Johnson, P. W., et al., Neuron. 3:377-385, 1989; Mukhopadhyay, G., et al., Neuron. 13:757-767, 1994; de Bellard, M. E., et al., Mol. Cell. Neurosci. 7:89-101, 1996; Cai, D., et al., J. Neurosci. 21:4731-4739, 2001). These findings suggest that immature neurons may have intrinsic mechanisms that confer resistance to the inhibitory molecules.

As used herein, “TAT PTD domain” or “PTD domain” are used interchangeably to refer to the amino acid sequence of an amino terminus of a TAT protein of human immune deficiency virus (HIV), which has an action to promote introduction of proteins. Representatively, this sequence includes, but is not limited to, YGRKKRRQRRR (SEQ ID NO. 20). This sequence can be fused with any active agent (e.g., p21, Pep5, or the like). As used herein, PTD domain can be referred to as “TAT”.

As used herein, “nerve regeneration agent” refers to an agent involved in nerve regeneration, such as the p75 signal transduction pathway or the like, which has an action of nerve regeneration (e.g., promotion of nerve regeneration, blockade of nerve inhibition, or the like). Examples of such an agent include, but are not limited to, the Pep5 polypeptide of the present invention, a nucleic acid molecule encoding the Pep5 polypeptide, an agent capable of specifically interacting with the p75 polypeptide, an agent capable of specifically interacting with the nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, a Rho GDI polypeptide, a nucleic acid molecule encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic acid molecule encoding the p21 polypeptide, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof, and the like.

The terms “silencing” and “silence” are used herein interchangeably to refer to disruption of the interaction between p75^{NTR} and Rho GDI. The term “silencer” refers to an agent which disrupts the interaction between p75^{NTR} and Rho GDI.

(Definition of Terms)

Hereinafter, the definitions of the terms as used herein are described.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a complex of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. Gene products of the present invention are ordinarily in the form of polypeptides. Such gene products of the present invention in the polypeptide form are useful for compositions of the present invention for diagnosis, prophylaxis, treatment or prognosis.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081(1991); Ohtsuka et al., J. Biol. Chem. 260:2605-2608 (1985); Rossolini et al., Mol. Cell. Probes. 8:91-98(1994)). Genes of the present invention are ordinarily in the form of the above-described polynucleotides. Such genes or gene products of the present invention in the nucleotide form are useful for compositions of the present invention for diagnosis, prophylaxis, treatment or prognosis.

As used herein, “nucleic acid molecule” is also used interchangeably with nucleic acid, oligonucleotide and polynucleotide, including cDNA, mRNA, genomic DNA, and the like. As used herein, nucleic acid and nucleic acid molecule may be included by the concept of the term “gene”. A nucleic acid molecule encoding the sequence of a given gene includes “splice variant”. Similarly, a particular protein encoded by a nucleic acid encompasses any protein encoded by a splice variant of that nucleic acid. “Splice variants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing (of exons. Alternative polypeptides derived from the same nucleic acid by read-through transcription are also encompassed by this definition. Any products of a splicing reaction, including recombinant forms of the splice products, are included in this definition. Therefore, the gene of the present invention may include the splice variants herein.

As used herein, “gene” refers to an agent defining a genetic trait. A gene is typically arranged in a given sequence on a chromosome. A gene which regulates the expression of a structural gene is called a regulatory gene (e.g., promoter). Genes herein include structural genes and regulatory genes unless otherwise specified. Therefore, Pep5, p75, Rho GDI, MAG, p21, Rho, Rho kinase genes and the like ordinarily include the structural genes of the gene of the present invention as well as the regulatory sequences (e.g., promoters) for transcription and/or translation, etc. In the present invention, it will be understood that in addition to structural genes, regulatory sequences for transcription and/or translation, etc. are useful for nerve regeneration, and diagnosis, treatment, prophylaxis and prognosis for neurological diseases, and the like. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understand what a gene product is, according to the context.

As used herein, “homology” of a gene (e.g., a nucleic acid sequence, an amino acid sequence, or the like) refers to the proportion of identity between two or more gene sequences. As used herein, the identity of a sequence (a nucleic acid sequence, an amino acid sequence, or the like) refers to the proportion of the identical sequence (an individual nucleic acid, amino acid, or the like) between two or more comparable sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other. As used herein, “similarity” of a gene (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using FASTA (sequence analyzing tool) with the default parameters.

As used herein, “amino acid” may refer to a naturally-occurring or nonnaturally-occurring amino acid as long as it satisfies the purpose of the present invention. The term “amino acid derivative” or “amino acid analog” refers to an amino acid which is different from a naturally-occurring amino acid and has a function similar to that of the original amino acid. Such an amino acid derivative and amino acid analog are well known in the art. The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers, although embodiments using D-amino acids are within the scope of the present invention. The term “nonnaturally-occurring amino acid” refers to an amino acid which is ordinarily not found in the nature. Examples of nonnaturally-occurring amino acids include norleucine, para-nitrophenylalanine, homophenylalanine, para-fluorophenylalanine, 3-amino-2-benzil propionic acid, D- or L-homoarginine, and D-phenylalanine. The term “amino acid analog” refers to a molecule having a physical property and/or function similar to that of amino acids, but not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid refers to an amino acid in a given protein molecule or polypeptide molecule, which has, or is anticipated to have, a function similar to that of a predetermined amino acid in a protein or polypeptide as a reference for comparison. Particularly, in the case of enzyme molecules, the term refers to an amino acid which is present at a similar position in an active site and similarly contributes to catalytic activity. For example, in the case of antisense molecules, the term refers to a similar portion in an ortholog corresponding to a particular portion of the antisense molecule.

As used herein, the term “corresponding” gene refers to a gene in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to mouse Pep5, p75, Rho GDI, MAG, p21, Rho, Rho kinase genes and the like can be found in other animals (human, rat, pig, cattle, and the like). Such a corresponding gene can be identified by a technique well known in the art. Therefore, for example, a corresponding gene in a given animal can be found by searching a sequence database of the animal (e.g., human, rat) using the sequence of a reference gene (e.g., mouse Pep5, p75, Rho GDI, MAG, p21, Rho, Rho kinase genes, and the like) as a query sequence.

As used herein, the term “exogenous” refers to a nucleotide or amino acid sequence which is a different or non-corresponding sequence, or a sequence derived from a different species. For example, a nucleotide or amino acid sequence of mouse MAG is exogenous to a nucleotide or amino acid sequence of human MAG, and a nucleotide or amino acid sequence of human MAG is exogenous to a nucleotide or amino acid sequence of human albumin.

As used herein, the term “nucleotide” may be either naturally-occurring or nonnaturally-occurring. The term “nucleotide derivative” or “nucleotide analog” refers to a nucleotide which is different from a naturally-occurring nucleotide and has a function similar to that of the original nucleotide. Such a nucleotide derivative and nucleotide analog are well known in the art. Examples of such a nucleotide derivative and nucleotide analog include, but are not limited to, phosphorothioate, phosphoramidate, methylphosphonate, chiral-methylphosphonate, 2-O-methyl ribonucleotide, and peptide-nucleic acid (PNA).

As used herein, the term “fragment” refers to a polypeptide or polynucleotide having a sequence length ranging- from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification. The length of a useful fragment may be determined depending on whether or not at least one function is maintained among the functions of a full-length protein which is a reference of the fragment.

As used herein, the term “specifically interact with” indicates that a first substance or agent interacts with a second substance or agent with higher affinity than that to substances or agents other than the second substance or agent (particularly, other substances or agents in a sample containing the second substance or agent). Examples of a specific interaction with reference to a substance or agent include, but are not limited to, hybridization of nucleic acids, antigen-antibody reaction, ligand-receptor reaction, enzyme-substrate reaction, a reaction between a transcriptional agent and a binding site of the transcriptional agent when both a nucleic acid and a protein are involved, a protein-lipid interaction, a nucleic acid-lipid interaction, and the like. Therefore, when both the first and second substances or agents are nucleic acids, “specifically interact with” means that the first substance or agent is at least partially complementary to the second substance or agent. Alternatively, when both the first and second substances or agents are proteins, “specifically interact with” includes, but is not limited to, an interaction due to antigen-antibody reaction, an interaction due to receptor-ligand reaction, an enzyme-substrate interaction, and the like. When the two substances or agents are a protein and a nucleic acid, “specifically interact with” includes an interaction between a transcriptional agent and a biding region of a nucleic acid molecule targeted by the transcriptional agent.

As used herein, the term “agent capable of specifically interacting with” a biological agent, such as a polynucleotide, a polypeptide or the like, refers to an agent which has an affinity to the biological agent, such as a polynucleotide, a polypeptide or the like, which is representatively higher than or equal to an affinity to other non-related biological agents, such as polynucleotides, polypeptides or the like (particularly, those with identity of less than 30%), and preferably significantly (e.g., statistically significantly) higher. Such an affinity can be measured with, for example, a hybridization assay, a binding assay, or the like. As used herein, the “agent” may be any substance or other agent (e.g., energy, such as light, radiation, heat, electricity, or the like) as long as the intended purpose can be achieved. Examples of such a substance include, but are not limited to, proteins, polypeptides, oligopeptides, peptides, polynucleotides, oligonucleotides, nucleotides, nucleic acids (e.g., DNA such as cDNA , genomic DNA , or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like), and the like), and combinations of these molecules. Examples of an agent specific to a polynucleotide include, but are not limited to, representatively, a polynucleotide having complementarity to the sequence of the polynucleotide with a predetermined sequence homology (e.g., 70% or more sequence identity), a polypeptide such as a transcriptional agent binding to a promoter region, and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme, and the like.

As used herein, the term “compound” refers to any identifiable chemical substance or molecule, including, but not limited to, a low molecular weight molecule, a peptide, a protein, a sugar, a nucleotide, or a nucleic acid. Such a compound may be a naturally-occurring product or a synthetic product.

As used herein, the term “transduction agent” in the p75 signal transduction pathway refers to a molecule playing a role in transferring a signal in the p75 signal transduction pathway. Such a molecule includes, but is not limited to, MAG, GT1b, p75, Rho GDI, Rho, p21, Rho kinase, and the like.

As used herein, the terms “suppression” and “inhibition” of the p75 signal transduction pathway means that the whole or a part of the p75 signal transduction pathway is blocked, and as a result, a signal is not completely transferred (preferably, no signal transferred). As used herein, the terms “suppression” and “inhibition” of a transduction agent in the p75 signal transduction pathway similarly mean that the function of the transduction agent in the signal transduction pathway is partially or fully impaired (preferably, fully impaired). Such a mechanism of suppression or inhibition includes, but is not limited to, mutation, suppression, inhibition, or extinction of MAG, GT1b, p75, Rho GDI, Rho, p21, Rho kinase, and the like.

As used herein, the term “low molecular weight organic molecule” refers to an organic molecule having a relatively small molecular weight. Usually, the low molecular weight organic molecule refers to a molecular weight of about 1,000 or less, or may refer to a molecular weight of more than 1,000. Low molecular weight organic molecules can be ordinarily synthesized by methods known in the art or combinations thereof. These low molecular weight organic molecules may be produced by organisms. Examples of the low molecular weight organic molecule include, but are not limited to, hormones, ligands, information transfer substances, synthesized by combinatorial chemistry, pharmaceutically acceptable low molecular weight molecules (e.g., low molecular weight ligands and the like), and the like.

As used herein, the term “contact” refers to direct or indirect placement of a compound physically close to the polypeptide or polynucleotide of the present invention. Polypeptides or polynucleotides may be present in a number of buffers, salts, solutions, and the like. The term “contact” includes placement of a compound in a beaker, a microtiter plate, a cell culture flask, a microarray (e.g., a gene chip) or the like containing a polypeptide encoded by a nucleic acid or a fragment thereof.

As used herein, the term “antibody” encompasses polyclonal antibodies, monoclonal antibodies, human antibodies, humanized antibodies, polyfunctional antibodies, chimeric antibodies, and anti-idiotype antibodies, and fragments thereof (e.g., F(ab′)2 and Fab fragments), and other recombinant conjugates. These antibodies may be fused with an enzyme (e.g., alkaline phosphatase, horseradish peroxidase, α-galactosidase, and the like) via a covalent bond or by recombination.

As used herein, the term “monoclonal antibody” refers to an antibody composition having a group of homologous antibodies. This term is not limited by the production manner thereof. This term encompasses all immunoglobulin molecules and Fab molecules, F(ab′)2 fragments, Fv fragments, and other molecules having an immunological binding property of the original monoclonal antibody molecule. Methods for producing polyclonal antibodies and monoclonal antibodies are well known in the art, and will be more sufficiently described below.

Monoclonal antibodies are prepared by using the standard technique well known in the art (e.g., Kohler and Milstein, Nature (1975) 256:495) or a modification thereof (e.g., Buck et al. (1982) In Vitro 18:377). Representatively, a mouse or rat is immunized with a protein bound to a protein carrier, and boosted. Subsequently, the spleen (and optionally several large lymph nodes) is removed and dissociated into single cells. If desired, the spleen cells may be screened (after removal of nonspecifically adherent cells) by applying a cell suspension to a plate or well coated with a protein antigen. B-cells that express membrane-bound immunoglobulin specific for the antigen bind to the plate, and are not rinsed away with the rest of the suspension. Resulting B-cells, or all dissociated spleen cells, are then induced to fuse with myeloma cells to form hybridomas. The hybridomas are used to produce monoclonal antibodies.

As used herein, the term “antigen” refers to any substrate to which an antibody molecule may specifically bind. As used herein, the term “immunogen” refers to an antigen capable of initiating activation of the antigen-specific immune response of a lymphocyte.

As used herein, the term “single chain antibody” refers to a single chain polypeptide formed by linking a heavy chain fragment and the light chain fragment of the Fv region via peptide crosslinker.

As used herein, the term “composite molecule” refers to a molecule in which a plurality of molecules, such as polypeptides, polynucleotides, lipids, sugars, low molecular weight molecules, and the like, are linked together. Examples of such a composite molecule include, but are not limited to, glycolipids, glycopeptides, and the like. These composite molecules can be used herein as nucleic acid molecules encoding Pep5, p75, Rho GDI, MAG, p21, Rho, Rho kinase, variants or fragments thereof, and the like, products thereof, GT1b, or the agent of the present invention as long as they have a function similar to that of the nucleic acid molecules encoding Pep5, p75, Rho GDI, MAG, p21, Rho, Rho kinase, variants or fragments thereof, and the like, products thereof, GT1b, or the agent of the present invention.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intened nucleic acid; and in the case of proteins, agents other than proteins and proteins having an amino acid sequence other than an intened protein). The “isolated” nucleic acid and protein include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a part of naturally accompanying agents is removed. Therefore, ordinarily, the purity of the biological agent of a purified biological agent is higher than the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

As used herein, the term “expression” of a gene product, such as a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

Therefore, as used herein, the term “reduction” of “expression” of a gene, a polynucleotide, a polypeptide, or the like indicates that the level of expression is significantly reduced in the presence of the action of the agent of the present invention as compared to when the action of the agent is absent. Preferably, the reduction of expression includes a reduction in the amount of expression of a polypeptide (e.g., Pep5, p75, Rho GDI, MAG, p21, Rho, Rho kinase, or variants or fragments thereof, and the like). As used herein, the term “increase” of “expression” of a gene, a polynucleotide, a polypeptide, or the like indicates that the level of expression is significantly increased in the presence of the action of the agent of the present invention as compared to when the action of the agent is absent. Preferably, the increase of expression includes an increase in the amount of expression of a polypeptide (e.g., Pep5, p75, Rho GDI, MAG, p21, Rho, Rho kinase, or variants or fragments thereof, and the like). As used herein, the term “induction” of “expression” of a gene indicates that the amount of expression of the gene is increased by applying a given agent to a given cell. Therefore, the induction of expression includes allowing a gene to be expressed when expression of the gene is not otherwise observed, and increasing the amount of expression of the gene when expression of the gene is observed. The increase or reduction of these genes or gene products (polypeptides or polynucleotides) may be useful in treatment embodiments, prognosis embodiments or prophylaxis embodiments of the present invention.

As used herein, the term “specifically expressed” in the case of genes indicates that a gene is expressed in a specific site or in a specific period of time at a level different from (preferably higher than) that in other sites or periods of time. The term “specifically expressed” includes that a gene may be expressed only in a given site (specific site) or may be expressed in other sites. Preferably, the term “specifically expressed” indicates that a gene is expressed only in a given site. Therefore, according to an embodiment of the present invention, Pep5, p75, Rho GDI, MAG, p21, Rho, Rho kinase, or variants or fragments thereof, and the like may be expressed specifically or locally in an affected portion (e.g., nerve).

As used herein, term “biological activity” refers to activity prossessed by an agent (e.g., a polynucleotide, a protein, etc.) within an organism, including activities exhibiting various functions (e.g., transcription promoting activity). For example, when two agents interact with each other (e.g., Pep5 and p75, p75 and Rho GDI, MAG and p75, GT1b and p75, or the like), the biological activity includes binding of the two molecules and a biological change due to the binding. For example, when one molecule is precipitated using antibodies, another molecule may also precipitate. In this case, it is determined that the two molecules are bound together. Therefore, observation of such coprecipitation provides a determination method, for example. In addition, neurite outgrowth may be used as an indicator to infer that a given molecule is functionally associated with another molecule. Specifically, the term “biological activity” includes the observation that MAG, GT1b, p75, and Rho GDI inhibit neurite outgrowth in association with one another, while Pep5 and p21 block this action. For example, when a given agent is an enzyme, the biological activity thereof includes the emzymatic activity thereof. In another example, when a given agent is a ligand, the biological activity thereof includes binding of the agent to a receptor for the ligand. Such biological activity can be measured with a technique well known in the art.

As used herein, the term “activity” refers to various measurable indicators which indicate or clarify binding (either directly or indirectly); or affect a response (i.e., having a measurable influence on response to some exposure or stimuli), including the affinity of a compound directly binding to the polypeptide or polynucleotide of the present invention, the amount of an upstream or downstream protein after some stimuli or events, or other similar functional scales. Such an activity may be measured by an assay, such as competitive inhibition of MAG binding to GTb1. For example, non-labeled soluble MAG is added to an assay at an increasing concentration, and the binding of MAG to p75-GTb1 expressed on the surface of a CHO cell is inhibited. As another example, an ability of a neuron to extend across a lesion caused by nerve injury may be evaluated (Schnell and Schwab (1990), Nature 343, 269-272).

As used herein, the term “interaction” with reference to two substances means that one substance influences the other substance via forces (e.g., intermolecular forces (Van der Waals force), hydrogen bonding, hydrophobic interactions, or the like). Typically, two substances interacting with each other are in the form of association or binding.

As used herein, the term “binding” means the physical or chemical interaction between two proteins or compounds or associated proteins or compounds or combinations thereof. Binding includes ionic, non-ionic, hydrogen, Van der Waals, hydrophobic interactions, etc. A physical interaction (binding) can be either direct or indirect. Indirect interactions may be through or due to the effects of another protein or compound. Direct binding refers to interactions that do not take place through, or due to, the effect of another protein or compound, but instead are without other substantial chemical intermediates.

As used herein, the term “modulate” or “modify” refers to an increase or decrease or maintenance in a specific activity, or the amount, quality or effect of a protein.

As used herein, the term “antisense (activity)” refers to activity which permits specific suppression or reduction of expression of a target gene. The antisense activity is ordinarily achieved by a nucleic acid sequence having a length of at least 8 contiguous nucleotides, which is complementary to the nucleic acid sequence of a target gene (e.g., Pep5, p75, Rho GDI, MAG, p21, Rho, Rho kinase, or variants or fragments thereof, and the like). Such a nucleic acid sequence preferably has a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 30 contiguous nucleotides, a length of at least 40 contiguous nucleotides, and a length of at least 50 contiguous nucleotides. A molecule having such a nucleic acid sequence is herein referred to as “antisense molecule”, “antisense nucleic acid molecule”, or “antisense nucleic acid”, which are interchangeably used. These nucleic acid sequences include nucleic acid sequences having at least 70% homology thereto, more preferably at least 80%, even more preferably at least 90%, and still even more preferably at least 95%. The antisense activity is preferably complementary to a 5′ terminal sequence of the nucleic acid sequence of a target gene. Such an antisense nucleic acid sequence includes the above-described sequences having one or several, or at least one, nucleotide substitutions, additions, and/or deletions.

Given a nucleic acid sequence disclosed herein (e.g., SEQ ID NO. 1, 3, 5, 7, 9, 11, 13, or the like), antisense nucleic acids of the present invention can be designed according to the rules of Watson and Crick or Hoogsteen base pairing. The antisense nucleic acid molecule can be complementary to the entire coding region of mRNA of a p75 signal transduction agent, but more preferably is an oligonucleotide that is antisense to only a portion of the coding or noncoding region of the mRNA of the p75 signal transduction agent. For example, the antisense oligonucleotide can be complementary to the region surrounding the translation start site of the mRNA of the p75 signal transduction agent. An antisense oligonucleotide can be, for example, about 5, 10, 15, 20, 25, 30, 35, 40, 45 or 50 nucleotides in length. An antisense nucleic acid of the present invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used. Examples of modified nucleotides that can be used to generate the antisense nucleic acid include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

As used herein, the term “RNAi” is an abbreviation of RNA interference and refers to a phenomenon that an agent for causing RNAi, such as double-stranded RNA (also called dsRNA), is introduced into cells and mRNA homologous thereto is specifically degraded, so that synthesis of gene products is suppressed, and a technique using the phenomenon. As used herein, RNAi may have the same meaning as that of an agent which causes RNAi.

As used herein, the term “an agent causing RNAi” refers to any agent capable of causing RNAi. As used herein, “an agent causing RNAi for a gene” indicates that the agent causes RNAi relating to the gene and the effect of RNAi is achieved (e.g., suppresson of expression of the gene, and the like). Examples of such an agent causing RNAi include, but are not limited to, a sequence having at least about 70% homology to the nucleic acid sequence of a target gene or a sequence hybridizable under stringent conditions, RNA containing a double-stranded portion having a length of at least 10 nucleotides or variants thereof. Here, this agent may be preferably DNA containing a 3′ protruding end, and more preferably the 3′ protruding end has a length of 2 or more nucleotides (e.g., 2-4 nucleotides in length).

Though not wishing to be bound by any theory, a mechanism which causes RNAi is considered as follows. When a molecule which causes RNAi, such as dsRNA, is introduced into a cell, an RNase III-like nuclease having a helicase domain (called dicer) cleaves the molecule on about a 20 base pair basis from the 3′ terminus in the presence of ATP in the case where the RNA is relatively long (e.g., 40 or more base pairs). As used herein, the term “siRNA” is an abbreviation of short interfering RNA and refers to short double-stranded RNA of 10 or more base pairs which are artificially chemically or biochemically synthesized, synthesized in the organism body, or produced by double-stranded RNA of about 40 or more base pairs being degraded within the body. siRNA typically has a structure having 5′-phosphate and 3′-OH, where the 3′ terminus projects by about 2 bases. A specific protein is bound to siRNA to form RISC (RNA-induced-silencing-complex). This complex recognizes and binds to mRNA having the same sequence as that of siRNA and cleave mRNA at the middle of siRNA due to RNase III-like enzymatic activity. It is preferable that the relationship between the sequence of siRNA and the sequence of mRNA to be cleaved as a taget is a 100% match. However, base mutation at a site away from the middle of siRNA does not completely remove the cleavage activity by RNAi, leaving partial activity, while base mutation in the middle of siRNA has a large influence and the mRNA cleavage activity by RNAi is considerably lowered. By utilizing such a nature, only mRNA having a mutation can be specifically degraded. Specifically, siRNA in which the mutation is provided in the middle thereof is synthesized and is introduced into a cell. Therefore, in the present invention, siRNA per se as well as an agent capable of producing siRNA (e.g., representatively dsRNA of about 40 or more base pairs) can be used as an agent capable of eliciting RNAi.

Also, though not wishing to be bound by any theory, apart from the above-described pathway, the antisense strand of siRNA binds to mRNA and siRNA functions as a primer for RNA-dependent RNA polymerase (RdRP), so that dsRNA is synthesized. This dsRNA is a substrate for a dicer again, leading to production of new siRNA. It is intended that such an action is amplified. Therefore, in the present invention, siRNA per se as well as an agent capable of producing siRNA are useful. In fact, in insects and the like, for example, 35 dsRNA molecules can substantially completely degrade 1000 or more copies of intracellular mRNA, and therefore, it will be understood that siRNA per se as well as an agent capable of producing siRNA are useful.

In the present invention, double-stranded RNA having a length of about 20 bases (e.g., representatively about 21 to 23 bases) or less than about 20 bases, which is called siRNA, can be used. Expression of siRNA in cells can suppress expression of a pathogenic gene targeted by the siRNA. Therefore, siRNA can be used for treatment, prophylaxis, prognosis, and the like of diseases.

The siRNA of the present invention may be in any form as long as it can elicit RNAi.

In another embodiment, an agent capable of causing RNAi may have a short hairpin structure having a sticky portion at the 3′ terminus (shRNA; short hairpin RNA). As used herein, the term “shRNA” refers to a molecule of about 20 or more base pairs in which a single-standed RNA partially contains a palindromic base sequence and forms a double-strand structure therein (i.e., a hairpin structure). shRNA can be artificially chemically synthesized. Alternatively, shRNA can be produced by linking sense and antisense strands of a DNA sequence in reverse directions and synthesizing RNA in vitro with T7 RNA polymerase using the DNA as a template. Though not wishing to be bound by any theory, it should be understood that after shRNA is introduced into a cell, the shRNA is degraged in the cell into a length of about 20 bases (e.g., representatively 21, 22, 23 bases), and causes RNAi as with siRNA, leading to the treatment effect of the present invention. It should be understood that such an effect is exhibited in a wide range of organisms, such as insects, plants, animals (including mammals), and the like. Thus, shRNA elicits RNAi as with siRNA and therefore can be used as an effective component of the present invention. shRNA may preferably have a 3′ protruding end. The length of the double-stranded portion is not particularly limited, but is preferably about 10 or more nucleotides, and more preferably about 20 or more nucleotides. Here, the 3′ protruding end may be preferably DNA, more preferably DNA of at least 2 nucleotides in length, and even more preferably DNA of 2-4 nucleotides in length.

An agent capable of causing RNAi used in the present invention may be artificially synthesized (chemically or biochemically) or naturally occurring. There is substantially no difference therebetween in terms of the effect of the present invention. A chemically synthesized agent is preferably purified by liquid chromatography or the like.

An agent capable of causing RNAi used in the present invention can be produced in vitro. In this synthesis system, T7 RNA polymerase and T7 promoter are used to synthesize antisense and sense RNAs from template DNA. These RNAs are annealed and thereafter are introduced into a cell. In this case, RNAi is caused via the above-described mechanism, thereby achieving the effect of the present invention. Here, for example, the introduction of RNA into cell can be carried out by a calcium phosphate method.

Another example of an agent capable of causing RNAi according to the present invention is a single-stranded nucleic acid hybridizable to mRNA or all nucleic acid analogs thereof. Such agents are useful for the method and composition of the present invention.

As used herein, “polynucleotides hybridizing under stringent conditions” refers to conditions commonly used and well known in the art. Such a polynucleotide can be obtained by conducting colony hybridization, plaque hybridization, Southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A or T. “Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of DNA encoding a polypeptide having an amino acid sequence specifically herein disclosed, preferably a polynucleotide having a homology of at least 80%, and more preferably a polynucleotide having a homology of at least 95%.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.0015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual (2nd ed., Cold Spring Harbor Laboratory, N.Y., 1989); Anderson et al., Nucleic Acid Hybridization: A Practical Approach Ch. 4 (IRL Press Limited) (Oxford Express). More stringent conditions (such as higher temperature, lower ionic strength, higher formamide, or other denaturing agents) may be optionally used. Other agents may be included in the hybridization and washing buffers for the purpose of reducing non-specific and/or background hybridization. Examples are 0.1% bovine serum albumin, 0.1% polyvinylpyrrolidone, 0.1% sodium pyrophosphate, 0.1% sodium dodecylsulfate (NaDodSO₄ or SDS), Ficoll, Denhardt's solution, sonicated salmon sperm DNA (or another noncomplementary DNA), and dextran sulfate, although other suitable agents can also be used. The concentration and types of these additives can be changed without substantially affecting the stringency of the hybridization conditions. Hybridization experiments are ordinarily carried out at pH 6.8-7.4; however, at typical ionic strength conditions, the rate of hybridization is nearly independent of pH. See Anderson et al., Nucleic Acid Hybridization: A Practical Approach Ch. 4 (IRL Press Limited, Oxford UK).

Agents affecting the stability of DNA duplex include base composition, length, and degree of base pair mismatch. Hybridization conditions can be adjusted by those skilled in the art in order to accommodate these variables and allow DNAs of different sequence relatedness to form hybrids. The melting temperature of a perfectly matched DNA duplex can be estimated by the following equation:

Tm(° C.)=81.5+16.6(log[Na⁺])+0.41(% G+C)−600/N−0.72(% formamide)

where N is the length of the duplex formed, [Na⁺] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, “moderately stringent conditions” of 50° C. in 0.015 M sodium ion will allow about a 21% mismatch.

It will be appreciated by those skilled in the art that there is no absolute distinction between “highly stringent conditions” and “moderately stringent conditions”. For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, those skilled in the art can simply lower the temperature or raise the ionic strength.

A good estimate of the melting temperature in 1 M NaCl for oligonucleotide probes up to about 20 nucleotides is given by:

Tm=(2° C. per A−T base pair)+(4° C. per G−C base pair).

Note that the sodium ion concentration in 6× salt sodium citrate (SSC) is 1 M. See Suggs et al., Developmental Biology Using Purified Genes 683 (Brown and Fox, eds., 1981).

A naturally-occurring nucleic acid encoding a protein (e.g., Pep5, p75, Rho GDI, MAG, p21, Rho, Rho kinase, or variants or fragments thereof, or the like) may be readily isolated from a cDNA library having PCR primers and hybridization probes containing part of a nucleic acid sequence indicated by, for example, SEQ ID NO. 1, 3, 5, 7, 9, 11, 13, 15, 16 or the like. A preferable nucleic acid encoding Pep5, p75, Rho GDI, MAG, p21, Rho, Rho kinase, or variants or fragments thereof, or the like is hybridizable to the whole or part of a sequence as set forth in SEQ ID NO. 1, 3, 5, 7, 9, 11, 13, 15 or 16 under low stringent conditions defined by hybridization buffer essentially containing 1% bovine serum alubumin (BSA); 500 mM sodium phosphate (NaPO₄); 1 mM EDTA; and 7% SDS at 42° C., and wash buffer essentially containing 2×SSC (600 mM NaCl; 60 mM sodium citrate); and 0.1% SDS at 50° C., more preferably under low stringent conditions defined by hybridization buffer essentially containing 1% bovine serum alubumin (BSA); 500 mM sodium phosphate (NaPO₄); 15% formamide; 1 mM EDTA; and 7% SDS at 50° C., and wash buffer essentially containing 1×SSC (300 mM NaCl; 30 mM sodium citrate); and 1% SDS at 50° C., and most preferably under low stringent conditions defined by hybridization buffer essentially containing 1% bovine serum alubumin (BSA); 200 mM sodium phosphate (NaPO₄); 15% formamide; 1 mM EDTA; and 7% SDS at 50° C., and wash buffer essentially containing 0.5×SSC (150 mM NaCl; 15 mM sodium citrate); and 0.1% SDS at 65° C.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a usual probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90%, or at least 95%.

As used herein, the term “search” indicates that a given nucleic acid base sequence is utilized to find other nucleic acid base sequences having a specific function and/or property electronically or biologically, or other methods. Examples of electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of biological search include, but are not limited to, a macroarray in which genomic. DNA is attached to a nylon membrane or the like or a microarray (microassay) in which genomic DNA is attached to a glass plate under stringent hybridization, PCR and in situ hybridization, and the like. It is herein intended that Pep5, p75, Rho GDI, MAG, p21, Rho, Rho kinase, and the like used in the present invention include corresponding genes identified by such an electronic or biological search.

As used herein, the “percentage of (amino acid, nucleotide, or the like) sequence identity, homology or similarity” is determined by comparing two optimally aligned sequences over a window of comparison, wherein the portion of a polynucleotide or polypeptide sequence in the comparison window may comprise additions or deletions (i.e. gaps), as compared to the reference sequences (which does not comprise additions or deletions (if the other sequence includes an addition, a gap may occur)) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e. the window size) and multiplying the results by 100 to yield the percentage of sequence identity. When used in a search, homology is evaluated by an appropriate technique selected from various sequence comparison algorithms and programs well known in the art. Examples of such algorithms and programs include, but are not limited to, TBLASTN, BLASTP, FASTA, TFASTA and CLUSTALW (Pearson and Lipman, 1988, Proc. Natl. Acad. Sci. USA 85(8):2444-2448, Altschul et al., 1990, J. Mol. Biol. 215(3):403-410, Thompson et al., 1994, Nucleic Acids Res. 22(2):4673-4680, Higgins et al., 1996, Methods Enzymol. 266:383-402, Altschul et al., 1990, J. Mol. Biol. 215(3):403-410, Altschul et al., 1993, Nature Genetics 3:266-272). In a particularly preferable embodiment, the homology of a protein or nucleic acid sequence is evaluated using a Basic Local Alignment Search Tool (BLAST) well known in the art (e.g., see Karlin and Altschul, 1990, Proc. Natl. Acad. Sci. USA 87:2267-2268, Altschul et al., 1990, J. Mol. Biol. 215:403-410, Altschul et al., 1993, Nature Genetics 3:266-272, Altschul et al., 1997, Nuc. Acids Res. 25:3389-3402). Particularly, 5 specialized-BLAST programs may be used to perform the following tasks to achieve comparison or search:

(1) comparison of an amino acid query sequence with a protein sequence database using BLASTP and BLAST3;

(2) comparison of a nucleoride query sequence with a nucleotide sequence database using BLASTN;

(3) comparison of a conceptually translated product in which a nucleotide query sequence (both strands) is converted over 6 reading frames with a protein sequence database using BLASTX;

(4) comparison of all protein query sequences converted over 6 reading frames (both strands) with a nucleotide sequence database using TBLASTN; and

(5) comparison of nucleotide query sequences converted over 6 reading frames with a nucleotide sequence database using TBLASTX.

The BLAST program identifies homologous sequences by specifying analogous segments called “high score segment pairs” between amino acid query sequences or nucleic acid query sequences and test sequences obtained from preferably a protein sequence database or a nucleic acid sequence database. A large number of the high score segment pairs are preferably identified (aligned) using a scoring matrix well known in the art. Preferably, the scoring matrix is the BLOSUM62 matrix (Gonnet et al., 1992, Science 256:1443-1445, Henikoff and Henikoff, 1993, Proteins 17:49-61). The PAM or PAM250 matrix may be used, although they are not as preferable as the BLOSUM62 matrix (e.g., see Schwartz and Dayhoff, eds., 1978, Matrices for Detecting Distance Relationships: Atlas of Protein Sequence and Structure, Washington: National Biomedical Research Foundation). The BLAST program evaluates the statistical significance of all identified high score segment pairs and preferably selects segments which satisfy a threshold level of significance independently defined by a user, such as a user set homology. Preferably, the statistical significance of high score segment pairs is evaluated using Karlin's formula (see Karlin and Altschul, 1990, Proc. Natl. Acad. Sci. USA 87:2267-2268).

As used herein, the term “primer” refers to a substance required for initiation of a reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and at least 95%. An appropriate sequence as a primer may vary depending on the property of a sequence to be synthesized (amplified). Those skilled in the art can design an appropriate primer depending on a sequence of interest. Such a primer design is well known in the art and may be performed manually or using a computer program (e.g., LASERGENE, Primer Select, DNAStar).

As used herein, the term “epitope” refers to an antigenic determinant whose structure is clear. Therefore, the term “epitope” includes a set of amino acid residues which is involved in recognition by a particular immunoglobulin, or in the context of T cells, those residues necessary for recognition by T cell receptor proteins and/or Major Histocompatibility Complex (MHC) receptors. This term is also used interchangeably with “antigenic determinant” or “antigenic determinant site”. In the field of immunology, in vivo or in vitro, an epitope is the features of a molecule (e.g., primary, secondary and tertiary peptide structure, and charge) that form a site recognized by an immunoglobulin, T cell receptor or HLA molecule. An epitope including a peptide comprises 3 or more amino acids in a spatial conformation which is unique to the epitope. Generally, an epitope consists of at least 5 such amino acids, and more ordinarily, consists of at least 6, 7, 8, 9 or 10 such amino acids. The greater the length of an epitope, the more the similarity of the epitope to the original peptide, i.e., longer epitopes are generally preferable. This is not necessarily the case when the conformation is taken into account. Methods of determining the spatial conformation of amino acids are known in the art, and include, for example, X-ray crystallography and 2-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23: 709 (technique for identifying peptides with high affinity for a given antibody). Antibodies that recognize the same epitope can be identified in a simple immunoassay. Thus, methods for determining an epitopes including a peptide are well known in the art. Such an epitope can be determined using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and 25 amino acids. Epitopes may be linear or conformational.

(Modification of genes, protein molecules, nucleic acid molecules, and the like)

In a given protein molecule (e.g., Pep5, p75, Rho GDI, MAG, p21, Rho, Rho kinase, etc.), a given amino acid contained in a sequence may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness.

When the above-described modifications are designed, the hydrophobicity indices of amino acids may be taken into consideration. The hydrophobic amino acid indices play an important role in providing a protein with an interactive biological function, which is generally recognized in the art (Kyte. J and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5)).

It is well known that if a given amino acid is substituted with another amino acid having a similar hydrophobicity index, a resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on the hydrophobicity is efficient. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, the conservative substitution is carried out between amino acids having a hydrophilicity or hydrophobicity index of within ±2, preferably within ±1, and more preferably within ±0.5. Examples of the conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an allelic variant ordinarily has a sequence the same as or highly similar to that of the corresponding allele, and ordinarily has almost the same biological activity, though it rarely has different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of largely affecting the higher-order structure of a polypeptide. Examples of a method for such modification of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500(1983)). Modification can be performed using methods ordinarily used in the field of molecular biology.

In order to prepare functionally equivalent polypeptides, amino acid additions, deletions, or modifications can be performed in addition to amino acid substitutions. Amino acid substitution(s) refers to the replacement of at least one amino acid of an original peptide with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, truncation,lipidation,alkylation, glycosylation, phosphorylation, hydroxylation, acylation (e.g., acetylation), and the like. Amino acids to be substituted or added may be naturally-occurring or nonnaturally-occurring amino acids, or amino acid analogs. Naturally-occurring amino acids are preferable.

As used herein, the term “peptide analog” or “peptide derivative” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same as the function of the original peptide (e.g., a similar pKa value, a similar functional group, a similar binding manner to other molecules, a similar water-solubility, and the like). Such a peptide analog can be prepared using a technique well known in the art. Therefore, a peptide analog may be a polymer containing an amino acid analog.

A chemically-modified polypeptide composition in which a polypeptide of the present invention is attached to a polymer is included within the scope of the present invention. This polymer may be water soluble so that the protein does not precipitate in an aqueous environment (e.g., a physiological environment). An appropriate water soluble polymer may be selected from the group consisting of: polyethylene glycol (PEG), monomethoxy-polyethylene glycol, dextran, cellulose, or other carbohydrate based polymers, poly-(N-vinylpyrrolidone) polyethylene glycol, propylene glycol homopolymers, a polypropylene oxide/ethylene oxide co-polymer, polyoxyethylated polyols (e.g., glycerol) and polyvinyl alcohol. The selected polymer is typically modified to have a single reactive group (e.g., active ester for acylation or aldehyde for alkylation). As a result, the degree of polymerization may be controlled. The polymer may be of any molecular weight, and may be branched or unbranched. Included within the scope of suitable polymers is a mixture of polymers. When the chemically modified polymer of the present invention is used in therapeutic applications, a pharmaceutically acceptable polymer is selected.

When the polymer is modified by an acylation reaction, the polymer should have a single reactive ester group. Alternatively, when the polymer is modified by reducing alkylation, the polymer should have a single reactive aldehyde group. A preferable reactive aldehyde is, for example, polyethylene glycol, propionaldehyde (which is water stable), or mono C1-C10 alkoxy or aryloxy derivatives thereof (see U.S. Pat. No. 5,252,714, which is herein incorporated by reference in its entity).

Pegylation of the polypeptide of the present invention may be carried out by any of the pegylation reactions known in the art, as described for example in the following references: Focus on Growth Factors, 3, 4-10 (1992); EP 0 154 316; EP 0 401 384, which are herein incorporated by reference in their entity). Preferably, pegylation may be carried out via an acylation reaction or an alkylation reaction with a reactive polyethylene glycol molecule (or an analogous reactive water-soluble polymer). Polyethylene glycol (PEG) is a water-soluble polymer suitable for use in pegylation of the polypeptide of the present invention (e.g., MAG, p75, p21, Pep5, Rho, Rho GDI, and the like). As used herein, the term “polyethylene glycol” is meant to encompass any of the forms of PEG that have been used to derivatize proteins (e.g., mono(C1-C10) alkoxy-polyethylene glycol or mono(C1-C10) aryloxy-polyethylene glycol (PEG)).

Chemical derivatization of the polypeptide of the present invention may be performed under any suitable conditions that can be used to react a biologically active substance with an activated polymer molecule. Methods for preparing pegylated polypeptides of the present invention will generally comprise the steps of (a) reacting the polypeptide with polyethylene glycol (such as a reactive ester or aldehyde derivative of PEG) under conditions whereby a transduction agent in the p75 signal transduction pathway becomes attached to one or more PEG groups, and (b) obtaining the reaction product (s). The optimal reaction conditions or the acylation reactions are easily selected by those skilled in the art based on known parameters and the desired result.

Generally, conditions may be alleviated or modulated by the administration of the pegylated polypeptide of the present invention. However, the polypeptide derivative of the polypeptide molecule of the present invention disclosed herein may have additional activities, enhanced or reduced biological activity, or other characteristics (e.g., increased or decreased half-life), as compared to the nonderivatized molecules. The polypeptide of the present invention, and fragments, variants and derivatives thereof may be used singly or in combination, or in combination with other pharmaceutical compositions, such as cytokines, proliferating agents, antigens, anti-inflammatory agents and/or chemotherapeutics, which are suitable for treatment of symptoms.

Similarly, the term “polynucleotide analog” or “nucleic acid analog” refers to a compound which is different from a polynucleotide or a nucleic acid but has at least one chemical function or biological function equivalent to that of a polynucleotide or a nucleic acid. Therefore, a polynucleotide analog or a nucleic acid analog includes one that has at least one nucleotide analog or nucleotide derivative addition or substitution with respect to the original peptide.

Nucleic acid molecules as used herein includes one in which a part of the sequence of the nucleic acid is deleted or is substituted with other base(s), or an additional nucleic acid sequence is inserted, as long as a polypeptide expressed by the nucleic acid has substantially the same activity as that of the naturally-occurring polypeptide, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function as that of that polypeptide. Such a gene is known in the art and can be used in the present invention.

The above-described nucleic acid can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute with respect to the original polypeptide or polynucleotide. This is achieved by techniques well known in the art, including a site-specific mutagenesis technique and the like. A polypeptide or a polynucleotide may have any number (>0) of substitutions, additions, or deletions. The number can be as large as a variant having such a number of substitutions, additions or deletions maintains an intended function (e.g., the information transfer function of hormones and cytokines, etc.). For example, such a number may be one or several, and preferably within 20% or 10% of the full length, or no more than 100, no more than 50, no more than 25, or the like.

(General Techniques)

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-Interscience; Ausubel, F. M. (1989), Short Protocols in Molecular Biology: A Compendium of Methods from Current Protocols in Molecular Biology, Greene Pub. Associates and Wiley-Interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Experimental Method for Gene Introduction & Expression Analysis”, Yodo-sha, 1997; and the like. Relevant portions (or possibly the entirety) of each of these publication are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approac, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

(Genetic Engineering)

Pep5, p75, Rho GDI, MAG, p21, Rho, Rho kinase and the like, and fragments and variants thereof as used herein can be produced by genetic engineering techniques.

When a gene is mentioned herein, the term “vector” or “recombinant vector” refers to a vector capable of transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for cloning is referred to as “cloning vector”. Such a cloning vector ordinarily contains a multiple cloning site containing a plurality of restriction sites. Restriction sites and multiple cloning sites are well known in the art and may be appropriately or optionally used depending on the purpose. The technology is described in references as described herein (e.g., Sambrook et al. (supra)).

Preferred vectors include, but are not limited to, plasmids, phages, cosmids, episomes, viral particles or viruses, and integratable DNA fragments (i.e., fragments which can be integrated into a host genome by homologous recombination). Preferred viral particles include, but are not limited to, adenoviruses, baculoviruses, parvoviruses, herpesviruses, poxviruses, adeno-associated viruses, Semliki Forest viruses, vaccinia viruses, and retroviruses.

One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers. It is well known to those skilled in the art that the type of an organism (e.g., a plant) expression vector and the type of a regulatory element may vary depending on the host cell.

As used herein, a “recombinant vector” for prokaryotic cells includes, for example, pcDNA 3(+), pBluescript-SK(+/−), pGEM-T, pEF-BOS, pEGFP, pHAT, pUC18, pFT-DEST™, 42GATEWAY (Invitrogen), and the like.

As used herein, a “recombinant vector” for animal cells includes, for example, pcDNA I/Amp, pcDNA I, pCDM8 (all commercially available from Funakoshi, Tokyo, Japan), pAGE107 [Japanese Laid-Open Publication No. 3-229 (Invitrogen)], pAGE103 [J. Biochem., 101, 1307 (1987)], pAMo, pAMoA [J. Biol. Chem., 268, 22782-22787 (1993)], retroviral expression vectors based on Murine Stem Cell Virus (MSCV), pEF-BOS, pEGFP, and the like.

As used herein, the term “terminator” refers to a sequence which is located downstream of a protein-encoding region of a gene and which is involved in the termination of transcription when DNA is transcribed into mRNA, and the addition of a poly A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. Therefore, a portion of a given gene which functions as a promoter is herein referred to as a “promoter portion”. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon.

As used herein, the term “origin of replication” refers to a specific region on a chromosome from which DNA replication starts. An origin of replication may be provided either by construction of the vector so that an endogenous origin is contained therein or by the chromosomal replication mechanism of a host cell. When the vector is integrated into a chromosome in the host cell, the latter may be sufficient. Alternatively, instead of using a vector containing a viral origin of replication, a mammalian cell may be transformed by those skilled in the art using a method of co-transforming a selectable marker and the DNA of the present invention. Examples of an appropriate selectable marker include dihydrofolate reductase (DHFR) or thymidine kinase (U.S. Pat. No. 4,399,216).

For example, by expressing a nucleic acid using a tissue-specific regulatory element, a recombinant mammalian expression vector is capable of directing expression of the nucleic acid preferentially in a particular cell type. Tissue-specific regulatory elements are known in the art. Non-limiting examples of suitable tissue-specific promoters include developmentally-regulated promoters (e.g., the murine hox promoters (Kessel and Gruss (1990) Science 249, 374-379) and the a-fetoprotein promoter (Campes and Tilghman (1989) Genes Dev 3, 537-546); the albumin promoter (liver-specific; Pinkert et al. (1987) Genes Dev 1, 268-277), lymphoid-specific promoters (Calame and Eaton (1988) Adv Immunol 43, 235-275), in particular promoters of T cell receptors (Winoto and Baltimore (1989) EMBO J. 8, 729-733) and immunoglobulins (Banerji et al. (1983) Cell 33, 729-740; Queen and Baltimore (1983) Cell 33, 741-748), neuron-specific promoters (e.g., the neurofilament promoter; Byrne and Ruddle (1989) Proc. Natl. Acad. Sci. USA 86, 5473-5477), pancreas-specific promoters (Edlund et al. (1985) Science 230, 912-916), and mammary gland-specific promoters (e.g., milk whey promoter; U.S. Pat. No. 4,873,316 and European Application Publication No. 264,166).

As used herein, the term “enhancer” refers to a sequence which is used so as to enhance the expression efficiency of a gene of interest. Such an enhancer is well known in the art. One or more enhancers may be used, or no enhancer may be used.

As used herein, the term “operatively linked” indicates that a desired sequence is located such that expression (operation) thereof is under control of a transcription and translation regulatory sequence (e.g., a promoter, an enhancer, and the like) or a translation regulatory sequence. In order for a promoter to be operatively linked to a gene, typically, the promoter is located immediately upstream of the gene. A promoter is not necessarily adjacent to a structural gene.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Method for Gene Introduction & Expression Analysis”, Yodo-sha, 1997; and the like. Gene introduction can be confirmed by method as described herein, such as Northern blotting analysis and Western blotting analysis, or other well-known, common techniques.

Any of the above-described methods for introducing DNA into cells can be used as an vector introduction method, including, for example, transfection, transduction, transformation, and the like (e.g., a calcium phosphate method, a liposome method, a DEAE dextran method, an electroporation method, a particle gun (gene gun) method, and the like).

As used herein, the term “transformant” refers to the whole or a part of an organism, such as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic cell is used herein for genetic operations or the like, the prokaryotic cell may be of, for example, genus Escherichia, genus Serratia, genus Bacillus, genus Brevibacterium, genus Corynebacterium, genus Microbacterium, genus Pseudomonas, or the like. Specifically, the prokaryotic cell is, for example, Escherichia coli XL1-Blue, Escherichia coli XL2-Blue, Escherichia coli DH1, or the like.

Examples of an animal cell as used herein include a mouse myeloma cell, a rat myeloma cell, a mouse hybridoma cell, a Chinese hamster overy (CHO) cell, a baby hamster kidney (BHK) cell, an African green monkey kidney cell, a human leukemic cell, HBT5637 (Japanese Laid-Open Publication No. 63-299), a human colon cancer cell line, and the like. The mouse myeloma cell includes ps20, NSO, and the like. The rat myeloma cell includes YB2/0 and the like. A human embryo kidney cell includes HEK293 (ATCC:CRL-1573) and the like. The human leukemic cell includes BALL-1 and the like. The African green monkey kidney cell includes COS-1, COS-7, and the like. The human colon cancer cell line includes HCT-15, and the like. A human neuroblastoma includes SK-N-SH, SK-N-SH-5Y, and the like. A mouse neuroblastoma includes Neuro2A, and the like.

Any method for introduction of DNA can be used herein as a method for introduction of a recombinant vector, including, for example, a calcium chloride method, an electroporation method (Methods. Enzymol., 194, 182 (1990)), a lipofection method, spheroplast method (Proc.Natl.Acad.Sci.USA,84,1929(1978)), a lithium acetate method (J.Bacteriol.,153,163(1983)), a method described in Proc. Natl. Acad. Sci. USA, 75, 1929 (1978), and the like.

A retrovirus infection method as used herein is well known in the art as described in, for example, Current Protocols in Molecular Biology (supra) (particularly, Units 9.9-9.14), and the like. Specifically, for example, embryonic stem cells are trypsinized into a single-cell suspension, followed by co-culture with the culture supernatant of virus-producing cells (packaging cell lines) for 1-2 hours, thereby obtaining a sufficient amount of infected cells.

The transient expression of Cre enzyme, DNA mapping on a chromosome, and the like, which are used herein in a method for removing a genome, a gene locus, or the like, are well known in the art, as described in Kenichi Matsubara and Hiroshi Yoshikawa, editors, Saibo-Kogaku [Cell Engineering], special issue, “Experiment Protocol Series “FISH Experiment Protocol From Human Genome Analysis to Chrmosome/Gene diagnosis”, Shujun-sha (Tokyo), and the like.

Gene expression (e.g., mRNA expression, polypeptide expression) may be. “detected” or “quantified” by an appropriate method, including mRNA measurement and immunological measurement method. Examples of the molecular biological measurement method include a Northern blotting method, a dot blotting method, a PCR method, and the like. Examples of the immunological measurement method include an ELISA method, an RIA method, a fluorescent antibody method, a Western blotting method, an immunohistological staining method, and the like, where a microtiter plate may be used. Examples of a quantification method include an ELISA method, an RIA method, and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. The protein array is described in detail in Nat Genet. 2002 Dec; 32 Suppl:526-32. Examples of a method for analyzing gene expression include, but are not limited to, an RT-PCR method, a RACE method, an SSCP method, an immunoprecipitation method, a two-hybrid system, an in vitro translation method, and the like in addition to the above-described techniques. Other analysis methods are described in, for example, “Genome Analysis Experimental Method, Yusuke Nakamura's Labo-Manual, edited by Yusuke Nakamura, Yodo-sha (2002), and the like. All of the above-described publications are herein incorporated by reference.

As used herein, the term “amount of expression” refers to the amount of a polypeptide or mRNA expressed in a subject cell. The amount of expression includes the amount of expression at the protein level of a polypeptide of the present invention evaluated by any appropriate method using an antibody of the present invention, including immunological measurement methods (e.g., an ELISA method, an RIA method, a fluorescent antibody method, a Western blotting method, an immunohistological staining method, and the like, or the amount of expression at the mRNA level of a polypeptide of the present invention evaluated by any appropriate method, including molecular biological measurement methods (e.g., a Northern blotting method, a dot blotting method, a PCR method, and the like). The term “change in the amount of expression” indicates that an increase or decrease in the amount of expression at the protein or mRNA level of a polypeptide of the present invention evaluated by an appropriate method including the above-described immunological measurement method or molecular biological measurement method.

As used herein, the term “upstream” in reference to a polynucleotide means that the position is closer to the 5′ terminus than a specific reference point.

As used herein, the term “downstream” in reference to a polynucleotide means that the position is closer to the 3′ terminus than a specific reference point.

As used herein, the term “base paired” and “Watson & Crick base paired” have the same meaning and refer to nucleotides which can be bound together by hydrogen bonds based on the sequence identity that an adenine residue is bound to a thymine residue or a uracil residue via two hydrogen bonds and a cytosine residue is bound to a guanine reside via three hydrogen bonds, as seen in double-stranded DNA (see Stryer, L., Biochemistry, 4th edition, 1995).

As used herein, the term “complementary” or “complement” refers to a polynucleotide sequence such that the whole complementary region thereof is capable of Watson-Crick base paring with another specific polynucleotide. In the present invention, when each base of a first polynucleotide pairs with a corresponding complementary base, the first polynucleotide is regard as being complementary to a second polynucleotide. Complementary bases are generally A and T (or A and U) or C and G. As used herein, the term “complement” is used as a synonym for the terms “complementary polynucleotide”, “complementary nucleic acid” and “complementary nucleotide sequence”. These terms are applied to a pair of polynucleotides based on the sequence, but not a specific set of two polynucleotides which are virtually bound together.

(Polypeptide Production Method)

A transformant derived from an microorganism, an animal cell, or the like, which possesses a recombinant vector into which DNA encoding a polypeptide of the present invention (e.g., Pep5, p75, Rho GDI, MAG, p21, Rho, Rho kinase or the like) is incorprated, is cultured according to an ordinary culture method. The polypeptide of the present invention is produced and accumulated. The polypeptide of the present invention is collected from the culture, thereby making it possible to produce the polypeptide of the present invention.

The transformant of the present invention can be cultured on a culture medium according to an ordinary method for use in culturing host cells. A culture medium for a transformant obtained from a prokaryote (e.g., E. coli) or a eukaryote (e.g., yeast) as a host may be either a naturally-occurring culture medium or a synthetic culture medium as long as the medium contains a carbon source, a nitrogen source, inorganic salts, and the like which an organism of the present invention can assimilate and the medium allows efficient culture of the transformant.

The carbon source includes any one that can be assimilated by the organism, such as carbohydrates (e.g, glucose, fructose, sucrose, molasses containing these, starch, starch hydrolysate, and the like), organic acids (e.g., acetic acid, propionic acid, and the like), alcohols (e.g., ethanol, propanol, and the like), and the like.

The nitrogen source includes ammonium salts of inorganic or organic acids (e.g., ammonia, ammonium chloride, ammonium sulfate, ammonium acetate, ammonium phosphate, and the like), and other nitrogen-containing substances (e.g., peptone, meat extract, yeast extract, corn steep liquor, casein hydrolysate, soybean cake, and soybean cake hydrolysate, various fermentation bacteria and digestion products thereof), and the like.

Salts of inorganic acids, such as potassium (I) phosphate, potassium (II) phosphate, magnesium phosphate, magnesium phosphate, sodium chloride, iron (I) sulfate, manganese sulfate, copper sulfate, calcium carbonate, and the like, can be used. Culture is performed under aerobic conditions for shaking culture, deep aeration agitation culture, or the like.

Culture temperature is preferably 15 to 40° C., culture time is ordinarily 5 hours to 7 days. The pH of culture medium is maintained at 3.0 to 9.0. The adjustment of pH is carried out using inorganic or organic acid, alkali solution, urea, calcium carbonate, ammonia, or the like. An antibiotic, such as ampicillin, tetracycline, or the like, may be optionally added to culture medium during-cultivation.

When culturing an microorganism which has been transformed using an expression vector containing an inducible promoter, culture medium may be optionally supplemented with an inducer. For example, when a microorganism, which has been transformed using an expression vector containing a lac promoter, is cultured, isopropyl-β-D-thiogalactopyranoside or the like may be added to the culture medium. When a microorganism, which has been transformed using an expression vector containing a trp promoter, is cultured, indole acrylic acid or the like may be added to culture medium. A cell or an organ into which a gene has been introduced can be cultured in a large volume using a jar fermenter.

For example, when an animal cell is used, a culture medium of the present invention for culturing the cell includes a commonly used RPMI1640 culture medium (The Journal of the American Medical Association, 199, 519 (1967)), Eagle's MEM culture medium (Science,122,501(1952)), DMEM culture medium (Virology, 8, 396 (1959)), 199 culture medium (Proceedings of the Society for the Biological Medicine, 73, 1 (1950)) or these culture media supplemented with fetal bovine serum or the like.

Culture is normally carried out for 1 to 7 days under conditions such as pH 6 to 8, 25 to 40° C., 5% CO₂. An antibiotic, such as kanamycin, penicillin, streptomycin, or the like may be optionally added to culture medium during cultivation.

A polypeptide of the present invention can be isolated or purified from a culture of a transformant, which has been transformed with a nucleic acid sequence encoding the polypeptide, using an ordinary method for isolating or purifying enzymes, which are well known and commonly used in the art. For example, when a polypeptide of the present invention is secreted outside a transformant for producing the polypeptide, the culture is subjected to centrifugation or the like to obtain a soluble fraction. A purified specimen can be obtained from the soluble fraction by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When a polypeptide (e.g., Pep5, p75, Rho GDI, MAG, p21, Rho, Rho kinase, or variants or fragments thereof, and the like) of the present invention is accumulated in a dissolved form within a transformant cell for producing the polypeptide, the culture is subjected to centrifugation to collect cells in the culture. The cells are washed, followed by pulverization of the cells using a ultrasonic pulverizer, a French press, MANTON GAULIN homogenizer, Dinomil, or the like, to obtain a cell-free extract solution. A purified specimen can be obtained from a supernatant obtained by centrifuging the cell-free extract solution or by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When the polypeptide of the present invention has been expressed and formed insoluble bodies within cells, the cells are harvested, pulverized, and centrifuged. From the resulting precipitate fraction, the polypeptide of the present invention is collected using a commonly used method. The insoluble polypeptide is solubilized using a polypeptide denaturant. The resulting solubilized solution is diluted or dialyzed into a denaturant-free solution or a dilute solution, where the concentration of the polypeptide denaturant is too low to denature the polypeptide. The polypeptide of the present invention is allowed to form a normal three-dimensional structure, and the purified specimen is obtained by isolation and purification as described above.

Purification can be carried out in accordance with a commonly used protein purification method (J. Evan. Sadler et al.: Methods in Enzymology, 83, 458). Alternatively, the polypeptide of the present invention can be fused with other proteins to produce a fusion protein, and the fusion protein can be purified using affinity chromatography using a substance having affinity to the fusion protein (Akio Yamakawa, Experimental Medicine, 13, 469-474 (1995)). For example, in accordance with a method described in Lowe et al., Proc. Natl. Acad. Sci., USA, 86, 8227-8231 (1989), Genes Develop., 4, 1288(1990)), a fusion protein of the polypeptide of the present invention with protein A is produced, followed by purification with affinity chromatography using immunoglobulin G.

A fusion protein of the polypeptide of the present invention with a FLAG peptide is produced, followed by purification with affinity chromatography using anti-FLAG antibodies (Proc. Natl. Acad. Sci., USA, 86, 8227(1989), Genes Develop., 4, 1288 (1990)). For such a fusion protein, in fusion expression vectors, a proteolytic cleavage site is introduced at the junction of a fusion moiety and a recombinant protein to enable separation of the recombinant protein from the fusion moiety subsequent to purification of the fusion protein. Such enzymes, and their cognate recognition sequences, include Factor Xa, thrombin, and enterokinase. Typical fusion expression vectors include pGEX (Pharmacia Biotech Inc; Smith and Johnson (1988) Gene 67, 31-40), pMAL (New England Biolabs. Beverly, Mass.) and pRIT5 (Pharmacia, Piscataway. N.J.) that fuse glutathione S-transferase (GST), maltose E binding protein, or protein A, respectively, to the target recombinant protein.

The polypeptide of the present invention can be purified with affinity chromatography using antibodies which bind to the polypeptide. The polypeptide of the present invention can be produced using an in vitro transcription/translation system in accordance with a known method (J. Biomolecular NMR, 6, 129-134; Science, 242, 1162-1164; J. Biochem., 110, 166-168 (1991)).

The polypeptide of the present invention can also be produced by a chemical synthesis method, such as the Fmoc method (fluorenylmethyloxycarbonyl method), the tBoc method (t-buthyloxycarbonyl method), or the like, based on the amino acid information thereof. The peptide can be chemically synthesized using a peptide synthesizer (manufactured by Advanced ChemTech, Applied Biosystems, Pharmacia Biotech, Protein Technology Instrument, Synthecell-Vega, PerSeptive, Shimazu, or the like).

The structure of the purified polypeptide of the present invention can be carried out by methods commonly used in protein chemistry (see, for example, Hisashi Hirano. “Protein Structure Analysis for Gene Cloning”, published by Tokyo Kagaku Dojin, 1993). The physiological activity of a polypeptide of the present invention can be measured in accordance with a known measurement method.

Production of a soluble polypeptide useful in the present invention may be achieved by various methods known in the art. For example, the polypeptide may be derived from an intact transmembrane p75 polypeptide molecule by protein degradation which is carried out by exopeptidase, Edman degradation or a combination of both using specific endopeptidase. The intact p75 polypeptide molecule may be purified from naturally occurring sources using conventional methods. Alternatively, the intact p75 polypeptide may be produced by recombinant DNA technology using well known techniques for cDNA, expression vectors, and recombinant gene expression.

Preferably, a soluble polypeptide useful in the present invention may be directly produced. Therefore, the necessity of using the whole p75 peptide as a starting material is eliminated. This may be achieved by conventional chemical synthesis techniques or well known recombinant DNA techqniques (here, expression is carried out in a host in which only a DNA sequence encoding a desired peptide is transformed). For example, a gene encoding a desired soluble p75 polypeptide may be synthesized by chemical means using an oligonucleotide synthesizer. Such an oligonucleotide is designed based on the amino acid sequence of the desired soluble p75 polypeptide. A specific DNA sequence encoding a desired peptide may be derived from the full-length DNA sequence by isolation of a specific restriction endonuclease fragment or PCR synthesis of a specific region of cDNA.

(Method for Producing Mutant Polypeptide)

Amino acid deletion, substitution or addition (including fusion) of the polypeptide of the present invention (e.g., Pep5, p75, Rho GDI, MAG, p21, Rho, Rho kinase, and the like) can be carried out by a site-specific mutagenesis method which is a well known technique. One or several amino acid deletions, substitutions or additions can be carried out in accordance with methods described in Molecular Cloning, A Laboratory Manual, Second Edition, Cold Spring Harbor Laboratory Press (1989); Current Protocols in Molecular Biology, Supplement 1 to 38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci USA, 82, 488 (1985); Proc. Natl. Acad. Sci., USA, 81, 5662 (1984); Science, 224, 1431 (1984); PCT WO85/00817(1985); Nature, 316, 601 (1985); and the like.

(Immunochemistry)

Preparation of antibodies which recognize the polypeptide of the present invention (e.g., Pep5, p75, Rho GDI, MAG, p21, Rho, Rho kinase, variants or fragments thereof, and the like) are also well known in the art. For example, preparation of polyclonal antibodies can be carried out by administering a purified speciment of the whole or a partial fragment of an obtained polypeptide or a peptide having a part of the amino acid sequence of the protein of the present invention, as an antigen, to an animal.

To produce antibodies, a rabbit, a goat, a rat, a mouse, a hamster, or the like can be used as an animal to which an antigen is administered. The dose of the antigen is preferably 50 to 100 μg per animal. When a peptide is used as an antigen, the peptide is preferably coupled via covalent bond to a carrier protein, such as keyhole limpet haemocyanin, bovine thyroglobulin, or the like. A peptide used as an antigen can be synthesized using a peptide synthesizer. The antigen is administered every 1 to 2 weeks after a first administration a total 3 to 10 times. 3 to 7 days after each administration, blood is collected from the venous plexus of eye grounds, and whether or not the serum reacts with the antigen which has been used for immunization is determined by an enzyme immunoassay (Enzyme Immunoassay (ELISA): published by Igaku-syoin 1976; Antibodies—A Laboratory Manual, Cold Spring Harbor Lavoratory (1988); and the like).

Serum is obtained from a non-human mammal whose serum exhibits a sufficient antibody titer to an antigen. From the serum, polyclonal antibodies can be isolated and purified using well known techniques. Production of monoclonal antibodies is also well known in the art. In order to prepare antibody secreting cells, a rat whose serum exhibits a sufficient antibody titer for fragments of a polypeptide of the present invention which has been used for immunization, is used as a source for antibody secreting cells, which are fused with myeloma cells to prepare hybridomas. Thereafter, a hybridoma specifically reacting with the fragments of the polypeptide of the present invention is selected using enzyme immunoassays. A monoclonal antibody secreted by the thus-obtained hybridoma can be used for various purposes.

Such an antibody can be used for an immunological method of detecting the polypeptide of the present invention, for example. Examples of an immunological method of detecting the polypeptide of the present invention using the antibody of the present invention include an ELISA method using microtiter plates, a fluorescent antibody method, a Western blotting method, an immunohistological method, and the like.

Further, the antibody of the present invention can be used for immunological methods for quantifying the polypeptide of the present invention polypeptide. Examples of the immunological methods for quantifying the polypeptide of the present invention include a sandwich ELISA method using two monoclonal antibodies for different epitopes of the polypeptide of the present invention, which react with the polypeptide of the present invention; a radioimmunoassay using the polypeptide of the present invention labeled with a radioactive isotope, such as 126I or the like, and antibodies which recognize the polypeptide of the present invention; and the like.

Methods for quantifying mRNA for the polypeptide of the present invention polypeptide are well known in the art. For example, the above-described oligonucleotides prepared from the polynucleotide or DNA of the present invention can be used to quantify the amount of expression of DNA encoding the polypeptide of the present invention based on the mRNA level using Northern hybridization or PCR. Such a technique is well known in the art and is described in literature described herein.

The polynucleotides may be obtained, and the nucleotide sequence of the polynucleotides determined, by any method known in the art. For example, if the nucleotide sequence of an antibody is known, a polynucleotide encoding the antibody may be assembled from chemically synthesized oligonucleotides (e. g., as described in Kutmeier ef al., BioTechniques 17: 242 (1994)), which, briefly, involves the synthesis of overlapping oligonucleotides containing portions of the sequence encoding the antibody, annealing and ligation of those oligonucleotides, and then amplification of the ligated oligonucleotides by PCR.

Alternatively, a polynucleotide encoding an antibody can be produced from a nucleic acid from a suitable source. If a clone containing a nucleic acid encoding a particular antibody is not available, but the sequence of the antibody molecule is known, a nucleic acid encoding the immunoglobulin may be obtained from a suitable source (e. g., an antibody cDNA library, or a cDNA library generated from any tissue or cells expressing the antibody (e.g., hybridoma cells selected to express an antibody of the present invention), or nucleic acids (preferably poly A+RNA) isolated therefrom) by PCR amplification using synthetic primers hybridizable to the 3′ and 5′ ends of the sequence or by cloning using an oligonucleotide probe specific for the particular gene sequence to identify, for example, a cDNA clone from a cDNA library that encodes the antibody. Amplified nucleic acids produced by PCR may be cloned into replicable cloning vectors using any method well known in the art.

Once the nucleotide sequence and corresponding amino acid sequence of an antibody is determined, the nucleotide sequence of the antibody may be manipulated using methods well known in the art for the manipulation of nucleotide sequences (e. g., recombinant DNA techniques, site directed mutagenesis, PCR, etc. (see, for example, the techniques described in Sambrook et al., 1990, Molecular Cloning, A Laboratory Manual, 2nd Ed., Cold Spring Harbor Laboratory, Cold Spring Harbor, N.Y. and Ausubel el al., eds., 1998, Current Protocols in Molecular Biology, John Wiley & Sons, NY, which are both incorporated by reference herein in their entireties), to produce antibodies having a different amino acid sequence, for example, to create amino acid substitutions, deletions, and/or insertions.

In a specific embodiment, the amino acid sequence of heavy and/or light chain variable domains may be inspected to identify the sequences of the complementarity determining regions (CDRs) by methods that are well know in the art (e. g., by comparison to known amino acid sequences of other heavy and light chain variable regions to determine the regions of sequence hypervariability). Using routine recombinant DNA techniques, one or more of the CDRs may be inserted within framework regions (e. g., into human framework regions to humanize a non-human antibody) as described above. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the present invention. Preferably, as discussed above, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314: 452-454) by splicing genes from a mouse antibody molecule of appropriate antigen specificity together with genes from a human antibody molecule of appropriate biological activity can be used. As described above, a chimeric antibody is a molecule in which different portions are derived from different animal species. Such a molecule has a variable region derived from a murine mAb and a human immunoglobulin constant region (e.g., humanized antibodies).

Known techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

(Methods of Producing Antibodies)

The antibodies of the present invention can be produced by any method known in the art for the synthesis of antibodies, by chemical synthesis, or preferably, by recombinant expression techniques.

Recombinant expression of an antibody of the present invention, or fragment, derivative or analog thereof (e.g., a heavy or light chain of an antibody of the present invention) requires construction of an expression vector containing a polynucleotide that encodes the antibody. Once a polynucleotide encoding an antibody molecule or a heavy or light chain of an antibody, or portion thereof (preferably containing the heavy or light chain variable domain), of the present invention has been obtained, a vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art may be used to construct expression vectors containing antibody coding sequences and appropriate transcriptional and translational control signals. These methods include, for example, in vitro recombinant DNA techniques, synthetic techniques, and in viva genetic recombination. The present invention, thus, provides replicable vectors comprising a nucleotide sequence encoding an antibody molecule of the present invention, or a heavy or light chain thereof, or a heavy or light chain variable domain, operably linked to a promoter. Such vectors may include the nucleotide sequence encoding the constant region of the antibody molecule (see, e. g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the present invention. Thus, the present invention includes host cells containing a polynucleotide encoding an antibody of the present invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

In embodiments related to the present invention, pharmaceutical compositions (e.g., vaccine compositions) may be provided for prophylactic or therapeutic applications. Such compositions generally include immunogenic polypeptides or polynucleotides and immune stimulating agents (e.g., adjuvants) of the present invention.

An antibody of the present invention (e.g., monoclonal antibody) can be used to isolate a polypeptide of the present invention by standard techniques (e.g., affinity chromatography or immunoprecipitation). An antibody specific to a given agent can facilitate the purification of a natural agent from cells and of a recombinantly produced agent expressed in host cells. Moreover, such an antibody can be used to detect a protein of the present invention (e.g., in a cellular lysate or cell supernatant) in order to evaluate the abundance and pattern of expression of the protein of the present invention. Such an antibody can be used diagnostically to monitor protein levels in tissue as part of a clinical testing procedure to, for example, determine the efficacy of a given treatment regimen. Detection can be facilitated by (physically) coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S or ³H. The present invention is not so limited.

In another aspect, the present invention relates to a method for inducing an immune response to a polynucleotide of the present invention by administering a polypeptide to an animal in an amount sufficient to induce the immune response. This amount varies depending on the type, size or the like of the animal, but can be determined by those skilled in the art.

(Screening)

As used herein, the term “screening” refers to selection of a target, such as an organism, a substance, or the like, a given specific property of interest from a population containing a number of elements using a specific operation/evaluation method. For screening, an agent (e.g., an antibody), a polypeptide or a nucleic acid molecule of the present invention can be used. Screening may be performed using libraries obtained in vitro, in vivo, or the like (with a system using a real substance) or alternatively in silico (with a system using a computer). It will be understood that the present invention encompasses compounds having desired activity obtained by screening. The present invention is also intended to provide drugs which are produced by computer modeling based on the disclosures of the present invention.

In one embodiment, the present invention provides an assay for screening candidate compounds or test compounds for a protein or polypeptide of the present invention, or a compound capable of binding to a biologically active portion thereof or modulating the activity thereof. The test compounds of the present invention can be obtained using any of the numerous approaches in combinatorial library methods known in the art, including biological libraries, spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12: 145).

Examples of methods for the synthesis of molecular libraries can be found in the art as follows: DeWitt et al. (1993) Proc. Natl. Acad. Sci. USA 90: 6909; Erb et al. (1994) Proc. Natl. Acac. Sci. USA 91: 1 11422; Zuckermann et al. (1994) J. Med. Chem. 37: 2678; Cho et al. (1993) Science 261: 1303; Carrell et al. (1994) Angew. Chem. Int. Ed. Engl. 33: 2059; Carrell et al. (1994) Angew. Chem. Int. Ed. Engl. 33: 2061; and Gallop et al. (1994) J. Med Chem. 37: 1233.

Libraries of compounds may be presented in solution (e. g., Houghten (1992) Bio. Techniques 13: 412-421), or on beads (Lam (1991) Nature 354: 82-84), chips (Fodor (1993) Nature 364: 555-556), bacteria (Ladner, U.S. Pat. No. 5,223,409), spores (Landner, supra), plasmids (Cull et al. (1992) Proc. Natl. Acad. Sci. USA 89: 1865-1869), or phage (Scott and Smith (1990). Science 249: 386-390; Devlin (1990) Science 249. 404-406; Cwirla et al. (1990) Proc. Natl. Acad. Sci. USA 87: 6378-6382, and Felici (1991) J. Mol. Biol. 222: 301-310; Ladner supra).

(Nervous Diseases and Nerve Regeneration)

As used herein, the term “axon” refers to a long cellular protrusion from a neuron, whereby action potentials are conducted from or towards the cell body.

As used herein, the term “axonal growth” refers to an extension of the long process or axon, originating at the cell body and preceded by the growth cone.

As used herein, the term “growth cone” refers to a specialized region at the tip of a growing neurite that is responsible for sensing the local environment and moving the axon toward its appropriate synaptic target cell.

As used herein, the term “growth cone movement” refers to the extension or collapse of the growth cone toward a neuron's target cell.

As used herein, the term “neurite” refers to a process growing out of a neuron. As it is sometimes difficult to distinguish a dendrite from an axon in culture, the term neurite is used for both.

As used herein, the term “oligodendrocyte” refers to a neuroglial cell of the CNS whose function is to myelinate CNS axons.

The term “nervous disease” or “neurological disease” are used herein interchangeably to refer to the discontinuation, termination or disorder of a function, a structure, an organ, or the like of a nerve. The term typically refers to a lesion satisfying at least two of the following criteria: 1) the presence of a pathogenic substance; 2) the presence of a symptom and/or a syndrome capable of being clearly indicated; and 3) a corresponding anatomical change. Examples of nervous diseases include, but are not limited to, cerebrovascular disorders (e.g., cerebral hemorrhage, subarachnoid hemorrhage, cerebral infarction, transient (cerebral) ischemic attack (TIA), cerebral arteriosclerosis, Binswanger disease, cerebral sinus thrombosis/cerebral phlebothrombosis, hypertensive encephalopathy, temporal arteritis, transient global amnesia (TGA), moya-moya disease, fibromuscular hyperplasia internal carotid artery/cavernous sinus/fistula, chronic subdural hematoma, amyloid angiopathy (see Alzheimer disease), etc.); circulatory disorder of the spinal cords (e.g., spinal infact, transient spinal ischemia, spinal hemorrhage, circulatory deformity of the spinal cord, spinal subarachnoid hemorrhage, subacute necrotizing myelitis, etc.); infective and inflamational disorders (e.g., meningitis, encephalitis, Herpes simplex encephalitis (HSE), Japanese encephalitis, other encephalitises, rabies, slow virus disease (e.g., subacute sclerosing panencephalitis (SSPE), progressive multiforcal leukoencephalitis (PML), Creutzfeldt-Jakob disease (CJD), etc.), neural Behcet disease, chorea minor AIDS dementa syndrome, neuro syphilis, cerebral abscess, spinal epidural abscess, HTLV-I-associated myelopathy (HAM), poliomyelitis); demyelining diseases (multiple sclerosis (MS), acute disseminated encephalomyelitis (ADEM), Balo's concentric sclerosis, inflammatory universal sclerosis, leukodystrophy, metachromatic leukodystrophy, Krabbe's disease, adrenoleukodystrophy (ALD), Canavan's disease (leukodystrophy), Pelizaeus-Merzbacher diesese (leukodystrophy), Alexander's disease (leukodystrophy), etc.); dementia disease (Alzheimer's disease, senile dementia of Alzheimer type (SDAT), Pick's disease, cerebrovascular dementia, Creutzfeldt-Jakob disease (CJD), Parkinson-dementia complex, normal pressure hydrocephalus, pregressive supranuclear palsy (PSP), etc.); basal nuclei degenerative disease (e.g., Parkinson disease (PD), symptomatic parkinsonism, striatonigral denegeration (SNG), Parkinson-dementia complex, Huntington's disease (HD), essential tremer, athetosis, dystonia syndrome (e.g., idiopathic torsion distonia, local dystonia (spasmodic wryneck, writer's cramp, Meige's disease, etc.), symptomatic dystonia (Hallervorden-Spats disease, drug-induced dystonia, etc.), Gilles de la Tourette's syndrome, etc.); spinocerebellar degenerative disease (e.g., spinocerebellar degeneration (SCD) (Shy-Drager syndrome, Machado-Joseph disease (MJD), etc.), Louis-Bar syndrome, Bassen-Kornzweig syndrome, Refsum disease, other cerebellar ataxias, etc.); motor neuron diseases (MND) (e.g., amyotrophic lateral sclerosis (ALS), progressive bulbar amytrophy (see amyotrophic lateral sclerosis), familial amyotrophic lateral sclerosis, Werdnig-Hoffmann disease (WHD), Kugelberg-Welander (K-W) disease, bulbar spinal sclerosis, juvenile one upper limb muscular sclerosis, etc.); tumor diseases of brain and spinal cord (e.g., intracranial tumor, spinal abscess, meningeal carcinoma, etc.); functional diseases (e.g., epilepsy, chronic headache, syncope (see syncope), idiopathic endocranial increased infracranial pressure disease, Meniere disease, narcolepsy, Kleine-Levin syndorome, etc.); toxic and metabolic diseases (e.g., drug intoxication (phenothiazines-derived antipsychotic agent intoxication, sedatives and hypnotics intoxication, antibiotics intoxication, antiparkinson drug, antitumor drug intoxication, β-blocker intoxication, calcium antagonist intoxication, clofibrate intoxication, antiemetic drug intoxication, SMON diease, salicylic acid intoxication, digitalis intoxication, marcotic addiction, etc.), chronic alcoholism (Wernicke encephalopathy, Marchiafava-Bignami syndrome, central pontine myelinolysis, etc.), organic solvent poisoning and pesticide poisoning (e.g., organophosphate compounds poisoning, carbamates poisoning, chloropicrin poisoning, paraquat poisoning, etc.), organophosphate nerve gas poisoning, carbon monooxide poisoning, hydrogen sulfide poisoning, cyanide compound poisoning, mercurial poisoning (metallic mercurial poisoning, inorganomercurial poisoning, organomercurial poisoning, etc.), lead poisoning, tetraethyl lead poisoning, arsenic poisoning, cadmium poisoning, chrome poisoning, manganese poisoning, metal fume fever, sedatives and hypnotics intoxication, salicylic acid intoxication, digitalis intoxication, marcotic addiction, food poisoning (e.g., natural food poisoning (tetradotoxin poisoning, measles shell fish poison food poisoning, diarrhogenic shell fish poison food poisoning, ciguatera, mushroom poisoning, potato-plant poisoning, etc.), vitamin deficiency (vitamin A deficiency, vitamin B1 deficiency, vitamin B2 deficiency, pellagra, scurvy, vitamin dependency), lipidosis, Gaucher disease, Niemann-Pick disease, etc.), acquired disorders of amino acid metabolism, Wilson disease, amyloidosis, etc.); congenital deformity (Arnold-Chiari malformation, Klippel-Feil syndrome, basilar impression, syringomyelia); neurosis and dermatopathy (e.g., phacomatosis, von-Recklinghausen, tuberous sclerosis, Sturge-Weber, von Hippel Lindau, etc.); spinal diseases (deformity of the spine herniated intervertebral discs, lateral axial band osteosis, etc.), and the like.

As used herein, the term “nervous disorder” refers to a disorder of a function, structure, or both of a nerve caused by hereditary relating to development, defects in development, or exogenous factors (e.g., toxins, traumas, diseases, etc.). Examples of nervous disorders include, but are not limited to, peripheral nervous disorders, diabetic nervous disorder, and the like. The peripheral nerve is disordered by various causes. Irrespective of causes, peripheral nervous disorders are collectively called “neropathy”. Examples of causes for nervous disorders include hereditary, infection, poisoning, metabolic disorders, allergy, collagen diseases, cancer, vascular disorders, traumas, mechanical pressure, tumor, and the like. No cause for a nervous disorder may be identified in clinical situations. The present invention encompasses nervous disorders having unknown causes as subjects to be treated. Examples of nervous disorders include, but are not limited to, parenchymatous neuropathy and intestitial neuropathy. Parenchymatous neuropathy indicates that at least one of neuron, Schwann cell and medually sheath which substantially constitute the peripheral nerve is affected by a pathogen, and a lesion occurs therein. Intestitial neuropathy refers to disorders in which stroma is affected. Examples of intestitial neuropathy include, but are not limited to, physical pressure, vascular lesion (periarteritis nodosa (PAN), collagen diseases, etc.), inflammation, and granulation tissue (e.g., leproma, sarcoidosis, etc.). If the metabolism of the whole neuron is disordered, the peripheral portion of a neuron is degenerated; the degeneration progresses toward the cell body; and eventually the nerve cell shrinks (antidromic necrotizing neuropathy). Examples of syndromes of nervous disorders include, but are not limited to, motor disorders, sensory disorders, loss of muscle strength, muscular atrophy, loss of reflex, autonomic disorders, combinations thereof, and the like. The present invention is effective for treatment, prophylaxis and the like of such nervous disorders.

As used herein, the term “nervous condition” refers to the degree of the health of a nerve. Such a condition can be represented by various parameters. The present invention makes it possible to determine the condition of a nerve by measuring Pep5, p75, Rho GDI, GT1b, MAG, p21, or the like.

As used herein, the term “central nervous system disorder” refers to any pathological condition associated with abnormal function of the central nervous system (CNS). The term includes, but is not limited to, altered CNS function resulting from physical trauma to cerebral tissue, viral infection, autoimmune mechanism, and genetic mutation.

As used herein, the term “demyelinating disease” refers to a pathological disorder characterized by the degradation of the myelin sheath of the oligodendrocyte cell membrane.

Illustrative examples of diseases, disorders or injuries (conditions) capable of being treated by a molecule or method of the present invention include brain injury, spinal cord injury, stroke, demyelinating diseases (monophasic demyelination), encephalomyelitis, multifocal leukoencephalopathy, panencephalitis, Marchiafava-Bignami disease, Spongy degeneration, Alexander's disease, Canavan's disease, metachromatic leukodystrophy and Krabbe's disease.

As used herein, the term “regeneration” refers to the recovery of injured tissue or organ to the original condition, and is also called pathological regeneration. The body of an organism may lose a part of organs or may be heavily injured by traumas or diseases in its life time. In this case, whether or not the injured organ can regenerate varies among organs (or among animal species). The branch of medicine that permits organs (or tissue), which cannot naturally regenerate, to regenerate so as to recover the function, is regeneration medicine. Whether or not tissue has regenerated, can be determined based on whether or not the function is improved. Mammals have capability of regenerating tissue and organs to some degree (e.g., regeneration of skin, liver, and blood). However, the tissue of certain organs or the central nervous system, such as heart, lung, brain, and the like has poor ability to regenerate. It has been believed that once such tissue is injured, the function cannot be recovered. Therefore, conventionally, when such an organ is injured, organ transplant is substantially the only measure for the treatment of the organ. In the case of the central nervous system to which transplant is not applicable, substantially no treatment is available.

As used herein, the term “nerve regeneration” refers to the recovery of an injured or extinguished nerve. Conventionally, it is believed that nerves, particularly the central nervous system, cannot regenerate in the adult. Once nerves lose their function, it is difficult to regenerate it. Whether or not a nerve has regenerated can be confirmed by assessing motor or sensory ability, axonal regeneration in tissue, or the like.

As used herein, the terms “prophylaxis”, “prophylactic” and “prevent” refer to the reduction of the possibility that an organism contract a disease or an abnormal condition occurs in an organism.

As used herein, the terms “treatment” and “treat” refer to a therapeutic effect and partial alleviation or suppression of an abnormal condition of an organism.

As used herein, the term “therapeutic effect” refers to an inhibition or activation agent capable of causing or contributing to an abnormal condition. A therapeutic effect relaxes at least one symptom in an abnormal condition to some extent. A therapeutic effect with reference to the treatment of an abnormal condition may refers to at least one of the following items: (a) increasing the proliferation, growth, and/or differentiation of cells; (b) inhibiting cell death (i.e., delaying or arresting cell death); (c) inhibiting degeneration; (d) relaxing at least one symptom associated with an abnormal condition; and (e) enhancing the function of an affected cell population. A compound exhibiting efficacy to an abnormal condition may be identified as described herein.

As used herein, the term “abnormal condition” refers to a function of a cell or tissue of an organism which departs from the normal condition. An abnormal condition may be associated with cell proliferation, cell differentiation, cell signal transduction, or cell survival. An abnormal condition may also include an abnormality in nerve transmission, obesity, diabetic complication (e.g., retina degeneration), irregular glucose intake or metabolism, and irregular fatty acid intake or metabolism.

Examples of abnormal cell proliferation include abnormal proliferation of neurons, cancer (e.g., fibrosis and mesangium disorder), abnormal angiogenesis and angiopoiesis, wound healing, psoriasis, diabetic, and inflammation.

Examples of abnormal differentiation include nerve degeneration disorder, the slow rate of wound healing, and the slow rate of healing of tissue graft.

Examples of abnormal cell signal transduction include psychiatric disorders including excessive neurotransmitters.

Abnormal cell survival is related to activation or suppression of an apoptosis (programmed cell death) pathway. A number of protein kinases are associated with the apoptosis pathway. An abnormality in a function of one of the protein kinases may lead to the immortality of a cell or unmatured cell death.

The present invention provides both a prophylactic method and a therapeutic method for treating a subject having (or suspected of having) a neurological disease, disorder or abnormal condition, or a subject having above-described disorders.

Diseases and disorders that are characterized by increased (relative to a subject not suffering from the disease or disorder) levels of biological activity may be treated with therapeutics that antagonize (i.e., reduce or inhibit) activity. Therapeutics that antagonize activity may be administered in a therapeutic or prophylactic manner. Therapeutics that may be utilized include, but are not limited to, (i) a transduction agent (e.g., a polypeptide) in the p75 signal transduction pathway, or analogs, derivatives, fragments or homologs thereof; (ii) antibodies to a transduction agent in the p75 signal transduction pathway; (iii) nucleic acids encoding a transduction agent in the p75 signal transduction pathway (where the agent is a polypeptide); (iv) administration of antisense nucleic acid and nucleic acids that are “dysfunctional” (i.e., due to a heterologous insertion within the coding sequences of coding sequences to a transduction agent (polypeptide) in the p75 signal transduction pathway) (e.g. RNAi) are utilized to “knockout” endogenous function of a transduction agent in the p75 signal transduction pathway by homologous recombination (see, e. g., Capecchi (1989) Science 244: 1288-1292); or (v) modulators (i.e., inhibitors, agonists and antagonists, including additional peptide mimetic of the present invention or antibodies specific to a peptide of the present invention) that modulates the interaction between a transduction agent in the p75 signal transduction pathway and its binding partner.

Diseases and disorders that are characterized by decreased (relative to a subject not suffering from the disease or disorder) levels of biological activity may be treated with therapeutics that increase (i. e., are agonists to) activity. Therapeutics that upregulate activity may be administered in a therapeutic or prophylactic manner. Therapeutics that may be utilized include, but are not limited to, a transduction agent in the p75 signal transduction pathway, or analogs, derivatives, fragments or homologs thereof ; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a transduction agent in the p75 signal transduction pathway). Methods that are well known in the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

The present invention provides a method for preventing abnormal expression of a transduction agent in the p75 signal transduction pathway or a disease or condition associated with the activity of a transduction agent in the p75 signal transduction pathway by administering a drug capable of modulating the expression of the transduction agent in the p75 signal transduction pathway or the activity of the transduction agent in the p75 signal transduction pathway. A subject having a risk of a diasese caused or contributed by abnormal expression of a transduction agent in the p75 signal transduction pathway or the activity of a transduction agent in the p75 signal transduction pathway, may be identified using either a diagnosis assay or a prognosis assay as described herein or a combination thereof. A prophylactic agent may be administered before appearance of a symptom characteristic to an abnormality in a transduction agent in the p75 signal transduction pathway. As a result, a disease or disorder can be prevented or its progression is delayed. In accordance with the type of an abnormality in a transduction agent in the p75 signal transduction pathway, for example, an agonist or antagonist agent for a transduction agent in the p75 signal transduction pathway may be used to treat a subject. An appropriate drug may be determined based on screening assays described herein.

The present invention also relates to a method for modulating the expression or activity of a transduction agent in the p75 signal transduction pathway for therapeutic purposes. The modulation method of the present invention comprises a step of contacting cells with a drug capable of modulating the activity of at least one transduction agent in the p75 signal transduction pathway associated with the cell. A drug for modulating the activity of a transduction agent in the p75 signal transduction pathway may be a drug as described herein, such as a nucleic acidnecleic acid or a protein, naturally-occurring cognate ligands and peptides of a transduction agent in the p75 signal transduction pathway, peptide mimics of a transduction agent in the p75 signal transduction pathway, or other small molecules. In one embodiment, a drug may stimulate at least one transduction agents in the p75 signal transduction pathway. Examples of such a stimulant include a nucleic acid encoding a transduction agent in the active p75 signal transduction pathway and a nucleic acid encoding a transduction agent in the p75 signal transduction pathway, which is introduced into cells. In another embodiment, a drug inhibits at least one transduction agent activities in the p75 signal transduction pathway. Examples of such an inhibitor include an antisense for a nucleic acid encoding a transduction agent in the p75 signal transduction pathway and an antibody against a transduction agent in the p75 signal transduction pathway. These modulation method may be carried out in vitro (e.g., culturing cells with a drug) or in vivo (e.g., administering a drug into a subject). Thus, the present invention provides a method for treating a subject suffering from a disease or disorder characterized by the abnormal expression or abnormal activity of a nucleic acid molecule encoding a transduction agent (e.g., a polypeptide) in the p75 signal transduction pathway. In one embodiment, the method comprises a step of administering a combination of a drug (e.g., a drug identified by a screening assay described herein) and a drug capable of modulating (e.g., upregulating or downregulating) the expression or activity of a transduction agent in the p75 signal transduction pathway. In another embodiment, the method comprises a step of administering a transduction agent in the p75 signal transduction pathway or a necleic acid molecule encoding it in order to compensate for reduced or abnormal expression or activity of the transduction agent in the p75 signal transduction pathway or the necleic acid molecule encoding it.

(Gene Therapy)

In a specific embodiment, a nucleic acid containing the nucleic acid sequence of a normal gene of the present invention, or a sequence encoding an antibodyor or a functional derivative thereof is administered for the purposes of gene therapy for treating, inhibiting, or preventing diseases or disorders associated with the abnormal expression and/or activity of a polypeptide of the present invention. Gene therapy refers to a therapy performed by administrating a nucleic acid, which has been expressed or is capable of being expressed, into subjects. In this embodiment of the present invention, a nucleic acid produces a protein encoded thereby and the protein mediates a therapeutic effect.

Any method available in the art for gene therapy may be used in accordance with the present invention. Illustrateive methods are described below.

See the following review articles for gene therapy: Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); and May, TIBTECH 11(5):155-215(1993). Generally known recombinant DNA techniques used for gene therapy are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

Therefore, in the present invention, gene therapy using a nucleic acid molecule encoding Pep5, p75, Rho GDI, MAG, p21, Rho, Rho kinase, or a variant or fragment thereof, or the like may be useful.

As used herein, the terms “trait” and “phenotype” are used interchangeably to refer to a observable trait, a detectable trait or other measurable traits of organisms. An example of a trait is a symptom of a disease or sensitivity to a disease. The term “trait” or “phenotype” may be used herein typically to refer to symptoms of breast-related diseases (e.g., breast cancer), obesity or obesity-related disorders, particularly atherosclerosis, insulin resistance, hypertention, microangiopathy in an obesity individual with type II diabetic, ocular lesion associated with microangiopathy in an obesity individual with type II diabetic, or renal lesion associated with microangiopathy in an obesity individual with type II diabeticor, or the morbidity thereof.

As used herein, the term “genotype” refers to a genetic structure of an individual organism, and often refers to an allele present in an individual or sample. The term “determine the genotype” of a sample or individual encompasses analysis of the sequence of a specific gene of the individual.

As used herein, the term “polymorphism” refers to the occurrence of at least two selective genomic sequences or alleles between different genomes or individuals. The term “polymorphism (polymorphic)” refers to a state having the possibility that at least two mutants are found in a specific genomic sequence in individuals. The term “polymorphic site” refers to a gene locus at which such a mutation occurs. Single nucleotide polymorphisms (SNPs) indicate that a nucleotide is replaced with another nucleotide at a polymorphic site. A single nucleotide deletion or insertion can lead to a single nucleotide polymorphism. As used herein, the term “single nucleotide polymorphism” preferably refers to a single nucleotide substitution. In general, two different nucleotides may share a polymorphic site between different individuals. In the present invention, polymorphisms of Pep5, p75, Rho GDI, MAG, Rho, Rho kinase, and the like are considered to be associated with nervous diseases. In one embodiment, alleles identified by such polymorphism analysis may be effective for regeneration, prophylaxis, diagnosis, treatment, or prognosis.

As used herein, the term “synthesis” or “synthesize” refers to a chemical substance (e.g., a polynucleotide, a polypeptide, or the like) which is purely chemically produced in contrast to enzymatic methods. Therefore, a “globally” synthesized chemical substance (e.g., a polynucleotide, a polypeptide, or the like) includes one that is globally produced by chemical means, while a “partially” synthesized chemical substance (e.g., a polynucleotide, a polypeptide, or the like) includes one that is only partially produced by chemical means.

As used herein, the term “region” refers to a physically contiguous portion of the first-order structure of a biomolecule. In the case of a protein, a region is defined by a portion having a contiguous amino acid sequence. As used herein, the term “domain” refers to a structural portion of a biomolecule which contributes to a known or inferred function of the biomolecule. A domain may have the same range as a region or a portion thereof. A domain may comprise a portion of a biomolecule, which is distinguished from a specific region, in addition to the whole or a part of the region. Examples of a domain of a protein in the p75 signal transduction according to the present invention include, but are not limited to, a signal peptide, an extracellular (i.e., N-terminal) domain, and a leucine rich repeated domain.

(Demonstration of Therapeutic Activity or Prophylactic Activity)

The compounds or pharmaceutical compositions of the present invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art (including, but not limited to, cell lysis assays). In accordance with the present invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

(Therapeutic/Prophylactic Administration and Composition)

The present invention provides methods of treatment, inhibition and prophylaxis by administration to a subject of an effective amount of a compound or pharmaceutical composition of the present invention. In a preferred aspect, the compound is substantially purified (e. g., substantially free from substances that limit its effect or produce undesired side-effects).

As used herein, term “amount effective for diagnosis, prophylaxis, treatment, or prognosis” refers to an amount which is recognized as therapeutically effective for diagnosis, prophylaxis, treatment (or therapy), or prognosis. Such an amount can be determined by those skilled in the art using techniques well known in the art with reference to various parameters.

Animals targeted by the present invention include any organism as long as it has a nervous system or its analogous system (e.g., animals (e.g., vertebrates, invertebrate)). Preferably, the animal is a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivore, insectivore, probbscidea, perissodactyla, artiodactyla, tubulidentata, pholidota, sirenia, cetacean, primates, rodentia, lagomorpha, etc.). Illustrative examples of a subject include, but are not limited to, animals, such as cattle, pig, horse, chicken, cat, dog, and the like. More preferably, cells derived from Primates (e.g., chimpanzee, Japanese monkey, human) are used. Most preferably, cells derived from a human are used.

When a nucleic acid molecule or polypeptide of the present invention is used as a medicament, the medicament may further comprise a pharmaceutically acceptable carrier. Any pharmaceutically acceptable carrier known in the art may be used in the medicament of the present invention.

Examples of a pharmaceutical acceptable carrier or a suitable formulation material include, but are not limited to, antioxidants, preservatives, colorants, flavoring agents, diluents, emulsifiers, suspending agents, solvents, fillers, bulky agents, buffers, delivery vehicles, and/or pharmaceutical adjuvants. Represetatively, a medicament of the present invention is administered in the form of a composition comprising a polypeptide or a polynucleotide, such as Pep5, p75, Rho GDI, MAG, p21, Rho, Rho kinase or a variant or fragment thereof, or a variant or derivative thereof with at least one physiologically acceptable carrier, exipient or diluent. For example, an appropriate vehicle may be injection solution, physiological solution, or artificial cerebrospinal fluid, which can be supplemented with other substances which are commonly used for compositions for parenteral delivery.

Acceptable carriers, excipients or stabilizers used herein preferably are nontoxic to recipients and are preferably inert at the dosages and concentrations employed, and preferably include phosphate, citrate, or other organic acids; ascorbic acid, α-tocopherol; low molecular weight polypeptides; proteins (e.g., serum albumin, gelatin, or immunoglobulins); hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

Hereinafter, commonly used preparation methods of the medicament of the present invention will be described. Note that animal drug compositions, quasi-drugs, marine drug compositions, food compositions, cosmetic compositions, and the like are prepared using known preparation methods.

The polypeptide, polynucleotide and the like of the present invention can be mixed with a pharmaceutically acceptable carrier and can be orally or parenterally administered as solid formulations (e.g., tablets, capsules, granules, abstracts, powders, suppositories, etc.) or liquid formulations (e.g., syrups, injections, suspensions, solutions, spray agents, etc.). Examples of pharmaceutically acceptable carriers include exicipients, lubricants, binders, disintegrants, disintegration inhibitors, absorption promoters, adsorbers, moisturizing agents, solubilizing agents, stabilizers and the like in solid formulations; and solvents, solubilizing agents, suspending agents, isotonic agents, buffers, soothing agents and the like in liquid formulations. Additives for formulations, such as antiseptics, antioxidants, colorants, sweeteners, and the like can be optionally used. The composition of the present invention can be mixed with substances other than the polynucleotide, polypeptide, and the like of the present invention. Examples of parenteral routes of administration include, but are not limited to, intravenous injection, intramuscular injection, intranasal, rectum, vagina, transdermal, and the like.

Examples of exicipients in solid formulations include glucose, lactose, sucrose, D-mannitol, crystallized cellulose, starch, calcium carbonate, light silicic acid anhydride, sodium chloride, kaolin, urea, and the like.

Examples of lubricants in solid formulations include, but are not limited to, magnesium stearate, calcium stearate, boric acid powder, colloidal silica, talc, polyethylene glycol, and the like.

Examples of binders in solid formulations include, but are not limited to, water, ethanol, propanol, saccharose, D-mannitol, crystallized cellulose, dextran, methylcellulose, hydroxypropylcellulose, hydroxypropylmethylcellulose, carboxymethylcellulose, starch solution, gelatin solution, polyvinylpyrrolidone, calcium phosphate, potassium phosphate, shellac, and the like.

Examples of disintegrants in solid formulations include, but are not limited to, starch, carboxymethylcellulose, carboxymethylcellulose calcium, agar powder, laminarin powder, croscarmellose sodium, carboxymethyl starch sodium, sodium alginate, sodium hydrocarbonate, calcium carbonate, polyoxyethylene sorbitan fatty acid esters, sodium lauryl sulfate, starch, monoglyceride stearate, lactose, calcium glycolate cellulose, and the like.

Examples of disintegration inhibitors in solid formulations include, but are not limited to, hydrogen-added oil, saccharose, stearin, cacao butter, hydrogenated oil, and the like.

Examples of absorption promoters in solid formulations include, but are not limited to, quaternary ammonium salts, sodium lauryl sulfate, and the like.

Examples of absorbers in solid formulations include, but are not limited to, starch, lactose, kaolin, bentonite, colloidal silica, and the like.

Examples of moisturizing agents in solid formulations include, but are not limited to, glycerin, starch, and the like.

Examples of solubilizing agents in solid formulations include, but are not limited to, arginine, glutamic acid, aspartic acid, and the like.

Examples of stabilizers in solid formulations include, but are not limited to, human serum albumin, lactose, and the like.

When tablets, pills, and the like are prepared as solid formulations, they may be optionally coated with film of a substance dissolvable in the stomach or the intestine (saccharose, gelatin, hydroxypropylcellulose, hydroxypropylmethylcellulose phthalate, etc.). Tablets include those optionally with a typical coating (e.g., dragees, gelatin coated tablets, enteric coated tablets, film coated tablets or double tablets, multilayer tablets, etc.). Capsules include hard capsules and soft capsules. When tablets are molded into the form of suppository, higher alcohols, higher alcohol esters, semi-synthesized glycerides, in addition to the above-described additives. The present invention is not so limited.

Preferable examples of solutions in liquid formulations include injection solutions, alcohols, propyleneglycol, macrogol, sesami oil, corn oil, and the like.

Preferrable examples of solubilizing agents in liquid formulations include, but are not limited to, polyethyleneglycol, propyleneglycol, D-mannitol, benzyl benzoate, ethanol, trisaminomethane, cholesterol, triethanolamine, sodium carbonate, sodium citrate, and the like.

Preferable examples of suspending agents in liquid formulations include surfactants (e.g., stearyltriethanolamine, sodium lauryl sulfate, lauryl amino propionic acid, lecithin, benzalkonium chloride, benzethonium chloride, glycerin monostearate, etc.), hydrophilic macromolecule (e.g., polyvinyl alcohol, polyvinylpyrrolidone, carboxymethylcellulose sodium, methylcellulose, hydroxymethylcellulose, hydroxyethylcellulose, hydroxyethylcellulose, hydroxypropylcellulose, etc.), and the like.

Preferable examples of isotonic agents in liquid formulations include, but are not limited to, sodium chloride, glycerin, D-mannitol, and the like.

Preferable examples of buffers in liquid formulations include, but are not limited to, phosphate, acetate, carbonate, citrate, and the like.

Preferable examples of soothing agents in liquid formulations include, but are not limited to, benzyl alcohol, benzalkonium chloride, procaine hydrochloride, and the like.

Preferable examples of antiseptics in liquid formulations include, but are not limited to, parahydroxybenzoate ester, chlorobutanol, benzyl alcohol, 2-phenylethylalcohol, dehydroacetic acid, sorbic acid, and the like.

Preferable examples of antioxidants in liquid formulations include, but are not limited to, sulfite, ascorbic acid, α-tocopherol, cysteine, and the like.

When liquid agents and suspensions are prepared as injections, they are sterilized and are preferably isotonic with the blood. Typically, these agents are made aseptic by filtration using a bacteria-contained filter or the like, mixing with a bactericideor, irradiation, or the like. Following these treatment, these agents may be made solid by lyophilization or the like. Immediately before use, sterile water or sterile injection diluent (lidocaine hydrochloride aqueous solution, physiological saline, glucose aqueous solution, ethanol or a mixure solution thereof, etc.) may be added.

The medicament composition of the present invention may further comprises a colorant, a presertive, a flavor, an aroma chemical, a sweetener, or other drugs.

The medicament of the present invention may be administered orally or parenterally. Alternatively, the medicament of the present invention may be administered intravenously or subcutaneously. When systemically administered, the medicament for use in the present invention may be in the form of a pyrogen-free, pharmaceutically acceptable aqueous solution. The preparation of such pharmaceutically acceptable compositions, with due regard to pH, isotonicity, stability and the like, is within the skill of the art. Administration methods may be herein oral, parenteral administration (e.g., intravenous, intramuscular, subcutaneous, intradermal, to mucosa, intrarectal, vaginal, topical to an affected site, to the skin, etc.). A prescription for such administration may be provided in any formulation form. Such a formulation form includes liquid formulations, injections, sustained preparations, and the like.

The medicament of the present invention may be prepared for storage by mixing a sugar chain composition having the desired degree of purity with optional physiologically acceptable carriers, excipients, or stabilizers (Japanese Pharmacopeia ver. 14 or the latest version; Remington's Pharmaceutical Sciences, 18th Edition, A. R. Gennaro, ed., Mack Publishing Company, 1990; and the like), in the form of lyophilized cake or aqueous solutions.

Various delivery systems are known and can be used to administer a compound of the present invention (e.g., liposomes, microparticles, microcapsules). Methods of introduction include, but are not limited to, intradermal, intramuscular, intraperitoneal, intravenous, subcutaneous, intranasal, epidural, and oral routes. The compounds or compositions may be administered by any convenient route (e.g., by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the present invention into the central nervous system by any suitable route (including intraventricular and intrathecal injection; intraventricular injection may be facilitated by an intraventricular catheter, for example, attached to a reservoir, such as an Ommaya reservoir). Pulmonary administration can also be employed, e.g., by use of an inhaler or nebulizer, and formulation with an aerosolizing agent.

In a specific embodiment, it may be desirable to administer a polypeptide, polynucleotide or composition of the present invention locally to the area in need of treatment (e.g., the central nervous system, the brain, etc.); this may be achieved by, for example, and not by way of limitation, local infusion during surgery, topical application (e.g., in conjunction with a wound dressing after surgery), by injection, by means of a catheter, by means of a suppository, or by means of an implant (the implant being of a porous, non-porous, or gelatinous material, including membranes, such as sialastic membranes, or fibers). Preferably, when administering a protein, including an antibody, of the present invention, care must be taken to use materials to which the protein does not absorb.

In another embodiment, the compound or composition can be delivered in a vesicle, in particular a liposome (see Langer, Science 249: 1527-1533. (1990); Treat et al., Liposomes in the Therapy of Infectious Disease and Cancer, Lopez-Berestein and Fidler (eds.), Liss, New York, pp. 353-365 (1989); Lopez-Berestein, ibid., pp. 317-327; 'see generally ibid.)

In yet another embodiment, the compound or composition can be delivered in a controlled release system. In one embodiment, a pump may be used (see Langer, supra; Sefton, CRC Crit. Ref. Biomed. Eng. 14: 201 (1987); Buchwald et al., Surgery 88: 507 (1980); Saudek et al., N. Engl. J. Med. 321: 574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23: 61 (1983); see also Levy et al., Science 228: 190 (1985); During et al., Ann. Neurol. 25: 351 (1989); Howard et al., J. Neurosurg. 71: 105 (1989)).

In yet another embodiment, a controlled release system can be placed in proximity to the therapeutic target, i. e., the brain, thus requiring only a fraction of the systemic dose (see, e. g., Goodson, in Medical Applications of Controlled Release, supra, vol. 2, pp. 115-138 (1984)).

Other controlled release systems are discussed in the review by Langer (Science 249: 1527-1533 (1990)).

The amount of a compound used in the treatment method of the present invention can be easily determined by those skilled in the art with reference to the purpose of use, a target disease (type, severity, and the like), the patient's age, weight, sex, and case history, the form or type of the cells, and the like. The frequency of the treatment method of the present invention which is applied to a subject (patient) is also determined by the those skilled in the art with respect to the purpose of use, a target disease (type, severity, and the like), the patient's age, weight, sex, and case history, the progression of the therapy, and the like. Examples of the frequency include once per day to several months (e.g., once per week to once per month). Preferably, administration is performed once per week to month with reference to the progression.

The doses of the polypeptides, polynucleotides or the like of the present invention vary depending on the subject's age, weight and condition or an administration method, or the like, including, but not limited to, ordinarily 0.01 mg to 10 g per day for an adult in the case of oral administration, preferably 0.1 mg to 1 g, 1 mg to 100 mg, 0.1 mg to 10 mg, and the like; in the parenteral administration, 0.01 mg to 1 g, preferably 0.01 mg to 100 mg, 0.1 mg to 100 mg, 1 mg to 100 mg, 0.1 mg to 10 mg, and the like. The present invention is not so limited.

As used herein, the term “administer” means that the polypeptides, polynucleotides or the like of the present invention or pharmaceutical compositions containing them are incorporated into a cell tissue of an organism either alone or in combination with other therapeutic agents. Combinations may be administered either concomitantly (e.g., as an admixture), separately but simultaneously or concurrently; or sequentially. This includes presentations in which the combined agents are administered together as a therapeutic mixture, and also procedures in which the combined agents are administered separately but simultaneously (e.g., as through separate intravenous lines into the same individual). “Combination” administration further includes the separate administration of one of the compounds or agents given first, followed by the second.

Abnormal conditions may be prevented or treated by administering a compound into cells having abnormality in a signal transduction pathway for an organism and then monitoring an effect of the administration of the compound on a biological function. The organism is preferably a mouse, a rat, a rabbit, or a goat, more preferably a monkey or an ape, and most preferably a human.

As used herein, “instructions” describe a method of administering a medicament of the present invention, a method for diagnosis, or the like for persons who administer, or are administered, the medicament or the like or persons who diagnose or are diagnosed (e.g, physicians, patients, and the like). The instructions describe a statement indicating an appropriate method for administrating a diagnostic, medicament, or the like of the present invention. The instructions are prepared in accordance with a format defined by an authority of a country in which the present invention is practiced (e.g., Health, Labor and Welfare Ministry in Japan, Food and Drug Administration (FDA) in U.S., and the like), explicitly describing that the instructions are approved by the authority. The instructions are so-called package insert and are typically provided in paper media. The instructions are not so limited and may be provided in the form of electronic media (e.g., web sites and electronic mails provided on the Internet).

The judgment of termination of treatment with a method of the present invention may be supported by a result of a standard clinical laboratory using commercially available assays or instruments or extinction of a clinical symptom characteristic to a disease (e.g., a neurological disease) associated with Pep5, p75, Rho GDI, MAG, GT1b, p21, Rho, Rho kinase, or the like. Treatment can be resumed by the relapse of a disease (e.g., a neurological disease) associated with Pep5, p75, Rho GDI, MAG, GT1b, p21, Rho, Rho kinase, or the like.

The present invention also provides a pharmaceutical package or kit comprising one or more containers loaded with one or more pharmaceutical compositions. A notice in a form defined by a government agency which regulates the production, use or sale of pharmaceutical products or biological products may be arbitrarily attached to such a container, representing the approval of the government agency relating to production, use or sale with respect to administration to human.

The plasma half-life and internal body distribution of a drug or a metabolite in the plasma, tumor and major organs may be determined so as to facilitate the selection of the most appropriate drug for inhibiting disorders. Such a measurement may be carried out by, for example, HPLC analysis of the plasma of an animal treated by a drug. The location of a radiolabeled compound may be determined using a detection method, such as X-ray, CAT scan, or MRI. A compound which exhibits strong inhibition activity in screening assays but has insufficient pharamacokinetic characteristics may be optimized by changing or retesting the chemical structure thereof. In this regard, a compound having satisfactory pharmacokinetic characteristics may be used as a model.

Toxicity studies may be carried out by measuring blood cell composition. For example, a toxicity study may be carried out in the following appropriate animal model: (1) a compound is administered into mice (an untreated control mouse should also be used); (2) a blood sample is periodically obtained from a mouse in each treatment group via the tail vein; and (3) the sample is analyzed for the numbers of erythrocytes and leukocytes, the blood cell composition, and the ratio of lymphocytes and polymorphonuclear cells. Comparison of the result of each drug regimen with the control shows whether or not toxicity is present.

At the end of each toxicity study, a further study may be carried out by sacrificing the animal (preferably, in accordance with American Veterinary Medical Association guidelines Report of the American Veterinary Medical Assoc. Panel on Euthanasia, (1993) J. Am. Vet. Med. Assoc. 202: 229-249). Thereafter, a representative animal from each treatment group may be tested by viewing the whole body for direct evidence of transitions, abnormal diseases or toxicity. A global abnormality in tissue is described and the tissue is hisotologically tested. A compound causing a reduction in weight or a reduction in blood components is not preferably as are compounds having an adverse action to major organs. In general, the greater the adverse action, the less preferable the compound.

### DETAILED DESCRIPTION

The present inventors' studies demonstrated that the association of p75^{NTR} with Rho GDI is enhanced by MAG and Nogo. As p75^{NTR} has an ability to release RhoA from Rho GDI in vitro, activation of RhoA by MAG and Nogo through p75^{NTR} may be attributable, at least partly, to Rho GDI displacement. The release of Rho from Rho GDI is an important step allowing the activation by guanine nucleotide exchange agents and membrane association of the GTP-bound form of Rho. As p75^{NTR} itself may not mediate the process of guanine nucleotide exchange, some Rho guanine nucleotide exchange agents might co-operate with p75^{NTR}, which is one of the issues to be addressed in the future. It is noted that another Rho GDI displacement agent, ezrin/radixin/moesin, also induces activation of RhoA in Swiss 3T3 cells, which is similar to our findings that p75^{NTR} activates RhoA.

There is growing evidence that p75^{NTR} has a key role in axon guidance or growth during the developmental stage (Dechant, G. & Barde, Y. A. Nat Neurosci. 5, 1131-1136 (2002)). Axon outgrowth from spinal motor neurons or forelimb motor neurons in mice carrying a mutation in p75^{NTR} is significantly retarded in vivo (Yamashita, T., Tucker, K. L. & Barde, Y. A. Neuron 24, 585-593 (1999); Bentley, C. A. & Lee K. F., J Neurosci. 20, 7706-7715 (2000)). This phenotype may be attributable to ligand binding to p75^{NTR}, as the chick ciliary neurons, which express p75^{NTR} but not TrkA, extend neurites in response to NGF. Contrary to these observations, abberant axonal elongation is observed in myelin-rich areas where these axons would normally not grow in mice carrying a mutation in p75^{NTR} (Walsh, G. S., Krol, K. M., Crutcher, K. A. & Kawaja, M. D., J. Neurosci. 19, 4155-4168 (1999)). In line with this finding, all the myelin-derived inhibitors of neurite outgrowth identified so far inhibit growth that is dependent on p75^{NTR} (Yamashita, T., Higuchi, H. & Tohyama, M., J. Cell Biol. 157, 565-570 (2002); Wang, K. C. & Kim, J. A., Sivasankaran, R., Segal, R. & He, Z., Nature 420, 74-78 (2002); Wong, S. T. et al., Nat Neurosci. 5, 1302-1308 (2002)). Our findings suggest that these effects may result from the Rho GDI displacement activity of p75^{NTR}. In addition, axon pathfinding errors of p75^{NTR}-expressing neurons are prominent among the phenotypes observed in mice carrying a mutation in p75^{NTR}, including mistargeting of sympathetic and cortical subplate axons (Lee, K. F, Bachman, K., Landis, S. & Jaenisch, R., Science 263, 1447-1449 (1994); McQuillen, P. S., DeFreitas, M. F., Zada, G. & Shatz, C. J., J. Neurosci. 22, 3580-3593 (2000)). As Rho seems to be involved in the regulation of axon pathfinding in the developmental stages, it is possible that the mistargeting in the absence of p75^{NTR} may be attributable to the failure of appropriate regulation of Rho activity. Interestingly, a recent report suggests a role of Rho GDI in spatial and temporal activation of the downstream pathway of Racl (Del Pozo, M. A. et al., Nat Cell Biol. 4, 232-239 (2002)). Although Rho GDI associates with Racl and blocks effector binding, release of Racl from Rho GDI at specific regions where integrin localizes allows Racl to bind its effectors. Thus, Rho GDI is suggested to confer spatially restricted regulation of Rho GTPases-effectors interaction. In future studies, it will be interesting to test the hypothesis that spatial control of Rho signaling regulated by Rho GDI may participate in the axon pathfindings.

A short isoform of p75^{NTR} has been found which lacks three of the four cysteine-rich repeats in the extracellular ligand-binding domain but has the intact intracellular domain (von Schack et al., Nat Neurosci. 4, 977-978 (2001)). The cells from mice bearing a targeted disruption of the third exon of the p75^{NTR} gene express this short isoform of p75^{NTR} (Lee, K. F. et al. Cell 69. 737-749 (1992)), but are insensitive to inhibitory molecules (Yamashita, T., Higuchi, H. & Tohyama, M. J. Cell Biol. 157, 565-570(2002); Wang, K. C. & Kim, J. A., Sivasankaran, R., Segal, R. & He, Z., Nature 420, 74-78 (2002); Wong, S. T. et al., Nat Neurosci. 5, 1302-1308 (2002)). As our data show that Pep5 did not affect the neurite outgrowth of the neurons which express the short isoform but not the full-length p75^{NTR} (B in FIG. 5), the short isoform might not act as a regulator of the neurite outgrowth.

As such a short isoform is a component constituting an interacellular domain, p75 comprising a component containing an extracellular domain may be used in a prefered embodiment.

It is now well established that axons of the adult central nervous system are capable of only a limited amount of regrowth after injury, and that an unfavorable environment plays major a role in the lack of regeneration. Much of the axon growth inhibitory effects are associated with myelin. Identification of the myelin-derived inhibitors led to our increase in the present inventors' knowledge about the molecular mechanisms of the biological activities. Therefore, it is now an important issue to explore strategies to overcome the inhibitory signals. The present inventors note that. Pep5 seems to specifically inhibit the action mediated by myelin-derived inhibitors, as Pep5 did not inhibit the NGF-induced promotion of the neurite outgrowth from hippocampal neurons (data not shown) or the cell death of superior cervical ganglion neurons treated with 100 ng/ml BDNF (data not shown). Specific inhibition of myelin-associated inhibitor effects may provide a practical therapeutic agent for injuries to the central nervous system.

Best Mode for Carrying Out the Present Invention

Hereinafter, embodiments of the present invention will be described. Embodiments provided below are provided for better understanding of the present invention. It will be understood that the scope of the present invention is not limited to the following description. Therefore, it is apparent that those skilled in the art can appropriately modify the present invention without departing from the spirit or scope of the present invention by referencing the description of the specification.

(Pep5 in the Polypeptide Form)

In one aspect, the present invention provides a composition comprising a Pep5 polypeptide for regenerating nerves, and a composition comprising a Pep5 polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by Pep5). The effect of nerve regeneration by blocking of a signal transduction pathway has not been conventionally known. Therefore, the present invention provides an effect more excellent than the prior art.

In one embodiment of the present invention, Pep5 or fragments or variants thereof comprise (a) a polypeptide encoded by a nucleic acid sequence as set forth in SEQ ID NO. 1 or a fragment thereof; (b) a polypeptide having an amino acid sequence as set forth in SEQ ID NO. 2 or a fragment thereof; (c) a polypeptide having an amino acid sequence as set forth in SEQ ID NO. 2 having at least one mutation selected from the group consisting of one or more amino acid substitutions, additions and deletions, and having biological activity; or (d) a polypeptide having an amino acid sequence having at least 70% homology to any one of the polypeptides described in (a) to (c), and having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of Pep5).

In another preferred embodiment, the biological activity possessed by the variant polypeptide described in (d) above includes, but is not limited to, for example, an interaction with an antibody specific to the polypeptide having the amino acid sequence as set forth in SEQ ID NO. 2 or a fragment thereof; an interaction with the p75 polypeptide; and the like.

In a preferred embodiment, the above-described homology to any one of the polypeptides described in (a) to (c) above may be at least about 80%, more preferably at least about 90%, even more preferably at least about 98%, and most preferably at least about 99%.

The polypeptide of the present invention typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, 30, and the like). The upper limit of the length of the polypeptide of the present invention may be greater than or equal to the full length of the sequence as set forth in SEQ ID NO. 2 as long as the peptide is capable of interacting with a given agent.

In one embodiment, the Pep5 polypeptide or fragments or variants thereof comprise the whole amino acid sequence as set forth in SEQ ID NO. 2. More preferably, the Pep5 or fragments or variants thereof consist of the whole amino acid sequence as set forth in SEQ ID NO. 2.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(Pep5 in the Nucleic Acid Form)

In one aspect, the present invention provides a composition comprising a nucleic acid molecule encoding the Pep5 polypeptide for regenerating nerves, and a composition comprising a nucleic acid molecule encoding the Pep5 polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by Pep5). The effect of nerve regeneration by blocking of a signal transduction pathway has not been conventionally known. Therefore, the present invention provides an effect more excellent than the prior art.

In one embodiment of the present invention, the nucleic acid molecule encoding Pep5 or fragments or variants thereof comprise (a) a polynucleotide having the base sequence as set forth in SEQ ID NO. 1 or a fragment thereof; (b) a polynucleotide encoding a polypeptide having an amino acid sequence (CFFRGGFFNHNPRYC as set forth in SEQ ID NO. 2) or a fragment thereof; (c) a polynucleotide encoding a variant polypeptide having the amino acid sequence as set forth in SEQ ID NO. 2 having at least one mutation selected from the group consisting of one or more amino acid substitutions, additions and deletions, and having biological activity; (d) a polynucleotide hybridizable to any one of the polynucleotides described in (a) to (c) above under stringent conditions and encoding a polypeptide having biological activity; or (e) a polynucleotide having a base sequence having at least 70% identity to any one of the polynucleotides described in (a) to (c) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of Pep5).

In another preferred embodiment, the biological activity possessed by the above-described variant polypeptide includes, but is not limited to, for example, an interaction with an antibody specific to the polypeptide having the amino acid sequence as set forth in SEQ ID NO. 2 or a fragment thereof; an interaction with p75; modulation of the functional regulation of Rho GDI by p75; and the like. These activities can be measured by, for example, immunological assays, phosphorylation quantification, or the like.

In a preferred embodiment, the identity to any one of the polynucleotides described in (a) to (c) above or a complementary sequence thereof may be at least about 80%, more preferably at least about 90%, even more preferably at least about 98%, and most preferably at least about 99%.

In a preferred embodiment, the nucleic acid molecule of the present invention encoding Pep5 or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22 30, and the like). The upper limit of the length of the polypeptide of the present invention may be greater than or equal to the full length of the sequence as set forth in SEQ ID NO. 1 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the nucleic acid molecule encoding Pep5 or fragments or variants thereof comprise the whole nucleic acid sequence as set forth in SEQ ID NO. 1. More preferably, the nucleic acid molecule encoding Pep5 or fragments or variants thereof consist of the whole nucleic acid sequence as set forth in SEQ ID NO. 1.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(Agent Capable of Specifically Interacting with p75 in the Polypeptide Form)

In one aspect, the present invention provides a composition comprising an agent capable of specifically interacting with a p75 polypeptide for regenerating nerves, and a composition comprising an agent capable of specifically interacting with a p75 polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting with p75). The effect of nerve regeneration by blocking of a signal transduction pathway has not been conventionally known. Therefore, the present invention provides an effect more excellent than the prior art.

In one embodiment of the present invention, the agent of the present invention may be an agent capable of specifically interacting with (a) a polypeptide having an amino acid sequence as set forth in SEQ ID NO. 4 or a fragment thereof; (b) a polypeptide having an amino acid sequence as set forth in SEQ ID NO. 4 having at least one mutation selected from the group consisting of one or more amino acid substitutions, additions and deletions, and having biological activity; (c) a polypeptide encoded by a splice variant or allelic variant of a base sequence as set forth in SEQ ID NO. 3 or 16; (d) a polypeptide which is a species homolog of the amino acid sequence as set forth in SEQ ID NO. 4; or (e) a polypeptide having an amino acid sequence having at least 70% homology to any one of the polypeptides described in (a) to (d), and having, biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the p75 gene).

In another preferred embodiment, the allelic variant described in (c) above preferably has at least 99% homology to the amino acid sequence as set forth in SEQ ID NO. 4.

In another preferred embodiment, the above-described species homolog can be identified as described above and preferably has at least about 30% homology to the amino acid sequence as set forth in SEQ ID NO. 4.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 4 or a fragment thereof; an interaction with the Rho GDI polypeptide; and the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide with which the agent of the present invention specifically interacts typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 4 as long as the peptide is capable of interacting with a given agent.

In a preferred embodiment, the agent of the present invention is selected from the group consisting of a nucleic acid molecule, a polypeptide, a lipid, a sugar chain, an organic small molecule and a composite molecule thereof. More preferably, the agent of the present invention is antibody or a derivative thereof (e.g., a single chain antibody). Therefore, the agent of the present invention can be used as a probe and/or an inhibitor.

In one embodiment, the p75 polypeptide or fragments or variants thereof comprise amino acids 273 to 427 of SEQ ID NO. 4 or amino acids 275 to 425 of SEQ ID NO. 17. More preferably, the p75 or fragments or variants thereof consist of amino acids 393 to 408 of SEQ ID NO. 4 or amino acids 391 to 406 of SEQ ID NO. 17.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In a preferred embodiment, the agent of the present invention may be advantageously labeled or capable of being bound to a label. When labeled, various states which can be measured using the agent of the present invention can be directly and/or readily measured. Any label can be used as long as it can be identified. Examples of a label include, but are not limited to, a fluorescent label, a chemically light emitting label, a radiolabel, and the like. Alternatively, when the agent is capable of interacting with an antibody or the like in an immune reaction, a system which is commonly used in an immune reaction, such as biotin-streptavidin.

(Agent Capable of Interacting with p75 Polypeptide in the Nucleic Acid Form)

In one aspect, the present invention provides a composition comprising an agent capable of specifically interacting with a nucleic acid molecule encoding the p75 polypeptide for regenerating nerves, and a composition comprising an agent capable of specifically interacting with a nucleic acid molecule encoding the p75 polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting with p75). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with a polynucleotide encoding (a) a polynucleotide having the base sequence as set forth in SEQ ID NO. 3 or 16 or a fragment thereof; (b) a polynucleotide encoding a polypeptide having an amino acid sequence as set forth in SEQ ID NO. 4 or a fragment thereof; (c) a polynucleotide encoding a variant polypeptide having the amino acid sequence as set forth in SEQ ID NO. 4 having at least one mutation selected from the group consisting of one or more amino acid substitutions, additions and deletions, and having biological activity; (d) a polynucleotide which is a splice variant or allelic variant of the base sequence as set forth in SEQ ID NO. 3 or 16; (e) a polynucleotide encoding a species homolog of the polypeptide having the amino acid sequence as set forth in SEQ ID NO. 4; (f) a polynucleotide hybridizable to any one of the polynucleotides described in (a) to (e) above under stringent conditions and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the p75 gene).

In another preferred embodiment, the biological activity possessed by the above-described variant polypeptide includes, but is not limited to, for example, an interaction with an antibody specific to the polypeptide having the amino acid sequence as set forth in SEQ ID NO. 4 or a fragment thereof; an interaction with p75; modulation of the functional regulation of Rho GDI by p75; and the like. These activities can be measured by, for example, immunological assays, phosphorylation quantification, or the like.

In another preferred embodiment, the allelic variant described in (c) above adventurously has at least 99% homology to the nucleic acid sequence as set forth in SEQ ID NO. 3 or 16.

The above-described species homolog can be identified by searching a gene sequence database for the species of the species homolog using the p75 of the present invention as a query sequence, if such a database is available. Alternatively, the species homolog can be identified by using the whole or part of p75 of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 3 or 16.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding p75 or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22, . . . 30, and the like). The upper limit of the length of the polynucleotide of the present invention may be greater than or equal to the full length of the sequence as set forth in SEQ ID NO. 3 or 16 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the nucleic acid molecule encoding p75 or fragments or variants thereof comprise amino acids 114 to 1397 of the nucleic acid sequence as set forth in SEQ ID NO. 3 or amino acids 114 to 1391 of the nucleic acid sequence as set forth in SEQ ID NO. 16. More preferably, the nucleic acid molecule encoding p75 or fragments or variants thereof consist of amino acids 1 to 3386 of the nucleic acid sequence as set forth in SEQ ID NO. 3 or amino acids 16 to 3259 of the nucleic acid sequence as set forth in SEQ ID NO. 16.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In a preferred embodiment, the agent of the present invention is selected from the group consisting of a nucleic acid molecule, a polypeptide, a lipid, a sugar chain, an organic small molecule and a composite molecule thereof.

In a preferred embodiment, the agent of the present invention is a nucleic acid molecule. When the agent of the present invention is a nucleic acid molecule, such a nucleic acid molecule may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22, . . . 30, and the like). The upper limit of the length of the polynucleotide of the present invention may be greater than or equal to the full length of the sequence as set forth in SEQ ID NO. 3 or 16 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

Therefore, in an illustrative embodiment, the agent of the present invention may be a nucleic acid molecule sequence having a sequence complementary to any of the nucleic acid sequences of the polynucleotides (a) to (g) or a sequence having at least 70% identity thereto.

In another illustrative embodiment, the agent of the present invention may be a nucleic acid molecule hybridizable to any of the nucleic acid sequences of the polynucleotides (a) to (g).

In another preferred embodiment, the agent of the present invention is an antisense or RNAi. RNAi may be either siRNA or shRNA, for example, double-stranded RNA having a length of about 20 bases (e.g., representatively about 21 to 23 bases) or less than about 20, preferably having a structure having 5′-phosphate and 3′-OH, where the 3′ terminus projects by about 2 bases. Preferably, shRNA may have 3′ terminus projects. The length of the double-stranded portion is about 10 nucleotides, more preferably about 20 or more nucleotides, but is not particularly limited. Here, the 3′ protruding end is preferably DNA, more preferably DNA of 2 nucleotides in length, even more preferably 2 to 4 nucleotides in length.

(p75 Extracellular Domain in the Polypeptide Form)

In one aspect, the present invention provides a composition comprising a p75 extracellular domain polypeptide for regenerating nerves, and a composition comprising a p75 extracellular domain polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the p75 extracellular domain). The effect of nerve regeneration by blocking of a signal transduction pathway has not been conventionally known. Therefore, the present invention provides an effect more excellent than the prior art.

In one embodiment, the p75 extracellular domain of the present invention comprises (a) a polypeptide encoded by nucleotides 198 to 863 or nucleotides 201 to 866 of the nucleic acid sequence as set forth in SEQ ID NO. 3 or 16 or a fragment thereof; (b) a polypeptide having amino acids 29 to 250 or 30 to 251 of the amino acid sequence as set forth in SEQ ID NO. 4 or a fragment thereof; (c) a variant polypeptide having amino acids 29 to 250 or 30 to 251 of the amino acid sequence as set forth in SEQ ID NO. 4 having at least one mutation selected from the group consisting of one or more amino acid substitutions, additions and deletions, and having biological activity; (d) a polypeptide encoded by a splice variant or allelic variant of nucleotides 198 to 863 or 201 to 866 of the base sequence as set forth in SEQ ID NO. 3 or 16, respectively; (e) a polypeptide which is a species homolog of a polypeptide having amino acids 29 to 250 or 30 to 251 of the amino acid sequence as set forth in SEQ ID NO. 4, respectively; or (f) a polypeptide consisting of an amino acid sequence having at least 70% identity to any one of the polypeptides described in (a) to (e), and having biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the p75 gene).

In another preferred embodiment, the allelic variant described in (c) above preferably has at least 99% homology to the amino acid sequence as set forth in SEQ ID NO. 4.

In another preferred embodiment, the above-described species homolog can be identified as described above and preferably has at least about 30% homology to the amino acid sequence as set forth in SEQ ID NO. 4.

The above-described species homolog can be identified by searching a gene sequence database for the species of the species homolog using the p75 of the present invention as a query sequence, if such a database is available. Alternatively, the species homolog can be identified by using the whole or part of p75 of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 3 or 16 or the amino acid sequence as set forth in SEQ ID NO. 4.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 4 or a fragment thereof; an interaction with the Pep5 polypeptide; an interactin with Rho, an interactin with GT1b, an interactin with MAG, an interactin with NgR, an interactin with Nogo, an interactin with OMgp, the modulation of the functional regulation of Rho GDI by p75; and the like. These interactions can be measured by immunoassays, phophorylation quantification, and the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide of the present invention typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 4 as long as the peptide is capable of interacting with a given agent.

In one embodiment, the p75 extracellular domain polypeptide or fragments or variants thereof comprise amino acids 29 to 250 or 30 to 251 of SEQ ID NO. 4 or 17, respectively. More preferably, the p75 extracellular domain polypeptide or fragments or variants thereof consist of amino acids 29 to 250 or 30 to 251 of SEQ ID NO. 4 or 17, respectively.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In another embodiment, the p75 extracellular domain of the present invention is preferably soluble. Such a soluble peptide can be prepared by removing the whole or a part of the transmembrane domain using genetic engineering or synthesis.

(p75 Extracellular Domain Polypeptide in the Nucleic Acid Form)

In one aspect, the present invention provides a composition comprising a nucleic acid molecule encoding the p75 extracellular domain polypeptide for regenerating nerves, and a composition comprising a nucleic acid molecule encoding the p75 extracellular domain polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the p75 extracellular domain). The effect of nerve regeneration by blocking of a signal transduction pathway has not been conventionally known. Therefore, the present invention provides an effect more excellent than the prior art.

In one embodiment, the p75 extracellular domain of the present invention comprise a polynucleotide selected from the group consisting of (a) a polynucleotide having nucleotides 198 to 863 or nucleotides 201 to 866 of the base sequence as set forth in SEQ ID NO. 3 or 16, respectively, or a fragment thereof; (b) a polynucleotide encoding amino acids 29 to 250 or 30 to 251 of the amino acid sequence as set forth in SEQ ID NO. 4 or 17 or a fragment thereof; (c) a polynucleotide encoding a variant polypeptide having amino acids 29 to 250 or 30 to 251 of the amino acid sequence as set forth in SEQ ID NO. 4 or 17 having at least one mutation selected from the group consisting of one or more amino acid substitutions, additions and deletions, and having biological activity; (d) a polynucleotide which is a splice variant or allelic variant of nucleotides 198 to 863 or 201 to 866 of the base sequence as set forth in SEQ ID NO. 3 or 16, respectively; (e) a polynucleotide encoding a species homolog of a polypeptide having amino acids 29 to 250 or 30 to 251 of the amino acid sequence as set forth in SEQ ID NO. 4; (f) a polynucleotide hybridizable to any one of the polynucleotide described in (a) to (e), and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the p75 gene).

In another preferred embodiment, the biological activity possessed by the above-described variant polypeptide includes, but is not limited to, for example, an interaction with an antibody specific to the polypeptide having the amino acid sequence as set forth in SEQ ID NO. 4 or 17 or a fragment thereof; an interaction with the Pep5 polypeptide; an interactin with Rho, an interactin with GT1b, an interactin with MAG, an interactin with NgR, an interactin with Nogo, an interactin with OMgp; modulation of the functional regulation of Rho GDI by p75; and the like. These activities can be measured by, for example, immunological assays, phosphorylation quantification, or the like.

In another preferred embodiment, the allelic variant described in (c) above adventurously has at least 99% homology to the nucleic acid sequence as set forth in SEQ ID NO. 3 or 16.

The above-described species homolog can be identified by searching a gene sequence database for the species of the species homolog using the p75 extracellular domain of the present invention as a query sequence, if such a database is available. Alternatively, the species homolog can be identified by using the whole or part of the p75 extracellular domain of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 3 or 16.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding the p75 extracellular domain or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22, . . . 30, and the like). The upper limit of the length of the polynucleotide of the present invention may be greater than or equal to the full length of the sequence as set forth in SEQ ID NO. 3 or 16 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the nucleic acid molecule encoding the p75 extracellular domain or fragments or variants thereof comprise nucleotides 198 to 863 or 201 to 866 of the nucleic acid sequence as set forth in SEQ ID NO. 3 or 16, respectively. More preferably, the nucleic acid molecule encoding the p75 extracellular domain or fragments or variants thereof consist of nucleotides 198 to 863 or 201 to 866 of the nucleic acid sequence as set forth in SEQ ID NO. 3 or 16, respectively.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In another embodiment, the p75 extracellular domain peptide of the present invention is preferably soluble. Such a soluble peptide can be prepared by removing the whole or a part of the transmembrane domain using genetic engineering or synthesis.

(Rho in the Polypeptide Form)

In one aspect, the present invention provides a composition comprising a Rho polypeptide for regenerating nerves, and a composition comprising a Rho polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by Rho). The effect of nerve regeneration by blocking of a signal transduction pathway has not been conventionally known. Therefore, the present invention provides an effect more excellent than the prior art.

In one embodiment, the Rho polypeptide of the present invention comprise (a) a polypeptide having an amino acid sequence as set forth in SEQ ID NO. 5 or a fragment thereof; (b) a polypeptide having an amino acid sequence as set forth in SEQ ID NO. 6; (c) a variant polypeptide having an amino acid sequence as set forth in SEQ ID NO. 6 having at least one mutation selected from the group consisting of one or more amino acid substitutions, additions and deletions, and having biological activity; (d) a polypeptide encoded by a splice variant or allelic variant of a base sequence as set forth in SEQ ID NO. 5; (e) a polypeptide which is a species homolog of the amino acid sequence as set forth in SEQ ID NO. 6; or (f) a polypeptide having an amino acid sequence having at least. 70% identity to any one of the polypeptides described in (a) to (e), and having biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho or RhoA gene).

In another preferred embodiment, the allelic variant described in (c) above preferably has at least 99% homology to the amino acid sequence as set forth in SEQ ID NO. 6.

In another preferred embodiment, the above-described species homolog can be identified as described above and preferably has at least about 30% homology to the amino acid sequence as set forth in SEQ ID NO. 6.

The above-described species homolog can be identified by searching a gene sequence database for the species of the species homolog using the Rho or RhoA of the present invention as a query sequence, if such a database is available. Alternatively, the species homolog can be identified by using the whole or part of Rho or RhoA of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 5 or the amino acid sequence as set forth in SEQ ID No. 6.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 6 or a fragment thereof; an interaction with Pep5; an interaction with p75; an interaction with GT1b; an interaction with MAG; an interaction with Rho GDI; and the like. These activities can be measured by, for example, immunological assays, phosphorylation quantification, or the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide of the present invention typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 6 as long as the peptide is capable of interacting with a given agent.

In one embodiment, the Rho polypeptide or fragments or variants thereof comprise amino acids 29 to 250 or 30 to 251 of SEQ ID No. 6. More preferably, the Rho polypeptide or fragments or variants thereof consist of amino acids 29 to 250 or 30 to 251 of SEQ ID No. 6.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In another embodiment, the Rho polypeptide of the present invention is. preferably soluble. Such a soluble peptide can be prepared by removing the whole or a part of the transmembrane domain using genetic engineering or synthesis.

(Rho Polypeptide in the Nucleic Acid Form)

In one aspect, the present invention provides a composition comprising a nucleic acid molecule encoding the Rho polypeptide for regenerating nerves, and a composition comprising a nucleic acid molecule encoding the Rho polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio ogawa, Chugai-Igaku 1994). In the present invention, it was revealed that regeneration of nerves occurs due to inhibition of neurite outgrowth being disrupted by blocking of the p75 signal transduction pathway (by modulation of the Rho polypeptide). The effect of nerve regeneration by blocking of a signal transduction pathway has not been conventionally known. Therefore, the present invention provides an effect more excellent than the prior art.

In one embodiment of the present invention, the nucleic acid molecule encoding the Rho polypeptide or fragments or variants thereof comprise a polynucleotide selected from the group consisting of (a) a polynucleotide having the base sequence as set forth in SEQ ID NO. 5 or a fragment thereof; (b) a polynucleotide encoding an amino acid sequence as set forth in SEQ ID NO. 6 or a fragment thereof; (c) a polynucleotide encoding a variant polypeptide having the amino acid sequence as set forth in SEQ ID NO. 6 having at least one mutation selected from the group consisting of one or more amino acid substitutions, additions and deletions, and having biological activity; (d) a polynucleotide which is a splice variant or allelic variant of the base sequence as set forth in SEQ ID NO. 5; (e) a polynucleotide encoding a species homolog of the polypeptide having the amino acid sequence as set forth in SEQ ID NO. 6; (f) a polynucleotide hybridizable to any one of the polynucleotides described in (a) to (e) above under stringent conditions and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho gene).

In another preferred embodiment, the biological activity possessed by the above-described variant polypeptide includes, but is not limited to, for example, an interaction with an antibody specific to the polypeptide having the amino acid sequence as set forth in SEQ ID NO. 6 or a fragment thereof; an interaction with Pep5; an interaction with p75; an interaction with GT1b; an interaction with MAG; modulation of the functional regulation of Rho GDI; and the like. These activities can be measured by, for example, immunological assays, phosphorylation quantification, or the like.

In another preferred embodiment, the allelic variant described in (c) above has at least 99% homology to the nucleic acid sequence as set forth in SEQ ID NO. 5.

The above-described species homolog can be identified by searching a gene sequence database for the species of the species homolog using the Rho or RhoA of the present invention as a query sequence, if such a database is available. Alternatively, the species homolog can be identified by using the whole or part of the Rho of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 5.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding the Rho polypeptide or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22, . . . 30, and the like). The upper limit of the length of the polynucleotide of the present invention may be greater than or equal to the full length of the sequence as set forth in SEQ ID NO. 5 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the nucleic acid molecule encoding the Rho polypeptide or fragments or variants thereof comprise the whole nucleic acid sequence as set forth in SEQ ID NO. 5. More preferably, the nucleic acid molecule encoding Rho or fragments or variants thereof consist of the whole nucleic acid sequence as set forth in SEQ ID NO. 5.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In another embodiment, the Rho polypeptide of the present invention is preferably bound to the PTD domain. Such a nucleic acid molecule encoding the PTD domain-bound polypeptide can be prepared by adding a nucleic acid sequence encoding the PTD domain using genetic engineering or synthesis.

(Agent Capable of Specifically Interacting with Rho GDI Polypeptide)

In one aspect, the present invention provides a composition comprising an agent capable of specifically interacting with a Rho GDI polypeptide for regenerating nerves, and a composition comprising an agent capable of specifically interacting with a Rho GDI polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting with the Rho GDI polypeptide). The effect of nerve regeneration by blocking of a signal transduction pathway has not been conventionally known. Therefore, the present invention provides an effect more excellent than the prior art.

In one embodiment of the present invention, the agent of the present invention may be an agent capable of specifically interacting with (a) a polypeptide encoded by the nucleic acid sequence as set forth in SEQ ID NO. 5 or a fragment thereof; (b) a polypeptide having an amino acid sequence as set forth in SEQ ID NO. 6; (c) a variant polypeptide having an amino acid sequence as set forth in SEQ ID NO. 6 having at least one mutation selected from the group consisting of one or more amino acid substitutions, additions and deletions, and having biological activity; (d) a polypeptide encoded by a splice variant or allelic variant of the base sequence as set forth in SEQ ID NO. 5; (e) a polypeptide which is a species homolog of the amino acid sequence as set forth in SEQ ID NO. 6; or (f) a polypeptide having an amino acid sequence having at least 70% identity to any one of the polypeptides described in (a) to (e), and having biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho GDI gene).

In another preferred embodiment, the allelic variant described in (c) above preferably has at least 99% homology to the amino acid sequence as set forth in SEQ ID NO. 4.

In another preferred embodiment, the above-described species homolog can be identified as described above and preferably has at least about 30% homology to the amino acid sequence as set forth in SEQ ID NO. 6.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 6 or a fragment thereof; an interaction with the p75 polypeptide; and the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide with which the agent of the present invention specifically interacts typically has a sequence of at least 3 contiguous amino acids. The amino acid length of the polypeptide of the present invention may be short as long as the polynucleotide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 6 as long as the peptide is capable of interacting with a given agent.

In a preferred embodiment, the agent of the present invention is selected from the group consisting of a nucleic acid molecule, a polypeptide, a lipid, a sugar chain, an organic small molecule and a composite molecule thereof. More preferably, the agent of the present invention is antibody or a derivative thereof (e.g., a single chain antibody). Therefore, the agent of the present invention can be used as a probe and/or an inhibitor.

In one embodiment, the Rho GDI polypeptide or fragments or variants thereof comprise the whole amino acid sequence as set forth in SEQ ID NO. 6. More preferably, the Rho GDI or fragments or variants thereof consist of the whole amino acid sequence as set forth in SEQ ID NO. 6.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(Agent Capable of Interacting with a Nucleic Acid Molecule Encoding the Rho GDI Polypeptide)

In one aspect, the present invention provides a composition comprising an agent capable of specifically interacting with a nucleic acid molecule encoding the Rho GDI polypeptide for regenerating nerves, and a composition comprising an agent capable of specifically interacting with a nucleic acid molecule encoding the Rho GDI polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting with the Rho GDI polypeptide). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with a polynucleotide encoding (a) a polynucleotide having the base sequence as set forth in SEQ ID NO. 5 or a fragment thereof; (b) a polynucleotide encoding a polypeptide having an amino acid sequence as set forth in SEQ ID NO. 6 or a fragment thereof; (c) a polynucleotide encoding a variant polypeptide having the amino acid sequence as set forth in SEQ ID NO. 6 having at least one mutation selected from the group consisting of one or more amino acid substitutions, additions and deletions, and having biological activity; (d) a polynucleotide which is a splice variant or allelic variant of the base sequence as set forth in SEQ ID NO. 5; (e) a polynucleotide encoding a species homolog of the polypeptide having the amino acid sequence as set forth in SEQ ID NO. 6; (f) a polynucleotide hybridizable to any one of the polynucleotides described in (a) to (e) above under stringent conditions and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho GDI gene).

In another preferred embodiment, the biological activity possessed by the above-described variant polypeptide includes, but is not limited to, for example, an interaction with an antibody specific to the polypeptide having the amino acid sequence as set forth in SEQ ID NO. 6 or a fragment thereof; an interaction with p75; modulation of the functional regulation of Rho GDI by p75; and the like. These activities can be measured by, for example, immunological assays, phosphorylation quantification, or the like.

In another preferred embodiment, the allelic variant described in (c) above has at least 99% homology to the nucleic acid sequence as set forth in SEQ ID NO. 5.

The above-described species homolog can be identified by searching a gene sequence database for the species of the species homolog using the Rho GDI of the present invention as a query sequence, if such a database is available. Alternatively, the species homolog can be identified by using the whole or part of the Rho GDI of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 5.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding Rho GDI or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22, 30, and the like). The upper limit of the length of the polynucleotide of the present invention may be greater than or equal to the full length of the sequence as set forth in SEQ ID NO. 5 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the Rho GDI polypeptide or fragments or variants thereof comprise the whole amino acid sequence as set forth in SEQ ID NO. 6. More preferably, the Rho GDI or fragments or variants thereof consist of the whole amino acid sequence as set forth in SEQ ID NO. 6.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In a preferred embodiment, the agent of the present invention is selected from the group consisting of a nucleic acid molecule, a polypeptide, a lipid, a sugar chain, an organic small molecule and a composite molecule thereof.

In a preferred embodiment, the agent of the present invention is a nucleic acid molecule. When the agent of the present invention is a nucleic acid molecule, such a nucleic acid molecule may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22, 30, and the like). The upper limit of the length of the polynucleotide of the present invention may be greater than or equal to the full length of the sequence as set forth in SEQ ID NO. 5 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

Therefore, in an illustrative embodiment, the agent of the present invention may be a nucleic acid molecule sequence having a sequence complementary to any of the nucleic acid sequences of the polynucleotides (a) to (g) or a sequence having at least 70% identity thereto.

In another illustrative embodiment, the agent of the present invention may be a nucleic acid molecule hybridizable to any of the nucleic acid sequences of the polynucleotides (a) to (g).

In another preferred embodiment, the agent of the present invention is an antisense or RNAi. RNAi may be either siRNA or shRNA, for example, double-stranded RNA having a length of about 20 bases (e.g., representatively about 21 to 23 bases) or less than about 20, preferably having a structure having 5′-phosphate and 3′-OH, where the 3′ terminus projects by about 2 bases. Preferably, shRNA may have 31 terminus projects. The length of the double-stranded portion is about 10 nucleotides, more preferably about 20 or more nucleotides, but is not particularly limited. Here, the 3′ protruding end is preferably DNA, more preferably DNA of 2 nucleotides in length, even more preferably 2 to 4 nucleotides in length.

(Agent Capable of Specifically Interacting with MAG in the Polypeptide Form)

In one aspect, the present invention provides a composition comprising an agent capable of specifically interacting with a MAG polypeptide for regenerating nerves, and a composition comprising an agent capable of specifically interacting with a MAG polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting (e.g., inhibiting or suppressing) with MAG). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with (a) a polypeptide having an amino acid sequence as set forth in SEQ ID NO. 7 or a fragment thereof; (b) a polypeptide having an amino acid sequence as set forth in SEQ ID NO. 8; (c) a variant polypeptide having the amino acid sequence as set forth in SEQ ID NO. 8 having at least one mutation selected from the group consisting of one or more amino acid substitutions, additions and deletions, and having biological activity;. (d) a polypeptide encoded by a splice variant or allelic variant of a base sequence as set forth in SEQ ID NO. 7; (e) a polypeptide which is a species homolog of the amino acid sequence as set forth in SEQ ID NO. 8; or (f) a polypeptide having an amino acid sequence having at least 70% identity to any one of the polypeptides described in (a) to (e), and having biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the MAG gene).

In another preferred embodiment, the allelic variant described in (c) above preferably has at least 99% homology to the amino acid sequence as set forth in SEQ ID NO. 4.

In another preferred embodiment, the above-described species homolog can be identified as described above and preferably has at least about 30% homology to the amino acid sequence as set forth in SEQ ID NO. 8.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 8 or a fragment thereof; an interaction with p75 polypeptide; and the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide with which the agent of the present invention specifically interacts typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 8 as long as the peptide is capable of interacting with a given agent.

In a preferred embodiment, the agent of the present invention is selected from the group consisting of a nucleic acid molecule, a polypeptide, a lipid, a sugar chain, an organic small molecule and a composite molecule thereof. More preferably, the agent of the present invention is antibody or a derivative thereof (e.g., a single chain antibody). Therefore, the agent of the present invention can be used as a probe and/or an inhibitor.

In one embodiment, the MAG polypeptide or fragments or variants thereof comprise amino acids 1 to 626 of SEQ ID NO. 8. More preferably, the MAG or fragments or variants thereof consist of the whole amino acid sequence of SEQ ID NO. 8.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(Agent Capable of Interacting with a Nucleic Acid Molecule Encoding a MAG Polypeptide)

In one aspect, the present invention provides a composition comprising an agent capable of specifically interacting with a nucleic acid molecule encoding the MAG polypeptide for regenerating nerves, and a composition comprising an agent capable of specifically interacting with a nucleic acid molecule encoding the MAG polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting with the MAG polypeptide). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with a polynucleotide encoding (a) a polynucleotide having the base sequence as set forth in SEQ ID NO. 7 or a fragment thereof; (b) a polynucleotide encoding a polypeptide having an amino acid sequence as set forth in SEQ ID NO. 8 or a fragment thereof; (c) a polynucleotide encoding a variant polypeptide having the amino acid sequence as set forth in SEQ ID NO. 8 having at least one mutation selected from the group consisting of one or more amino acid substitutions, additions and deletions, and having biological activity; (d) a polynucleotide which is a splice variant or allelic variant of the base sequence as set forth in SEQ ID NO. 7; (e) a polynucleotide encoding a species homolog of the polypeptide having the amino acid sequence as set forth in SEQ ID NO. 8; (f) a polynucleotide hybridizable to any one of the polynucleotides described in (a) to (e) above under stringent conditions and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the MAG gene).

In another preferred embodiment, the biological activity possessed by the above-described variant polypeptide includes, but is not limited to, for example, an interaction with an antibody specific to the polypeptide having the amino acid sequence as set forth in SEQ ID NO. 8 or a fragment thereof; an interaction with p75; modulation of the functional regulation of MAG by p75; and the like. These activities can be measured by, for example, immunological assays, phosphorylation quantification, or the like.

In another preferred embodiment, the allelic variant described in (c) above adventurously has at least. 99% homology to the nucleic acid sequence as set forth in SEQ ID NO. 7.

The above-described species homolog can be identified by searching a gene sequence database for the species of the species homolog using the MAG of the present invention as a query sequence, if such a database is available. Alternatively, the species homolog can be identified by using the whole or part of the MAG of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 7.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding MAG or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22 30, and the like). The upper limit of the length of the polynucleotide of the present invention may be greater than or equal to the full length of the sequence as set forth in SEQ ID NO. 7 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the MAG polypeptide or fragments or variants thereof comprise the whole amino acid sequence as set forth in SEQ ID NO. 7. More preferably, the MAG or fragments or variants thereof consist of the whole amino acid sequence as set forth in SEQ ID NO. 7.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In a preferred embodiment, the agent of the present invention is selected from the group consisting of a nucleic acid molecule, a polypeptide, a lipid, a sugar chain, an organic small molecule and a composite molecule thereof.

In a preferred embodiment, the agent of the present invention is a nucleic acid molecule. When the agent of the present invention is a nucleic acid molecule, such a nucleic acid molecule may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22 30, and the like). The upper limit of the length of the polynucleotide of the present invention may be greater than or equal to the full length of the sequence as set forth in SEQ ID NO. 7 as long as the polynucleotide can be used for the intended purpose (e.g. antisense, RNAi, marker, primer, probe) capable of interacting with a given agent. Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

Therefore, in an illustrative embodiment, the agent of the present invention may be a nucleic acid molecule sequence having a sequence complementary to any of the nucleic acid sequences of the polynucleotides (a) to (g) or a sequence having at least 70% identity thereto.

In another illustrative embodiment, the agent of the present invention may be a nucleic acid molecule hybridizable to any of the nucleic acid sequences of the polynucleotides (a) to (g). Stringency may be high, moderate, or low, which can be determined by those skilled in the art depending on the situation.

In another preferred embodiment, the agent of the present invention is an antisense or RNAi. RNAi may be either siRNA or shRNA, for example, double-stranded RNA having a length of about 20 bases (e.g., representatively about 21 to 23 bases) or less than about 20, preferably having a structure having 5′-phosphate and 3′-OH, where the 3′ terminus projects by about 2 bases. Preferably, shRNA may have 3′ terminus projects. The length of the double-stranded portion is about 10 nucleotides, more preferably about 20 or more nucleotides, but is not particularly limited. Here, the 3′ protruding end is preferably DNA, more preferably DNA of 2 nucleotides in length, even more preferably 2 to 4 nucleotides in length.

(Agent Capable of Specifically Interacting with Rho in the Polypeptide Form)

In one aspect, the present invention provides a composition comprising an agent capable of specifically interacting with a nucleic acid molecule encoding a Rho polypeptide for regenerating nerves, and a composition comprising an agent capable of specifically interacting with a nucleic acid molecule encoding a Rho polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting (e.g., inhibiting or suppressing) with Rho). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with (a) a polypeptide having an amino acid sequence as set forth in SEQ ID NO. 11 or a fragment thereof; (b) a polypeptide having an amino acid sequence as set forth in SEQ ID NO. 12; (c) a variant polypeptide having the amino acid sequence as set forth in SEQ ID NO. 11 having at least one mutation selected from the group consisting of one or more amino acid substitutions, additions and deletions, and having biological activity; (d) a polypeptide encoded by a splice variant or allelic variant of a base sequence as set forth in SEQ ID NO. 11; (e) a polypeptide which is a species homolog of the amino acid sequence as set forth in SEQ ID NO. 12; or (f) a polypeptide having an amino acid sequence having at least 70% identity to any one of the polypeptides described in (a) to (e), and having biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho gene).

In another preferred embodiment, the allelic variant described in (c) above preferably has at least 99% homology to the amino acid sequence as set forth in SEQ ID NO. 4.

In another preferred embodiment, the above-described species homolog can be identified as described above and preferably has at least about 30% homology to the amino acid sequence as set forth in SEQ ID NO. 12.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 12 or a fragment thereof; an interaction with p75 polypeptide; and the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide with which the agent of the present invention specifically interacts typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 12 as long as the peptide is capable of interacting with a given agent.

In a preferred embodiment, the agent of the present invention is selected from the group consisting of a nucleic acid molecule, a polypeptide, a lipid, a sugar chain, an organic small molecule and a composite molecule thereof. More preferably, the agent of the present invention is antibody or a derivative thereof (e.g., a single chain antibody). Therefore, the agent of the present invention can be used as a probe and/or an inhibitor.

In one embodiment, the Rho polypeptide or fragments or variants thereof comprise amino acids 1 to. 193 of SEQ ID NO. 12. More preferably, the Rho or fragments or variants thereof consist of the whole amino acid sequence of SEQ ID NO. 12.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(Agent Capable of Interacting with a Nucleic Acid Molecule Encoding a Rho Polypeptide)

In one aspect, the present invention provides a composition comprising an agent capable of specifically interacting with a nucleic acid molecule encoding a Rho polypeptide for regenerating nerves, and a composition comprising an agent capable of specifically interacting with a nucleic acid molecule encoding a Rho polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting with the Rho polypeptide). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with a polynucleotide encoding (a) a polynucleotide having the base sequence as set forth in SEQ ID NO. 11 or a fragment thereof; (b) a polynucleotide encoding a polypeptide having an amino acid sequence as set forth in SEQ ID NO. 12 or a fragment thereof; (c) a polynucleotide encoding a variant polypeptide having the amino acid sequence as set forth in SEQ ID NO. 12 having at least one mutation selected from the group consisting of one or more amino acid substitutions, additions and deletions, and having biological activity; (d) a polynucleotide which is a splice variant or allelic variant of the base sequence as set forth in SEQ ID NO. 11; (e) a polynucleotide encoding a species homolog of the polypeptide having the amino acid sequence as set forth in SEQ ID NO. 12; (f) a polynucleotide hybridizable to any one of the polynucleotides described in (a) to (e) above under stringent conditions and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho gene).

In another preferred embodiment, the biological activity possessed by the above-described variant polypeptide includes, but is not limited to, for example, an interaction with an antibody specific to the polypeptide having the amino acid sequence as set forth in SEQ ID NO. 12 or a fragment thereof; an interaction with p75; modulation of the functional regulation of Rho by p75 or Rho GDI; and the like. These activities can be measured by, for example, immunological assays, phosphorylation quantification, or the like.

In another preferred embodiment, the allelic variant described in (c) above adventurously has at least 99% homology to the nucleic acid sequence as set forth in SEQ ID NO. 11.

The above-described species homolog can be identified by searching a gene sequence database for the species of the species homolog using the Rho of the present invention as a query sequence, if such a database is available. Alternatively, the species homolog can be identified by using the whole or part of the Rho of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 11.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding Rho or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22 30, and the like). The upper limit of the length of the polynucleotide of the present invention may be greater than or equal to the full length of the sequence as set forth in SEQ ID NO. 11 as long as the polynucleotide can be used for the intended purpose (e.g. antisense, RNAi, marker, primer, probe, capable of interacting with a given agent). The upper limit of the length of the polynucleotide of the present invention may be greater than or equal to the full length of the sequence as set forth in SEQ ID NO. 11 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the Rho polypeptide or fragments or variants thereof comprise amino acids 1 to 579 of SEQ ID NO. 11. More preferably, the Rho or fragments or variants thereof consist of the whole amino acid sequence as set forth in SEQ ID NO. 11.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In a preferred embodiment, the agent of the present invention is selected from the group consisting of a nucleic acid molecule, a polypeptide, a lipid, a sugar chain, an organic small molecule and a composite molecule thereof.

In a preferred embodiment, the agent of the present invention is a nucleic acid molecule. When the agent of the present invention is a nucleic acid molecule, such a nucleic acid molecule may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22, . . . 30, and the like). The upper limit of the length of the polynucleotide of the present invention may be greater than or equal to the full length of the sequence as set forth in SEQ ID NO. 11 as long as the polynucleotide can be used for the intended purpose (e.g. antisense, RNAi, marker, primer, probe) capable of interacting with a given agent. The upper limit of the length of the polynucleotide of the present invention may be greater than or equal to the full length of the sequence as set forth in SEQ ID NO. 11 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

Therefore, in an illustrative embodiment, the agent of the present invention may be a nucleic acid molecule sequence having a sequence complementary to any of the nucleic acid sequences of the polynucleotides (a) to (g) or a sequence having at least 70% identity thereto.

In another illustrative embodiment, the agent of the present invention may be a nucleic acid molecule hybridizable to any of the nucleic acid sequences of the polynucleotides (a) to (g). Stringency may be high, moderate, or low, which can be determined by those skilled in the art depending on the situation. Stringency may be high, moderate, or low, which can be determined by those skilled in the art depending on the situation.

In another preferred embodiment, the agent of the present invention is an antisense or RNAi. RNAi may be either siRNA or shRNA, for example, double-stranded RNA having a length of about 20 bases (e.g., representatively about 21 to 23 bases) or less than about 20, preferably having a structure having 51-phosphate and 3′-OH, where the 3′ terminus projects by about 2 bases. Preferably, shRNA may have 3′ terminus project. The length of the double-stranded portion is about 10 nucleotides, more preferably about 20 or more nucleotides, but is not particularly limited. Here, the 3′ protruding end is preferably DNA, more preferably DNA of 2 nucleotides in length, even more preferably 2 to 4 nucleotides in length.

(Agent Capable of Specifically Interacting with Rho Kinase in the Polypeptide Form)

In one aspect, the present invention provides a composition comprising an agent capable of specifically interacting with a nucleic acid molecule encoding a Rho kinase polypeptide for regenerating nerves, and a composition comprising an agent capable of specifically interacting with a nucleic acid molecule encoding a Rho polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting (e.g., inhibiting or suppressing) with the Rho kinase polypeptide). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with (a) a polypeptide having an amino acid sequence as set forth in SEQ ID NO. 18 or a fragment thereof; (b) a polypeptide having an amino acid sequence as set forth in SEQ ID NO. 19; (c) a variant polypeptide having the amino acid sequence as set forth in SEQ ID NO. 19 having at least one mutation selected from the group consisting of one or more amino acid substitutions, additions and deletions, and having biological activity; (d) a polypeptide encoded by a splice variant or allelic variant of a base sequence as set forth in SEQ ID NO. 18; (e) a polypeptide which is a species homolog of the amino acid sequence as set forth in SEQ ID NO. 19; or (f) a polypeptide having an amino acid sequence having at least 70% identity to any one of the polypeptides described in (a) to (e), and having biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho kinase gene).

In another preferred embodiment, the allelic variant described in (c) above preferably has at least 99% homology to the amino acid sequence as set forth in SEQ ID NO. 4.

In another preferred embodiment, the above-described species homolog can be identified as described above and preferably has at least about 30% homology to the amino acid sequence as set forth in SEQ ID NO. 19.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 19 or a fragment thereof; an interaction with p75 polypeptide; and the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide with which the agent of the present invention specifically interacts typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 19 as long as the peptide is capable of interacting with a given agent.

In a preferred embodiment, the agent of the present invention is selected from the group consisting of a nucleic acid molecule, a polypeptide, a lipid, a sugar chain, an organic small molecule and a composite molecule thereof. More preferably, the agent of the present invention is antibody or a derivative thereof (e.g., a single chain antibody). Therefore, the agent of the present invention can be used as a probe and/or an inhibitor.

In one embodiment, the Rho kinase polypeptide or fragments or variants thereof comprise amino acids 1 to 1388 of SEQ ID NO. 19. More preferably, the Rho kinase or fragments or variants thereof consist of the whole amino acid sequence of SEQ ID NO. 19.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(Agent Capable of Interacting with a Nucleic Acid Molecule Encoding a Rho Kinase Polypeptide)

In one aspect, the present invention provides a composition comprising an agent capable of specifically interacting with a nucleic acid molecule encoding a Rho kinase polypeptide for regenerating nerves, and a composition comprising an agent capable of specifically interacting with a nucleic acid molecule encoding a Rho kinase polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by the agent capable of specifically interacting with the Rho kinase polypeptide). The effect of nerve regeneration by blocking of a signal transduction pathway has not been conventionally known. Therefore, the present invention provides an effect more excellent than the prior art.

In one embodiment of the present invention, the agent may be an agent capable of specifically interacting with a polynucleotide encoding (a) a polynucleotide having the base sequence as set forth in SEQ ID NO. 18 or a fragment thereof; (b) a polynucleotide encoding a polypeptide having an amino acid sequence as set forth in SEQ ID NO. 19 or a fragment thereof; (c) a polynucleotide encoding a variant polypeptide having the amino acid sequence as set forth in SEQ ID NO. 19 having at least one mutation selected from the group consisting of one or more amino acid substitutions, additions and deletions, and having biological activity; (d) a polynucleotide which is a splice variant or allelic variant of the base sequence as set forth in SEQ ID NO. 18; (e) a polynucleotide encoding a species homolog of the polypeptide having the amino acid sequence as set forth in SEQ ID NO. 19; (f) a polynucleotide hybridizable to any one of the polynucleotides described in (a) to (e) above under stringent conditions and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8, or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the Rho kinase gene).

In another preferred embodiment, the biological activity possessed by the above-described variant polypeptide includes, but is not limited to, for example, an interaction with an antibody specific to the polypeptide having the amino acid sequence as set forth in SEQ ID NO. 19 or a fragment thereof; an interaction with p75; modulation of the functional regulation of Rho by p75 or Rho kinase GDI; and the like. These activities can be measured by, for example, immunological assays, phosphorylation quantification, or the like.

In another preferred embodiment, the allelic variant described in (c) above adventurously has at least 99% homology to the nucleic acid sequence as set forth in SEQ ID NO. 18.

The above-described species homolog can be identified by searching a gene sequence database for the species of the species homolog using the Rho kinase of the present invention as a query sequence, if such a database is available. Alternatively, the species homolog can be identified by using the whole or part of the Rho kinase of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 18.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding Rho kinase or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers. (e.g., 9, 11, 12, 13, 14, 16, and the like) or above the above-specified numbers (e.g., 21, 22 30, and the like). The upper limit of the length of the polynucleotide of the present invention may be greater than or equal to the full length of the sequence as set forth in SEQ ID NO. 18 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, a nucleic acid encoding the Rho kinase polypeptide or fragments or variants thereof comprise porition 1-4164 of the nucleic acid sequence as set forth in SEQ ID NO. 18. More preferably, the Rho kinase or fragments or variants thereof consist of the whole nucleic acid sequence as set forth in SEQ ID NO. 18.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

In a preferred embodiment, the agent of the present invention is selected from the group consisting of a nucleic acid molecule, a polypeptide, a lipid, a sugar chain, an organic small molecule and a composite molecule thereof.

In a preferred embodiment, the agent of the present invention is a nucleic acid molecule. When the agent of the present invention is a nucleic acid molecule, such a nucleic acid molecule may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22 30, and the like). The upper limit of the length of the polynucleotide of the present invention may be greater than or equal to the full length of the sequence as set forth in SEQ ID NO. 18 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

Therefore, in an illustrative embodiment, the agent of the present invention may be a nucleic acid molecule sequence having a sequence complementary to any of the nucleic acid sequences of the polynucleotides (a) to (g) or a sequence having at least 70% identity thereto.

In another illustrative embodiment, the agent of the present invention may be a nucleic acid molecule hybridizable to any of the nucleic acid sequences of the polynucleotides (a) to (9). Stringency may be high, moderate, or low, which can be determined by those skilled in the art depending on the situation. Stringency may be high, moderate, or low, which can be determined by those skilled in the art depending on the situation.

In another preferred embodiment, the agent of the present invention is an antisense or RNAi. RNAi may be either siRNA or shRNA, for example, double-stranded RNA having a length of about 20 bases (e.g., representatively about 21 to 23 bases) or less than about 20, preferably having a structure having 5′-phosphate and 3′-OH, where the 3′ terminus projects by about 2 bases. Preferably, shRNA may have 3′ terminus projects. The length of the double-stranded portion is about 10 nucleotides, more preferably about 20 or more nucleotides, but is not particularly limited. Here, the 3′ protruding end is preferably DNA, more preferably DNA of 2 nucleotides in length, even more preferably 2 to 4 nucleotides in length.

(p21 in the Polypeptide Form)

In one aspect, the present invention provides a composition comprising a p21 polypeptide for regenerating nerves, and a composition comprising a p21 polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by p21). The effect of nerve regeneration by blocking of a signal transduction pathway has not been conventionally known. Therefore, the present invention provides an effect more excellent than the prior art.

In one embodiment of the present invention, p21 used in the present invention or fragments or variants thereof comprise (a) a polypeptide having an amino acid sequence as set forth in SEQ ID NO. 14 or 23 or a fragment thereof; (b) a polypeptide having the amino acid sequence as set forth in SEQ ID NO. 14 or 23 having at least one mutation selected from the group consisting of one or more amino acid substitutions, additions and deletions, and having biological activity; (c) a polypeptide encoded by a splice variant or allelic variant of a base sequence as set forth in SEQ ID NO. 13 or 22; (d) a polypeptide which is a species homolog of the amino acid sequence as set forth in SEQ ID NO. 14 or 23; or (e) a polypeptide having an amino acid sequence having at least 70% identity to any one of the polypeptides described in (a) to (d), and having biological activity.

In one preferred embodiment, the number of substitutions, additions and deletions described in (b) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the p21 gene).

In another preferred embodiment, the allelic variant described in (c) above preferably has at least 99% homology to the amino acid sequence as set forth in SEQ ID NO. 14 or 23.

In another preferred embodiment, the above-described species homolog can be identified as described above and preferably has at least about 30% homology to the amino acid sequence as set forth in SEQ ID NO. 14 or 23.

In another preferred embodiment, the biological activity possessed by the variant polypeptide described in (e) above includes, but is not limited to, for example, an interaction with an antibody specific to the polypeptide having the amino acid sequence as set forth in SEQ ID NO. 14 or 23 or a fragment thereof; an interaction with Rho GTP or Rho kinase; and the like.

In a preferred embodiment, the above-described homology to any one of the polypeptides described in (a) to (d) above may be at least about 80%, more preferably at least about 90%, even more preferably at least about 98%, and most preferably at least about 99%.

The polypeptide of the present invention typically has a sequence of at least 3 contiguous amino acids. The amino acid length of the polypeptide of the present invention may be short as long as the peptide is suitable for an intended application, but preferably a longer sequence may be used. Therefore, the amino acid length may be preferably at least 4, more preferably at least 5, at least 6, at least 7, at least 8, at least 9 and at least 10, even more preferably at least 15, and still even more preferably at least 20. These lower limits of the amino acid length may be present between the above-specified numbers (e.g., 11, 12, 13, 14, 16, and the like) or above the above-specified numbers (e.g., 21, 22, . . . 30, and the like). The upper limit of the length of the polypeptide of the present invention may be greater than or equal to the full length of the sequence as set forth in SEQ ID NO. 14 or 23 as long as the peptide is capable of interacting with a given agent.

In one embodiment, the p21 polypeptide or fragments or variants thereof comprise amino acids. 1 to 140 or 1 to 164 of SEQ ID NO. 14 or 23. More preferably, the p21 peptide or fragments or variants thereof consist of amino acids 1 to 140 or the whole of SEQ ID NO. 14 or 23. In another preferred embodiment, the p21 polypeptide or fragments or variants thereof advantageously comprise 1 to 140 (ΔNLS region) of SEQ ID NO. 14 or 23, and is free of amino acids 141 or later of SEQ ID NO. 14 or 23 (herein referred to as ΔNLS p21). ΔNLS is an abbriviation of nuclear locomotion signal. By inserting a mutation which does not permit the nuclear locomotion signal to function, p21 or fragments or variants thereof can be caused to reside in the cytoplasm, thereby making it possible to suppress or inhibit the p75 signal transduction mechanism. The effect of the present invention can be more advantageously achieved.

In a preferred embodiment, the p21 polypeptide contained in a composition of the present invention may advantageously comprise a PTD domain. A representative sequence of the PTD domain includes, but is not limited to, YGRKKRRQRRR (SEQ ID NO. 20) and a fragment thereof. The PTD domain may be located at any position relative to a nerve regeneration agent (e.g., p21 polypeptide). In a preferred embodiment, the PTD domain may be advantageously located at the N or C terminus of the p21 polypeptide.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(p21 in the Nucleic Acid Form)

In one aspect, the present invention provides a composition comprising a nucleic acid molecule encoding a p21 polypeptide for regenerating nerves, and a composition comprising a nucleic acid molecule encoding a p21 polypeptide for treatment, prophylaxis, diagnosis or prognosis of nervous diseases, nervous disorders or nervous conditions. An effective amount of the composition for regeneration, diagnosis, prophylaxis, treatment, or prognosis can be determined by those skilled in the art based on the disclosures of the present specification using techniques well known in the art with reference to various parameters. For example, such an amount can be determined by those skilled in the art with reference to the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (by p21). The effect of nerve regeneration by blocking of a signal transduction pathway has not been conventionally known. Therefore, the present invention provides an effect more excellent than the prior art.

In one embodiment, the nucleic acid molecule encoding p21 used in the present invention or fragments or variants thereof comprise (a) a polynucleotide having the base sequence as set forth in SEQ ID NO. 13 or 22 or a fragment thereof; (b) a polynucleotide encoding a polypeptide having an amino acid sequence as set forth in SEQ ID NO. 14 or 23 or a fragment thereof; (c) a polynucleotide encoding a variant polypeptide having the amino acid sequence as set forth in SEQ ID NO. 14 or 23 having at least one mutation selected from the group consisting of one or more amino acid substitutions, additions and deletions, and having biological activity; (d) a polynucleotide which is a splice variant or allelic variant of the base sequence as set forth in SEQ ID NO. 13 or 22; (e) a polynucleotide encoding a species homolog of the polypeptide having the amino acid sequence as set forth in SEQ ID NO. 14 or 23; (f) a polynucleotide hybridizable to any one of the polynucleotides described in (a) to (e) above under stringent conditions and encoding a polypeptide having biological activity; and (g) a polynucleotide having a base sequence having at least 70% identity to any one of the polynucleotides described in (a) to (e) or a complementary sequence thereof and encoding a polypeptide having biological activity.

In one preferred embodiment, the number of substitutions, additions and deletions described in (c) above may be limited to, for example, preferably 50 or less, 40 or less, 30 or less, 20 or less, 15 or less, 10 or less, 9 or less, 8 or less, 7 or less, 6 or less, 5 or less, 4 or less, 3 or less, or 2 or less. The number of substitutions, additions and deletions is preferably small, but may be large as long as the biological activity is maintained (preferably, the activity is similar to or substantially the same as that of a product of the p21 gene).

In another preferred embodiment, the biological activity possessed by the above-described variant polypeptide includes, but is not limited to, for example, an interaction with an antibody specific to the polypeptide having the amino acid sequence as set forth in SEQ ID NO. 14 or 23 or a fragment thereof; an interaction with Rho kinase; modulation of the functional regulation of Rho GTP; and the like. These activities can be measured by, for example, immunological assays, phosphorylation quantification, or the like.

In another preferred embodiment, the allelic variant described in (c) above adventurously has at least 99% homology to the nucleic acid sequence as set forth in SEQ ID NO. 13 or 22.

The above-described species homolog can be identified by searching a gene sequence database for the species of the species homolog using the p21 of the present invention as a query sequence, if such a database is available. Alternatively, the species homolog can be identified by using the whole or part of the p21 of the present invention as a probe or a primer to screen gene libraries of the species. Such an identification method is well known in the art and is described in references as described herein. The species homolog preferably has at least about 30% homology to the nucleic acid sequence as set forth in SEQ ID NO. 13 or 22.

In a preferred embodiment, the identity to any one of the polynucleotides described in (a) to (e) above or a complementary sequence thereof may be at least about 80%, more preferably at least about 90%, even more preferably at least about 98%, and most preferably at least about 99%.

In a preferred embodiment, the nucleic acid molecule of the present invention encoding p21 or fragments and variants thereof may have a length of at least 8 contiguous nucleotides. The appropriate nucleotide length of the nucleic acid molecule of the present invention may vary depending on the purpose of use of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the nucleotide length may be present between the above-specified numbers (e.g., 9, 11, 12, 13, 14, 16, and the like) or above the above-specified numbers (e.g., 21, 22 30, and the like). The upper limit of the length of the polynucleotide of the present invention may be greater than or equal to the full length of the sequence as set forth in SEQ ID NO. 13 or 22 as long as the polynucleotide can be used for the intended purpose (e.g. antisense, RNAi, marker, primer, probe, capable of interacting with a given agent). Alternatively, when the nucleic acid molecule of the present invention is used as a primer, the nucleic acid molecule typically may have a nucleotide length of at least about 8, preferably a nucleotide length of about 10. When used as a probe, the nucleic acid molecule typically may have a nucleotide length of at least about 15, and preferably a nucleotide length about 17.

In one embodiment, the nucleic acid molecule encoding p21 or fragments or variants thereof comprise nucleotides 1 to 420 or 1 to 492 of SEQ ID NO. 13 or 22. More preferably, the nucleic acid molecule encoding p21 or fragments or variants thereof consist of nucleotides 1 to 420 or the whole of SEQ ID NO. 13 or 22.

In one embodiment, the p21 polnucleotide or fragments or variants thereof comprise nucleotides 1 to 420 or 1 to 492 of SEQ ID NO. 13 or 22. More preferably, the p21 polynucleotide or fragments or variants thereof consist of nucleotides 1 to 420 or 1 to 492 of SEQ ID NO. 13 or 22. In another preferred embodiment, the p21 polynucleotide or fragments or variants thereof advantageously comprise nucleotides 1 to 420 of SEQ ID NO. 14 or 23 and are free of 421 or later of SEQ ID NO. 13 or 22 (herein referred to as ΔNLS p21). ΔNLS is an abbriviation of nuclear locomotion signal. By inserting a mutation which does not permit the nuclear locomotion signal to function, p21 or fragments or variants thereof can be caused to reside in the cytoplasm, thereby making it possible to suppress or inhibit the p75 signal transduction mechanism. The effect of the present invention can be more advantageously achieved.

In a preferred embodiment, the p21 polypeptide contained in a composition of the present invention may advantageously comprise a PTD domain. A representative sequence of the PTD domain includes, but is not limited to, YGRKKRRQRRR (SEQ ID NO. 20) and a fragment thereof. The PTD domain may be located at any position relative to a nerve regeneration agent (e.g., p21 polypeptide). In a preferred embodiment, the PTD domain may be advantageously located at the N or C terminus of the p21 polypeptide.

In one embodiment, nervous diseases, disorders or conditions to be treated are exemplified herein elsewhere and include, for example, Alzheimer's disease, spinal cord injury, cerebrovascular disorder, brain injury, and the like. Preferably, a nervous disease, disorder or condition intended to be treated by the composition of the present invention may be Alzheimer's disease. In another preferred embodiment, nervous diseases, disorders or conditions intended to be treated by the composition of the present invention may be spinal cord injury, cerebrovascular disorder, and brain injury.

(Action of PTD Domain on Nerve Regeneration)

In another aspect, the present invention provides a composition for regenerating nerves, comprising a TAT PTD domain and a nerve regeneration agent. Here, the TAT PTD domain includes, but is not limited to, representatively an amino acid sequence represented by YGRKKRRQRRR (SEQ ID NO. 20) or variants thereof (e.g., having one or several amino acid substitutions, additions and/or deletions). A nerve regeneration agent used for the composition of the present invention may be selected from the Pep5 polypeptide, the nucleic acid molecule encoding the Pep5 polypeptide, an agent capable of specifically interacting with the p75 polypeptide, an agent capable of specifically interacting with the nucleic acid molecule encoding the p75 polypeptide, a p75 extracellular domain polypeptide, a nucleic acid molecule encoding the p75 extracellular domain polypeptide, an agent capable of specifically interacting with the Rho GDI polypeptide, an agent capable of specifically interacting with an nucleic acid molecule encoding the Rho GDI polypeptide, the Rho GDI polypeptide, the nucleic acid molecule encoding the Rho GDI polypeptide, an agent capable of specifically interacting with the MAG polypeptide, an agent capable of specifically interacting with a nucleic acid molecule encoding the MAC polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with a Rho kinase, an agent capable of specifically interacting with a nucleic acid molecule encoding the Rho kinase, and variants and fragments thereof. The present invention is not so limited.

Therefore, in another aspect, the present invention provides a composition for disrupting inhibition of neurite outgrowth.

(Use of PTD Domain as a Medicament or an Auxiliary for Nerve Regeneration)

In another aspect, the present invention provides a composition for regenerating nerves, comprising a PTD domain and a nerve regeneration agent. The PTD domain has an action of promoting introduction of protein into cells and has been used to introduce a molecule into cells, which is otherwise difficult to introduce into cells, but has not been used for nerve regeneration. Therefore, the present invention provides a novel application of the PTD domain (i.e., an improver for nerve regeneration compositions). Such PTD includes, but is not limited to, representatively, the amino acid sequence YGRKKRRQRRR (SEQ ID NO. 20) or variants or fragments thereof.

Any nerve regeneration agent contained in the regeneration composition comprising the PTD domain of the present invention may be used, preferably an agent which inhibits the p75 signal transduction pathway. Such an agent may include, but is not limited to, a polypeptide, a polynucleotide, an antibody, an antisense, RNAi, and the like.

In another preferred embodiment, the nerve regeneration agent contained in the nerve regeneration composition comprising the PTD domain of the present invention includes, but is not limited to, a transduction agent in the p75 signal transduction pathway or variant or fragments thereof, an agent capable of specifically interacting with the transduction agent in the p75 signal transduction pathway, and the like. Such variants and fragments may advantageously be functionally identical to the original transduction agent or maintain at least one function. The present invention is not so limited. It is optionally preferable that a function is removed from such a variant or fragment.

In another preferred embodiment, the transduction agent in the p75 signal transduction pathway of the nerve regeneration composition comprising the PTD domain of the present invention includesat least one transduction agent selected from the group consisting of MAG, GT1b, p75, Rho GDI, Rho, p21, and Rho kinase. More preferably, an agent active in cells is advantageous. Such an agent active in cells includes, but is not limited to, Rho GDI, Rho, and Rho kinase. Examples of inhibitors against Rho GDI, Rho, and Rho kinase include, but are not limited to, p21 or variants or fragments thereof, and Pep5 or variants or fragments thereof. It was elucidated that a combination of such an agent and the PTD domain noticeably enhances the nerve regeneration effect which was first found in the present invention. Such an effect had not been conventionally found and can be said to be surprising.

In another preferred embodiment, in the nerve regeneration composition comprising the PTD domain of the present invention, the nerve regeneration agent may have at least one action selected from the group consisting of inhibition of the interaction between MAG and GT1b, inhibition of the interaction between GT1b and p75, inhibition of the interaction between p75 and Rho, inhibition of the interaction between p75 and Rho GDI, maintenance and enhancement of the interaction between Rho and Rho GDI, inhibition of transformation of Rho GDP to Rho GTP, inhibition of the interaction between Rho and Rho kinase, and inhibition of activity of Rho kinase. Such an action can be observed by preparing two or more related molecules, contacting the molecules with the composition of the present invention, and determining whether or not the interaction between the molecules is changed.

In another preferred embodiment, in the nerve regeneration composition comprising the PTD domain of the present invention, the nerve regeneration agent may include, but is not limited to, at least one selected from the group consisting of agent capable of inhibiting or extinguishing the interaction between MAG and GT1b, agent capable of inhibiting or extinguishing the interaction between GT1b and p75, agent capable of inhibiting or extinguishing the interaction between p75 and Rho GDI, agent capable of inhibiting or extinguishing the interaction between p75 and Rho, an agent capable of maintaining or enhancing the interaction between Rho and Rho GDI, agent capable of inhibiting the conversion from Rho GDP to Rho GTP, agent capable of inhibiting the interaction between Rho and Rho kinase, and agent capable of inhibiting the activity of Rho kinase. Such an agent may be, but is not limited to, a polypeptide, a polynucleotide, a low molecular weight molecule, an antibody, RNAi, an antisense, or the like. Such an agent is described in detail elsewhere herein.

In another preferred embodiment, in the nerve regeneration composition of the PTD domain of the present invention, the nerve regeneration agent may include an agent selected from the group consisting of the Pep5 polypeptide, the nucleic acid molecule encoding the Pep5 polypeptide, an agent capable of specifically interacting with the p75 polypeptide, an agent capable of specifically interacting with the nucleic acid molecule encoding the p75 polypeptide, a p75 extracellular domain polypeptide, a nucleic acid molecule encoding the p75 extracellular domain polypeptide, an agent capable of specifically interacting with the Rho GDI polypeptide, an agent capable of specifically interacting with an nucleic acid molecule encoding the Rho GDI polypeptide, the Rho GDI polypeptide, the nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with a Rho kinase, an agent capable of specifically interacting with a nucleic acid molecule encoding the Rho kinase, and variants and fragments thereof. Such an agent may be, but is not limited to, a polypeptide, a polynucleotide, a low molecular weight molecule, an antibody, RNAi, an antisense, or the like. Such an agent is described in detail elsewhere herein.

In another preferred embodiment, the PTD domain may have the amino acid sequence YGRKKRRQRRR or the sequence having one or several substitutions, additions and/or deletions. In this case, it is preferable that the activity of introduction into cytoplasm is not lost due to such substitutions, additions and/or deletions. Such introduction activity can be found by determining where a desired polypeptide is expressed within a cell which has been transformed with a nucleic acid molecule encoding the polypeptide comprising the domain.

In a preferred embodiment, the PTD domain may be advantageously located at the C-terminus or the N-terminus of the nerve regeneration agent. This is because such location provides a desired activity (i.e., introduction into cytoplasm) without impairing the activity of the nerve regeneration agent. Therefore, preferably, the nerve regeneration agent contained in the nerve regeneration composition comprising the PTD of the present invention may reside in the cytoplasm. The residence time may be, for example, at least several hours, several days, or several months, though the residence time may be shorter or longer as long as the nerve regeneration effect is exhibited. Such a composition can be used in the present invention.

(Use of PTD in the Nucleic Acid Form as a Nerve Regeneration Medicament or an Auxiliary)

In another aspect, the present invention provides a composition for regenerating nerves comprising the PTD domain and nerve regeneration agent in the nucleic acid form. Therefore, the present invention provides a composition for regenerating nerves comprising a nucleic acid molecule having a nucleic acid sequence encoding the PTD domain and a nucleic acid sequence encoding a nerve regeneration agent. Such a nucleic acid molecule achieves the improved nerve regeneration effect as with the above-described protein molecules. Therefore, this form of the present invention can also achieve an unexpected, suprising effect. The present invention also provides a novel application of the nucleic acid molecule encoding the PTD domain (i.e., an improver for the nerve regeneration composition). Such a PTD includes, but is not limited to, representatively, a nucleic acid sequence encoding the amino acid sequence indicated by YGRKKRRQRRR (SEQ ID NO. 20) or variants or fragments thereof. Alternatively, such a nucleic acid molecule may be derived from a nucleic acid sequence (SEQ ID NO. 21) of HIV TAT.

Any nucleic acid sequence encoding a nerve regeneration agent contained in the nerve regeneration composition comprising a nucleic acid sequence of the PTD domain of the present invention may be used, but preferably, a nucleic acid sequence encoding a nerve regeneration agent inhibiting the p75 signal transduction pathway may be advantageous.

The nucleic acid sequence encoding a nerve regeneration agent contained in the nerve regeneration composition comprising a nucleic acid sequence of the PTD domain of the present invention preferably encodes a transduction agent in the p75 signal transduction pathway or a variant or fragment thereof, or an agent capable of specifically interacting with a transduction agent in the p75 signal transduction pathway. Such an agent may be, but is not limited to, a polypeptide, an antibody, or the like. Such an agent is described in detail elsewhere herein.

The transduction agent in the p75 signal transduction pathway targeted by the nerve regeneration composition comprising a nucleic acid sequence encoding the PTD domain of the present invention may include at least one transduction agent selected from the group consisting of MAG, GT1b, p75, Rho GDI, Rho, p21, and Rho kinase. It was revealed that the combination of the agent inhibiting the p75 signal transduction pathway and the PTD domain noticeably enhances the nerve regeneration effect which was first found in the present invention. Such an effect had not been conventionally found and can be said to be surprising.

In the nerve regeneration composition comprising a nucleic acid sequence encoding the PTD domain of the present invention, the nerve regeneration agent may have at least one action selected from the group consisting of inhibition of the interaction between MAG and GT1b, inhibition of the interaction between GT1b and p75, inhibition of the interaction between p75 and Rho, inhibition of the interaction between p75 and Rho GDI, maintenance and enhancement of the interaction between Rho and Rho GDI, inhibition of transformation of Rho GDP to Rho GTP, inhibition of the interaction between Rho and Rho kinase, and inhibition of activity of Rho kinase. Such an action can be observed by preparing two or more related molecules, contacting the molecules with the composition of the present invention, and determining whether or not the interaction between the molecules is changed.

In the nerve regeneration composition comprising a nucleic acid sequence encoding the PTD domain of the present invention, the nerve regeneration agent may include, but is not limited to, at least one selected from the group consisting of agent capable of inhibiting or extinguishing the interaction between MAG and GT1b, agent capable of inhibiting or extinguishing the interaction between GT1b and p75, agent capable of inhibiting or extinguishing the interaction between p75 and Rho GDI, agent capable of inhibiting or extinguishing the interaction between p75 and Rho, an agent capable of maintaining or enhancing the interaction between Rho and Rho GDI, agent capable of inhibiting the conversion from Rho GDP to Rho GTP, agent capable of inhibiting the interaction between Rho and Rho kinase, and agent capable of inhibiting the activity of Rho kinase. Such an agent may be advantageously capable of be linked to PTD.

In the nerve regeneration composition comprising a nucleic acid sequence encoding the PTD domain of the present invention, the nerve regeneration agent may include an agent selected from the group consisting of the Pep5 polypeptide, an agent capable of specifically interacting with the p75 polypeptide, an agent capable of specifically interacting with the nucleic acid molecule encoding the p75 polypeptide, a p75 extracellular domain polypeptide, the Rho GDI polypeptide, an agent capable of specifically interacting with the MAG polypeptide, an agent capable of specifically interacting with a nucleic acid molecule encoding the MAG polypeptide, the p21 polypeptide, an agent capable of specifically interacting with the Rho polypeptide, an agent capable of specifically interacting with a nucleic acid molecule encoding the Rho polypeptide, an agent capable of specifically interacting with a Rho kinase, an agent capable of specifically interacting with a nucleic acid molecule encoding the Rho kinase, and variants and fragments thereof. Such an agent may be advantageously capable of be linked to PTD.

In the nerve regeneration composition comprising a nucleic acid sequence encoding the PTD domain of the present invention, the PTD domain may have the amino acid sequence YGRKKRRQRRR or the sequence having one or several substitutions, additions and/or deletions.

In the nerve regeneration composition comprising a nucleic acid sequence encoding the PTD domain of the present invention, the PTD domain may be advantageously located at the C-terminus or the N-terminus of the nerve regeneration agent. This is because such location provides a desired activity (i.e., introduction into cytoplasm) without impairing the activity of the nerve regeneration agent. Therefore, preferably, the nerve regeneration agent contained in the nerve regeneration composition comprising the PTD of the present invention may reside in the cytoplasm. The residence time may be, for example, at least several hours, several days, or several months, though the residence time may be shorter or longer as long as the nerve regeneration effect is exhibited. Such a composition can be used in the present invention.

(Method for Nerve Regeneration)

In another aspect, the present invention provides a method for regenerating nerves. This method comprises a step of inhibiting the p75 signal transduction pathway. In the present invention, it was unexpectedly found that inhibition of the p75 signal transduction pathway leads to nerve regeneration. This fact had not been expected from the conventional art and can be said to be an unexpected effect. Therefore, the mechanism of nerve regeneration by inhibiting the p75 signal transduction pathway can be used for various treatments, such as treatment, prophylaxis, diagnosis, prognosis, and the like for nervous disease, disorder, or abnormal condition. The present invention is not so limited. Preferably, the p75 signal transduction pathway is present in neurons at a site desired for nerve regeneration. When the p75 signal transduction pathway in the target neurons are inhibited or suppressed, nerve blocking is reduced or inhibited (disrupted), so that nerve regeneration can be advantageously produced at a desired site.

In one embodiment, the inhibition of the p75 signal transduction pathway may be achieved by providing a transduction agent in the p75 signal transduction pathway or a variant or fragment thereof (preferably, such a variant or fragment has a function similar to that of the transduction agent), or an agent capable of specifically interacting with a transduction agent in the p75 signal transduction pathway in an amount effective for nerve regeneration.

In another embodiment, the transduction agent in the p75 signal transduction pathway may include, but is not limited to, at least one transduction agent selected from the group consisting of MAG, GT1b, p75, Rho GDI, Rho, p21, and Rho kinase. A method for inhibiting or suppressing such a transduction agent includes, but is not limited to, a method of administering or providing an agent capable of specifically interacting with the transduction agent or a nucleic acid molecule encoding the transduction agent; a method of reducing, suppressing, or inhibiting the expression of the transduction agent; a method of introducing a mutation which inhibits the function of the transduction agent; and the like.

In another embodiment, inhibition of the p75 signal transduction pathway may be selected from the group consisting of inhibition of the interaction between MAG and GT1b, inhibition of the interaction between GT1b and p75, inhibition of the interaction between p75 and Rho, inhibition of the interaction between p75 and Rho GDI, maintenance or enhancement of the interaction between Rho and Rho GDI, inhibition of the conversion from Rho GDP to Rho GTP, inhibition of the interaction between Rho and Rho kinase, and inhibition of the activity of Rho kinase. The present invention is not so limited. The inhibition of the interaction may be achieved by administering an inhibitor, providing a specifically interactive agent, or the like. The maintenance or enhancement of the interaction may be achieved by eliminating an agent weakening the interaction, increasing the amount of related molecules, or the like. The present invention is not so limited.

In another embodiment, the inhibition of the p75 signal transduction pathway may be achieved by providing, in an amount effective for nerve regeneration, at least one agent selected from the group consisting of agent capable of inhibiting or extinguishing the interaction between MAG and GT1b, agent capable of inhibiting or extinguishing the interaction between GT1b and p75, agent capable of inhibiting or extinguishing the interaction between p75 and Rho GDI, agent capable of inhibiting or extinguishing the interaction between p75 and Rho, an agent capable of maintaining or enhancing the interaction between Rho and Rho GDI, agent capable of inhibiting the conversion from Rho GDP to Rho GTP, agent capable of inhibiting the interaction between Rho and Rho kinase, and agent capable of inhibiting the activity of Rho kinase.

In the nerve regeneration method of the present invention, nerve regeneration may be carried out in vivo or in vitro. In the case of in vivo, therapeutic or prophylactic treatments or the like may be carried out directly within the body. In the case of in vitro, a nerve population can be prepared. In one embodiment, nerves are in a condition, including spinal cord injury, cerebrovascular disorder or brain injury. Alternatively, a nerve to be treated may be in a condition of any nervous disease, nervous disorder or abnormal condition illustrated elsewhere herein. Such a disease, disorder or condition includes, but is not limited to, brain injury, spinal cord injury, stroke, demyelinating diseases (monophasic demyelination), encephalomyelitis, multifocal leukoencephalopathy, panencephalitis, Marchiafava-Bignami disease, Spongy degeneration, Alexander's disease, Canavan's disease, metachromatic leukodystrophy and Krabbe's disease.

In another embodiment, the step of inhibiting the p75 signal transduction pathway in the nerve regeneration method of the present invention may be achieved by a step of providing, in an amount effective for nerve regeneraton, to a desired nerve, a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase, an agent capable of specifically interacting with a nucleic acid molecule encoding the Rho kinase, and variants and fragments thereof.

In the nerve regeneration method of the present invention, an agent for nerve regeneration may be provided in linkage with the PTD domain.

In the nerve regeneration method of the present invention, an amount effective for nerve regeneration can be determined by those skilled in the art using techniques well known in the art with reference to various parameters, including the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (e.g., via an agent related to the p75 signal transduciton pathway). The effect of nerve regeneration by blocking of the signal transduction pathway has not been conventionally known. Therefore, the present invention provides an effect more excellent than the conventional art.

In one embodiment, the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof can be in forms as described above. In the present invention, it was revealed that regeneration of nerves occurs due to inhibition of neurite outgrowth being disrupted by blocking of the p75 signal transduction pathway. The effect of nerve regeneration by blocking of the signal transduction pathway has not been conventionally known. Therefore, the present invention provides an effect more excellent than the conventional art. Particularly, the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho. GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof may be preferably used in combination. In this case, various combinations may be used. Preferably, two, three or four polypeptides, polynucleotides and/or agents may be used. In another preferred embodiment, a plurality of molecules may be advantageously inhibited on the pathway.

In another aspect, the present invention also provides a composition for regenerating nerves. This composition comprises an agent capable of inhibiting the p75 signal transduction pathway in an amount effective for regeneration. Such a composition can be prepared using techniques well known in the art as described herein.

In one embodiment, the agent capable of inhibiting the p75 signal transduction pathway may be a transduction agent in the p75 signal transduction pathway or a variant or fragment thereof (preferably, such a variant or fragment has a function similar to that of the transduction agent), or an agent capable of specifically interacting with the transduction agent in the p75 signal transduction pathway. The agent may be contained in a composition of the present invention in an amount effective for regeneration.

In another embodiment, the transduction agent in the p75 signal transduction pathway comprises at least one transduction agent selected from the group consisting of MAG, GT1b, p75, Rho GDI, Rho, p21, and Rho kinase.

In another embodiment, the agent capable of inhibiting the p75 signal transduction pathway may have at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In another embodiment, the agent capable of inhibiting the p75 signal transduction pathway may be at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase. Such an agent is present in an amount effective for regeneration.

In another embodiment, the agent capable of inhibiting the p75 signal transduction pathway comprises, in an amount effective for nerve regeneration, at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. Such an amount effective for nerve regeneration can be determined by those skilled in the art using techniques well known in the art with reference to various parameters, including the purpose of use, a target disease (type, severity, and the like), the patient's age, weight, sex and case history, the form or type of the cells, and the like.

In another embodiment, the agent capable of inhibiting the p75 signal transduction pathway comprises, in an amount effective for diagnosis, prophylaxis, treatment or prognosis, at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. Such an amount effective for diagnosis, prophylaxis, treatment or prognosis, can be determined by those skilled in the art using techniques well known in the art with reference to various parameters, including the purpose of use, a target disease (type, severity, and the like), the patient's age, weight, sex and case history, the form or type of the cells, and the like.

In another aspect, the present invention provides a composition for regenerating nerves, comprising a plurality of elements selected from the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. In this case, various combinations may be used. Preferably, two, three or four polypeptides, polynucleotides and/or agents can be used. In another preferred embodiment, a substance inhibiting a plurality of molecules on the pathway may be advantageously used.

An agent used in a composition of the present invention may comprise the PTD domain.

The present invention also relates to a nerve regeneration kit comprising the above-described composition. Such a kit may comprise instructions describing an administration method in addition to a composition of the present invention. The instructions are described elsewhere herein.

The present invention also relates to use of an agent capable of inhibiting the p75 transduction pathway for preparation of a nerve regeneration medicament.

(Diagnosis, Prophylaxis, Treatment or Prognosis for Neurological Diseases, Disorders or Conditions)

In another aspect, the present invention provides a method for diagnosis, prophylaxis, treatment or prognosis for neurological diseases, disorders or conditions. This method comprises a step of inhibiting the p75 signal transduction pathway. In the present invention, it was unexpectedly found that inhibition of the p75 signal transduction pathway can be utilized in diagnosis, prophylaxis, treatment or prognosis for neurological diseases, disorders or conditions. This fact had not been expected from the conventional art and can be said to be an unexpected effect.

Preferably, the p75 signal transduction pathway is present in neurons at a site desired for diagnosis, prophylaxis, treatment or prognosis for neurological diseases, disorders or conditions. When the p75 signal transduction pathway in the target neurons are inhibited or suppressed, nerve blocking is reduced or inhibited (disrupted), so that nerve regeneration can be advantageously produced at a desired site.

In one embodiment, the inhibition of the p75 signal transduction pathway may be achieved by providing a transduction agent in the p75 signal transduction pathway or a variant or fragment thereof (preferably, such a variant or fragment has a function similar to that of the transduction agent), or an agent capable of specifically interacting with a transduction agent in the p75 signal transduction pathway in an amount effective for nerve regeneration.

In another embodiment, the transduction agent in the p75 signal transduction pathway may include, but is not limited to, at least one transduction agent selected from the group consisting of MAG, GT1b, p75, Rho GDI, Rho, p21, and Rho kinase. A method for inhibiting or suppressing such a transduction agent includes, but is not limited to, a method of administering or providing an agent capable of specifically interacting with the transduction agent or a nucleic acid molecule encoding the transduction agent; a method of reducing, suppressing, or inhibiting the expression of the transduction agent; a method of introducing a mutation which inhibits the function of the transduction agent; and the like.

In another embodiment, inhibition of the p75 signal transduction pathway may be selected from the group consisting of inhibition of the interaction between MAG and GT1b, inhibition of the interaction between GT1b and p75, inhibition of the interaction between p75 and Rho, inhibition of the interaction between p75 and Rho GDI, maintenance or enhancement of the interaction between Rho and Rho GDI, inhibition of the conversion from Rho GDP to Rho GTP, inhibition of the interaction between Rho and Rho kinase, and inhibition of the activity of Rho kinase. The present invention is not so limited. The inhibition of the interaction may be achieved by administering an inhibitor, providing a specifically interactive agent, or the like. The maintenance or enhancement of the interaction may be achieved by eliminating an agent weakening the interaction, increasing the amount of related molecules, or the like. The present invention is not so limited.

In another embodiment, the inhibition of the p75 signal transduction pathway may be achieved by providing, in an amount effective for nerve regeneration, at least one agent selected from the group consisting of agent capable of inhibiting or extinguishing the interaction between MAG and GT1b, agent capable of inhibiting or extinguishing the interaction between GT1b and p75, agent capable of inhibiting or extinguishing the interaction between p75 and Rho GDI, agent capable of inhibiting or extinguishing the interaction between p75 and Rho, an agent capable of maintaining or enhancing the interaction between Rho and Rho GDI, agent capable of inhibiting the conversion from Rho GDP to Rho GTP, agent capable of inhibiting the interaction between Rho and Rho kinase, and agent capable of inhibiting the activity of Rho kinase.

In the method for diagnosis, prophylaxis, treatment or prognosis for neurological diseases, disorders or conditions according to the present invention, nerve regeneration may be carried out in vivo or ex vivo. In the case of in vivo therapeutic or prophylactic treatments or the like may be carried out directly within the body. In the case of ex vivo, a nerve population is prepared and the population can be prepared for each patient or subject.

In one embodiment, nerves are in a condition, including spinal cord injury, cerebrovascular disorder or brain injury. Alternatively, a nerve to be treated may be in a condition of any nervous disease, nervous disorder or abnormal condition illustrated elsewhere herein. Such a disease, disorder or condition includes, but is not limited to, brain injury, spinal cord injury, stroke, demyelinating diseases (monophasic demyelination), encephalomyelitis, multifocal leukoencephalopathy, panencephalitis, Marchiafava-Bignami disease, Spongy degeneration, Alexander's disease, Canavan's disease, metachromatic leukodystrophy and Krabbe's disease.

In another embodiment, the step of inhibiting the p75 signal transduction pathway in the method for diagnosis, prophylaxis, treatment or prognosis for neurological diseases, disorders or conditions may be achieved by a step of providing, in an amount effective for nerve regeneraton, to a desired nerve, a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof.

In the method for diagnosis, prophylaxis, treatment or prognosis for neurological diseases, disorders or conditions according to the present invention, an agent for nerve regeneration may be provided in linkage with the PTD domain.

In one embodiment, the method for diagnosis, prophylaxis, treatment or prognosis for neurological diseases, disorders or conditions according to the present invention comprises a step of providing, in an amount effective for nerve regeneraton, to a desired nerve, a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. An amount effective for nerve regeneration can be determined by those skilled in the art using techniques well known in the art with reference to various parameters, including the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (e.g., via an agent related to the p75 signal transduciton pathway). The effect of nerve regeneration by blocking of the signal transduction pathway has not been conventionally known. Therefore, the present invention provides an effect more excellent than the conventional art.

In one embodiment, the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof can be in forms as described above. In the present invention, it was revealed that regeneration of nerves occurs due to inhibition of neurite outgrowth being disrupted by blocking of the p75 signal transduction pathway. The effect of nerve regeneration by blocking of the signal transduction pathway has not been conventionally known. Therefore, the present invention provides an effect more excellent than the conventional art. Particularly, the Peps polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho. GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof may be used in combination. In this case, various combinations may be used. Preferably, two, three or four polypeptides, polynucleotides and/or agents can be used. In another preferred embodiment, a plurality of molecules may be advantageously inhibited on the pathway.

In another aspect, the present invention provides a composition for diagnosis, prophylaxis, treatment or prognosis for neurological diseases, disorders or conditions. This composition comprises an agent capable of inhibiting the p75 signal transduction pathway in an amount effective for diagnosis, prophylaxis, treatment or prognosis. Such a composition can be prepared using techniques well known in the art as described herein.

In one embodiment, the agent capable of inhibiting the p75 signal transduction pathway may be a transduction agent in the p75 signal transduction pathway or a variant or fragment thereof (preferably, such a variant or fragment has a function similar to that of the transduction agent), or an agent capable of specifically interacting with the transduction agent in the p75 signal transduction pathway. The agent may be contained in a composition of the present invention in an amount effective for regeneration.

In another embodiment, the transduction agent in the p75 signal transduction pathway comprises at least one transduction agent selected from the group consisting of MAG, GT1b, p75, Rho GDI, Rho, p21, and Rho kinase.

In another embodiment, the agent capable of inhibiting the p75 signal transduction pathway may have at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In another embodiment, the agent capable of inhibiting the p75 signal transduction pathway may be at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase. Such an agent is present in an amount effective for diagnosis, prophylaxis, treatment or prognosis. In another embodiment, an agent capable of inhibiting the p75 signal transduction pathway comprises, in an amount effective for diagnosis, prophylaxis, treatment or prognosis, at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. Here, an amount effective for diagnosis, prophylaxis, treatment or prognosis can be determined by those skilled in the art using techniques well known in the art with reference to various parameters, including the purpose of use, a target disease (type, severity, and the like), the patient's age, weight, sex and case history, the form or type of the cells, and the like.

In another preferred embodiment, the present invention also provides a composition for diagnosis, prophylaxis, treatment or prognosis for neurological diseases, disorders or conditions, comprising a plurality of elements selected from the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. In this case, various combinations may be used. Preferably, two, three or four polypeptides, polynucleotides and/or agents can be used. In another preferred embodiment, a substance inhibiting a plurality of molecules on the pathway may be advantageously used.

An agent used in a composition of the present invention may comprise the PTD domain.

The present invention also relates to a kit comprising the above-described composition for diagnosis, prophylaxis, treatment or prognosis of nervous diseases, disorders and conditions. Such a kit may comprise instructions describing an administration method in addition to a composition of the present invention. The instructions are described elsewhere herein.

The present invention also relates to use of an agent capable of inhibiting the p75 transduction pathway for preparation of a medicament diagnosis, prophylaxis, treatment or prognosis of nervous diseases, disorders and conditions.

(Method for Disrupting or Reducing Inhibition of Neurite Outgrowth)

In another aspect, the present invention provides a method for disrupting or reducing inhibition of neurite outgrowth. This method comprises a step of inhibiting the p75 signal transduction pathway. In the present invention, it was unexpectedly found that inhibition of the p75 signal transduction pathway leads to nerve regeneration. This fact had not been expected from the conventional art and can be said to be an unexpected effect.

Preferably, the p75 signal transduction pathway is present in neurons at a site desired for disruption or reduction of inhibition of neurite outgrowth. When the p75 signal transduction pathway in the target neurons are inhibited or suppressed, nerve blocking is reduced or inhibited (disrupted), so that disruption or reduction of inhibition of neurite outgrowth can be advantageously produced at a desired site.

In one embodiment, the inhibition of the p75 signal transduction pathway may be achieved by providing a transduction agent in the p75 signal transduction pathway or a variant or fragment thereof (preferably, such a variant or fragment has a function similar to that of the transduction agent), or an agent capable of specifically interacting with a transduction agent in the p75 signal transduction pathway in an amount effective for nerve regeneration.

In another embodiment, the transduction agent in the p75 signal transduction pathway may include, but is not limited to, at least one transduction agent selected from the group consisting of MAG, GT1b, p75, Rho GDI, Rho, p21, and Rho kinase. A method for inhibiting or suppressing such a transduction agent includes, but is not limited to, a method of administering or providing an agent capable of specifically interacting with the transduction agent or a nucleic acid molecule encoding the transduction agent; a method of reducing, suppressing, or inhibiting the expression of the transduction agent; a method of introducing a mutation which inhibits the function of the transduction agent; and the like.

In another embodiment, inhibition of the p75 signal transduction pathway may be selected from the group consisting of inhibition of the interaction between MAG and GT1b, inhibition of the interaction between GT1b and p75, inhibition of the interaction between p75 and Rho, inhibition of the interaction between p75 and Rho GDI, maintenance or enhancement of the interaction between Rho and Rho GDI, inhibition of the conversion from Rho GDP to Rho GTP, inhibition of the interaction between Rho and Rho kinase, and inhibition of the activity of Rho kinase. The present invention is not so limited. The inhibition of the interaction may be achieved by administering an inhibitor, providing a specifically interactive agent, or the like. The maintenance or enhancement of the interaction may be achieved by eliminating an agent weakening the interaction, increasing the amount of related molecules, or the like. The present invention is not so limited.

In another embodiment, the inhibition of the p75 signal transduction pathway may be achieved by providing, in an amount effective for nerve regeneration, at least one agent selected from the group consisting of agent capable of inhibiting or extinguishing the interaction between MAG and GT1b, agent capable of inhibiting or extinguishing the interaction between GT1b and p75, agent capable of inhibiting or extinguishing the interaction between p75 and Rho GDI, agent capable of inhibiting or extinguishing the interaction between p75 and Rho, an agent capable of maintaining or enhancing the interaction between Rho and Rho GDI, agent capable of inhibiting the conversion from Rho GDP to Rho GTP, agent capable of inhibiting the interaction between Rho and Rho kinase, and agent capable of inhibiting the activity of Rho kinase.

In the method for disrupting or reducing inhibition of neurite outgrowth according to the present invention, nerve regeneration may be carried out in vivo or ex vivo. In the case of in vivo therapeutic or prophylactic treatments or the like may be carried out directly within the body. In the case of ex vivo, a nerve population is prepared and the population can be prepared for each patient or subject.

In one embodiment, nerves are in a condition, including spinal cord injury, cerebrovascular disorder or brain injury. Alternatively, a nerve to be treated may be in a condition of any nervous disease, nervous disorder or abnormal condition illustrated elsewhere herein. Such a disease, disorder or condition includes, but is not limited to, brain injury, spinal cord injury, stroke, demyelinating diseases (monophasic demyelination), encephalomyelitis, multifocal leukoencephalopathy, panencephalitis, Marchiafava-Bignami disease, Spongy degeneration, Alexander's disease, Canavan's disease, metachromatic leukodystrophy and Krabbe's disease.

In another embodiment, the step of inhibiting the p75 signal transduction pathway in the method for disrupting or reducing inhibition of neurite outgrowth may be achieved by providing, in an amount for nerve regeneration, a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof.

In the method for disrupting or reducing inhibition of neurite outgrowth according to the present invention, an agent for nerve regeneration may be provided in linkage with the PTD domain.

In one embodiment, the method for disrupting or reducing inhibition of neurite outgrowth according to the present inventioncomprising a step of providing, in an amount effective for nerve regeneraton, to a desired nerve, a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. Here, an amount effective for nerve regeneration can be determined by those skilled in the art using techniques well known in the art with reference to various parameters, including the purpose of use, a target disease (type, severity, and the like), the patient's age, weight, sex and case history, the form or type of the cells, and the like (see Shinkei-Naika Chiryo Gaido [Guidance to Treatments in Neurological Internal Medicine], Norio Ogawa, Chugai-Igaku 1994). In the present invention, it was revealed that regeneration of nerves occurs due to inhibition of neurite outgrowth being disrupted by blocking of the p75 signal transduction pathway (e.g., via an agent related to the p75 signal transduciton pathway). The effect of nerve regeneration by blocking of the signal transduction pathway has not been conventionally known. Therefore, the present invention provides an effect more excellent than the conventional art.

In one embodiment, the Pep5 polypeptide, a nucleic acid molecule encoding the Peps polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof can be in forms as described above. In the present invention, it was revealed that regeneration of nerves occurs due to inhibition of neurite outgrowth being disrupted by blocking of the p75 signal transduction pathway. The effect of nerve regeneration by blocking of the signal transduction pathway has not been conventionally known. Therefore, the present invention provides an effect more excellent than the conventional art. Particularly, the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof may be preferably used in combination. In this case, various combinations may be used. Preferably, two, three or four polypeptides, polynucleotides and/or agents can be used. In another preferred embodiment, a plurality of molecules may be advantageously inhibited on the pathway.

In another aspect, the present invention provides a comosition for disrupting or reducing inhibition of neurite outgrowth. This composition comprises an agent capable of inhibiting the p75 signal transduction pathway in an amount effective for regeneration. Such a composition can be prepared using techniques well known in the art as described herein.

In one embodiment, the agent capable of inhibiting the p75 signal transduction pathway may be a transduction agent in the p75 signal transduction pathway or a variant or fragment thereof (preferably, such a variant or fragment has a function similar to that of the transduction agent), or an agent capable of specifically interacting with the transduction agent in the p75 signal transduction pathway. The agent may be contained in a composition of the present invention in an amount effective for regeneration.

In another embodiment, the transduction agent in the p75 signal transduction pathway comprises at least one transduction agent selected from the group consisting of MAG, GT1b, p75, Rho GDI, Rho, p21, and Rho kinase.

In another embodiment, the agent capable of inhibiting the p75 signal transduction pathway may have at least one action selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

In another embodiment, the agent capable of inhibiting the p75 signal transduction pathway may be at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase. Such an agent is present in an amount effective for disruption or reduction of inhibition of neurite outgrowth.

In another embodiment, the agent capable of inhibiting the p75 signal transduction pathway comprises, in an amount effective for disruption or reduction of inhibition of neurite outgrowth, at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. Such an amount effective for disruption or reduction of inhibition of neurite outgrowth can be determined by those skilled in the art using techniques well known in the art with reference to various parameters, including the purpose of use, a target disease (type, severity, and the like), the patient's age, weight, sex and case history, the form or type of the cells, and the like.

In another preferred embodiment, the present invention also provides a composition for disrupting or reducing inhibition of neurite outgrowth, comprising a plurality of elements selected from the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. In this case, various combinations may be used. Preferably, two, three or four polypeptides, polynucleotides and/or agents can be used.

In another preferred embodiment, a substance inhibiting a plurality of molecules on the pathway may be advantageously used.

An agent used in a composition of the present invention may comprise the PTD domain.

The present invention also relates to a kit comprising the above-described composition for disruption or reduction of inhibition of neurite outgrowth, disorders and conditions. Such a kit may comprise instructions describing an administration method in addition to a composition of the present invention. The instructions are described elsewhere herein.

The present invention also relates to use of an agent capable of inhibiting the p75 transduction pathway for disruption or reduction of inhibition of neurite outgrowth, disorders and conditions.

(Construction of a Network of Neurons)

In another aspect, the present invention also provides a composition and method for constructing a network of neurons. The composition and method comprises an agent for inhibiting the p75 signal transduction pathway in neurons, or a step of inhibiting the p75 signal transduction pathway in neurons.

As used herein, construction of a network of neurons refers to interconnection between a plurality of neurons so that organic matter or information is transferred between the cells. Neurons forming such a network is also referred to as a neuron population. Examples of neurons forming such a network include, but are not limited to, a population of neurons interconnected via synapses, the brain, the spinal cord, the peripheral nerve, and the like.

In one embodiment, in the composition and method for constructing a network of neurons according to the present invention, the inhibition of the p75 signal transduction pathway may be achieved by providing a transduction agent in the p75 signal transduction pathway or a variant or fragment thereof, or an agent capable of specifically interacting with the p75 signal transduction pathway to the neurons in an amount effective for nerve regeneration.

In another embodiment, in the composition and method for constructing a network of neurons according to the present invention, the transduction agent in the p75 signal transduction pathway may include at least one transduction agent selected from the group consisting of MAG, GT1b, p75, Rho GDI, Rho, p21, and Rho kinase.

In another embodiment, in the composition and method for constructing a network of neurons according to the present invention, the inhibition of the p75 signal transduction pathway may be achieved by modulation of an interaction selected from the group consisting of inhibition of the interaction between MAG and GT1b, inhibition of the interaction between GT1b and p75, inhibition of the interaction between p75 and Rho, inhibition of the interaction between p75 and Rho GDI, maintenance or enhancement of the interaction between Rho and Rho GDI, inhibition of the conversion from Rho GDP to Rho GTP, inhibition of the interaction between Rho and Rho kinase, and inhibition of the activity of Rho kinase.

The composition for constructing a network of neurons comprises at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof. Here, an amount effective for construction of a network of neurons can be determined by those skilled in the art using techniques well known in the art with reference to various parameters, including the purpose of use, a target disease (type, severity, and the like), the patient's age, weight, sex and case history, the form or type of the cells, and the like. In the present invention, it was revealed that regeneration of nerves occurs due to inhibition of neurite outgrowth being disrupted by blocking of the p75 signal transduction pathway. The effect of nerve regeneration by blocking of the signal transduction pathway has not been conventionally known. Therefore, the present invention provides an effect more excellent than the conventional art.

The thus-obtained neurons (population) forming a network can be transplanted to organisms having a nervous disorder.

In one embodiment, the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof can be in forms as described above. In the present invention, it was revealed that regeneration of nerves occurs due to inhibition of neurite outgrowth being disrupted by blocking of the p75 signal transduction pathway. The effect of nerve regeneration by blocking of the signal transduction pathway has not been conventionally known. Therefore, the present invention provides an effect more excellent than the conventional art. Particularly, the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof may be preferably used in combination. In this case, various combinations may be used. Preferably, two, three or four polypeptides, polynucleotides and/or agents can be used. In another preferred embodiment, a plurality of molecules may be advantageously inhibited on the pathway.

In another aspect, the present invention provides a method for constructing a network of neurons. This method comprises a step of providing to the neurons, in an amount effective for network construction, a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof.

(Kit for Treatment of Nervous Diseases)

In another aspect, the present invention provides a kit for treatment of neurological diseases. This kit comprises (A) a population of cells regenerated using a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof, and (B) a container for preserving the cell population.

Alternatively, such a kit comprises (A) a composition comprising at least one molecule selected from the group consisting of the Peps polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p2l, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof; (B) neurons or cells capable of differentiating into neurons, and (C) a container for preserving the cell population.

The kit is effective for treatment of diseases (nervous diseases, nervous disorders, nervous abnormal conditions, and the like) which require neurons or a neuron population. The obtained neurons or neuron population may be in any condition, but preferably, a differentiation condition is suitable.

Instructions provided in the kit of the present invention may be in any form as long as the instruction can be conveyable, including paper, computer readable recording media (e.g., a flexible disk, CD-R, and the like), electric mail, web sites, and the like.

In another aspect, the present invention provides a method for treatment of neurological diseases. This method comprise the steps of (a) providing a cell population regenerated with a composition comprising at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof; and (b) transplanting the cell population to a patient.

Such a cell population is also referred to as a graft. As used herein, the term “graft” typically refers to homologous or exogenous tissue or cells to be inserted into a specific site of the body, which serve as a part of the body after insertion. Examples of conventional grafts include organs or part of the organ, blood vessel, blood vessel-like tissue, skin segments, cardiac valve, pericardium, dura, cornea segments, teeth, and the like. Therefore, the graft includes any material used for compensating an impaired portion by inserting into the portion. The graft is typically divided into the following groups depending on the type of the donor: autograft, allograft, and heterograft. As used herein, the term “immune reaction” refers to a reaction due to lack of coordination of immunological tolerance between a graft and a host, including, for example, hyperacute rejection (within several minutes immediately after transplant) (immune reaction due to β-Gal antibody or the like), acute rejection (reaction due to cell-mediated immunity 7 to 21 days after transplant), chronic rejection (rejection due to cell-mediated immunity after three months or more), and the like. Whether or not an immune reaction is elicited can be herein determined by histopathologically studying the type or number of cells (immune system) infiltrating into graft tissue by staining (e.g., HE staining or the like), immunostaining, or microscopic examination of tissue sections.

The provision of a cell population is described in detail in other portions in the specification. For transplant of cells into a patient, techniques well known in the art can be used. Such techniques are described in Hyojun-Gekagaku [Standard Surgery] (published by Igakushoin), Shin-Gekagaku-Taikei (New Complete Surgery (published by Nakayama-shoten), and the like. Preferably, when a graft of the present invention is transplanted, it may be noted that an excessive pressure should be avoided in the above-described general methods.

The graft or cell population of the present invention may comprise an immunosuppressant therein or therewith. Such an immunosuppressant is known in the art. For the purpose of immunosuppression, other methods for achieving immunosuppression may be used. Examples of immunosuppression methods for avoiding the above-described rejection include use of an immunosuppressant, surgical operations, radiation exposure, and the like. Major immunosuppressants include an adrenocortical steroid drug, cyclosporine, FK506, and the like. The adrenocortical steroid drug reduces the number of circulating T cells and inhibits the nucleic acid metabolism and cytokine secretion of lymphocytes to suppress the functions thereof and the migration and metabolism of macrophages. As a result, an immune reaction can be suppressed. Cyclosporine and FK506 have similar functions in which they bind to a receptor present on the membrane of helper T cells and enter cells, and then directly act on DNA to inhibit production of interleukin-2. Killer T cells eventually cannot function, resulting in immunosuppression. Side effects are a problem with use of these immunosuppressants. Particularly, steroids cause a number of side effects and cyclosporine is toxic to the liver and the kidney. FK506 is also toxic to the kidney. As a surgical operation, for example, lymphnodectomy, splenectomy, and thymectomy are illustrated, but the effect thereof has not been fully demonstrated. Among the surgical operations, thoracic duct funnel draws circulating lymphocytes to the outside of the body and its effectiveness has been confirmed, but it has a drawback such that a large volume of serum protein and lipid flow out nutritional disorder is likely to occur. Radiation exposure includes whole body radiation and graft radiation. The effect of radiation exposure is not reliable and the load of a recipient is large. Therefore, radiation exposure is used in conjunction with the above-described immunosuppressant. Any of the above-described methods is not very preferable for prevention of rejection.

(Screening)

The present invention also provides a screening method for identifying an agent inducing nerve regeneration. In this method, such an agent can be identified by determining whether or not the test agent has a significant effect (reduction, enhancement, extinction, or the like) on the interaction between at least one molecule selected from the group consisting of the Pep5 polypeptide, a nucleic acid molecule encoding the Pep5 polypeptide, an agent capable of specifically interacting with the p75 polypeptide, an agent capable of specifically interacting with a nucleic acid molecule encoding the p75 polypeptide, the p75 extracellular domain polypeptide, a nucleic acid molecule encoding the p75 extracellular domain polypeptide, an agent capable of specifically interacting with the Rho GDI polypeptide, an agent capable of specifically interacting with a nucleic acid molecule encoding the Rho GDI polypeptide, the Rho GDI polypeptide, a nucleic acid molecule encoding the Rho GDI polypeptide, an agent capable of specifically interacting with the MAG polypeptide, an agent capable of specifically interacting with a nucleic acid molecule encoding the MAG polypeptide, the p21 polypeptide, a nucleic acid molecule encoding p21, an agent capable of specifically interacting with the Rho polypeptide, an agent capable of specifically interacting with a nucleic acid molecule encoding the Rho polypeptide, an agent capable of specifically interacting with the Rho kinase and an agent capable of specifically interacting with a nucleic acid molecule encoding the Rho kinase, and variants and fragments thereof, and molecules capable of interacting therewith.

In one embodiment, the method comprises the steps of (a) contacting a first polypeptide having an amino acid sequence having at least 70% homology to SEQ ID NO. 4 or a fragment thereof and a second polypeptide having an amino acid sequence having at least 70% homology to SEQ ID NO. 6 or a fragment thereof in the presence of a test agent, and (b) comparing the binding level of the first polypeptide and the second polypeptide in the presence of the test agent with the binding level thereof in the absence of the test agent, where when the binding level is reduced in the presence of the test agent as compared to when the test agent is absent, the test agent is identified as an agent for nerve regeneration.

The above-described method for determining a test agent is well known in the art and the results can be analyzed using any statistical technique.

In the identification method of the present invention, presentation and selection of subjects or patients can be arbitrarily carried out. However, in the case of human subjects, it is preferable to previously obtain the consent of a human patient. Any subject having an abnormal nervous condition can be used.

In an administration step in the identification method of the present invention, any technique may be used. Preferably, a form of administration used in ordinary therapies, such as oral administration, intravenous injection, or the like, is advantageous.

The above-described screening or identification method is well known in the art. The screening or identification method can be carried out using a microtiter plate or a biomolecule array or chip having DNA, protein, or the like. An agent to be tested by screening may be contained in, for example, gene libraries, compound libraries synthesized by combinatorial libraries, and the like. The present invention is not so limited.

Therefore, the present invention is intended to provide a drug by computer modeling based on the disclosures of the present invention.

In other embodiments, the present invention includes compounds obtained by a quantitative structure activity relationship (QSAR) computer modeling technique as an instrument for screening for the regulatory activity of the compound of the present invention. Here, the computer technique includes some substrate templates prepared by a computer, pharmacophore, production of homologous models of the active site of the present invention, and the like. In general, a method for modeling an ordinary characteristic group of a substance capable of interacting with a given substance from data obtained in vitro can be carried out using a CATALYST™ pharmacophore method (Ekins et al., Pharmacogenetics,9:477-489, 1999; Ekins et al., J. Pharmacol. & Exp. Ther., 288:21-29, 1999; Ekins. et al., J. Pharmacol. & Exp. Ther., 290:429-438, 1999; Ekins et al., J. Pharmacol. & Exp. Ther., 291:424-433, 1999) and comparative molecular field analysis; COMFA) (Jones et al., Drug Metabolism & Disposition, 24:1-6, 1996), and the like. In the present invention, the computer modeling may be carried out using molecular modeling software (e.g., CATALYST™ version 4 (Molecular Simulations, Inc., San Diego, Calif.), etc.).

Fitting of a compound to an active site can be carried out using any computer modeling technique known in the art. Visual inspection and manual operation of a compound to an active site can be carried out using a program, such as QUANTA (Molecular Simulations, Burlington, Mass., 1992), SYBYL (Molecular Modeling Software, Tripos Associates, Inc., St. Louis, Mo., 1992), AMBER (Weiner et al., J. Am. Chem. Soc., 106:765-784, 1984), CHARMM (Brooks et al., J. Comp. Chem., 4:187-217, 1983), or the like. In addition, energy minimization can be carried out using a standard force field, such as CHARMM, AMBER, or the like. Other more specialized computer modelings include GRID (Goodford et al., J. Med. Chem., 28:849-857, 1985), MCSS (Miranker and Karplus, Function and Genetics, 11:29-34, 1991), AUTODOCK (Goodsell and Olsen, Proteins: Structure, Function and Genetics, 8:195-202, 1990), DOCK (Kuntz et al., J. Mol. Biol., 161:269-288, (1982)), and the like. Additional structures of compounds can be newly constructed to blank active sites, active sites of known low molecular weight compounds, or the like, using a computer program, such as LUDI (Bohm, J. Comp. Aid. Molec. Design, 6:61-78, 1992), LEGEND (Nishibata and Itai, Tetrahedron, 47:8985, 1991), LeapFrog (Tripos Associates, St.Louis, Mo.), or the like. Such computer modelings are well known in the art and commonly used. Those skilled in the art can appropriately design compounds within the scope of the present invention in accordance with the disclosures of the present specification.

In another aspect, the present invention provides a modulating agent which is identified by the above-described identification method of the present invention.

In another aspect, the present invention provides a pharmaceutical composition comprising the modulating agent of the present invention.

In another aspect, the present invention provides a method for prophylaxis or treatment of neurological diseases, disorders or conditions. This method comprises a step of administering a pharmaceutical composition comprising the modulating agent of the present invention to a subject. Preferably, the nerve-related conditions, disorders or diseases include, but are not limited to, abnormalities, disorders or diseases for which the present invention is determined to be effective, preferably Alzheimer's disease.

Nerve-related diseases, disorders and conditions have been believed to be difficult to cure completely. However, the above-described effect of the present invention allows early diagnosis which has been conventionally believed to be impossible, and is applicable to therapies. Therefore, the present invention can be said to have usefulness which cannot be achieved by conventional diagnostics or medicaments.

(Transgenic Animals)

In another aspect, the present invention also provides a vector comprising a nucleic acid molecule having the sequence of at least one nucleic acid molecule selected from the group consisting of a nucleic acid molecule encoding the Pep5 polypeptide, a nucleic acid molecule encoding the p75 polypeptide, and a nucleic acid molecule encoding the Rho GDI polypeptide. This vector can be used for various purposes, including, but limited to, production of transgenic animals, production of modified polypeptides, and the like.

Therefore, the present invention provides a cell, tissue, an organ, and an organism comprising the above-described vector. The present invention also provides a nerve-modified transgenic animal transformed using the vector. A method for producing an animal is known in the art.

In another aspect, the present invention provides a knockout animal in which a gene of the present invention is knocked out.

As used herein, the term “knock out” with reference to a gene refers to disruption (loss) or malfunctioning of the gene.

As used herein, the term “knockout animal” refers to an animal (e.g., mouse) in which a given gene is knocked out.

Any “animal” capable of being knocked out may be herein used.

Therefore, an animal includes a vertebrate and an invertebrate. An animal includes a mammal (e.g., mouse, dog, cat, rat, monkey, pig, cattle, sheep, rabbit, dolphin, whale, goat, horse, etc.), a bird (e.g., chicken, quail, etc.), an amphibian (e.g., frog, etc.), a reptile, an insect (e.g., Drosophilia, etc.), and the like. Preferably, an animal may be a mammal, more preferably an animal which is easy to knock out (e.g., mouse). In another preferred embodiment, an animal may be one that has been revealed to be appropriate as a model animal for humans (e.g., monkey). In some embodiments, an animal may not be a human. The present invention is not so limited.

The present invention also relates to use of the agent of the present invention (e.g., a polypeptide, etc.) for the purpose of the present invention (e.g., the therapy, diagnosis, prophylaxis, treatment, prognosis and the like of nervous diseases, disorders, and abnormal conditions) or use of the agent of the present invention for production of a medicament composition. Detail embodiments thereof are similar to those which are described above, and can be appropriately applied by those skilled in the art. Hereinafter, the present invention will be described by way of examples. The examples below are provided only for illustrative purposes. Therefore, the scope of the present invention is limited only by the accompanying claims but not the examples.

### EXAMPLES

The present invention will be described in greater detail below with reference to examples. The present invention is not limited to the examples below. The handling of animals complied with provisions defined by Osaka University, Japan.

### Example 1

### p75^{NTR} Transduces a Signal from Myelin-bound Protein to Rho

(Materials and Methods)

(Animals)

A strain of mice bearing a targeted disruption of the third exon of the p75^{NTR} gene (Lee et al., Cell 69:737-749, 1992) (the mouse strain was originally obtained from the Jackson Laboratory (Bar Harbor, Me.).) on a C57BL/6J background was used.

(Neurite Outgrowth Assay)

DRG were removed from adult mice and dissociated into single cells by incubation with 0.025% trypsin and 0.15% collagenase type 1 (Sigma Aldrich) for 30 min at 37° C. For cerebellar neurons, the cerebella from two animals was combined in 5 ml of 0.025% trypsin, triturated, and incubated for 10 min at 37° C. DMEM containing 10% FCS was added, and the cells were centrifuged at 800 rpm. Neurons were plated in Sato media (Cai et al., Neuron, 22:89-101, 1999) on poly-L-lysine coated chamber slides. For outgrowth assays, plated cells were incubated for 24 hours and were fixed in 4% (wt/vol) paraformaldehyde, and were immunostained with a monoclonal antibody (TuJ1) recognizing the neuron-specific β tubulin III protein. Then, the length of the longest neurite or the total process outgrowth for each β tubulin III-positive neuron was determined. Where indicated, recombinant rat MAG-Fc chimera (R&D Systems) was added to the medium after plating. The recombinant C3 transferase was introduced into the cytoplasm of the neurons before plating by trituration as described previously (Borasio et al, Neuron 2:1087-1096, 1989).

(Affinity-precipitation of GTP-RhoA)

293 cells were transfected with pcDNA3 vectors containing wild type RhoA whose NH₂-terminus is tagged with HA (Yamashita, T. et al., Neuron. 24:585-593, 1999) and/or full-length human p75^{NTR} by lipofection using Lipofectamine 2000 (Gibco BRL). Cerebellar neurons from P9 mice were isolated as described previously (Cai et al, Neuron, 22:89-101, 1999). Cells were lysed in 50 mM Tris (pH 7.5), 1% Triton X-100, 0.5% sodium deoxycholate, 0.1% SDS, 500 mM NaCl, 10 mM MgCl₂, with leupeptin and aprotinin, each at 10 μg/ml. Cell lysates were clarified by centrifugation at 13,000×g at 4° C. for 10 min, and the supernatants were incubated with the 20 μg of GST-Rho binding domain of Rhotekin beads (Upstate Biotech.) at 4° C. for 45 min. The beads were washed 4 times with washing buffer (50 mM Tris (pH 7.5) containing 1% Triton X-100, 150 mM NaCl, 10 mM MgCl₂,10 μg/ml each of leupeptin and aprotinin). Bound Rho proteins were detected by Western blotting using a monoclonal antibody against RhoA (Santa Cruz Biotechnology).

(MAG-Fc Binding and Immunocytochemistry)

DRG neuron cultures were fixed in 1% paraformaldehyde in PBS for 30 min. They were then blocked in PBS containing 2% FCS. To localize the MAG binding molecule, MAG-Fc (5 μg/ml) and anti-human IgG (1 μg/ml) were precomplexed for 30 min at room temperature, before being added to the fixed and blocked DRG neurons (Turnley and Bartlett, Int. J. Dev. Neurosci. 17: 109-119, 1999). To identify p75^{NTR}, cells were permeabilized with 0.2% Triton-X-100/PBS, then were incubated overnight with polyclonal antibody to p75^{NTR} (Promega), followed by an Alexa fluor™ 568 labeled anti-rabbit IgG (Molecular Probes) for 1 hour. The specificity of the antibodies was assessed by Western blot analysis of cells expressing the proteins, and control immunocytochemistry experiments were performed by leaving out the primary antibodies.

(Co-precipitation of Recombinant p75^{NTR} and GT1b)

Recombinant human p75^{NTR}-Fc chimera (1 μg; Genzyme-Techne) and 1 μg of purified ganglioside GT1b (>98% purity, Seikagaku Co.) were incubated in 200 μl 0.025% Tween20/PBS for 2 h, and p75^{NTR} was precipitated using proteinA sepharose (Amersham Pharmacia Biotech). The resultant precipitates were electrophoretically transferred to polyvinylidene difluoride membranes after SDS-PAGE with 7% gels and were immunoblotted with anti GT1b antibody (IgM, Seikagaku Co.) or anti p75^{NTR} antibody.

(Co-immunoprecipitation Experiments)

Cells were lysed on ice for 20 min with lysis buffer (10 mM Tris-HCl (pH 7.5)1 150 mM NaCl, 1% Triton™, 25 μg/ml leupeptin and 25 μg/ml aprotinin). The lysates were centrifuged at 13,000×g for 20 min, and the supernatants were collected. They were then incubated with the anti-GT1b antibody or an anti-HA antibody (for transfected HA-p75^{NTR}) overnight, followed by incubation with the anti mouse IgM antibody (for GT1b). Immunocomplex or MAG-Fc was collected with protein A sepharose (Amersham Pharmacia Biotech). The suspension was centrifuged at 1,000×g for 5 min. The pellets were washed 4 times with lysis buffer, and subjected to SDS-PAGE followed by immunoblot analysis.

### Example 1-1

### Inhibition of Neurite Outgrowth is Dependent on p75^{NTR}

The present inventors first asked if p75^{NTR} was associated with the effects of MAG on neurons. Neurite outgrowth of adult DRG neurons from mice carrying a mutation in the p75^{NTR} gene (Lee et al., Cell 69:737-749, 1992) or wild type mice was examined.

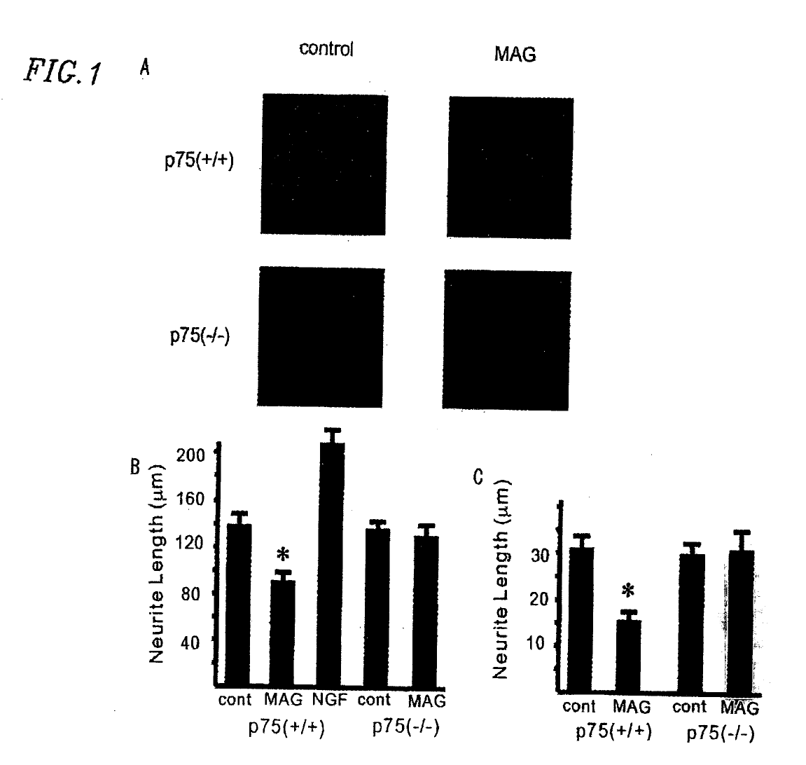

A soluble chimeric form of MAG, consisting of the extracellular domain of MAG fused to the Fc region of human IgG (MAG-Fc), was used. It was shown that soluble MAG was released in abundance from myelin and found in vivo, and MAG-Fc could potently inhibit axonal growth (Tang, S. et al., J. Cell Biol. 138:1355-1366, 1997a; Tang, S. et al., Mol. Cell. Neurosci. 9:333-346, 1997b). The present inventors compared the neurite length between MAG-treated and MAG-untreated neurons. MAG-Fc at the concentration of 25 μg/ml inhibited neurite outgrowth of DRG neurons from adult wild type mice (FIG. 1). Fc had no effect on the neurons (data not shown). Interestingly, the inhibitory effect of MAG could not be observed in DRG neurons from adult mice carrying a mutation in the p75^{NTR} gene. Exactly the same results were obtained whether total process outgrowth or length of the longest neurite was measured (data not shown).

Similar experiments with postnatal cerebellar neurons were performed. At a concentration of 25 μg/ml of MAG-Fc, neurite growth was significantly inhibited when cerebellar neurons from P9 wild type mice were used (C in FIG. 1). Again, no inhibition by MAG was observed in the neurons from P9 mice carrying a mutation in the p75^{NTR} gene. These results suggest that MAG inhibits neurite outgrowth by a p75^{NTR} dependent mechanism.

p75^{NTR} has been shown to be required for the inhibition of axonal growth and target innervation of peripheral neurons in vivo and in vitro (Kimpinski et al., Neuroscience 93:253-263, 1999; Kohn et al., J. Neurosci. 19:5393-5408, 1999), and for suppression of hyper-innervation of cholinergic neurons in vivo (Yeo, T. T. et al., J. Neurosci. 17:7594-7605, 1997). It was recently reported that the growth of sympathetic axons within the myelinated portions of the cerebellum was greater in NGF transgenic mice lacking expression of p75^{NTR} compared to those expressing p75^{NTR} in vivo (Walsh G. S. et al., J. Neurosci. 19:4155-4168, 1999). It may be a relevant finding supporting our data, as neurons carrying a mutation in the p75^{NTR} gene are suggested to be refractory to inhibitory factors.

### Example 1-2

### Signaling Mechanisms of MAG on the Neurons

Some neurons extend neurites rapidly when RhoA is inactivated, and neurite retraction occurs when RhoA is active (Davies, A. M., Curr. Biol. 10:R198-R200, 2000). Previous study shows that inactivation of RhoA promoted axonal regeneration in vivo (Lehmann, M. et al., J. Neurosci. 19:7537-7547, 1999). Thus, it was examined if activation of RhoA is necessary for modification of neurite outgrowth by MAG in our system.

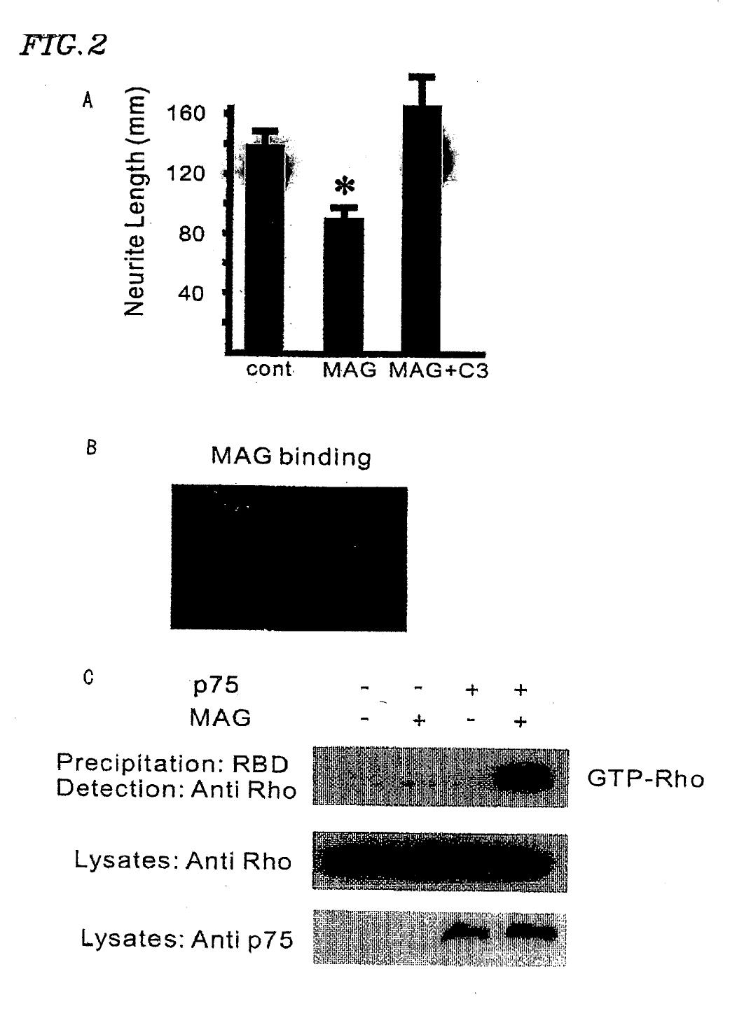

In order to examine if activation of RhoA is necessary for modification of neurite outgrowth by MAG in our system, the present inventors employed the exoenzyme C3 transferase from Clostridium botulinum, which ADP-ribosylates RhoA. The recombinant C3 transferase was introduced into the cytoplasm of DRG neurons by trituration. The C3 transferase completely abolished the effect of MAG on DRG neurons from wild type mice (A in FIG. 2). These data are consistent with the previous report suggesting RhoA is in the MAG signaling pathway (Lehmann, M. et al., J. Neurosci. 19:7537-7547, 1999).

The next hypothesis tested was that MAG regulates RhoA activity by a p75^{NTR} dependent mechanism. 293 cells, which express no p75^{NTR} endogenously, were used as MAG-Fc binding to the cell surface was diffusely observed (B in FIG. 2). Using the RhoA-binding domain of the effector protein Rhotekin (Ren, X. D. et al., EMBO J. 18:578-585, 1999), the GTP-bound form of RhoA can be affinity-precipitated. The direct measurement of RhoA activity in the cells can be done using this method. The assay revealed that within 30 minutes following the addition of soluble MAG (25 μg/ml), extracts of 293 cells transfected with p75^{NTR} and RhoA contained dramatically increased amounts of GTP-RhoA compared to the control (C in FIG. 2), though no change in the activity was observed by the addition of Fc (data not shown). However, no increase in GTP-RhoA content was observed in the cells untransfected with p75^{NTR} by the addition of MAG-Fc (C in FIG. 2).

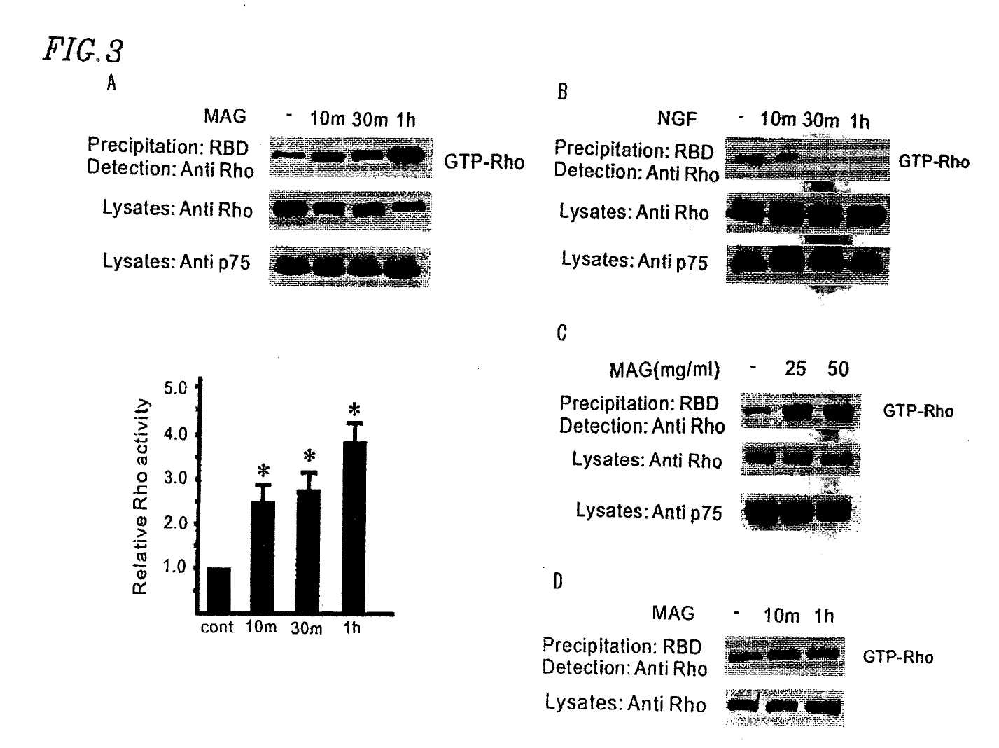

Regulation of RhoA activity when the proteins are artificially expressed may be difficult to detect in natural cells. Therefore, to see if RhoA activity is regulated by MAG in the cells expressing endogenous p75^{NTR}, postnatal cerebellar granule neurons were used, as these neurons also are sensitive to MAG with regard to neurite outgrowth. Consistent with the observation in transfected 293 cells, MAG-FC activates RhoA in cerebellar granule neurons from wild type mice (P9), which express abundant p75^{NTR} (A in FIG. 3). This rapid activation was in contrast with the effect of NGF on the neurons, which is also mediated by p75^{NTR}, as they do not express trkA (B in FIG. 3). RhoA activity (C in FIG. 3) as well as the effect of MAG on neurite outgrowth (data not shown) seems to be saturated by MAG at the concentration of 25 μg/ml. Activation of RhoA by MAG was lost in the neurons from mice carrying a mutation in the p75^{NTR} gene (D in FIG. 3). These data demonstrate that MAG activates RhoA by a p75^{NTR} dependent mechanism, thus inhibiting neurite outgrowth of postnatal cerebellar granule neurons.

Only the wild type of RhoA which is predominantly in a GDP-bound form, but not the constitutive active form of RhoA interacts with p75^{NTR} (Yamashita, T. et al., Neuron. 24:585-593, 1999). In transfected cells, overexpression of p75^{NTR} activated RhoA in a neurotrophin independent manner. Therefore, the GDP-bound form of RhoA may interact with the p75^{NTR} helical domain to be activated following exposure to MAG. More detailed structure-function analyses of p75^{NTR} should help to elucidate the precise mechanism of regulation of RhoA activity by p75^{NTR}.

### Example 1-3

### Colocalization of p75^{NTR} and MAG Binding

MAG binds to neurons in a sialic acid-dependent manner, but MAG's sialic acid binding site is distinct from its neurite inhibitory activity. The sialic acid-dependent binding to MAG is not sufficient or necessary for MAG's inhibitory effect (Tang, S. et al., J. Cell Biol. 138:1355-1366, 1997a). Therefore, it is possible that the binding partner and the signal transducing element for MAG may form a receptor complex. The present inventors assumed that the binding partner for MAG and p75^{NTR} might interact in a cis manner. To test this hypothesis, the localization of p75^{NTR} and MAG binding was assessed on the subcellular level.

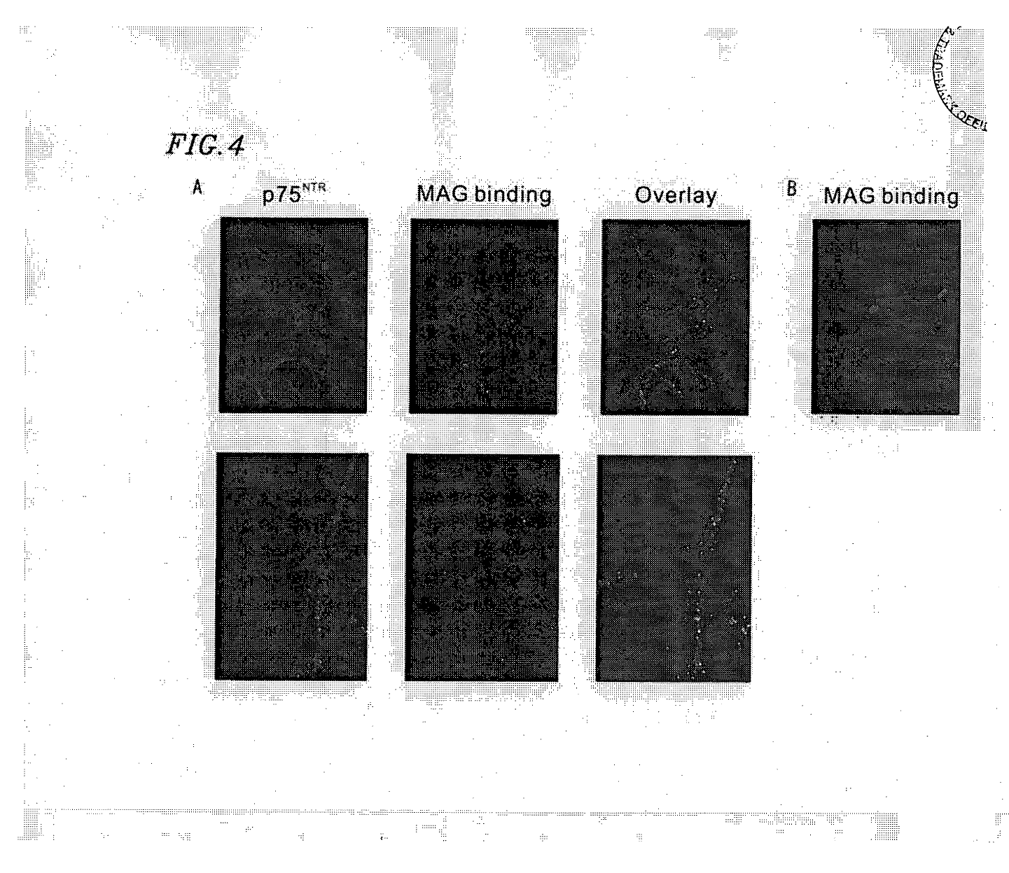

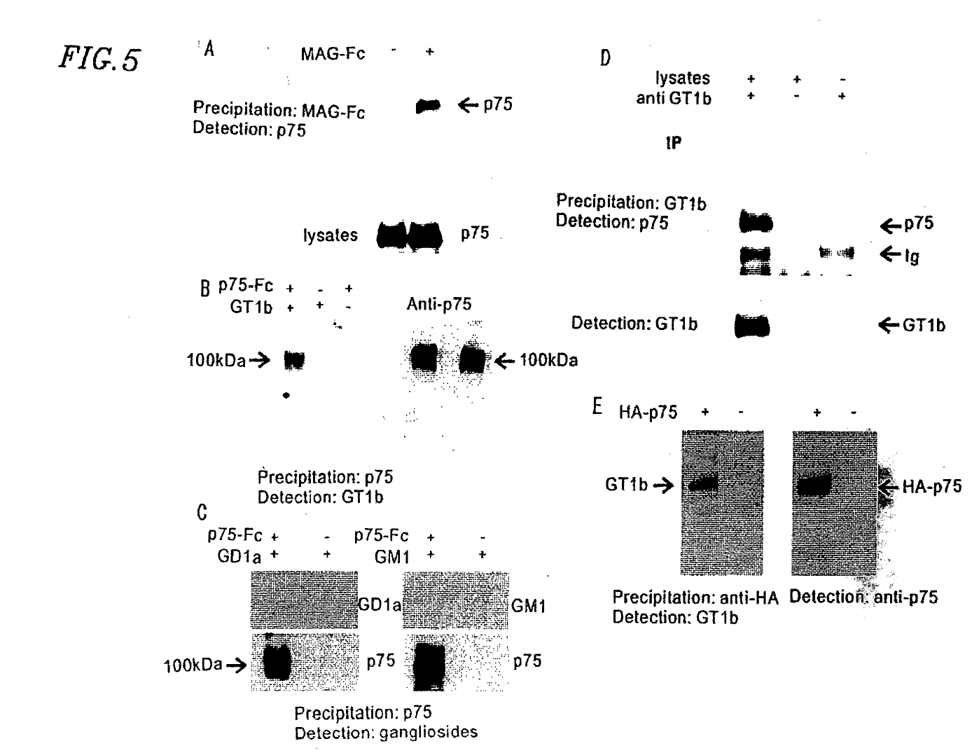

Binding of MAG-Fc was visualized by incubation with a fluorescent-tagged anti-human IgG. FIG. 4 shows binding of MAG-Fc to adult DRG neurons using confocal laser microscopy. MAG-Fc binding appears punctate. The same cells were stained with an anti-p75^{NTR} antibody, and the distribution was assessed. p75^{NTR} expression on the cell body was rather diffuse but that on the neurites showed fine speckled staining (A in FIG. 4, upper). The vast majority of puncta for p75^{NTR} immunoreactivity were colocalized with MAG binding. At high magnification, the colocalization was evident by the similar distribution of hot spots on the neuritic plasma membrane (A in FIG. 4, lower). Binding of MAG-Fc was still observed in DRG neurons from mice carrying a mutation in the p75^{NTR} gene (B in FIG. 4). These data demonstrate that the p75^{NTR} and MAG binding colocalize.

### Example 1-4

### p75^{NTR} Binds to Ganglioside GT1b

The present inventors next examined interaction of endogenous p75^{NTR} and MAG using lysates prepared from post-natal cerebellum from mice. In the MAG-Fc precipitates, the anti-p75^{NTR} antibody revealed the presence of a protein corresponding to p75^{NTR} (A in FIG. 5). However, as MAG-Fc did not precipitate recombinant p75^{NTR} protein in our preliminary experiments (data not shown), these data suggest indirect interaction of MAG with p75^{NTR}. Thus, p75^{NTR} may not be the binding partner, but the signal transducing element.

MAG binds to specific sialylated glycans and gangliosides present on the cell surface of neurons. The ability of MAG to bind specific gangliosides bearing terminal α-2-3 linked sialic acid has been well documented (Yang, L. J. et al., Proc. Natl. Acad. Sci. USA. 93:814-818, 1996). MAG was shown to bind GT1b and GD1a as well as the α-series gangliosides, and antibody cross-linking of cell surface GT1b, but not GD1a, mimics the effect of MAG (Vinson, M. et al., J. Biol. Chem. 276:20280-20285, 2001). The pathological features of the nervous system of the complex ganglioside knockout mice closely resemble those reported in mice with a disrupted gene for MAG (Sheikh, K. A. et al., Proc. Natl. Acad. Sci. USA. 96:7532-7537, 1999). These data prompted the present inventors to examine association of p75^{NTR} with these gangliosides, assuming that p75^{NTR} and gangliosides form a receptor complex for MAG.

Recombinant p75^{NTR} extracellular domain fused to Fc purified from Sf21 cells was used to precipitate gangliosides. In the p75^{NTR} precipitates, the anti-GT1b antibody revealed the presence of an approximately 100 kDa band (B in FIG. 5 left). To confirm that the positive band was p75^{NTR}, anti-GT1b antibody was stripped from the membrane, and the membrane was reprobed with the anti-p75^{NTR} antibody. The results showed the positive band to be p75^{NTR} (B in FIG. 5, right). It is not a non-specific interaction of GT1b, as association of extracellular domain of EGF receptor and GT1b was not observed (data not shown). Thus, GT1b binds to p75^{NTR} in a manner that is SDS-resistant. Though GD1a was also shown to associate with MAG (Vinson, M. et al., J. Biol. Chem. 276:20280-20285, 2001), the present inventors did not see any interaction of p75^{NTR} with GD1a (C in FIG. 5) Also, no interaction of p75^{NTR} with GM1 was found (C in FIG. 5), demonstrating specific interaction of p75^{NTR} with GT1b. Employing an anti-GT1b antibody, the present inventors examined interaction of endogenous p75^{NTR} and GT1b using lysates prepared from post-natal cerebellum from mice. Immunocytochemistry using the antibody confirmed the expression of GT1b on the surface of these neurons. In the GT1b immunoprecipitates, the anti-p75^{NTR} antibody revealed the presence of a protein corresponding to p75^{NTR} (D in FIG. 5). Preincubation of anti-GT1b antibody with synthetic GT1b abolished the detection of p75^{NTR} (data not shown). Finally, the present inventors assessed interaction of p75^{NTR} with GT1b using transfected 293 cells, which express abundant GT1b on the cell surface (data not shown). As expected, immunoprecipitaed p75^{NTR} was complexed with GT1b in an SDS-resistant manner (E in FIG. 5). These data suggest that GT1b and p75^{NTR} form a receptor complex for MAG.

According to the above-described results, the present inventors consider that p75^{NTR} is a molecule capable of eliciting dual signals. p75^{NTR} has been shown to bind more than just neurotrophins, such as CRNF (Fainzilber, M. et al., Science 274:1540-1543, 1996) or rabies virus glycoprotein (Tuffereau, C. et al., EMBO J. 17:7250-7259, 1998), but it is not known if these ligands trigger any signals through p75^{NTR} Thus, our findings demonstrating that p75^{NTR} is a signal transducer not only for neurotrophins but also for MAG are intriguing. More interestingly, neurotrophins binding to p75^{NTR} promotes axonal outgrowth of neurons presumably by inhibiting RhoA activity (Yamashita, T. et al., Neuron. 24:585-593, 1999), but MAG elicits the opposite effect via p75^{NTR} on neurons by activating RhoA. This implies that p75^{NTR} has dual signals as a transducing element. It is also important to note that essentially all adult neurons are sensitive to inhibition by MAG, whereas p75^{NTR} has a restricted distribution. The identification and characterization of MAG signals have shed light on a previously unrecognized mechanism by which neurons respond to extracellular inhibitory molecules.

### Example 2

### Cytoplasm p21^{Cip1/WAF1} Regulates Neurite Remodeling by Inhibiting Rho Kinase Activity

As shown in Example 1, it was found that p75^{NTR} induces bi-directional signals. The present inventors next analyzed the precise mechanism of regulation of Rho activity by p75^{NTR}.

(Materials and Methods)

(Animals)

The strain of mice bearing a targeted disruption of the third exon of the p75^{NTR} gene (Lee, K. F. et al., Cell 69. 737-749 (1992)) (the mouse strain was originally obtained from the Jackson Laboratory (Bar Harbor, Me.).) on a C57BL/6J background was used.

(Co-immunoprecipitation)

Amino-terminally FLAG-tagged human p75^{NTR} (SEQ ID NOs. 3 and 4) and/or HA-tagged RhoA (SEQ ID. NOs. 11 and 12) (Yamashita et al., Neuron 24, 585-593 (1999)) were transfected with 293T cells or N1E-115 cells by lipofection using Lipofectamine 2000 (Gibco BRL). Cells were lysed on ice for 20 min with lysis buffer (10 mM Tris-HCl (pH 7.5), 150 mM NaCl, 0.2% NP-40, 25 μg/ml leupeptin and 25 μg/ml aprotinin). The lysates were centrifuged at 13,000×g for 20 min, and the supernatants were collected. They were then incubated with the anti-FLAG antibody (for transfected FLAG-p75^{NTR}) or anti-p75 antibody (Chemicon) (for cerebellar neurons) for 3 hours. The immunocomplex was collected with protein A sepharose (Amersham Pharmacia). The suspension was centrifuged at 1,000×g for 5 min. The pellets were washed 4 times with lysis buffer, and subjected to SDS-PAGE, followed by immunoblot analysis using anti-Rho GDIα antibody (Sigma) or anti-RhoA antibody (Santa Cruz Biotechnology). Where indicated, recombinant rat MAG-Fc chimera (25 μg/ml, RD Systems Inc.), the Nogo peptide (4 μM, Alpha Diagnostic; SEQ ID NO. 10), TAT(PTD domain)-fused Pep5 (TAT-CFFRGGFFNHNPRYC) (SEQ ID NO. 2) or TAT(PTD domain)-fused control peptide (TAT-GGWKWWPGIF) (SEQ ID NO. 15) was used. The peptides were chemically synthesized and their composition was verified by amino acid analysis and mass spectrometry (Sigma Genosys). Amino-terminally FLAG-tagged human p75^{NTR} was cloned into pcDNA3.1 expression plasmid (Invitrogen).

(Co-precipitation of p75^{NTR} and Rho GDI)

p75N precipitated from the transfected 293T cells using anti-FLAG antibody and protein A sepharose, was incubated with recombinant human GST-Rho GDI (Cytoskeleton) or GST-RhoA (Cytoskeleton) in 200 μl buffer (20 mM Tris-HCl (pH 7.5), 100 mM NaCl, 10 mM EDTA, 0.025% Tween20) for 2 h, and washed. The resultant precipitates were electrophoretically transferred to polyvinylidene difluoride membranes after SDS/PAGE and were immunoblotted with the anti-GST antibody (Sigma). To examine the nucleotide dependency, GST-RhoA was preloaded with the appropriate nucleotide, and EDTA was replaced with 10 mM MgCl₂. Where indicated, Pep5 or the control peptide (GGWKWWPGIF (SEQ ID NO. 15)) was used.

(Production of Recombinant Proteins)

The p75^{NTR} ICD coding sequence, with or without the deletion, was cloned into the pGEX-5X bacterial expression vectors (Amersham Biosciences) to generate GST-fused proteins from E. coli. pGEX-GST-Rho GDI was provided by Dr. Y. Takai. After cell growth to an optical density at 600 nm (OD₆₀₀) of 1.0, 1 mM isopropyl-1-thio-β-D-galactopyranoside (IPTG) was added to induce protein synthesis, and cells were grown for another 16 hr at 25° C. Fusion proteins were purified employing glutathione-Sepharose 4B (Amersham Biosciences), and the GST moiety was removed to produce recombinant Rho GDI. Purity of the proteins was determined by SDS-PAGE and the concentration was measured. The deletion mutants of rat p75^{NTR} ICD are from residues 274 to 342, to 351, to 363, to 375, to 390, to 406 and to 425 (EMBO J. 16, 4999-5005 (1997)). Complex formation of GST-p75^{NTR} mutants with Rho GDI was assessed by precipitating the GST-p75^{NTR} mutants.

(Affinity-precipitation of GTP-RhoA)

Amino-terminally FLAG-tagged human p75^{NTR} or the deletion mutants of p75^{NTR} ICD were cloned into pcDNA3.1 expression plasmid, and were transfected with 293T cells. Cells were lysed in 50 mM Tris (pH 7.5), 1% Triton X-100, 0.5% sodium deoxycholate, 0.1% SDS, 500 mM NaCl, 10 mM MgCl₂, with leupeptin and aprotinin, each at 10 μg/ml (Ren, X. D., Kiosses, W. B. & Schwartz, M. A., EMBO J. 18, 578-585 (1999)). Cell lysates were clarified by centrifugation at 13,000×g at 4° C. for 10 min, and the supernatants were incubated with the 20 μg of GST-Rho binding domain of Rhotekin beads (Upstate Biotechnology) at 4° C. for 45 min. The beads were washed 4 times with washing buffer (50 mM Tris (pH 7.5) containing 1% Triton X-100, 150 mM NaCl, 10 mM MgCl₂, 10 μg/ml each of leupeptin and aprotinin). Bound Rho proteins were detected by Western blotting using a monoclonal antibody against RhoA (Santa Cruz Biotechnology).

(In vitro Nucleotide Exchange Assay)

Lipid-modified RhoA was purified from yeast membranes as described (Forget, M. A., Desrosiers, R. R., Gingras, D. & Beliveau, R., Biochem. J. 361, 243-54(2002)). [³H]GDP- or GDP-RhoA complexed with Rho GDI was obtained by first incubating GDP-RhoA with or without [³H]GDP, followed by incubation with Rho GDI for 30 min, as described previously (Takahashi, K. et al., J. Biol. Chem. 272, 23371-23375 (1997)). The sample, subjected to gel filtration, was equilibrated with 20 mM Tris-HCl (pH 7.5) containing 5 mM MgCl₂, 1 mM dithiothreitol and 0.1% CHAPS. The GDP dissociation and GTP binding assays were carried out by the filter binding method as described previously (Hart, M. J., Eva, A., Evans, T., Aaronson, S. A. & Cerione, R. A., Nature 354, 311-314 (1991)). In the [³H]GDP dissociation assay, 50 nM of the complex was incubated for 20 min with various concentrations of GST-fused proteins in a reaction mixture (50 μl) containing 30 mM Tris-HCl (pH 7.5), 5 mM or 0.5 μM MgCl₂, 1 (for low Mg) or 10 (for high Mg) mM EDTA, 0.1 mM GTP, 1 mM dithiothreitol, 0.12% CHAPS and 0.2 mg/ml bovine serum albumin. In the [³⁵S] GTPγS binding assay, the complex was incubated as described above except that 1 μM [³⁵S] GTPγS was used instead of 0.1 mM GTP. At the indicated time, an aliquot of the reaction sample was removed, and passed through nitrocellulose filters (IPVH 000, Millipore). The filters were washed and used for scintillation counting. GST protein or the buffer was used as a control. His-tagged catalytic domain of Dbl was used at the concentration of 90 nM.

(Neurite Outgrowth Assay (In Vitro))

Dorsal root ganglia were removed from adult mice and dissociated into single cells by incubation with 0.025% trypsin and 0.15% collagenase type 1 (Sigma) for 30 min at 37° C. For cerebellar neurons, the cerebella from two animals were combined in 5 ml of 0.025% trypsin, triturated, and incubated for 10 min at 37° C. DMEM containing 10% FCS was added, and the cells were centrifuged at 800 rpm. Neurons were plated in Sato media (Cai, D., Shen, Y., De Bellard, M., Tang, S. & Filbin, M. T., Neuron 22, 89-101 (1999)) on poly-L-lysine coated chamber slides. For outgrowth assays, plated cells were incubated for 24 hours and were fixed in 4% (wt/vol) paraformaldehyde, and were immunostained with a monoclonal antibody (TuJ1) recognizing the neuron-specific β-tubulin III protein. Then, the length of the longest neurite or the total process outgrowth for each β-tubulin III-positive neuron was determined. Where indicated, MAG-FC (25 μg/ml) or the Nogo peptide (4 μM) was added to the medium after plating. pEF-BOS-myc-Rho GDI plasmid, which was provided by Dr. Yoshimi Takai, or pEGFP plasmid, as a control, was used for the transfection. Twenty four hours after transfection by lipofection, the cells were replated and incubated for 24 hours. To determine the transfected cells, cells were permeabilized and immunostained with the anti-myc antibody (1:1000, Sigma).

(Nerve Regeneration Effect in Mammal of an Agent Capable of Disrupting the Interaction Between a Silencer and/or p75^{NTR} and Rho GDI)

200 g male Wistar rats were used. After the ninth thoracic vertebrae laminectomy was performed, the dorsal half of the spinal cord was dissected. A continuous osmotic pump was used to continuously administer either TAT(PTD domain)-fused Pep5 (TAT-CFFRGGFFNHNPRYC) (SEQ ID NO. 2) or TAT(PTD domain)-fused control peptide (TAT-GGWKWWPGIF) (SEQ ID NO. 15) to the injured site for 6 weeks (1 mg/weight/day). In this case, the tip of a tube connected to the pump was left in the medullary space. After spinal cord injury, the functional recovery was assessed using the BBB score. The animals were observed on day 7, 14, 21, 28, 35, and 42 after injury. These experiments were carried out using techniques described in Fournier A. E., Takizawa, B. T., Strittmatter, S. M., J. Neurosci. 2003, 23, 1416-1423.

Similar experiments were carried out using anti-p75^{NTR} antibodies, anti-Rho GDI antibodies, and the extracellular domain of p75^{NTR}. As a result, similar nerve regeneration effects were observed. These experiments were also carried out using techniques described in Fournier A. E., Takizawa, B. T., Strittmatter, S. M., J. Neurosci. 2003, 23, 1416-1423.

### Example 2-1

### p75^{NTR} Associates with Rho GDI

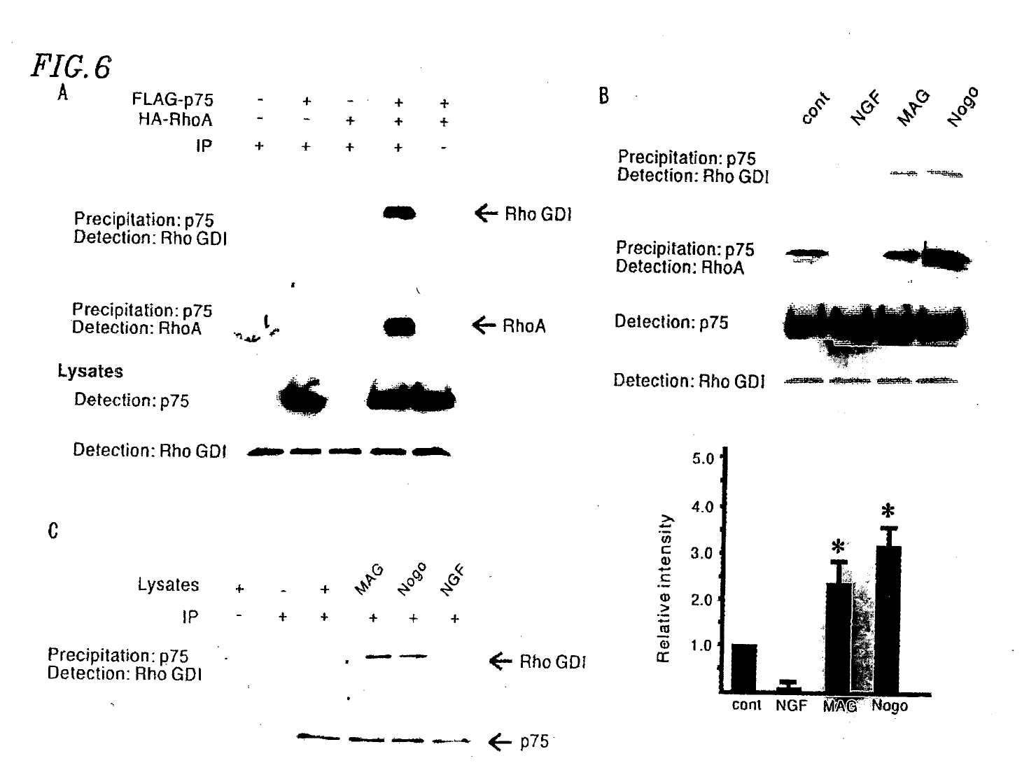

The present inventors first asked whether the complex of RhoA and Rho GDI associates with the intracellular domain of p75^{NTR} 293T cells, which express Rho GDI but not p75^{NTR} endogenously, were transfected with FLAG-tagged p75^{NTR} and HA-tagged wild-type RhoA. In the p75^{NTR} precipitates, the anti-Rho GDI antibody revealed the presence of a protein corresponding to Rho GDI (A in FIG. 6). As previously shown (Yamashita, T., Tucker, K. L. & Barde, Y. A., Neuron 24, 585-593 (1999)), RhoA was included in the complex. The present inventors next examined whether the interaction was strengthened by MAG or Nogo, which have been shown to activate RhoA through a p75^{NTR}-dependent mechanism. N1E-115 cells, which express the Nogo receptor endogenously (data not shown), were transfected with FLAG-tagged p75^{NTR}. The peptide corresponding to residues 31-55 of the extracellular fragment of Nogo (4 μM) (Fournier, A. E. et al., Nature 409, 341-346, 2001) and soluble MAG-Fc (25 μg/ml) significantly enhanced the interaction of p75^{NTR} with Rho GDI as well as RhoA (B in FIG. 6). In contrast, NGF (100 ng/ml), which inactivates RhoA by p75^{NTR}, abolished the interaction of p75^{NTR} with Rho GDI as well as RhoA. The present inventors previously noted that the interaction of endogenous p75^{NTR} with RhoA could not be observed in neurons (Yamashita, T., Tucker, K. L. & Barde, Y. A., Neuron 24, 585-593 (1999)) ). Therefore, the present inventors examined the interaction of endogenous p75^{NTR} with Rho GDI or RhoA using lysates prepared from post-natal cerebellar neurons from mice (P9). As shown in C of FIG. 6, an association of endogenous p75^{NTR} with RhoA and Rho GDI was observed only after stimulation with MAG or Nogo, suggesting that p75^{NTR} may not be a constitutive activator of RhoA in the cells expressing endogenous p75^{NTR} These findings demonstrate that Rho GDI in complex with RhoA interacts with p75^{NTR} and that the interaction is strengthened by MAG and Nogo.

### Example 2-2

### Direct Interaction of p75^{NTR} with Rho GDI

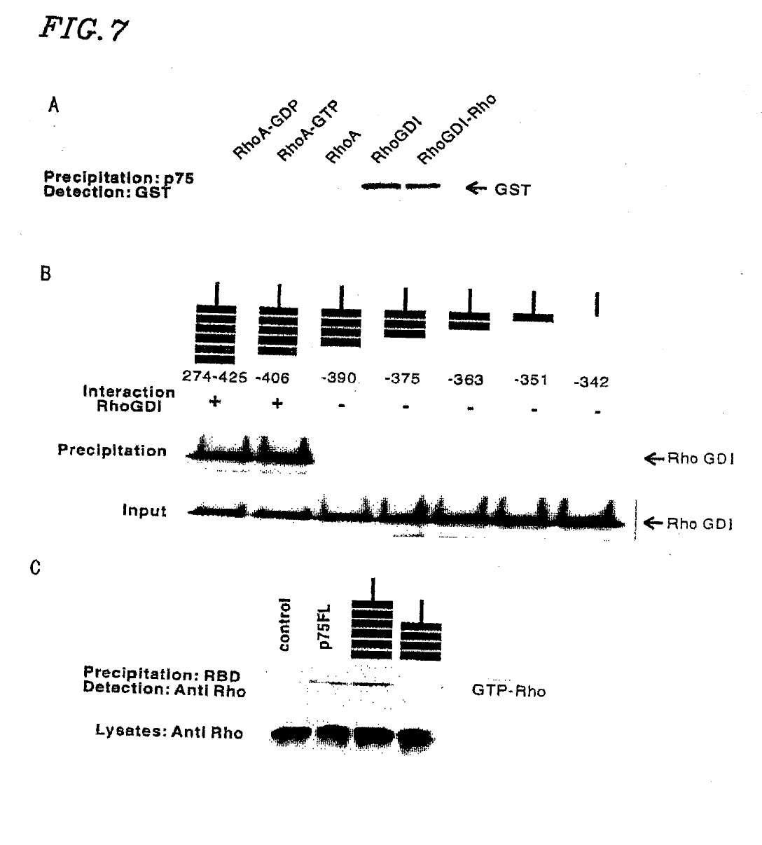

As RhoA was isolated as a p75^{NTR}-interacting protein by yeast two-hybrid screening, RhoA was suggested to bind directly to p75^{NTR} (Yamashita, T., Tucker, K. L. & Barde, Y. A., Neuron 24, 585-593 (1999)). However, the fact that endogenous Rho GDI in yeast is active on mammalian Rho family members leaves open an alternative possibility that RhoA in complex with yeast Rho GDI may be associated with p75^{NTR} in the yeast. Therefore, the present inventors next examined the direct physical interaction of p75^{NTR} with Rho GDI or RhoA using purified recombinant proteins. Bacterially produced RhoA, in the GDP-bound, GTP-bound or the nucleotide-depleted state, was incubated with p75^{NTR}, which was precipitated from transfected 293T cells. However, the present inventors observed no interaction between them in any nucleotide state (A in FIG. 7). Interestingly, recombinant Rho GDI bound to p75^{NTR}. When prenylated RhoA was complexed with Rho GDI, it associated with p75^{NTR}, suggesting that Rho GDI, but not RhoA, directly complexes with p75^{NTR}.

The present inventors determined the structural basis of the interaction between Rho GDI and p75^{NTR} The fifth of the six α-helices of the intracellular domain (ICD) of p75^{NTR} shows significant similarity with the 14-mer peptide mastoparan. Mastoparan is an amphiphilic component of wasp venom known to activate RhoA. Experiments with the deletion mutant of p75^{NTR} ICD show that the fifth helix is necessary for the interaction of p75^{NTR} with Rho GDI (B in FIG. 7). These results suggest that the activation of RhoA by MAG and Nogo may be dependent on the interaction of Rho GDI with the fifth helix of p75^{NTR} ICD. To test this hypothesis more directly, the present inventors employed 293T cells which express no p75^{NTR} endogenously. Affinity precipitation of the GTP-bound form of RhoA revealed that RhoA was activated by the overexpression of full-length p75^{NTR} or p75^{NTR} ICD, as shown previously (Yamashita, T., Tucker, K. L. & Barde, Y. A., Neuron 24, 585-593 (1999)). As expected, the deletion mutant that lacks the fifth helix failed to activate RhoA (C in FIG. 7), demonstrating that the fifth helix is necessary for the activation of RhoA by p75^{NTR}.

### Example 2-3

### Displacement Effect of p75^{NTR} that Releases RhoA from Rho GDI

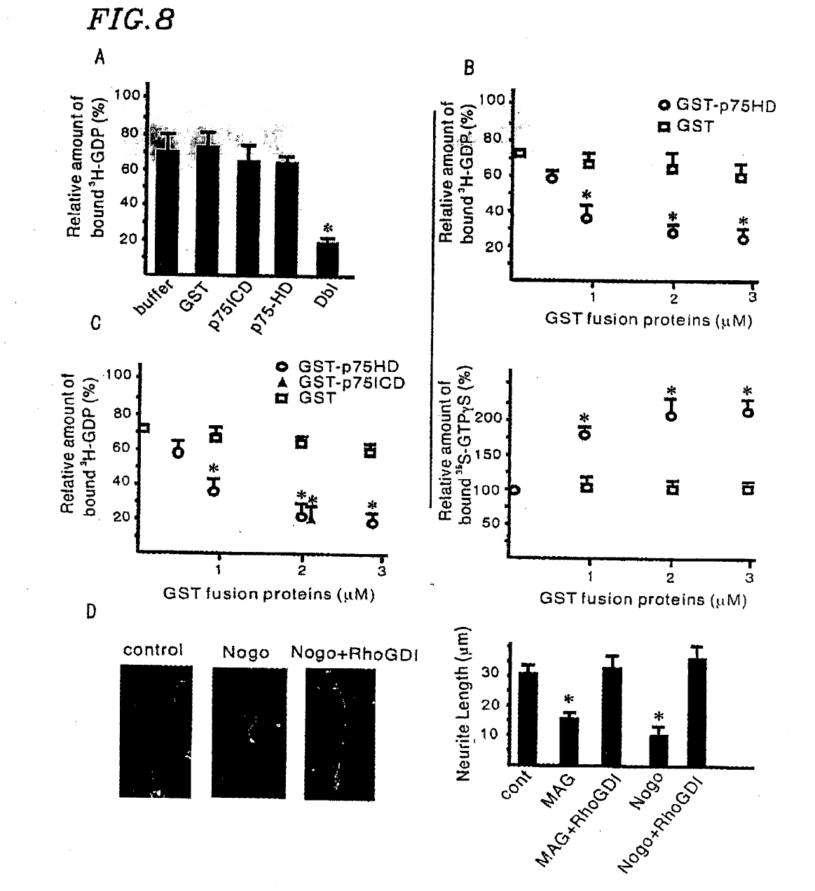

Experiments with bacterially expressed p75^{NTR} failed to indicate GDP/GTP exchange activity on recombinant RhoA in in vitro assays (A in FIG. 8). These results, in combination with the fact that RhoA does not directly associate with p75^{NTR}, raise the possibility that p75^{NTR} reduces the activity of Rho GDI, thus facilitating the release of RhoA from Rho GDI. This step allows for the activation by guanine nucleotide exchange factors and membrane association of the GTP-bound form of Rho proteins (Sasaki, T. & Takai, Y., Biochem Biophys Res Commun. 245, 641-645 (1998)). The present inventors first examined the effect of the interaction of Rho GDI with the helical domain (HD) of p75^{NTR} on its ability to inhibit the GDP/GTP exchange reaction of RhoA at low Mg²⁺ concentrations, as the inhibitory effect of Rho. GDI is more obvious at low Mg²⁺ concentrations (Takahashi, K. et al., J. Biol. Chem. 272, 23371-23375 (1997)). This reaction was estimated by measuring the dissociation of. [³H]GDP from [³H]GDP-RhoA complexed with Rho GDI and the binding of [³⁵S]GTPγS to GDP-RhoA complexed with Rho GDI. p75^{NTR} HD reduced this Rho GDI activity in a dose-dependent manner (B in FIG. 8). Under comparable conditions, glutathione S-transferase (GST) did not affect the Rho GDI activity (B. in FIG. 8). These results demonstrate that the p75^{NTR} HD has a potency to directly interact with Rho GDI and reduce its ability to inhibit the GDP/GTP exchange reactions of RhoA. The present inventors next examined the effect of p75^{NTR} HD on the Rho GDI ability to inhibit the Dbl stimulated GDP/GTP exchange reaction of RhoA at high Mg²⁺ concentrations. Rho guanine nucleotide exchange factors (Rho GEFs), such as Dbl, stimulate the GDP/GTP exchange reaction of. GDP-RhoA free of Rho GDI, but not that of GDP-RhoA complexed with Rho GDI at high Mg²⁺ concentrations (Yaku, H., Sasaki, T. & Takai, Y., Biochem Biophys Res Commun. 198, 811-817 (1994)). Dbl stimulated the dissociation of GDP from GDP-RhoA (A in FIG. 8), but the dissociation of GDP from GDP-RhoA complexed with Rho GDI was markedly reduced (C in FIG. 8). However, the dissociation of GDP was restored by p75^{NTR} HD. This inhibitory effect of p75^{NTR} HD on the Rho GDI activity was dose dependent. p75^{NTR} ICD showed the inhibitory effect to the same extent as p75^{NTR} HD (C in FIG. 8). These results demonstrate that the interaction of Rho GDI with p75 HD increases its activity in both the RhoGEF-independent and RhoGEF-dependent GDP/GTP exchange reactions of RhoA.

As p75^{NTR} has an ability to release RhoA from Rho GDI in vitro, activation of RhoA by MAG and Nogo through p75^{NTR} may be attributable to the activity that releases Rho from Rho GDI. Although MAG, as well as the Nogo peptide, significantly inhibited the neurite outgrowth from post-natal cerebellar neurons, over-expression of Rho GDI abolished these inhibitory effects (D in FIG. 8). These results are consistent with our suggestion that p75^{NTR} acts as a Rho GDI displacement factor.

### Example 2-4

### The Effect of Peptide Ligand on the Interaction of p75^{NTR} with Rho GDI

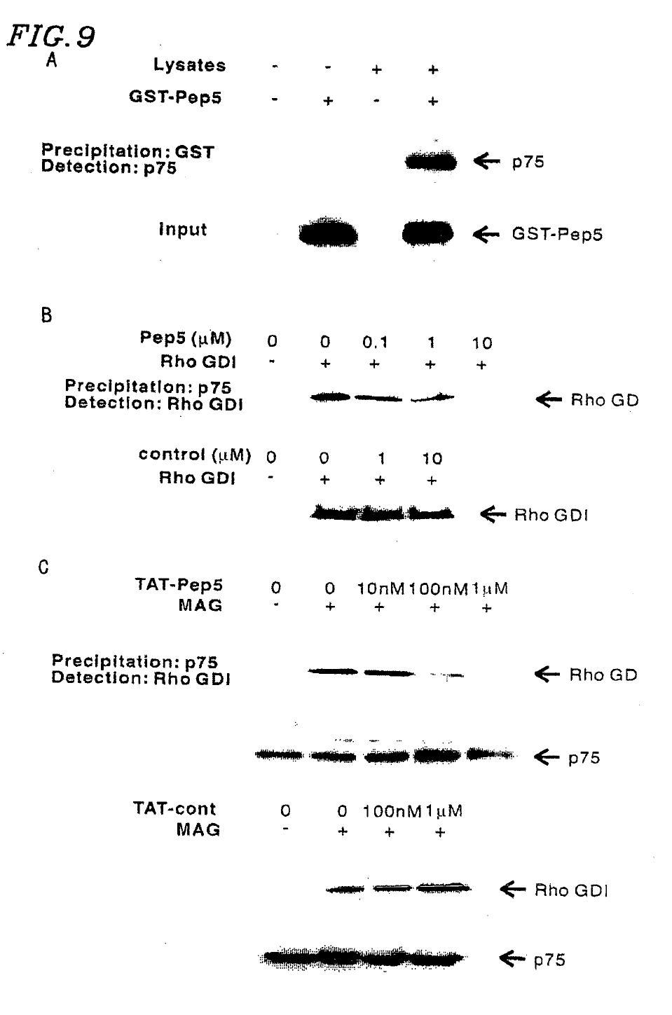

As all the myelin-derived inhibitors of axonal regeneration identified so far act on neurons through p75^{NTR}, intervening with p75^{NTR} signaling after injury to the central nervous system may alleviate myelin-dependent inhibition of axonal regeneration. Pinpointing the region of Rho GDI association allowed us to develop a strategy to specifically inhibit the function of p75^{NTR} The specific peptide ligand to the p75^{NTR} HD was previously obtained from a combinatorial library (Ilag, L. L. et al., Biochem Biophys Res Commun. 255, 104-109 (1999)). This ligand is a 15 amino acid residue peptide (Pep5; CFFRGGFFNHNPRYC (SEQ ID NO. 2)) and the binding site was mapped by nuclear magnetic resonance spectroscopy onto a hydrophobic patch framed by helices 5 and 6. Although the sequence of the peptide did not immediately suggest a protein that exists in mammals, the present inventors were interested in the possibility that it may play a role as a silencer that disrupts the recruitment of Rho GDI to p75^{NTR} HD. The present inventors first confirmed whether p75^{NTR} associates with Pep5. Glutathione S-transferase-fusion protein containing Pep5 (GST-Pep5) was incubated with lysates prepared from post-natal cerebellum that abundantly express p75^{NTR}. In the GST-Pep5 precipitates, the anti-p75^{NTR} antibody revealed the presence of a protein corresponding to p75^{NTR} (A in FIG. 9). Then, binding affinity was compared between Pep5 and Rho GDI. p75^{NTR}, immunoprecipitated and purified from the lysates of the transfected 293T cells, was incubated with 1 μM GST-Rho GDI and Pep5 at the indicated concentrations (B in FIG. 9). Pep5, but not the control peptide, inhibited the association of p75^{NTR} with Rho GDI dose dependently. Therefore, Pep5 has a potential to disrupt the signal mediated by p75^{NTR} in vitro. As the peptide ligand must gain entry into the cell if it is to act directly on the p75^{NTR} HD in vivo, the present inventors generated Pep5 fused with the amino-terminal 11 amino acid protein transduction domain (PTD domain) from the human immunodeficiency virus protein, TAT (TAT-Pep5) (Schwarze, S. R., Ho, A., Vocero-Akbani, A. & Dowdy, S. F., Science 285, 1569-1572 (1999)). The interaction of p75^{NTR} with Rho GDI induced by MAG-Fc in the dissociated cerebellar neurons was significantly inhibited by TAT-Pep5 in a competitive fashion, but not by TAT (PTD domain)-fused control peptide (C in FIG. 9). Thus, Pep5 may be used as an inhibitor of Rho GDI association with p75^{NTR}

### Example 2-5

### Pep5 Silences the Myelin Signal

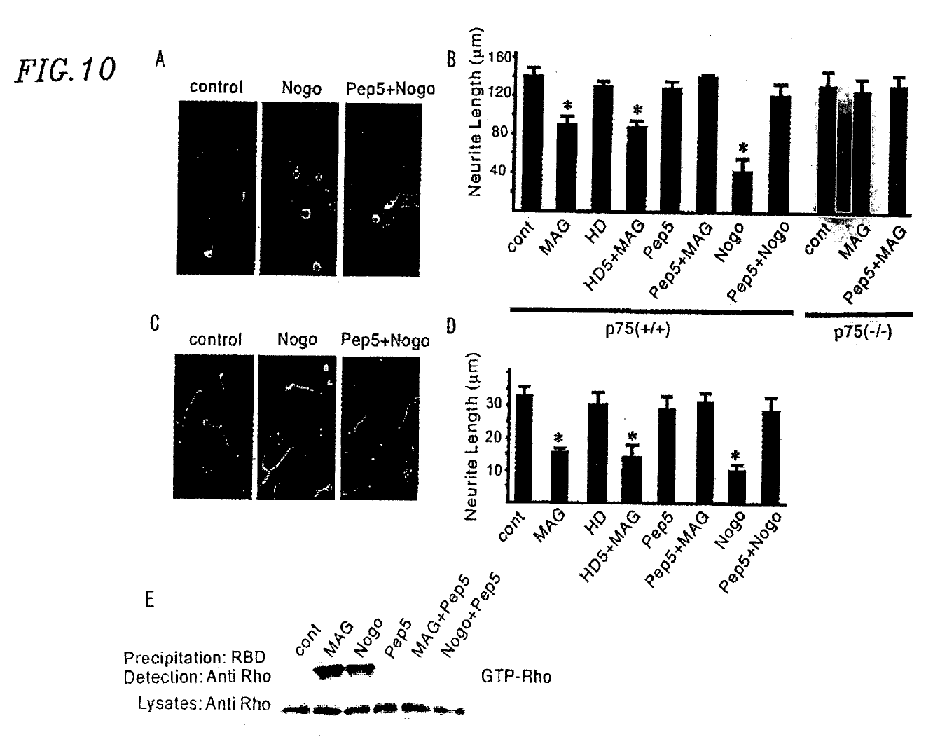

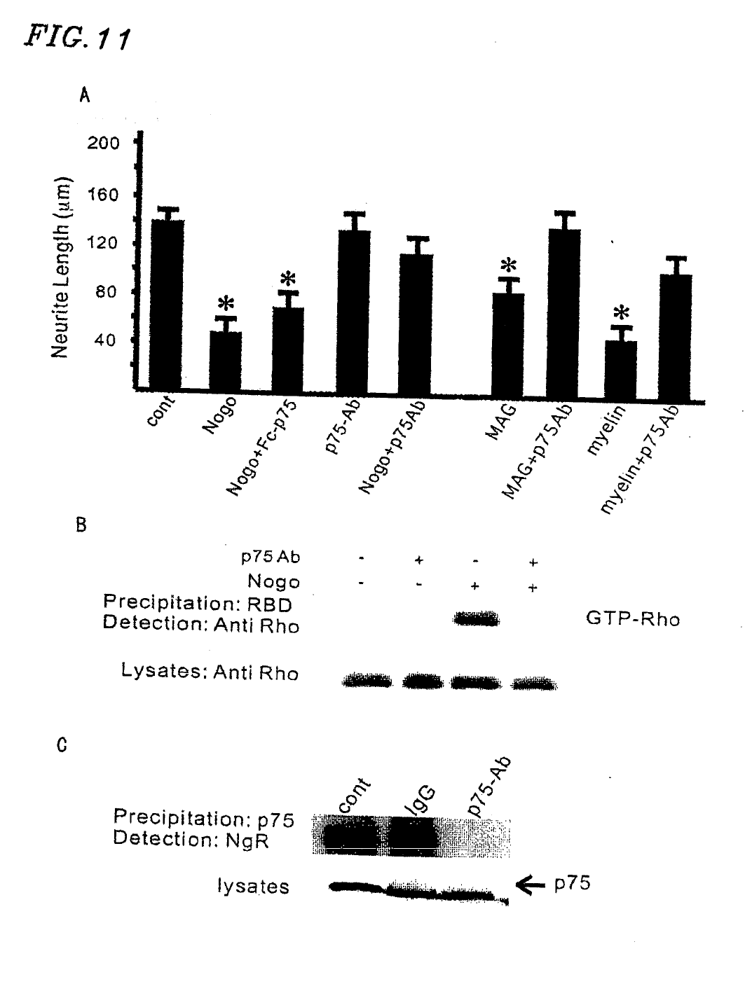

Next question the present inventors asked was if Pep5 inhibits the effect of MAG or Nogo. The present inventors employed the neurite growth assay to measure the effect of MAG or Nogo. The present inventors used another control peptide derived from rat p75^{NTR} corresponding to residue 368 to 381 of SEQ ID NO. 4. This peptide, at the concentration of 100 nM (B in FIG. 10) or 10 μM (data not shown), had no effect on neurite outgrowth of dorsal root ganglion (DRG) neurons, and it did not influence the action of MAG-Fc (B in FIG. 10) or the Nogo peptide (data not shown). However, TAT-Pep5, added exogenously to cultured neurons at the concentration of 100 nM, abolished their responsiveness to MAG (25 μg/ml) as well as the Nogo peptide (4 μM) (A and b FIG. 10). Post-natal cerebellar neurons were used to examine the effects of Pep5. As observed in DRG neurons, TAT-Pep5 efficiently silenced the inhibitory effect of MAG (25 μg/ml) and the Nogo peptide (4 μM) (C and D in FIG. 10). Finally, to show more clearly that the peptide acts as a silencer of p75^{NTR} signaling, the present inventors measured Rho activity by affinity precipitation. As expected, although RhoA was activated 30 min following the addition of MAG-Fc or the Nogo peptide to the post-natal cerebellar neurons, TAT-Pep5 inhibited the activation of RhoA induced by MAG-Fc or the Nogo peptide on these cells (E in FIG. 10). These findings strongly suggest that Pep5 inhibits the activation of RhoA through p75^{NTR} by inhibiting the association of Rho GDI with p75^{NTR}.

Similar results were observed when experiments were carried out using anti-p75^{NTR} antibodies, anti-Rho GDI antibodies, and the p75^{NTR} extracellular domain.

### Example 2-6

### In Vivo Nerve Regeneration Effect of an Agent Capable of Disrupting the Interaction Between a Silencer and/or p75^{NTR} and Rho GDI

200 g male Wistar rats were used. After the ninth thoracic vertebrae laminectomy was performed, the dorsal half of the spinal cord was dissected. A continuous osmotic pump was used to continuously administer either TAT-fused Pep5 or TAT(PTD domain)-fused control peptide to the injured site. As a result, nerve regeneration was significantly observed when TAT-Pep5 was used, as compared to when the control peptide was used.

Similar results were observed when anti-p75^{NTR} antibodies were used.

### Example 2-7

### Demonstration in Mouse

Similar experiments were carried out for mice as described above. As a result, nerve regeneration was similarly observed when TAT-fused Pep5 and anti-p75^{NTR} antibodies were used.

### Example 2-8

### Modified Amino Acid

Similar experiments were carried out using Pep5 in which alanine was added to the C terminus of the sequence (SEQ ID NO. 2), antibodies for the extracellular domain of p75, and p75 in which alanine was replaced with valine at amino acid 423 in positions 273-427 of SEQ ID NO. 4. As a result, nerve regeneration was similarly observed.

### Example 2-9

### Other Agents

Similar experiments were carried out using a nucleic acid molecule encoding the Peps polypeptide, an antibody as an agent capable of specifically interacting with the p75 polypeptide, an antisense and RNAi as an agent capable of specifically interacting with a nucleic acid molecule encoding the p75 polypeptide, the p75 extracellular domain polypeptide, a nucleic acid molecule encoding the p75 extracellular domain polypeptide, and an antibody as an agent capable of specifically interacting with the MAG polypeptide. As a result, neurite outgrowth was observed and in vitro nerve regeneration was observed.

Nerve-related diseases, disorders and conditions have been believed to be difficult to cure completely. However, the above-described effect of the present invention allows diagnosis which has been conventionally believed to be impossible, and is applicable to therapies. Therefore, the present invention can be said to have usefulness which cannot be achieved by conventional diagnostics or medicaments.

### Example 3

### Antibody Capable of Neutralizing p75^{NTR} Promotes Axon Regeneration in Injured CNS

The present inventors investigated the signal transduction pathway associated with p75^{NTR} in greater detail by analyzing an influence of antibodies for p75^{NTR} on the pathway.

(Materials and Methods)

Experiments were carried out using materials and methods essentially similar to those in Examples 1 and 2.

### Example 3-1

### Anti-p75^{NTR} Antibody is a Promising Drug Against the Myelin-bound Inhibitors

The present inventors employed the neurite growth assay to measure the effect of MAG, Nogo and myelin. MAG-Fc (25 μg/ml) and myelin, as well as the Nogo peptide (4 μM), corresponding to residues 31-55 of the extracellular fragment of Nogo (Fournier, A. E. et al., Nature 409, 341-346, 2001), significantly inhibited the neurite outgrowth from post-natal cerebellar neurons (A in FIG. 11). Although the recombinant p75^{NTR} extracellular domain fused to Fc, which was expected to act as a dominant negative form of p75^{NTR}, only partially inhibited the Nogo inhibitory effect, a polyclonal antibody to the extracellular domain of the p75^{NTR} (AB1554, Chemicon), which can be used to block the binding of NGF to the p75^{NTR}, effectively reduced the neurite inhibitory effect (A in FIG. 11). The antibody itself had no effect on the neurite outgrowth. This action is mediated by the inhibition of the signal transduction of the p75^{NTR}, as the activation of RhoA by the Nogo peptide was abolished by the antibody (B in FIG. 11). The inhibitory effect of the antibody may be dependent on the inhibition of association of the p75^{NTR} with the Nogo receptor, as the interaction of the Nogo receptor with the p75^{NTR} was reduced by the antibody (C in FIG. 11), as previously shown using the antibody to frog p75^{NTR} (Wong, S. T. et al., Nat. Neurosci. 5, 1302-1308, 2002). These results suggest the antibody to be a promising agent against the myelin-associated inhibitors.

### Example 3-2

### Anti-p75 Antibody Promotes Axon Regeneration in Injured CNS

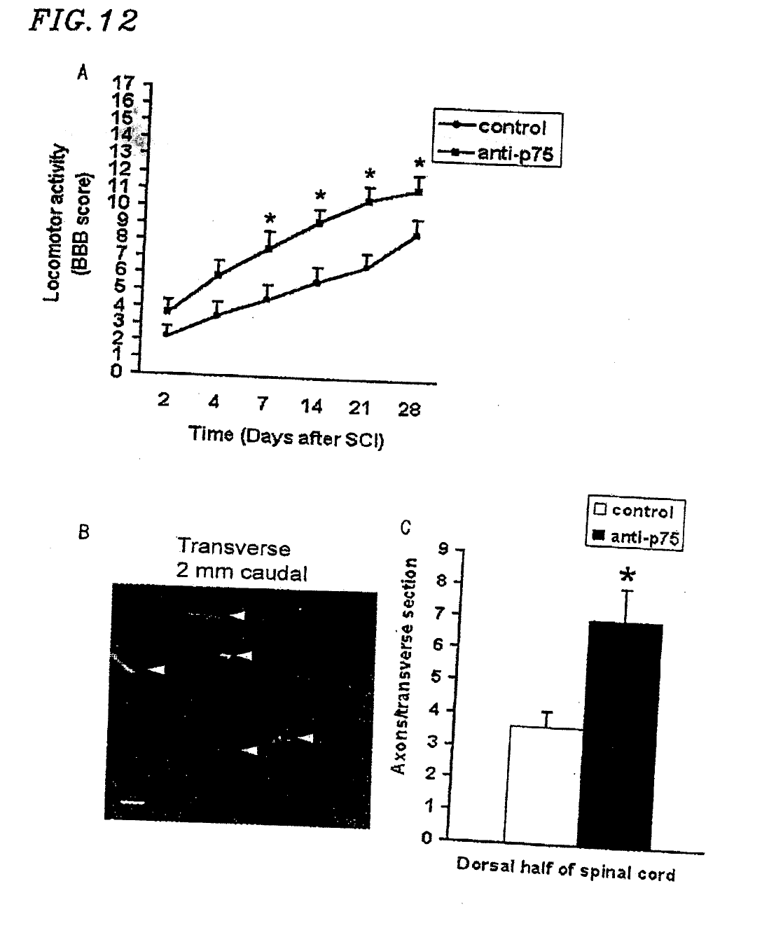

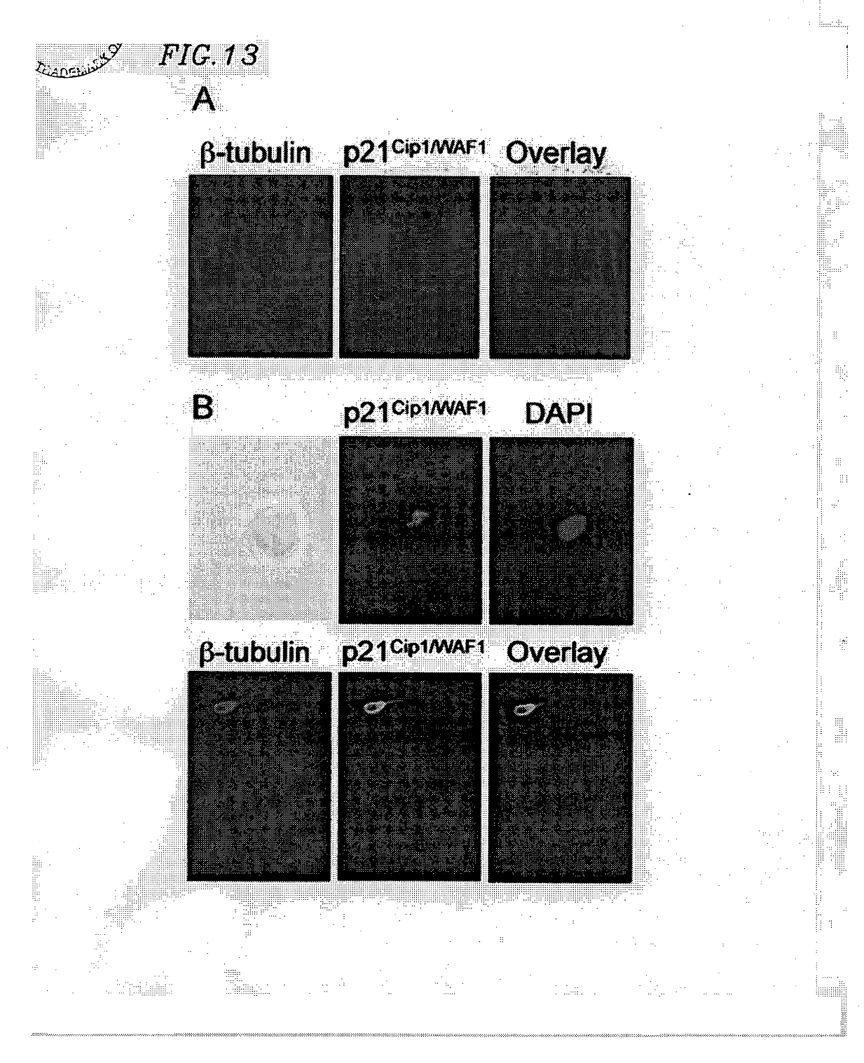

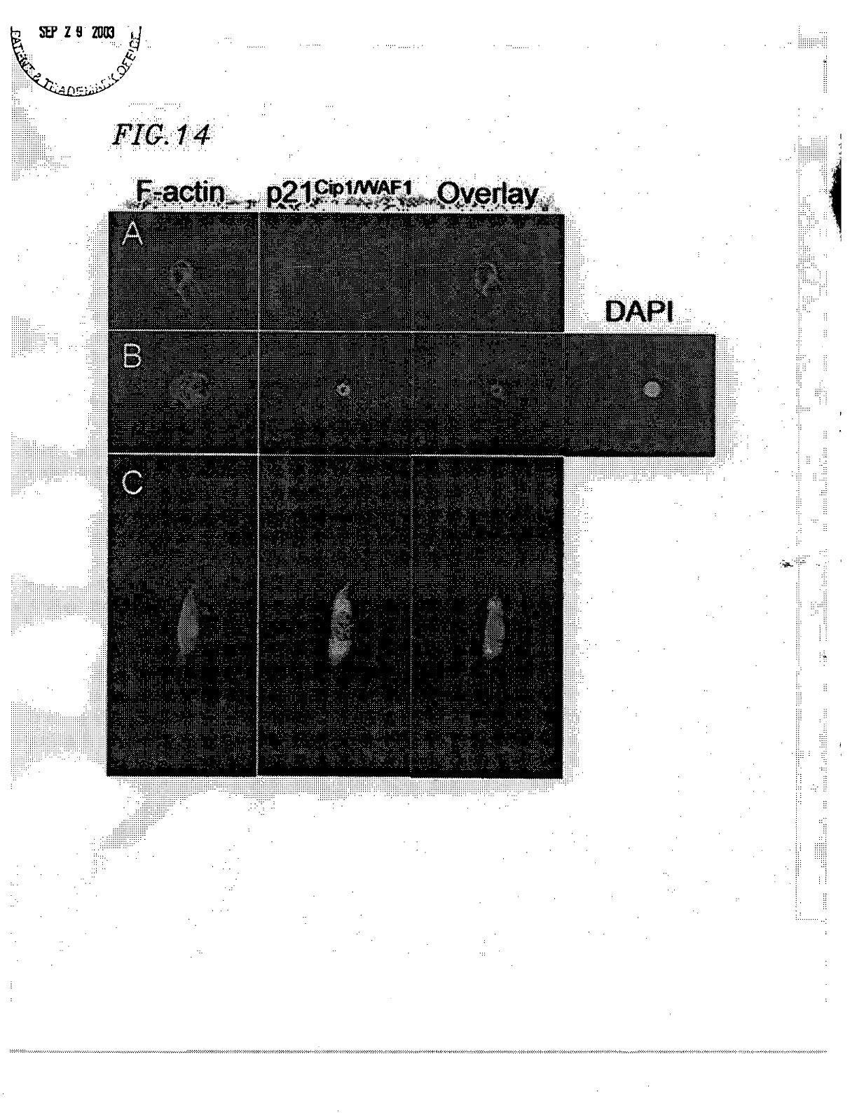

The present inventors next tested the ability of the antibody to promote the regeneration of cortico-spinal tract (CST) fibers after dorsal hemisection lesions at thoracic level T10/T11 in adult mice. The anti-p75 antibody or control antibody was delivered via an osmotic mini-pump (Alzet 1002, Durect Corp., Cupertino, Calif.; 100 μl solution at 0.25 μl per hour over 2 weeks) with catheters placed above the site of injury. The CST was anterogradely labeled by injection of the anterograde neuronal tracer BDA into the motor cortex (Fournier, A. E. et al., J. Neurosci. 23, 1416-1423, 2003). After injury, the recovery of locomotor behavior was assessed using the modified BBB scale (Dergham, P et al., J. Neurosci. 22, 6570-6577, 2002). Animals undergoing a dorsal hemisection at level T10/T11 finally regained partial functional recovery as assessed by the modified BBB scale (A in FIG. 12). Functional recovery of the anti-p75 antibody-treated mice was significantly higher than that of the control antibody-treated mice from seven days to 4 weeks after injury. In the anti-p75 antibody-treated mice, transverse sections 2 mm caudal to the injury site showed increased numbers of regenerating axons in the dorsal half of spinal cord (B in FIG. 12). The number of regenerating axons was increased twofold in the dorsal half of spinal cord (C in FIG. 12)

### Example 3-3

### Other Agents

Similar experiments were carried out using a nucleic acid molecule encoding the Pep5 polypeptide, an antibody as an agent capable of specifically interacting with the p75 polypeptide, an antisense and RNAi as an agent capable of specifically interacting with a nucleic acid molecule encoding the p75 polypeptide, the p75 extracellular domain polypeptide, a nucleic acid molecule encoding the p75 extracellular domain polypeptide, an antibody as an agent capable of specifically interacting with the Rho GDI polypeptide, an antisense and RNAi as an agent capable of specifically interacting with a nucleic acid molecule encoding the Rho GDI polypeptide, an antibody as an agent capable of specifically interacting with the MAG polypeptide, and an antisense and RNAi as an agent capable of specifically interacting with a nucleic acid molecule encoding the MAG polypeptide. As a result, neurite outgrowth was observed and in vitro nerve regeneration was observed.

### Example 4

### Cytoplasm p21^{Cip1/WAF1} Modulates Neurite Remodeling by Inhibiting Rho Kinase Activity

During the period of active neurogenesis, some neuroblasts enter the postmitotic state and then start migrating to their final destination. In the embryonic chick retina, ganglion cells are actively generated around embryonic day 5 (ES) (Frade J. M. et al., Development 124:3313-3320, 1997). The present inventors examined expression of p21^{Cip1/WAF1} in these cells to test whether p21^{Cip1/WAF1} was associated with differentiation and morphogenesis of these cells.

(Materials and Methods)

(Preparation of Chick Retina and Retinal Cells)

Whole chick ES embryos (White Leghorn) were fixed with 4% paraformaldehyde in PBS overnight and immersed in 30% sucrose. Cryosections (30 μm in thickness) of retinas were cut on the coronal plane, thaw-mounted onto slides and dried at room temperature. For retinal neuron culture, retinas from ES embryos were dissected free from the pigment epithelium and dissociated as described previously (Rodriguez-Tebar, A. et al., Dev. Biol. 136:296-303, 1989; de la Rosa, E. J. et al., Neuroscience. 58:347-352, 1994). Dissociated cells were plated (20,000 cells/cm²) on 4-well chamber slides (Nalge Nunc International K.K.), which were previously coated with poly-L-ornithine/laminin (Sigma) (Collins, F., Dev. Biol. 65:50-57, 1978). Cells were cultured in Dulbecco's modified Eagle's medium (DMEM)/F12 mixture (1:1) with N2 supplement (Bottenstein, J. E. & Sato, G. H., Proc. Natl. Acad. Sci. USA. 76:514-517, 1979), and maintained at 37° C. in a water saturated atmosphere containing 5% CO₂ for 12 hours and fixed with 4% paraformaldehyde in PBS.

(Plasmid Constructs)

pEGFP-full-p21 (aa 1-164) (SEQ ID NO. 23) and pEGFP-ΔNLS-p21 (aa 1-140 in SEQ ID NO. 23) are mammalian expression vectors for GFP fused proteins (Asada, M. et al., EMBO J. 18:1223-1234, 1999). Myc-Rho-kinase in pEF-BOS was kindly provided by Dr. K. Kaibuchi (Nagoya University, Japan).

(Cell Culture and Transfection)

NIH3T3 cells, N1E-115 cells and 293T cells were maintained in DMEM containing 10% fetal bovine serum. Lipofectamine 2000 (Invitrogen) was used for transfection. For the stress fiber formation assay, NIH3T3 cells were cultured in serum-free medium for 16 hours after transfection. Stress fiber formation was evoked by incubating the cells with 10% serum for 10 minutes. Hippocampal neurons were prepared from 18-day-old Sprague-Dawley rats, as previously described (Neumann, H. et al., Science. 269:549-552, 1995). Briefly, hippocampi were dissected and the meninges removed. The trimmed tissue was dissociated by trituration. The dissociated cells were plated on dishes pre-coated with poly-L-lysine (Sigma), and cultured in DMEM containing 10% fetal bovine serum for 24 hours. Then, the medium was replaced with DMEM with B27 supplement (Invitrogen), and the cells were transfected with GFP or GFP-ΔNLS-p21. Neuronal morphology was estimated at 24 hours after the transfection.

(Morphological Analysis of N1E-115 Cells

N1E-115 cells were transfected with GFP, GFP-full-p21 or GFP-ΔNLS-p21, and cultured in serum-starved condition for 5 hours. Then, the medium was replaced with DMEM containing 10% fetal bovine serum. The cells were fixed at 48 hours after transfection. The morphology of the cells was categorized into 3 groups; neurite positive cells, round cells and the other cells. The cells with longer neurites than their soma were defined as neurite positive cells. The other cells had various features including microspikes, ruffles and a flattened appearance.

(Co-immunoprecipitation of ΔNLS-p21 and Rho-kinase)

293T cells were transfected with myc-Rho-kinase in combination with GFP-full-p21 or GFP-ΔNLS-p21. At 48 hours after transfection, the cells were lysed with 1 ml of lysis buffer (50 mM Tris-HCl (pH 7.5), 150 mM NaCl, 10% glycerol, 0.5% Nonidet-P40 including protease inhibitor cocktail tablets (Roche). The cell lysates were centrifuged at 13,000×g for 20 minutes, and the supernatant was collected. Immunoprecipitations were performed for 2 hours at 4° C. using an anti-p21^{Cip1/WAF1} mouse monoclonal antibody (Santa Cruz Biotechnology) and 0.75 ml of the supernatant. The immunocomplexes were collected with protein G-Sepharose (Amersham Pharmacia Biotech) slurry (50% v/v), washed 4 times with lysis buffer, and subjected to SDS-PAGE. They were transferred to the polyvinylidene difluoride membranes and probed with the anti-myc rabbit polyclonal antibody (Santa Cruz Biotechnology). Interaction of endogenous proteins in N1E-115 cells was assessed in the same way using anti Rho-kinase antibody.

(In Vitro Binding Assay)

Recombinant full-length p21^{Cip1/WAF1} (1-164 in SEQ ID NO. 23, >98% purity, 1 nM, Santa Cruz) and purified GST fused protein of a fragment of Rho-kinase (GST-CAT; aa 6-553) were incubated in 1 ml of buffer (50 mM Tris-HCl (pH 7.5), 150 mM NaCl, 5 mM MgCl₂, 1 mM DTT and 1 mM EDTA including protease inhibitor cocktail tablets) for 2 hours, and GST-CAT was precipitated using glutathion sepharose (Amersham Pharmacia Biotech). The resultant precipitates were electrophoretically transferred to polyvinylidene difluoride membranes after SDS/PAGE with 10% gels and were immunoblotted with the anti-p21^{Cip1/WAF1} antibody.

(Kinase Assay)

The kinase reaction for Rho-kinase was carried out using a S6 Kinase Assay Kit (Upstate Biotechnology) according to the manufacturer's instructions. Briefly, for in vitro assay, 10 μl of assay dilution buffer (ADB: 20 mM MOPS (pH 7.2), 25 mM β-glycerol phosphate, 5 mM EGTA, 1 mM sodium orthovanadate and 1 mM dithiothreitol), 10 μl of substrate cocktail (250 μM substrate peptide (AKRRRLSSLRA (SEQ ID NO. 24)) in ADB), 10 μl of the inhibitor cocktail, 10 μl of the [γ-³²P] ATP mixture (Magnesium/ATP cocktail including 10 μCi of the [γ-³²P] ATP) and 20 mU of Rho kinase fragment (aa 1-543, Upstate Biotechnology) were mixed. After incubation with p21^{Cip1/WAF1} protein for 10 minutes at 30° C., the reaction mixtures were spotted onto the P81 phosphocellulose paper and quantified using a scintillation counter.

For the in vivo assay, 293T cells were co-transfected with myc-Rho-kinase in combination with GFP or p21^{Cip1/WAF1} constructs. Cells were lysed with lysis buffer (50 mM Tris-HCl (pH 7.5), 150 mM NaCl, 10% glycerol, 1% Nonidet-P40 and protease inhibitor cocktail). The kinase assay was carried out using the lysates.

(Immunostaining)

For immunohistochemistry, sections of chick retinas were permeabilized and blocked with the blocking buffer (0.1% Triton X-100, 0.1% BSA, and 5% goat serum in PBS) for 30 minutes at room temperature. For immunocytochemistry, cells were permeabilized and blocked with buffer containing 0.2% Triton X100. They were incubated overnight at 4° C. with the anti-p21^{Cip1/WAF1} antibody (1:1000) and an anti-β-tubulin class III rabbit polyclonal antibody (TuJ1) (1:2000, Research Diagnostic, Inc.), followed by incubation for 1 hour with Alexa 488-labeled goat anti-mouse IgG antibody (Molecular Probes) and Alexa 568-labeled goat anti-rabbit IgG antibody (Molecular Probes). Tetramethyl rhodamine isothiocyanate-labeled phalloidin (1:1000, Sigma) was used to detect F-actin in NIH3T3 cells and N1E-115 cells. Hippocampal neurons were immunostained with the anti-TuJ1 antibody. When necessary, DAPI (300 nM, Wako) was used to stain the nucleus. Samples were examined under a confocal laser-scanning microscope (Carl Zeiss).

### Example 4-1

### E5 Embryo-derived Chick Retinal Neuron Exhibits Cytoplasm p21^{Cip1/WAF1} Expression

Using immunohistochemistry it was found that retinal neurons immediately after neurogenesis were migrating into deep layers (A in FIG. 13). p21^{Cip1/WAF1} immunoreactivity was detected in the cells at the vitreous surface of the central neural retina using a monoclonal antibody against p21^{Cip1/WAF1} (A in FIG. 13). These p21^{Cip1/WAF1} positive cells were immature retinal neurons before migration. Therefore, it is suggested that p21^{Cip1/WAF1} is involved in the differentiation of retinal precursor cells in vivo.

Next, the present inventors isolated neural precursor cells from E5 retinas to assess more precisely the subcellular localization of p21^{Cip1/WAF1}. Dissociated retinal cells cultured on laminin-1 extended neurites rapidly (Frade, J. M. et al., Exp. Cell. Res. 222:140-149, 1996b). Cells were cultured on laminin-1 in a chemically-defined medium containing 1 μM insulin. Insulin used in the micromolar range is likely to be acting on insulin-like growth factor-I receptors, thus mimicking the differentiative effect of insulin-like growth factor-I on the E5 retinal cells (Frade, J. M. et al., Development. 122:2497-2506, 1996a). In almost all the immature cells devoid of immunoreactivity for β-tubulin, the expression of p21^{Cip1/WAF1} was predominantly seen in the nucleus (B in FIG. 13). p21^{Cip1/WAF1} in the nucleus may contribute to a change in the cell cycle in these cells. On the other hand, in most neurons that had relatively long neurites with immunoreactivity for neuron-specific β-tubulin, p21^{Cip1/WAF1} was mainly localized in the cytoplasm (B in FIG. 13). These findings suggest that cytoplasmic expression of p21^{Cip1/WAF1} is induced in the new neurons.

### Example 4-2

### In Vitro Differentiation of N1E-115 Cells is Associated with p21^{Cip1/WAF1} Expression in the Cytoplasm

The present inventors next used neuroblastoma N1E-115 cells to examine whether neuronal differentiation was associated with cytoplasmic expression of p21^{Cip1/WAF1} N1E-115 cells, which were induced to differentiate by DMSO were immunostained with the anti-p21^{Cip1/WAF1} antibody. After 24 hours of DMSO treatment, p21^{Cip1/WAF1} was induced in the nucleus (B in FIG. 14). However, after 4 days, a time point when the extensive neurite genesis was well evident, p21^{Cip1/WAF1} was mainly localized in the cytoplasm (C in FIG. 14). In this regard, the differentiation-associated cytoplasmic expression of p21^{Cip1/WAF1} is not restricted to chick retinal precursor cells.

### Example 4-3

### Ectopic Expression of p21^{Cip1/WAF1} Affects the Morphology of N1E-115 Cells

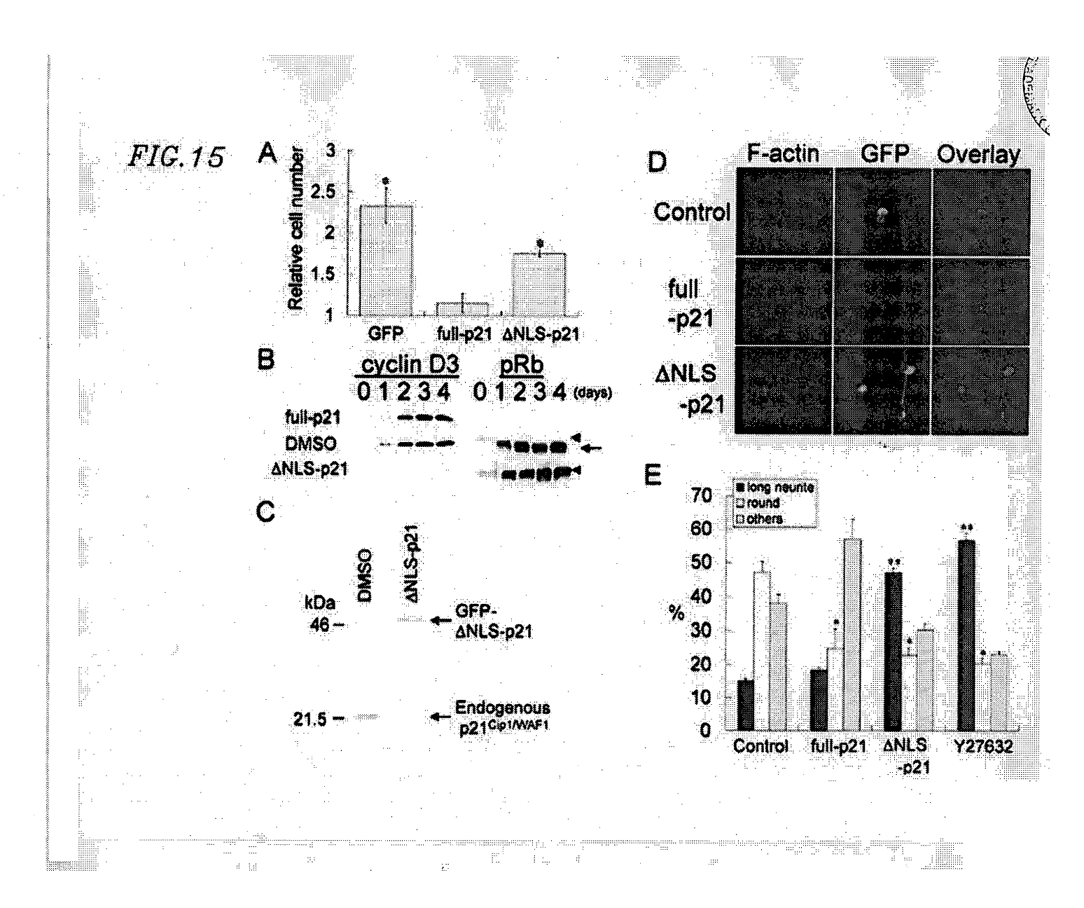

As the cells with cytoplasmic expression of p21^{Cip1/WAF1} extended long neurites, and those devoid of cytoplasmic p21^{Cip1/WAF1} did not (FIG. 13 and 14), the present inventors hypothesized that cytoplasmic p21^{Cip1/WAF1} was associated with neurite outgrowth. Therefore, the present inventors next asked if relocalization of p21^{Cip1/WAF1} to the cytoplasm elicited the extension of the neurites. To address this question, the mammalian expression vector for p21^{Cip1/WAF1} with loss of nuclear localization signal. (ΔNLS-p21; aa 1-140) as well as the full length p21^{Cip1/WAF1} (full-p21; aa 1-164) was made (Asada, M. et al., EMBO J. 18:1223-1234, 1999). The cells transfected with ΔNLS-p21 or green fluorescent protein (GFP) proliferated until 48 hours after transfection (A in FIG. 15), although those with full-p21 stopped proliferation. In the cells transfected with full-p21 or treated with DMSO, the protein level of cyclin D3 strongly increased (Kranenburg, O. et al., J. Cell. Biol. 131:227-234, 1995), whereas no change in the expression was found in those with ΔNLS-p21 (B in FIG. 15). Furthermore, although underphosphorylated pRb, retinoblastoma gene product, was induced and hyperphosphorylated pRb became undetectable by DMSO treatment, hyperphosphorylated pRb remained predominant in ΔNLS-p21 transfected cells during the observation period (B in FIG. 15). These data demonstrate that ΔNLS-p21 has no differentiation inducing activity in N1E-115 cells, as shown in U937 cells (Asada, M. et al., EMBO J. 18:1223-1234, 1999), thus enabling us to estimate the effects of p21^{Cip1/WAF1} without taking the differentiation effect on the cells into account. The expression level of ΔNLS-p21 in N1E-115 cells was comparable with that of endogenous p21^{Cip1/WAF1} in the cells with DMSO treatment for 4 days (C in FIG. 15). N1E-115 cells were transfected with these constructs and the morphological changes were assessed 48 hours later. The cells with the full-length p21^{Cip1/WAF1} expression showed a somewhat flattened and enlarged appearance and decreased cell rounding (D in FIG. 15) compared to those with GFP expression or no transfection, while there was no increase in the cell population that had long neurites (E in FIG. 15). These changes may be caused by the differentiation of N1E-115 cells expressing p21^{Cip1/WAF1} in the nucleus (Kranenburg, O. et al., J. Cell. Biol. 131:227-234, 1995), as the present inventors observed a similar phenotype when the cells were induced to differentiate by DMSO treatment (Kimhi, Y. et al., Proc. Natl. Acad. Sci. USA. 73:462-466, 1976) (data not shown). The cells with the full-length p21^{Cip1/WAF1} expression extended long neurites 4 days later, a time point when the signal for p21^{Cip1/WAF1} was also seen in the cytoplasm (data not shown). On the other hand, more than 45% of the cells transfected with ΔNLS-p21 extended long neurites (3.1-fold increase compared to the control) (E in FIG. 15). This result suggests that cytoplasmic p21^{Cip1/WAF1} regulates neurite remodeling in N1E-115 cells.

### Example 4-4

### Effects of Cytoplasmic p21^{Cip1/WAF1} on the Cytoskeletal Organization

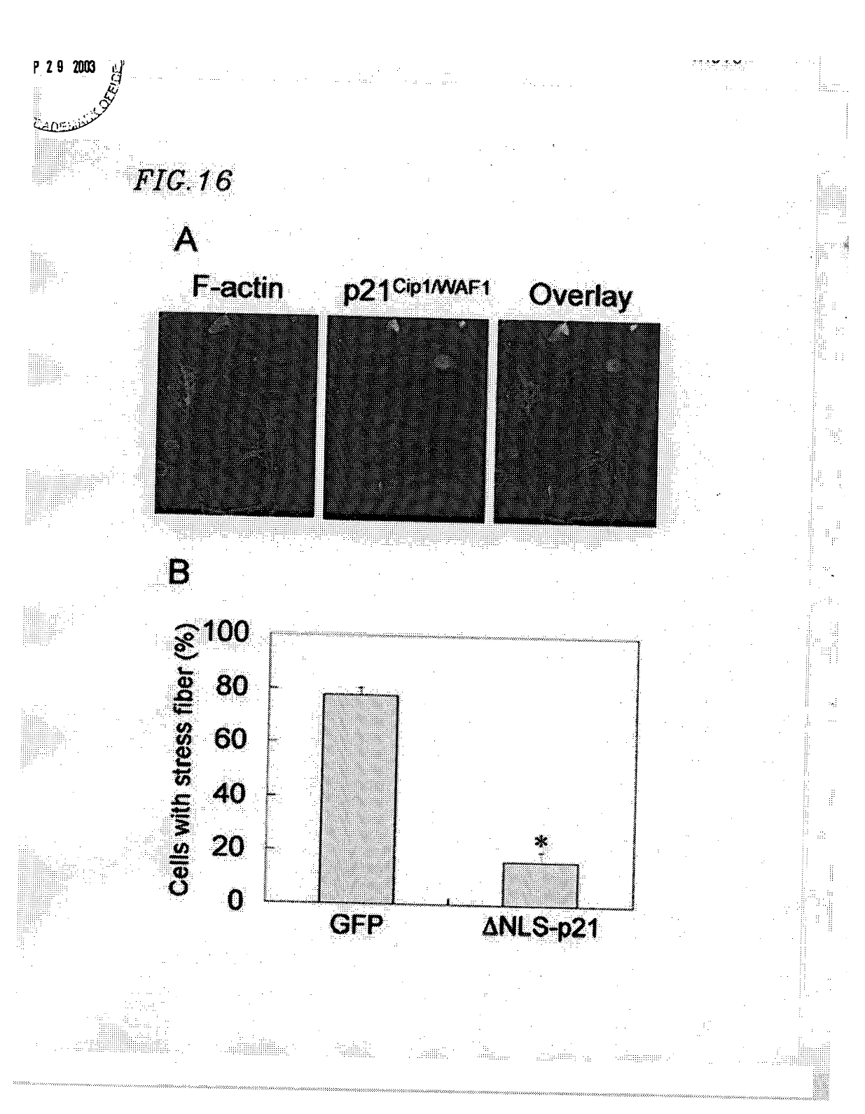

Overexpression of a dominant-active mutant of RhoA or p160ROCK, an isoform of Rho-kinase, induced cell rounding in N1E-115 cells (Hirose, M. et al., J. Cell. Biol. 141:1625-1636, 1998), but the expression of a dominant-negative mutant of p160ROCK or treatment with Y-27632 (E in FIG. 15), chemical compounds with specific inhibitory activity of Rho-kinase (Uehata, M. et al., Nature 389:990-994, 1997), induced significant neurite formation (Hirose, M. et al., J. Cell. Biol. 141:1625-1636, 1998). Our findings in N1E-115 cells in combination with these previous reports suggest that the neurite promoting activity of cytoplasmic p21^{Cip1/WAF1} may be associated with Rho/Rho-kinase. Therefore, the present inventors next used NIH3T3 cells to examine whether p21^{Cip1/WAF1} would regulate actin cytoskeleton mediated by Rho. NIH3T3 cells were transfected with ΔNLS-p21, and then were serum-starved for 16 hours. Incubation with serum for 10 minutes induced the formation of actin stress fibers, preferentially through activation of Rho (Ridley, A. J. & Hall, A., Cell 70:389-399, 1992). However, NIH3T3 cells transfected with ΔNLS-p21 had little stress fiber formation after the addition of serum, while prominent stress fibers were found in non-transfected cells (FIG. 16). Extensive actin stress fibers were observed in the cells with the full-length p21^{Cip1/WAF1} expression (data not shown). These results suggest that Rho-induced actin reorganization in NIH3T3 cells may be blocked by the cytoplasmic expression of p21^{Cip1/WAF1}

### Example 4-5

### p21^{Cip1/WAF1} Binds to Rho-kinase in the Cytoplasm

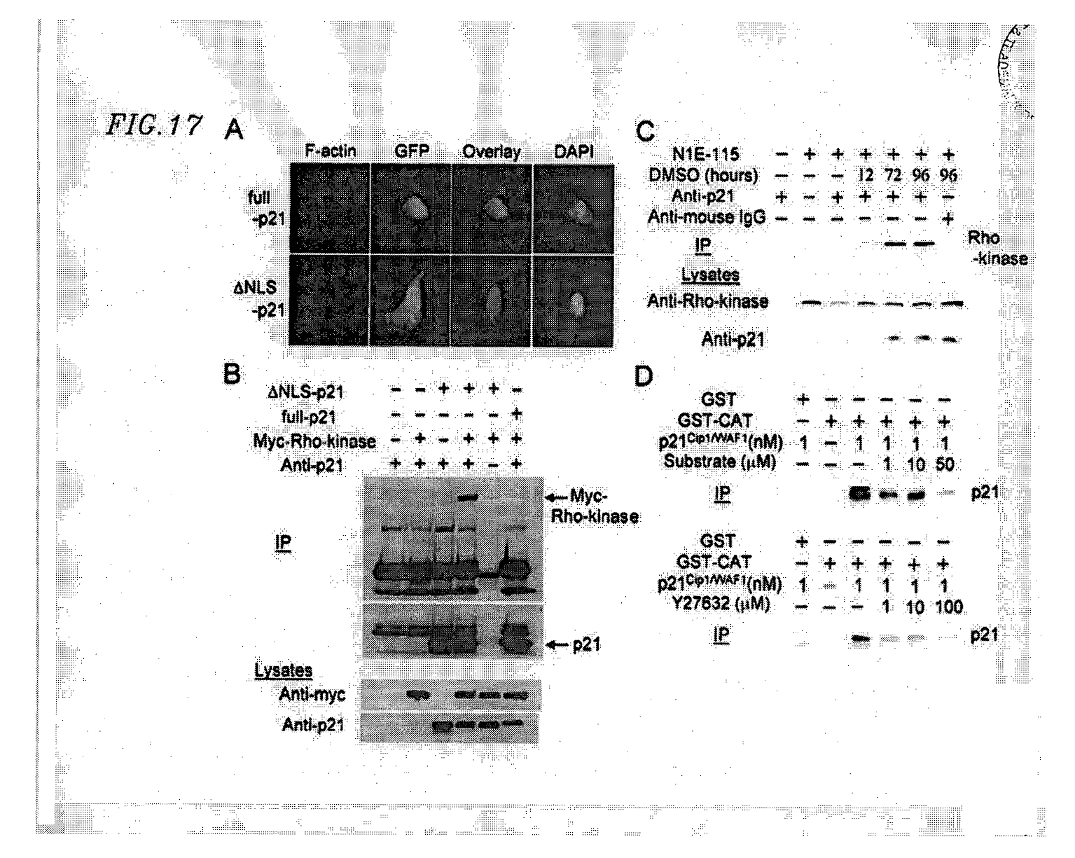

Rho-kinase was shown to work with mDial to elicit the Rho induced phenotype in fibroblast (Watanabe, N. et al., Nat. Cell Biol. 1:136-143, 1999). As serum is one of the most potent activators of Rho (Ridley, A. J. & Hall, A., Cell 70:389-399, 1992), loss of stress fiber formation by the expression of cytoplasmic p21^{Cip1/WAF1} in serum stimulated cells may result from the blockade of the downstream pathway of Rho. Morphological changes of N1E-115 cells by the expression of ΔNLS-p21 were comparable with those by Y-27632 (E in FIG. 15). Given that p21^{Cip1/WAF1} inhibits the activity of the apoptosis signal-regulating kinase 1 (Asada, M. et al., EMBO J. 18:1223-1234, 1999) as well as cyclin-Cdk kinases that are serine threonine kinases (for review, see Pines, J., Biochem. J. 308:697-711, 1995), the present inventors speculated that p21^{Cip1/WAF1} might inhibit the activity of Rho-kinase, which is also a serine threonine kinase. To test the possibility that cytoplasmic p21^{Cip1/WAF1} forms a complex with Rho-kinase in the cytoplasm, co-immunoprecipitation studies were performed using the 293T cells cotransfected with GFP-ΔNLS-p21 and myc-tagged Rho-kinase. Cytoplasmic expression was well evident in the 293T cells transfected with GFP-ΔNLS-p21 (A in FIG. 17). When the lysates were immunoprecipitated with the anti-p21^{Cip1/WAF1} antibody, p21^{Cip1/WAF1} efficiently precipitated myc-tagged Rho-kinase (B in FIG. 17). In an attempt to test if the interaction of ΔNLS-p21 with Rho-kinase depends on its cellular localization, the present inventors then tested the interaction of Rho-kinase with GFP-full-p21, which was expressed predominantly in the nucleus (A in FIG. 17). In contrast to ΔNLS-p21, only a faint signal could be detected (B in FIG. 17), despite comparable expression of the full-length and truncated forms of p21^{Cip1/WAF1} in the 293T cells.

Interaction of the artificially over-expressed proteins may be difficult to detect in natural cells. Employing the anti-p21 antibody, the present inventors examined the interaction of endogenous proteins using lysates prepared from differentiating N1E-115 cells. N1E-115 cells expressed p21^{Cip1/WAF1} in the cytoplasm after treatment with DMSO for 3 to 4 days (FIG. 14). In the p21 immunoprecipitates, the anti Rho-kinase antibody revealed the presence of a protein corresponding to Rho-kinase (C in FIG. 17).

The lack of an interaction of the full length-p21 with Rho-kinase may be attributable to the difference of the localization in the cells. Therefore, the present inventors tested the in vitro interaction of the recombinant full-length p21^{Cip1/WAF1} and Rho-kinase. These proteins bound to each other in vitro (D in FIG. 17). As glutathione S-transferase (GST) fused to the fragment of Rho kinase used here corresponds to the catalytic region of Rho-kinase (GST-CAT; aa 6-553), p21^{Cip1/WAF1} may directly bind to the catalytic region of Rho-kinase. This is substantiated by our finding that S6 kinase substrate peptide (AKRRRLSSLRA) as well as Y-27632 inhibited the interaction of p21^{Cip1/WAF1} with Rho-kinase in a dose dependent manner (D in FIG. 17). These results suggest that p21^{Cip1/WAF1} associates with Rho-kinase in the cytoplasm.

### Example 4-6

### p21^{Cip1/WAF1} Inhibits Rho-kinase Activity

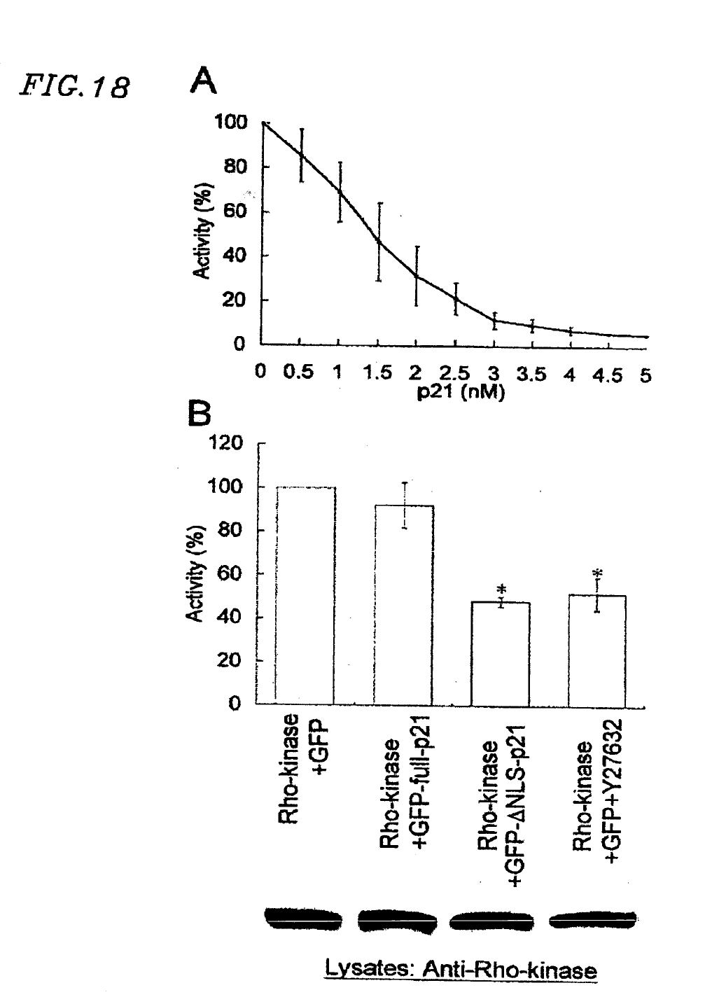

The present inventors next investigated whether p21^{Cip1/WAF1} could inhibit the activity of Rho-kinase in vitro. The kinase assay was carried out using S6 kinase substrate peptide and [γ-³²P] ATP. By using a scintillation counter, the quantity of ³²P-labeled substrate peptide on the phosphocellulose paper was determined. This kinetic analysis revealed that p21^{Cip1/WAF1} inhibited the Rho-kinase activity toward S6 kinase substrate peptide in a dose-dependent manner (A in FIG. 18), and the estimated IC₅₀ value was 1.43 nM.

These results prompted us to examine whether the Rho-kinase activity was inhibited by the expression of ΔNLS-p21 in vivo. 293 T cells were transfected with myc-Rho-kinase with or without ΔNLS-p21. The kinase assay was carried out using the lysates from the cells in the same method as the in vitro assay. The results show that the Rho-kinase activity was inhibited to 48.1% (compared to the original) on average in the cells expressing ΔNLS-p21 compared to the control (B in FIG. 18). This inhibitory effect was comparable with that of Y-27632 (51.9% (compared to the original) inhibition), although expression of the full-length p21^{Cip1/WAF1} had no significant effect. The present inventors' data clearly demonstrate that the activity of Rho-kinase was inhibited by p21^{Cip1/WAF1} in vivo as well as in vitro.

### Example 4-7

### Cytoplasmic p21^{Cip1/WAF1} Promotes Neurite Outgrowth and Branching of the Hippocampal Neurons

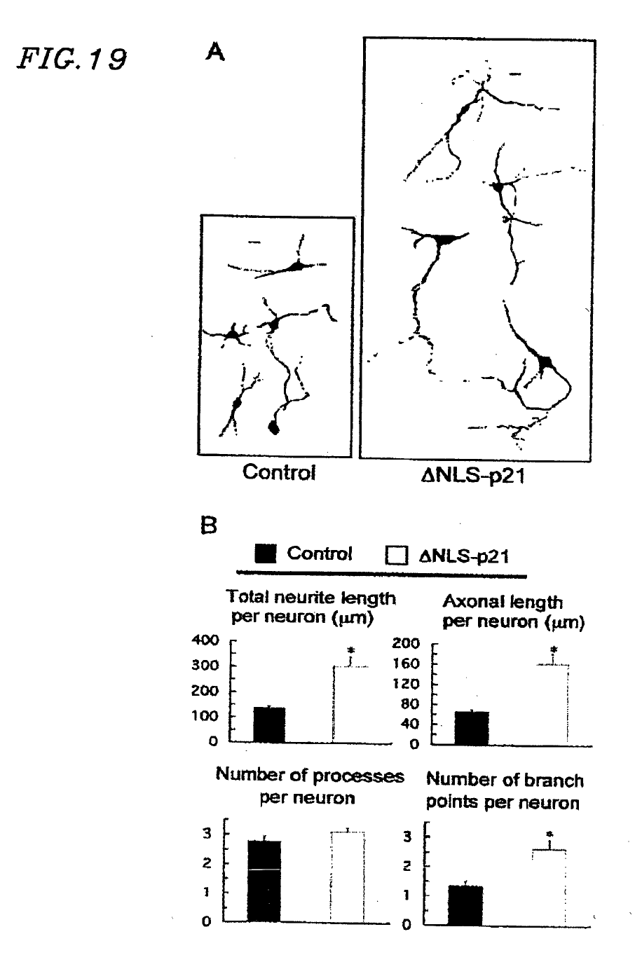

To investigate the relevance of our findings that the cytoplasmic p21^{Cip1/WAF1} acts on Rho-kinase, the present inventors assessed the effects on neurons. Cultures of the hippocampal neurons from rat E18 embryos were used. The present inventors chose these neurons, as they did not express enough endogenous p21^{Cip1/WAF1} to be detected by immunocytochemistry using the anti-p21^{Cip1/WAF1} antibody (data not shown). Dissociated hippocampal neurons were incubated for 48 hours and transfected with ΔNLS-p21. Twenty-four hours after transfection, the cells were fixed and immunolabeled with β-tubulin III. The total neurite length per neuron, the axonal length, defined as the length of the longest neurite per neuron, the number of primary processes originating from the neuronal somata, and the number of branch points per neuron were determined (Neumann, H. et al., J. Neurosci. 22:854-862, 2002). The neuronal morphology of the cells expressing ΔNLS-p21 was apparently different from the control cells without transfection or expressing GFP (A in FIG. 19). The cells with the ΔNLS-p21 expression extended longer neurites and had more branch points than the control cells (GFP expressing cells or no transfection). Ectopic expression of ΔNLS-p21 increased the total neurite length per neuron from 135.9 μm (±7.2 μm SEM) to 307.2 μm (±34.0 μm SEM), the axonal length from 66.3 μm (±3.2 μm SEM) to 162.9 μm (±18.6 μm SEM), and the number of branch points per neuron from 1.3 (±0.2 SEM) to 2.6 (±0.3 SEM). However, no change in the number of primary processes was found by overexpression of cytoplasmic p21^{Cip1/WAF1} (B in FIG. 19). These results indicate that cytoplasmic p21^{Cip1/WAF1} regulates neurite remodeling in the embryonic hippocampal neurons.

### Example 4-8

### Effects of TAT-bound p21

p21 was subjected to nerve regeneration experiments using 200 g male Wistar rats. As a result, an effect was not sufficiently observed.

Next, the present inventors prepared p21 to which a TAT PTD domain was bound and investigated the effect.

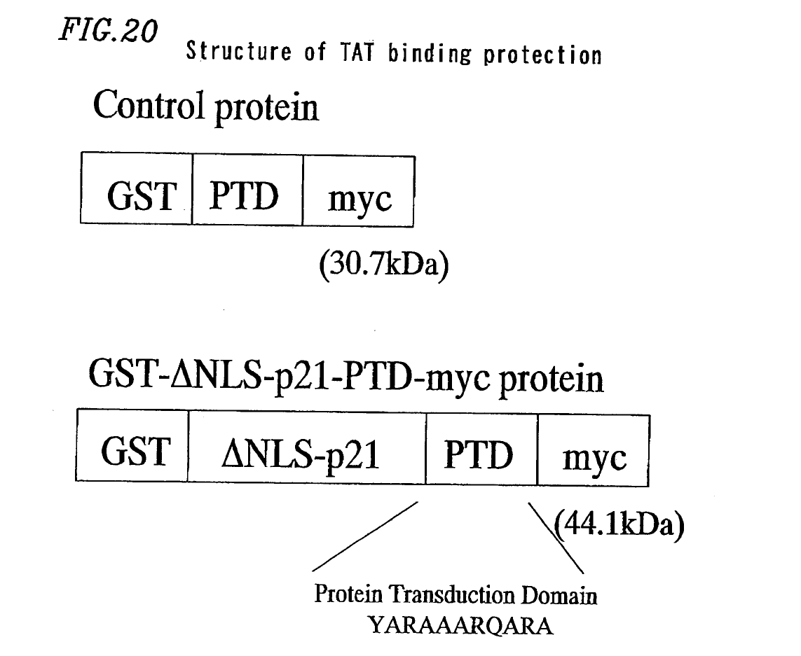

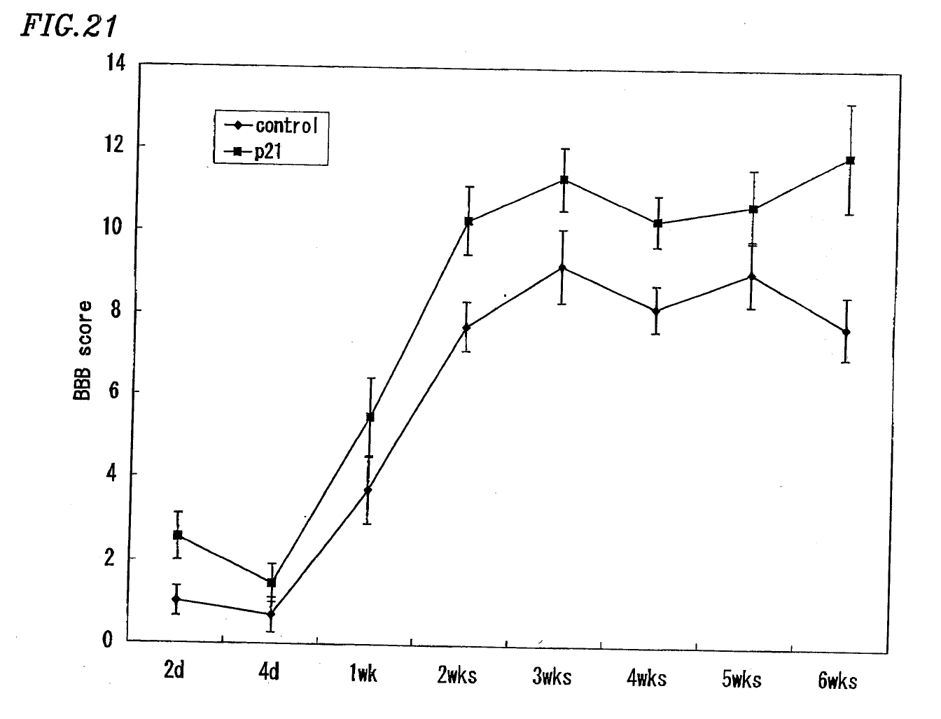

Initially, a nucleic acid sequence encoding p21 was fused with a nucleic acid sequence encoding GST; a nucleic acid sequence encoding an amino-terminally 11-amino acid protein-introduced domain (YGRKKRRQRRR SEQ ID NO. 20) derived from a human immunodeficiency virus protein; and a nucleic acid sequence encoding myc (FIG. 20). Also, a nucleic acid sequence without a p21-encoding sequence was prepared (FIG. 20). These sequences were expressed to produce polypeptides using a commonly used method. Whether or not these pepetides contribute to the functional recovery after spinal cord injury was investigated.

200 g male Wistar rats were used. After a ninth thoracic vertebrae laminectomy was performed, the dorsal half of the spinal cord was dissected. A continuous osmotic pump was used to continuously administer either TAT-bound p21 or a control protein to the injured site for 2 weeks. In this case, the tip of a tube connected to the pump was left in the medullary space.

After spinal cord injury, the functional recovery was assessed using the BBB score (Basso-Beattie-Bresnahan (BBB) Locomotor Rating; Basso, D. M., Beattie, M. S., Bresnahan, J. C., J. Neurotrauma 12(1):1-21 (1995)). Observation was carried out from day 2 after injury for 6 weeks. The results are shown in FIG. 21.

As shown in FIG. 21, in a group in which the TAT-bound p21 polypeptide was administered, a significant level of functional recovery was observed in the spinal cord, while in the control group substantially no such recovery was found. Therefore, it was revealed that the TAT-bound p21 of the present invention promotes the regeneration of the actual nerve system as well as the functional recovery.

Further, it was revealed that in p21, the TAT PTD domain is an active site. Thus, it was revealed that the TAT PTD domain has a significant effect of allowing a composition for nerve regeneration to function actually.

### Example 4-9

### Other Rho Kinase Inhibitors

Similar experiments were carried out using an antibody as an agent capable of specifically interacting with the Rho kinase. As a result, neurite outgrowth was observed and in vitro nerve regeneration was observed.

(Effects of the Invention)

Thus, the present invention provides a pharmaceutical composition and method for nerve regeneration and treatment of neurological diseases by nerve regeneration inhibition of neurite outgrowth. The present invention is based on the present inventors' findings on the relationship between p75^{NTR} involved in inhibition of neurite outgrowth and agents capable of interacting therewith.

As described above, the present invention is illustrated by way of the preferred embodiments. However, it will be understood that the scope of the present invention should be interpreted only by the accompanying claims. It will also be understood that the patents, patent applications and literature cited herein should be incorporated by reference as if set forth fully herein.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

The Sequence Listing is contained on separately submitted CD-ROM entitled 59150-8023-SEQLIST.TXT (148 KB) created Apr. 30, 2003, which is incorporated in entirety by reference herewith.

1. A method for regenerating nerves, comprising the step of:
inhibiting a p75 signal transduction pathway.

2. The method according to claim 1, wherein the p75 signal transduction pathway is present in a neuron at a site desired for nerve regeneration.

3. The method according to claim 1, wherein the inhibition of the p75 signal transduction pathway is achieved by providing a transduction agent in the p75 signal transduction pathway or a variant or fragment thereof, or an agent capable of specifically interacting with the transduction agent in the p75 signal transduction pathway in an amount effective for regeneration.

4. The method according to claim 3, wherein the transduction agent in the p75 signal transduction pathway is at least one transduction agent selected from the group consisting of MAG, GT1b, p75, Rho GDI, Rho, p21, and Rho kinase.

5. The method according to claim 1, wherein the inhibition of the p75 signal transduction pathway is selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase.

6. The method according to claim 1, wherein the inhibition of the p75 signal transduction pathway is achieved by providing at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase, in an amount effective for regeneration.

7. The method according to claim 1, wherein the nerve regeneration is carried out in vivo or in vitro.

8. The method according to claim 1, wherein the nerve is in a condition including spinal cord injury, cerebrovascular disorder, or brain injury.

9. The method according to claim 1, wherein the step of inhibiting the p75 signal transduction pathway comprises the step of:
providing a composition comprising at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase, an agent capable of specifically interacting with a nucleic acid molecule encoding the Rho kinase, and variants and fragments thereof, to the nerve in an amount effective for regeneration.

10. The method according to claim 4, wherein the agent is bound to a PTD domain.

11. A method for treatment, prophylaxis, diagnosis or prognosis of nervous diseases, nervous disorders and/or nervous conditions, comprising the step of:
modulating a p75 signal transduction pathway in a subject in need of or suspected of being in need of the treatment, prophylaxis, diagnosis or prognosis.

12. The method according to claim 11, wherein the step of modulating the p75 signal transduction pathway comprises the step of:
administering a transduction agent in the p75 signal transduction pathway or a variant or fragment thereof, or an agent capable of specifically interacting with the transduction agent in the p75 signal transduction pathway in an amount effective for regeneration to the subject in need of or suspected of being in need of the treatment, prophylaxis, diagnosis or prognosis.

13. The method according to claim 11, wherein the transduction agent in the p75 signal transduction pathway is at least one transduction agent selected from the group consisting of MAG, GT1b, p75, Rho GDI, Rho, p21, and Rho kinase.

14. The method according to claim 11, wherein the modulation of the p75 signal transduction pathway comprises at least one modulation selected from the group consisting of inhibition of an interaction between MAG and GT1b, inhibition of an interaction between GT1b and p75, inhibition of an interaction between p75 and Rho, inhibition of an interaction between p75 and Rho GDI, maintenance or enhancement of an interaction between Rho and Rho GDI, inhibition of conversion from Rho GDP to Rho GTP, inhibition of an interaction between Rho and Rho kinase, and inhibition of an activity of Rho kinase, in the subject in need of or suspected of being in need of the treatment, prophylaxis, diagnosis or prognosis.

15. The method according to claim 11, wherein the modulation of the p75 signal transduction pathway comprises the step of:
administering at least one agent selected from the group consisting of an agent capable of suppressing or extinguishing an interaction between MAG and GT1b, an agent capable of suppressing or extinguishing an interaction between GT1b and p75, an agent capable of suppressing or extinguishing an interaction between p75 and Rho GDI, an agent capable of suppressing or extinguishing an interaction between p75 and Rho, an agent capable of maintaining or enhancing an interaction between Rho and Rho GDI, an agent capable of inhibiting conversion from Rho GDP to Rho GTP, an agent capable of inhibiting an interaction between Rho and Rho kinase, and an agent capable of inhibiting an activity of Rho kinase, in an amount effective for regeneration to the subject in need of or suspected of being in need of the treatment, prophylaxis, diagnosis or prognosis.

16. The method according to claim 11, wherein the nerve regeneration is carried out in vivo or in vitro.

17. The method according to claim 11, wherein the nerve is in a condition including spinal cord injury, cerebrovascular disorder, or brain injury.

18. The method according to claim 11, wherein the step of modulating the p75 signal transduction pathway comprises the step of:
providing a composition comprising at least one molecule selected from the group consisting of a Pep5 polypeptide, a nucleic acid molecule encoding the Pep5 polypeptide, an agent capable of specifically interacting with a p75 polypeptide, an agent capable of specifically interacting with a nucleic acid molecule encoding the p75 polypeptide, a p75 extracellular domain polypeptide, a nucleic acid molecule encoding the p75 extracellular domain polypeptide, an agent capable of specifically interacting with a Rho GDI polypeptide, an agent capable of specifically interacting with a nucleic acid molecule encoding the Rho GDI polypeptide, the Rho GDI polypeptide, a nucleic acid encoding the Rho GDI polypeptide, an agent capable of specifically interacting with a MAG polypeptide, an agent capable of specifically interacting with a nucleic acid molecule encoding the MAG polypeptide, a p21 polypeptide, a nucleic molecule encoding p21, an agent capable of specifically interacting with a Rho polypeptide, an agent capable of specifically interacting with a nucleic acid molecule encoding the Rho polypeptide, an agent capable of specifically interacting with a Rho kinase and an agent capable of specifically interacting with a nucleic acid molecule encoding the Rho kinase, and variants and fragments thereof, in an amount effective for the diagnosis, prophylaxis, treatment or prognosis to the nerve.

19. The method according to claim 11, further comprising the step of:
providing one or more drugs.

20. The method according to claim 13, wherein the agent is bound to a PTD domain.

21-206 (Cancelled)
